(19) United States
(12) Patent Application Publication
(10) Pub. No.: US 2009/0221564 A1
(43) Pub. Date: Sep. 3, 2009

(54) Heterocyclic Compounds and Their Use in the Treatment of Cardiovascular Disease

(75) Inventors: Shouming Wang, Maidenhead (GB); Toby Jonathan Blench, London (GB); Frederic Marlin, Didcot (GB); Richard Beck, London (GB); Roger Peter Dickinson, Dover (GB)

Correspondence Address: KLARQUIST SPARKMAN, LLP, 121 SW SALMON STREET, SUITE 1600, PORTLAND, OR, 97204, US

(73) Assignee: TRIGEN LIMITED, Croydon (GB)

(21) Appl. No.: 12/224,265

(22) PCT Filed: Feb. 20, 2007
(86) PCT No.: PCT/EP2007/051626

§ 371 Date: Dec. 12, 2008

(30) Foreign Application Priority Data
Feb. 21, 2006 (GB) 0603374.0
Feb. 21, 2006 (GB) 0603376.5
Feb. 21, 2006 (GB) 0603378.1

(51) Int. Cl. A61K 31/541; C07D 333/58; A61K 31/381; C07D 409/12; C07D 409/10; A61K 31/4436; C07D 413/10; A61K 31/4245; A61K 31/496; A61K 31/506; C07D 417/12; A61K 31/428; A61K 31/422; A61K 31/4196; A61K 31/433; A61K 31/497; A61K 31/4725; C07D 413/12; A61K 31/5377; A61K 31/445; A61K 31/44; A61P 9/00

(52) U.S. Cl. 514/227.8; 549/51; 514/443; 546/281.1; 514/337; 548/131; 514/364; 544/376; 514/252.13; 544/333; 514/256; 548/159; 514/367; 548/247; 514/378; 548/266.4; 514/383; 514/361; 548/127; 544/405; 514/255.05; 546/143; 514/310; 544/146; 514/233.5; 546/202; 514/324; 514/300; 546/122; 546/171; 544/62

(57) ABSTRACT

Heterocyclic compounds of the formula (I) are provided: wherein ring A, ring B, R¹, R², R³, R⁴, Y, m, n and q are as identified herein. R¹ is in particular amidino. The invention further provides particular benzothiophene compounds. Compounds of the invention may be useful as inhibitors of Factor IXa and in the therapy of cardiovascular conditions and diseases, e.g. thrombosis.

### FIELD OF THE INVENTION

The present invention relates to heterocyclic compounds, their use, their formulation, their preparation, their synthetic intermediates and to other subject matter. The compounds may be useful in the treatment of cardiovascular diseases and conditions, in particular thrombosis.

### BACKGROUND TO THE INVENTION

Proteases are enzymes that catalyse the hydrolysis of covalent peptidic bonds. As examples of serine proteases may be mentioned thrombin (also called Factor IIa), Factor VIIa, Factor Xa, Factor IXa, Factor XIIa, plasmin, tissue kallikrein, pancreatic elastase, pancreatic elastase II, tissue plasminogen activator (also called tPA), Protein C (activated), and urokinase-type plasminogen activator (also called uPA or urokinase).

Control of coagulation serine protease activity is a major target in the development of pharmacological agents. Coagulation is a dynamic and complex process in which proteolytic enzymes such as thrombin play a key role. The blood coagulation cascade involves the conversion of zymogens into active enzymes which ultimately convert the soluble plasma protein fibrinogen into an insoluble matrix of highly cross-linked fibrin. Blood clots are composed of activated platelets and fibrin. Examples of such zymogens include Factor XII, Factor XI, Factor IX, Factor X, Factor VII, and prothrombin. These zymogens are activated to form the proteases Factor XIIa, Factor XIa, Factor IXa, Factor Xa, Factor VIIa, and thrombin.

There exists a need for effective therapeutic agents for the inhibition of serine proteases, for example in the regulation of hemostasis, and for the prevention and treatment of thrombus formation and other pathological processes in the vasculature induced by thrombin such as, for example, restenosis and inflammation. In particular, there exists a need for effective inhibitors of Factor IXa. There further exists a need for additional anti-tumour agents, including anti-tumour agents having anti-thrombotic activity.

Benzothiophene has the structure:

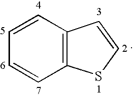

### SUMMARY OF THE INVENTION

The present invention provides compounds and compositions which may be useful in vitro or in vivo for inhibiting one or more serine proteases and/or for the prevention or therapy of conditions in mammals characterized by undesired serine protease activity, or by thrombosis. Also disclosed are compounds and compositions useful for inhibiting serine proteases, in particular Factor IXa.

In one aspect, the present invention relates to the use of compounds of Formula (I), including prodrugs, salts, isomers, hydrates and solvates thereof, for the inhibition of serine proteases, particularly in the therapy of diseases or conditions susceptible to treatment by inhibition of a serine protease, for example in the therapy of thrombosis and/or other cardiovascular diseases:

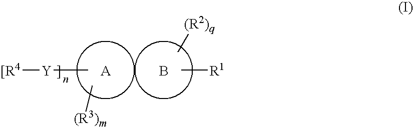

wherein
ring A is a 5-, 6- or 7-membered ring which is fused with ring B;
ring B is a 5-, 6- or 7-membered ring having at least one in-ring atom which is —O— or —S—;
each Y is independently a bond or a linker having 1 to 20 (e.g. 1 to 10) in-chain atoms (e.g. selected from C, N, O and S) and comprising, for example, one or more linkages selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C≡C—, carbocyclylene optionally substituted with 1, 2, 3, 4 or 5 R¹¹, and heterocyclylene optionally substituted with 1, 2, 3, 4 or 5 R¹¹ (in some embodiments said linkages may additionally be selected from —N(R⁶)—);
R¹ is hydrogen R¹¹, or a basic group;
R² and R³ are each independently an organic or inorganic substituent, for example and without limitation selected from R¹¹;
each R⁴ is independently hydrogen, except when Y is a bond; or is hydrocarbyl or heterocyclyl, either of which is optionally substituted, for example with 1, 2, 3, 4 or 5 R¹¹ (but the invention is not limited to substitution with 1, 2, 3, 4 or 5 R¹¹);
each R⁵ is independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
R⁶ and R⁷ are each independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃, with the proviso that R⁷ is not hydrogen;
R⁸, R⁹ and R¹⁰ are each independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
each R¹¹ is independently selected from halogen, hydroxy, trifluoromethyl, cyano, nitro, oxo, amidino, —B(OH)₂, ═NR¹², —OR¹², —SR¹², —C(O)R¹², —C(O)OR¹², —OC(O)R¹², —N(R¹²)R¹³, —C(O)N(R¹²)R¹³, —OC(O)N(R¹²)R¹³, —S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹², —NR¹³S(O)ₗR¹², —NR¹³C(O)NR¹²R¹³, —C(R¹²)₃ and R¹⁴ (of these R¹¹ moieties, one embodiment comprises halogen, hydroxy, trifluoromethyl, cyano, nitro, oxo, amidino, —B(OH)₂, ═NR¹², —OR¹², —C(O)R¹²—C(O)OR¹², —OC(O)R¹², —N(R¹²)R¹³, C(O)N(R¹²)R¹³, —S(O)ₗR¹², —C(R¹²)₃ and R¹⁴);
R¹² and R¹³ are the same or different and are each hydrogen or are selected from C₁₋₆ acyclic aliphatic groups and particularly C₁₋₆ alkyl, carbocyclyl optionally substituted by a C₁₋₆ acyclic aliphatic group and bonded to the remainder of the molecule either directly or through a C₁₋₆ acyclic aliphatic group (particularly —(CH₂)ⱼ-carbocyclyl or —(CH₂)ⱼ-carbocyclyl(C₁-C₆)alkyl), and heterocyclyl optionally substituted by a C₁₋₆ acyclic aliphatic group and bonded to the remainder of the molecule either directly or through a C₁₋₆ acyclic aliphatic group (particularly —(CH₂)ⱼ-heterocyclyl or —(CH₂)ⱼ-heterocyclyl(C₁-C₆)alkyl), any of which is optionally substituted with 1, 2, 3, 4 or 5 substituents independently selected from halogen, hydroxy, trifluoromethyl, cyano, nitro, oxo, amidino, —B(OH)₂, ═NR^{u}, —OR^{v}, —SR^{v}, —C(O)R^{v}, —C(O)OR^{v}, —OC(O)R^{v}, —N(R^{u})R^{v}, —C(O)N(R^{u})R^{v}, —OC(O)N(R^{u})R^{v}, —S(O)ₗNR^{v}, —S(O)ₗNR^{u}R^{v}, —S(O)ₗNR^{u}C(O)R^{v}, —S(O)ₗNR^{u}C(O)OR^{v}, —NR^{u}C(O)R^{v}, NR^{u}C(O)OR^{v}, —NR^{u}S(O)₁R^{v}, —NR^{u}C(O)NR^{v}R^{u}, —C(R^{v})₃, and C₁₋₆ alkyl optionally substituted by 1, 2, 3, 4 or 5 halogens, where R^{u} is H, OH or C₁₋₆ alkyl optionally substituted by up to 5 halogens and R^{v} is H or C₁₋₆ alkyl optionally substituted by up to 5 halogens, e.g. R¹² and R¹³ are the same or different and are each hydrogen or are selected from C₁₋₆ alkyl, —(CH₂)ⱼ-carbocyclyl and —(CH₂)ⱼ-heterocyclyl, any of which is optionally substituted with 1, 2, 3, 4 or 5 substituents independently selected from halogen, hydroxy and C₁₋₆ alkyl;
R¹³ additionally may be hydroxy or C₁₋₆ alkoxy;
R¹⁴ is selected from C₁₋₆ acyclic aliphatic groups and particularly C₁₋₆ alkyl, C₁₋₆ acyclic aliphatic-oxy and particularly C₁₋₆ alkoxy, —(CH₂)ᵢ—O—(CH₂)ⱼ-carbocyclyl, —(CH₂)ᵢ—O—(CH₂)ⱼ-heterocyclyl, —(CH₂)ᵢ—O—(CH₂)ⱼ-carbocyclyl(C₁-C₆)alkyl and —(CH₂)ᵢ—O—(CH₂)ⱼ-heterocyclyl(C₁-C₆)alkyl any of which is optionally substituted with 1, 2, 3, 4 or 5 substituents independently selected from halogen, hydroxy, trifluoromethyl, cyano, nitro, oxo, amidino, —B(OH)₂, ═NR¹², —OR², —SR², —C(O)R¹², —C(O)OR¹², —OC(O)R¹², —N(R¹²)R¹³, —C(O)N(R¹²)R¹³, —OC(O)N(R¹²)R¹³—S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹², —NR¹³S(O)ₗR¹², —NR¹³C(O)NR¹²R¹³, and —C(R¹²)₃ (of these R¹⁴ moieties, one embodiment comprises C₁₋₆ alkyl, C₁₋₆ alkoxy, —(CH₂)ⱼ-carbocyclyl and —(CH₂)ⱼ-heterocyclyl, any of which is optionally substituted with 1, 2, 3, 4 or 5 substituents independently selected from halogen, hydroxy, C₁₋₆ alkyl and C₁₋₆ alkoxy);
i is 0, 1, 2, 3, 4, 5 or 6
j is 0, 1, 2, 3, 4, 5 or 6;
k is 1, 2, 3, 4, 5 or 6;
l is 0, 1 or 2;
m is 0, 1, 2, 3 or 4;
n is 0, 1 or 2;
p is 0 or 1; and
q is 0, 1, 2, 3 or 4;
or a pharmaceutically acceptable salt or prodrug thereof.

The compounds of Formula (I) and the embodiments thereof described later in this specification themselves constitute an aspect of the invention.

In another aspect, the invention provides novel fused ring heterocyclic compounds including their pharmaceutically acceptable isomers, salts, hydrates, solvates and prodrugs. More particularly, the disclosure provides compounds of Formula (II):

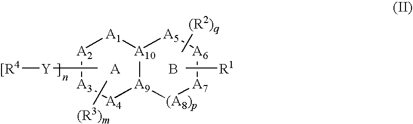

wherein
A₁, A₂, A₃, A₄, A₅, A₆, A₇ and A₈ may be the same or different and are each independently selected from —N═, —NH—, —O—, —S—, —CH═ and —CH₂—, wherein at least one of A₅, A₆, A₇ and A₈ is —O— or —S—;
A₉ and A₁₀ are each independently C, CH or N; and
other symbols are as defined in relation to Formula (I);
or a pharmaceutically acceptable salt or prodrug thereof.

In a further aspect the invention provides a benzothiophene compound, e.g. an amidinobenzothiophene compound, in particular a 2-amidinobenzothiophene compound, the compound being characterised in that it comprises a substituent at one or both of the 4- and 6-positions which substituent comprises a fragment independently selected from any of Formulae (i) to (vi):

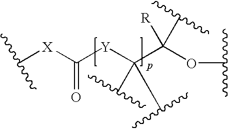

wherein
R is a moiety comprising an optionally substituted carbocyclic or heterocyclic group;
X and Y are each independently O or N;
p is 0 or 1; and
the oxygen atom on the right hand side of the fragment as drawn is bound directly to the 4- or 6-carbon atom of the benzothiophene ring;
   i.e. said substituent comprises a fragment independently selected from any of Formulae (i) to (vi):

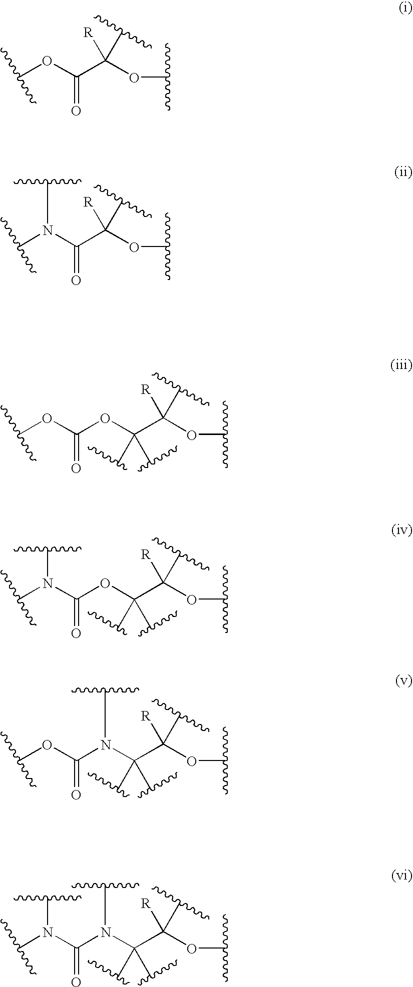

or a pharmaceutically acceptable salt or prodrug thereof.

In a further aspect the invention provides isosteres of the present compounds.

The invention includes also pharmaceutical formulations adapted to be administered to a patient and deliver a compound of the invention to the plasma of the patient.

The invention also provides formulations, pharmaceutical or otherwise, containing such compounds and includes compositions comprising excipients and diluents, as required. In particular, the disclosure also relates to pharmaceutically acceptable formulations of the compounds described herein, which may be useful as inhibitors of at least Factor IXa.

Where the context permits, substituents mentioned herein may be selected from (i) halogen; (ii) moieties having from 1 to 30 plural valent atoms (e.g. 1 to 20, for example 1 to 10, in particular 1, 2, 3 or 4, plural valent atoms), selected from C, N, O and S as well as monovalent atoms selected from hydrogen and halogen, e.g. selected from hydrogen, F, Cl and Br, for example hydrogen, F and Cl.

The invention includes in one embodiment compounds and groups in which any one or more hydrogen atoms bonded to a carbon are replaced by a halogen, e.g. F or Cl. Thus, reference to alkyl in this embodiment includes reference to such an alkyl group substituted by one or more halogens.

The compounds of the invention may be useful in the therapy (including treatment, prevention and the delay of progression) of cardiovascular diseases or conditions, in particular thrombosis.

The extent of protection includes counterfeit or fraudulent products which contain or purport to contain a compound of the invention irrespective of whether they do in fact contain such a compound and irrespective of whether any such compound is contained in a therapeutically effective amount.

Included in the scope of protection are packages which include a description or instructions which indicate that the package contains a species or pharmaceutical formulation of the invention and a product which is or comprises, or purports to be or comprise, such a formulation or species. Such packages may be, but are not necessarily, counterfeit or fraudulent.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

### DESCRIPTION OF VARIOUS EMBODIMENTS

### Definitions

### Hydrocarbyl

The term “hydrocarbyl” as used herein includes reference to a moiety consisting exclusively of hydrogen and carbon atoms; such a moiety may comprise an aliphatic and/or an aromatic moiety. It may additionally or alternatively comprise an alicyclic moiety. The hydrocarbyl moiety may comprise 1, 2, 3, 4, 5, 6, 7, 8, 9, 10, 11, 12, 13, 14, 15, 16, 17, 18, 19 or 20 carbon atoms, e.g. from 1 to 10 or from 1 to 6 carbon atoms. Examples of hydrocarbyl groups include C₁₋₆ alkyl (e.g. C₁, C₂, C₃ or C₄ alkyl, for example methyl, ethyl, propyl, isopropyl, n-butyl, sec-butyl or tert-butyl); C₁₋₆ alkyl substituted by aryl (e.g. benzyl) or by cycloalkyl (e.g cyclopropylmethyl); cycloalkyl (e.g. cyclopropyl, cyclobutyl, cyclopentyl or cyclohexyl); aryl (e.g. phenyl, naphthyl or fluorenyl) and the like.

### Aliphatic

The term “aliphatic” as used herein includes reference to acyclic or cyclic, saturated or unsaturated carbon moieties, excluding aromatic compounds. (Source: IUPAC Compendium of Chemical Terminology; http://goldbook.iupac.org/index.html). Aliphatic moieties are in particular acyclic hydrocarbyl moieties having, for example, 1, 2, 3, 4, 5 or 6 carbon atoms.

### Alkyl

The terms “alkyl” and “C₁₋₆ alkyl” as used herein include reference to a straight or branched chain alkyl moiety having 1, 2, 3, 4, 5 or 6 carbon atoms. This term includes reference to groups such as methyl, ethyl, propyl (n-propyl or isopropyl), butyl (n-butyl, sec-butyl or tert-butyl), pentyl, hexyl and the like. In particular, alkyl may have 1, 2, 3 or 4 carbon atoms. The term “lower alkyl” includes reference to alkyl groups having 1, 2, 3 or 4 carbon atoms.

### Alkenyl

The terms “alkenyl” and “C₂₋₆ alkenyl” as used herein include reference to a straight or branched chain alkyl moiety having 2, 3, 4, 5 or 6 carbon atoms and having, in addition, at least one double bond, of either E or Z stereochemistry where applicable. This term includes reference to groups such as ethenyl, 2-propenyl, 1-butenyl, 2-butenyl, 3-butenyl, 1-pentenyl, 2-pentenyl, 3-pentenyl, 1-hexenyl, 2-hexenyl and 3-hexenyl and the like. The term “lower alkenyl” includes reference to alkenyl groups having 1, 2, 3 or 4 carbon atoms.

### Alkynyl

The terms “alkynyl” and “C₂₋₆ alkynyl” as used herein include reference to a straight or branched chain alkyl moiety having 2, 3, 4, 5 or 6 carbon atoms and having, in addition, at least one triple bond. This term includes reference to groups such as ethynyl, 1-propynyl, 2-propynyl, 1-butynyl, 2-butynyl, 3-butynyl, 1-pentynyl, 2-pentynyl, 3-pentynyl, 1-hexynyl, 2-hexynyl and 3-hexynyl and the like. The term “lower alkynyl” includes reference to alkynyl groups having 1, 2, 3 or 4 carbon atoms.

### Alkoxy

The terms “alkoxy” and “C₁₋₆ alkoxy” as used herein include reference to —O-alkyl, wherein alkyl is straight or branched chain and comprises 1, 2, 3, 4, 5 or 6 carbon atoms. In one class of embodiments, alkoxy has 1, 2, 3 or 4 carbon atoms. This term includes reference to groups such as methoxy, ethoxy, propoxy, isopropoxy, butoxy, tert-butoxy, pentoxy, hexoxy and the like. The term “lower alkoxyl” includes reference to alkoxyl groups having 1, 2, 3 or 4 carbon atoms.

### Cycloalkyl

The term “cycloalkyl” as used herein includes reference to an alicyclic moiety having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

### Aryl

The term “aryl” as used herein includes reference to an aromatic ring system comprising 6, 7, 8, 9, 10, 11, 12, 13, 14, 15 or 16 ring carbon atoms. Aryl is often phenyl but may be a polycyclic ring system, having two or more rings, at least one of which is aromatic. This term includes reference to groups such as phenyl, naphthyl, fluorenyl, azulenyl, indenyl, anthryl and the like.

### Carbocyclyl

The term “carbocyclyl” as used herein includes reference to a saturated (e.g. cycloalkyl) or unsaturated (e.g. aryl) ring moiety having 3, 4, 5, 6, 7, 8, 9, 10, 11, 12, 13, 14, 15 or 16 carbon ring atoms. In particular, carbocyclyl includes a 3- to 10-membered non-aromatic ring or ring system and, in particular, a 5- or 6-membered non-aromatic ring, which may be fully or partially saturated. A carbocyclic moiety is, for example, selected from cyclopropyl, cyclobutyl, cyclopentyl, cyclohexyl, norbornyl, bicyclo[2.2.2]octyl, phenyl, naphthyl, fluorenyl, azulenyl, indenyl, anthryl and the like.

### Heterocyclyl

The term “heterocyclyl” as used herein includes reference to a saturated (e.g. heterocycloalkyl) or unsaturated (e.g. heteroaryl) heterocyclic ring moiety having from 3, 4, 5, 6, 7, 8, 9, 10, 11, 12, 13, 14, 15 or 16 ring atoms, at least one of which is selected from nitrogen, oxygen, phosphorus, silicon and sulphur. In particular, heterocyclyl includes a 3- to 10-membered non-aromatic ring or ring system and more particularly a 5- or 6-membered ring, which may be fully or partially saturated.

A heterocyclic moiety is, for example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl, chromanyl and the like.

### Heterocycloalkyl

The term “heterocycloalkyl” as used herein includes reference to a saturated heterocyclic moiety having 3, 4, 5, 6 or 7 ring carbon atoms and 1, 2, 3, 4 or 5 ring heteroatoms selected from nitrogen, oxygen, phosphorus and sulphur. The group may be a polycyclic ring system but more often is monocyclic. This term includes reference to groups such as azetidinyl, pyrrolidinyl, tetrahydrofuranyl, piperidinyl, oxiranyl, pyrazolidinyl, imidazolyl, indolizidinyl, piperazinyl, thiazolidinyl, morpholinyl, thiomorpholinyl, quinolizidinyl and the like.

### Heteroaryl

The term “heteroaryl” as used herein includes reference to an aromatic ring system having 5, 6, 7, 8, 9, 10, 11, 12, 13, 14, 15 or 16 ring atoms, at least one of which is selected from nitrogen, oxygen and sulphur. The group may be a polycyclic ring system, having two or more rings, at least one of which is aromatic, but is more often monocyclic. This term includes reference to groups such as pyrimidinyl, furanyl, benzo[b]thiophenyl, thiophenyl, pyrrolyl, imidazolyl, pyrrolidinyl, pyridinyl, benzo[b]furanyl, pyrazinyl, purinyl, indolyl, benzimidazolyl, quinolinyl, phenothiazinyl, triazinyl, phthalazinyl, 2H-chromenyl, oxazolyl, isoxazolyl, thiazolyl, isoindolyl, indazolyl, purinyl, isoquinolinyl, quinazolinyl, pteridinyl and the like.

### Halogen

The term “halogen” as used herein includes reference to F, Cl, Br or I. In a particular class of embodiments, halogen is F or Cl, of which F is more common.

Basic moieties

The term “basic moiety” or “basic group” as used herein includes reference to a moiety having an available pair of electrons capable of forming a covalent bond with a proton (i.e. a Brønsted base) or with the vacant orbital of another species (i.e. a Lewis base). A basic moiety is, for example, a moiety containing a basic nitrogen atom, such as an amino acid residue.

### Amidino

The term “amidino” as used herein includes reference to moieties of the general structure —C(NH)NH₂ and derivatives thereof, in particular, those in which a hydrogen is replaced by alkyl, (e.g. methyl or ethyl) or hydroxy. In embodiments, “amidino” means a group of the structure —C(NH)NH₂. Amidino may be in the form of a pharmaceutically acceptable salt or prodrug thereof.

### Substituted

The term “substituted” as used herein in reference to a moiety means that one or more, especially up to 5, more especially 1, 2 or 3, of the hydrogen atoms in said moiety are replaced independently of each other by the corresponding number of the described substituents.

It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Additionally, it will of course be understood that the substituents described herein may themselves be substituted by any substituent, subject to the aforementioned restriction to appropriate substitutions as recognised by the skilled man.

Where steric issues determine placement of substituents on a group, the isomer having the lowest conformational energy may be preferred.

### Optionally

Where a compound, moiety, process or product is described as “optionally” having a feature, the disclosure includes such a compound, moiety, process or product having that feature and also such a compound, moiety, process or product not having that feature. Thus, when a moiety is described as “optionally substituted”, the disclosure comprises the unsubstituted moiety and the substituted moiety.

### Independently

Where two or more moieties are described as being “each independently” selected from a list of atoms or groups, this means that the moieties may be the same or different. The identity of each moiety is therefore independent of the identities of the one or more other moieties.

### Pharmaceutically Acceptable

The term “pharmaceutically acceptable” as used herein includes reference to those compounds, materials, compositions, and/or dosage forms which are, within the scope of sound medical judgment, suitable for use in contact with the tissues of human beings or animals without excessive toxicity, irritation, allergic response, or other problem or complication, commensurate with a reasonable benefit/risk ratio. This term includes acceptability for both human and veterinary purposes.

### Prevention

The term “prevention” and cognate terms as applied to a disease, disorder or occurrence (by way of non-limiting example, thrombosis or thrombotic diseases or disorders) and the like refers to the primary and secondary prophylaxis thereof, unless the context requires otherwise. When a compound or composition is administered to prevent a disease, disorder or occurrence, it does not mean that the prevention will be completely successful in every patient for an indefinite period, since both curative and prophylactic (preventative) treatments may fail. Preventative or prophylactic treatment therefore includes reference to treatment which, within the scope of sound medical judgment, is appropriate to prevent the disease, disorder or occurrence in question, irrespective of the outcome of the treatment. The invention includes successful and unsuccessful methods of prevention.

### Thrombosis

The term “thrombosis” as used herein refers inter alia to atrophic thrombosis, arterial thrombosis, cardiac thrombosis, coronary thrombosis, creeping thrombosis, infective thrombosis, mesenteric thrombosis, placental thrombosis, propagating thrombosis, traumatic thrombosis and venous thrombosis.

### Thrombin Inhibitor

The term “thrombin inhibitor” as used herein includes reference to a compound or product which, within the scope of sound pharmacological judgement, is potentially or actually pharmaceutically useful as an inhibitor of thrombin, and includes reference to any substance which comprises a pharmaceutically active species and is described, promoted or authorised as a thrombin inhibitor. Such thrombin inhibitors may be selective, that is they are regarded, within the scope of sound pharmacological judgement, as selective towards thrombin in contrast to other proteases; the term “selective thrombin inhibitor” includes reference to substance which comprises a pharmaceutically active species and is described, promoted or authorised as a selective thrombin inhibitor.

### Factor IXa Inhibitor

The terms “Factor IXa” or “FIXa” as used herein include reference to a compound or product which, within the scope of sound pharmacological judgement, is potentially or actually pharmaceutically useful as an inhibitor of Factor IXa, and includes reference to any substance which comprises a pharmaceutically active species and is described, promoted or authorised as a Factor IXa inhibitor. Such Factor IXa inhibitors may be selective, that is they are regarded, within the scope of sound pharmacological judgement, as selective towards thrombin in contrast to other proteases; the term “selective Factor IXa inhibitor” includes reference to any substance which comprises a pharmaceutically active species and is described, promoted or authorised as a selective Factor IXa inhibitor.

### Product

The term “product” or “product of the invention” as used herein includes reference to any product containing a compound of the present invention. In particular the term product relates to compositions containing a compound of the present invention, such as a pharmaceutical composition, for example.

### Therapeutically Effective Amount

The term “therapeutically effective amount” as used herein refers to an amount of a drug, or pharmaceutical agent that, within the scope of sound pharmacological judgement, is calculated to (or will) provide a desired therapeutic response in a mammal (animal or human). The therapeutic response may for example serve to cure, delay the progression of or prevent a disease, disorder or condition.

### Isosteres

The term “isostere” as used herein includes reference to compounds, atoms or groups that have different molecular formulae but exhibit the same or similar properties. Examples of isosteric groups are given below.

(a) In-chain isosteres
(i) —CH₃, —NH₂, —OH, —F, Cl
(ii) —Cl, —SH, —PH₂
(iii) —Br, -iso-propyl
(iv) —CH₂, —NH—, —O—, —S—
(v) —C(O)CH₂—, —C(O)NH—, —C(O)O—, —C(O)S—
(vi) —HC═, —N═
   (b) In-ring isosteres
(i) —CH═CH—, —S—
(ii) —O—, —S—, —CH₂—, —NH—
(iii) —CH═, —N—

Other isosteres exist to mimic functional, or other, groups, for example, tetrazole is an isostere of carboxylic acid because it mimics the properties of carboxylic acid even though they both have very different molecular formulae.

Tetrazole is one of many possible isosteric replacements for carboxylic acid. Other carboxylic acid isosteres contemplated by the disclosure include:

—COOH, —SO₃H, —SO₂HNR′, —PO₂(R′)₂, —CN, —PO₃(R′)₂, —OR′, —SR′, —NHCOR′, —N(R′)₂, —CON(R′)₂, —CONH(O)R′, —CONHNHSO₂R′, —COHNSO₂R′, and —CONR′CN
where R′ is any atom or group which does not impair the carboxylic acid isosteric properties of the moiety or compound.

In addition, carboxylic acid isosteres can include 5- to 7-membered carbocycles or heterocycles containing any combination of CH₂, O, S, or N in any chemically stable oxidation state, where any of the atoms or groups of said ring structure are unsubstituted or substituted in one or more positions. Isosteres of the are often preferably bio-isosteres.

Preferably, any addition of chemical substituents to an isostere, such as a carboxylic acid isostere, retains the properties of the isostere.

The following are non-limiting examples of isosteres of the present invention:

### Mono-valent Moieties:

(i) CH₃, NH₂, OH, F, Cl
(ii) Cl, PH₂, SH
(iii) Br, i-Pr
(iv) I, t-Bu

### Bi-valent Moieties:

(i) —CH₂—, —NH—, —O—, —S—, —Se—
(ii) —COCH₂R—, —CONHR—, —CO₂R—, —COSR, —NRSO₂—

### Tri-valent Moieties:

(i) —CH═, —N═
(ii) —P═, —As═

### Tetra-valent Moieties:

(i) C, Si
(ii) ═C═, ═N⁺═, ═P⁺═

### Halogen Moieties:

F, Cl, Br, I, CF₃, CN, N(CN)₂, C(CN)₃

### Thioether Moieties:

—S—, —O—, —C(CN)₂— and —N(CN)—

### Thiourea Moieties:

—NHC(S)NH₂, —NHC(═NCN)NH₂, —NHC(═NNO₂)NH₂, —C(═NS(O)₂NH₂)NH₂

### In-ring Moieties:

(i) —CH═CH—, —S— (e.g. in benzene and thiophene)
(ii) —CH═, —N═ (e.g. in benzene, pyridine)
(iii) —O—, —S—, —CH₂—, —NH— (e.g. in tetrahydrofuran, tetrahydrothiophene, cyclopentane and pyrrolidine)

### Spacers:

—(CH₂)₃— and phenylene

### Azomethine:

—N═, —C(CN)═

### Hydrogen:

H, F

### Halogen Moieties:

F, Cl, Br, I, CF₃, CN, N(CN)₂, C(CN)₃

### Carbonyl Moieties:

—C(O)—, —C(S)—, —S(O)—, —C(═NOH)—, —C(═N—)-,
—C(O)N(−)-, —CH(CN)—, —C(═C(CN)₂))-, —S(O)₂N(−)-, —S(O)₂—

### Carboxylic Acid Moieties:

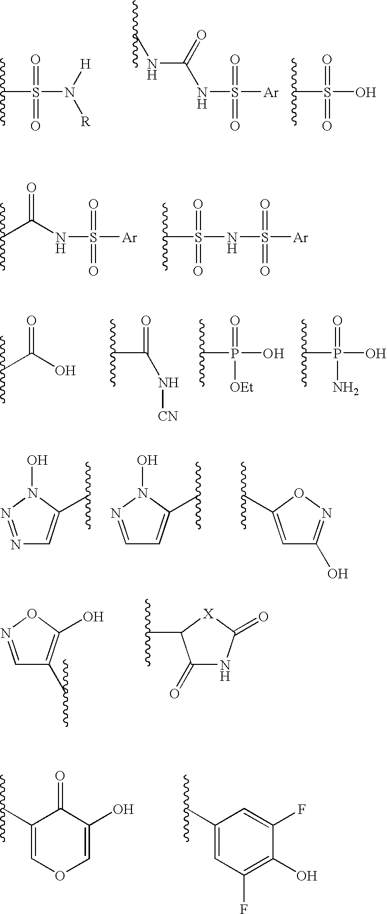

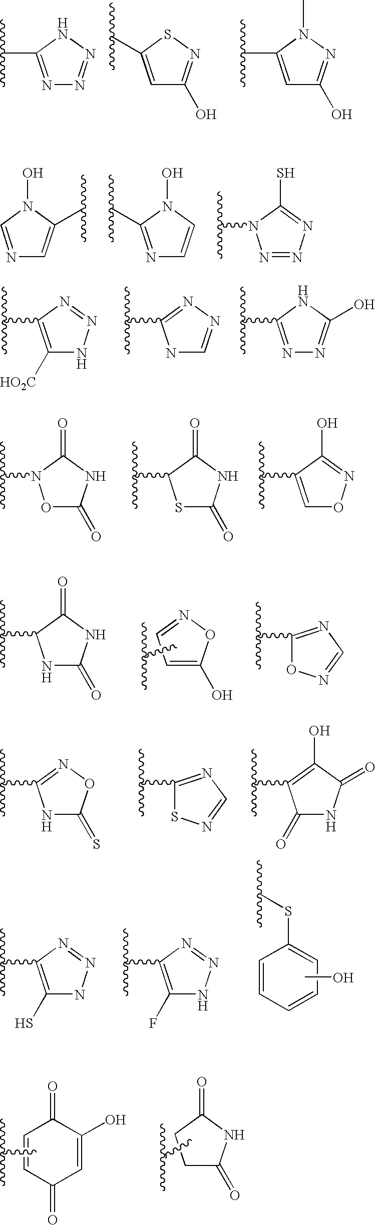

### Amide Moieties:

C(O)NH₂, —C(S)NH₂, —NHC(O)NH₂, —C(O)CH₂CH₃, —C(O)O—, —NHC(O)—, —NHC(O)O—, —NHS(O)O—

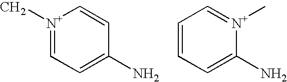

### Ester Moieties:

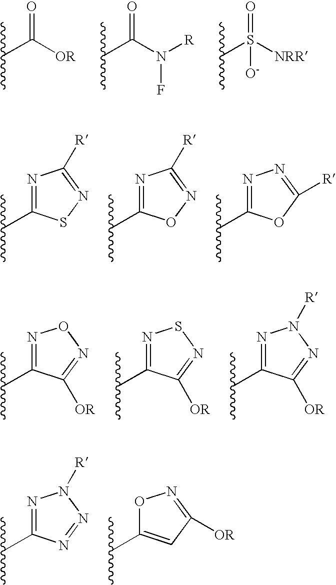

### Hydroxy Moieties:

—OH, —NHC(O)—, —NHS(O)₂—, —CH₂OH, —NH(CN)—, —CH(CN)₂, —NHC(O)NH₂

### Catechol Moieties:

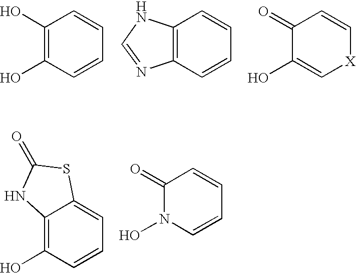

### Pyridine Moieties:

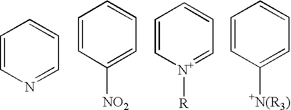

### Benzene Moieties:

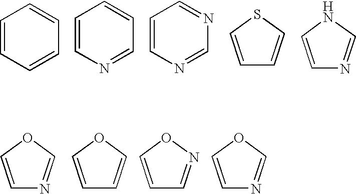

### Ring Equivalents:

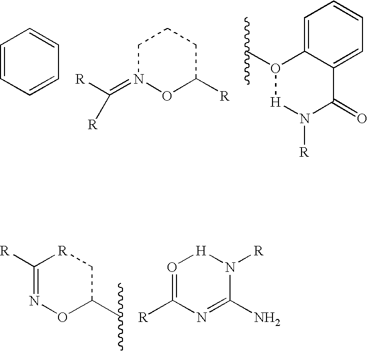

### Compounds

In one aspect, the present invention provides compounds of Formula (I) as previously defined. In embodiments, one or more of R¹¹, R¹², R¹³ and R¹⁴, e.g. one or both of R¹¹ and R¹⁴, are as described in the following paragraph.

One class of compounds (and the invention is not limited to this class of compounds) is of the following formula:

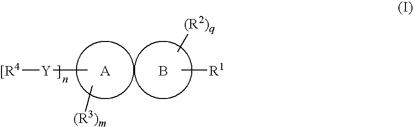

wherein
ring A is a 5-, 6- or 7-membered ring which is fused with ring B;
ring B is a 5-, 6- or 7-membered ring having at least one in-ring atom which is —O— or —S—;
each Y is independently a bond or a linker having 1 to 20 (e.g. 1 to 10) in-chain atoms (e.g. selected from C, N, O and S) and comprising, for example, one or more linkages selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C≡C—, carbocyclylene optionally substituted with 1, 2, 3, 4 or 5 R¹¹, and heterocyclylene optionally substituted with 1, 2, 3, 4 or 5 R¹¹;
R¹ is hydrogen or R¹¹, or a basic group;
R² and R³ are each independently selected from R¹¹;
each R⁴ is independently hydrogen, except when Y is a bond; or is hydrocarbyl or heterocyclyl, either of which is optionally substituted with 1, 2, 3, 4 or 5 R¹¹;
each R⁵ is independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
R⁶ and R⁷ are each independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃, with the proviso that R⁷ is not hydrogen;
R⁸, R⁹ and R¹⁰ are each independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
each R¹¹ is independently selected from halogen, hydroxy, trifluoromethyl, cyano, nitro, oxo, amidino, —B(OH)₂, ═NR¹², —OR¹², —C(O)R¹², —C(O)OR¹², —OC(O)R¹², —N(R¹²)R¹³, —C(O)N(R¹²)R¹³, —S(O)ₗR¹², —C(R¹²)₃ and R¹⁴;
R¹² and R¹³ are each independently hydrogen or are selected from C₁₋₆ alkyl, —(CH₂)ⱼ-carbocyclyl and —(CH₂)ⱼ-heterocyclyl, any of which is optionally substituted with 1, 2, 3, 4 or 5 substituents independently selected from halogen, hydroxy and C₁₋₆ alkyl;
R¹⁴ is selected from C₁₋₆ alkyl, C₁₋₆ alkoxy, —(CH₂)ⱼ-carbocyclyl and —(CH₂)ⱼ-heterocyclyl, any of which is optionally substituted with 1, 2, 3, 4 or 5 substituents independently selected from halogen, hydroxy, C₁₋₆ alkyl and C₁₋₆ alkoxy;
j is 0, 1, 2, 3, 4, 5 or 6;
k is 1, 2, 3, 4, 5 or 6;
l is 0, 1 or 2;
m is 0, 1, 2, 3 or 4;
n is 0, 1 or 2;
p is 0 or 1; and
q is 0, 1, 2, 3 or 4;
or a pharmaceutically acceptable salt or prodrug thereof.

A further class of compounds are of Formula (II):

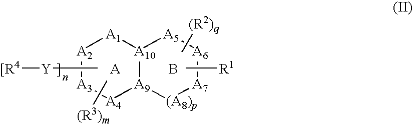

wherein
A₁, A₂, A₃, A₄, A₅, A₆, A₇ and A₈ may be the same or different and are each independently selected from —N═, —NH—, —O—, —S—, —CH═ and —CH₂—, wherein at least one of A₅, A₆, A₇ and A₈ is —O— or —S—;
A₉ and A₁₀ are each independently C, CH or N;
and other symbols are as defined previously in this specification, for example:
each Y is independently a bond or a linker having 1 to 20 (e.g. 1 to 10) in-chain atoms (e.g. selected from C, N, O and S) and comprising, for example, one or more linkages selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C≡C—, carbocyclylene optionally substituted with 1, 2, 3, 4 or 5 R¹¹, and heterocyclylene optionally substituted with 1, 2, 3, 4 or 5 R¹¹;
R¹ is hydrogen or R¹¹, for example a basic moiety;
R² and R³ are each independently selected from R¹¹;
each R⁴ is independently hydrogen, except when Y is a bond; or is hydrocarbyl or heterocyclyl, either of which is optionally substituted with 1, 2, 3, 4 or 5 R¹¹;
each R⁵ is independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
R⁶ and R⁷ are each independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃, with the proviso that R⁷ is not hydrogen;
R⁸, R⁹ and R¹⁰ are each independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
each R¹¹ is independently selected from halogen, hydroxy, trifluoromethyl, cyano, nitro, oxo, amidino, —B(OH)₂, ═NR¹², —OR¹², —C(O)R¹², —C(O)OR¹², —OC(O)R¹², —N(R¹²)R¹³, —C(O)N(R¹²)R¹³, —S(O)ₗR¹², —C(R¹²)₃ and R¹⁴;
R¹² and R¹³ are each independently hydrogen or are selected from C₁₋₆ alkyl, —(CH₂)ⱼ-carbocyclyl and —(CH₂)ⱼ-heterocyclyl, any of which is optionally substituted with 1, 2, 3, 4 or 5 substituents independently selected from halogen, hydroxy and C₁₋₆ alkyl;
R¹⁴ is selected from C₁₋₆ alkyl, C₁₋₆ alkoxy, —(CH₂)ⱼ-carbocyclyl and —(CH₂)ⱼ-heterocyclyl, any of which is optionally substituted with 1, 2, 3, 4 or 5 substituents independently selected from halogen, hydroxy, C₁₋₆ alkyl and C₁₋₆ alkoxy;
j is 0, 1, 2, 3, 4, 5 or 6;
k is 1, 2, 3, 4, 5 or 6;
l is 0, 1 or 2;
m is 0, 1, 2, 3 or 4;
n is 0, 1 or 2;
p is 0 or 1; and
q is 0, 1, 2, 3 or 4;
or a pharmaceutically acceptable salt or prodrug thereof.

Embodiments of the invention are described below. It will be appreciated that the features specified in each embodiment may be combined with other specified features, to provide further embodiments.

### Fused Ring AB

In Formula (I), ring A is a 5-, 6- or 7-membered ring which is fused with ring B. Ring B is a 5-, 6- or 7-membered ring having at least one in-ring heteroatom which is —O— or —S—.

In one embodiment, ring A is a 5- or 6-membered ring, in particular a 6-membered ring.

In a further embodiment, ring A is unsaturated.

In a further embodiment, ring A is aromatic.

In a further embodiment, ring A is heterocyclic.

In a further embodiment, ring A is aromatic and heterocyclic.

In a further embodiment, ring A is carbocyclic.

In a further embodiment, ring A is aromatic and carbocyclic.

In a further embodiment, ring B is a 5- or 6-membered ring, particularly a 5-membered ring.

In a further embodiment, ring B is unsaturated.

In a further embodiment, ring B contains a single in-ring heteroatom which is —O— or —S—.

In a further embodiment, ring B is contains a single in-ring heteroatom and is a 5-membered ring.

In a further embodiment, ring B contains two in-ring heteroatoms.

In a further embodiment, ring B contains three in-ring heteroatoms.

Typically, when two or more heteroatoms are present in ring B, they are separated by at least one in-ring carbon atom.

In a further embodiment, ring A is a 5 or 6-membered ring, and ring B is a 5 or 6-membered ring.

In a further embodiment, ring A is a 6-membered ring and ring B is a 5- or 6-membered ring.

In a further embodiment, ring A is a 6-membered ring and ring B is a 5-membered ring.

In a further embodiment, fused ring AB is as defined in Formula (II) below:

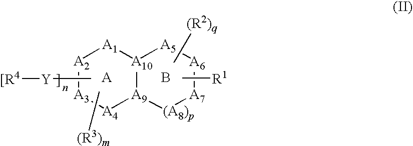

wherein
A₁, A₂, A₃, A₄, A₅, A₆, A₇ and A₈ are each independently selected from —N═, —NH—, —O—, —S—, —CH═ and —CH₂—, wherein at least one of A₅, A₆, A₇ and A₈ is —O— or —S—;
A₉ and A₁₀ are each independently C, CH or N; and
the remaining terms are as defined in relation to Formula (I).

With regard to formula (II) and related formulae, it will be appreciated that the solid lines forming rings A and B may represent single or double bonds depending on the identities of A₁ to A₁₀. Thus, each bond within a ring is a double bond or a single bond as required to satisfy valency requirements. As indicated, this applies not only to formula (II) but also, for example, formula (III) and the left hand ring of formula (IV) below, and the subsequently-described sub-classes of these embodiments.

In a further embodiment, A₁, A₂, A₃, A₄, A₉ and A₁₀ taken together form an aromatic ring. Thus, for example, A₁, A₂, A₃, A₄, A₉ and A₁₀ taken together may form a benzene ring.

In a further embodiment, at least two of A₁, A₂, A₃, A₄, A₉ and A₁₀ are each independently —CH═ and —CH₂—, and the one or more others are each —N═, —NH—.

In a further embodiment, A₁, A₂, A₃ and A₄ are each —CH═ (i.e. each comprise an sp² carbon atom).

In a further embodiment, A₁, A₂, A₃, A₄, A₉ and A₁₀ are each ═CH— or —CH₂— (i.e. each comprise and sp² or an sp³ carbon atom).

In a further embodiment, at least one, e.g. exactly one, of A₅, A₆, A₇ and A₈ (if present) is S.

In a further embodiment, only A₇ is a heteroatom, in particular —S— or —O—.

In a further embodiment, p is 0, i.e. the compound is of the Formula (III):

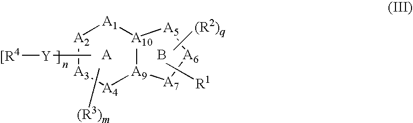

Particular embodiments of compounds of the invention include those of Formulae (IV) to (VII) below, and pharmaceutically acceptable salts or prodrugs thereof:

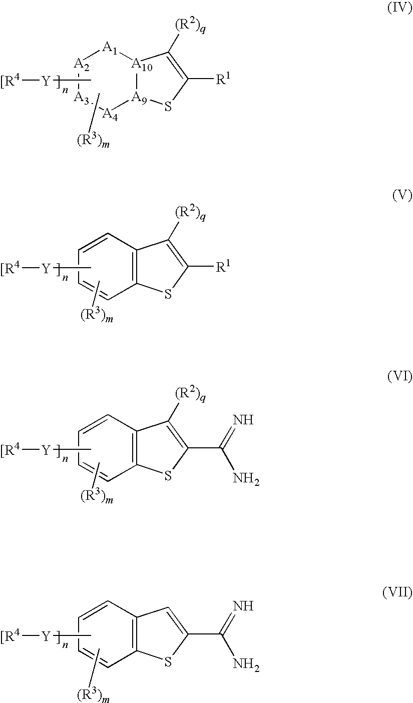

In another class of compounds, fused ring AB is one of the following ring systems:

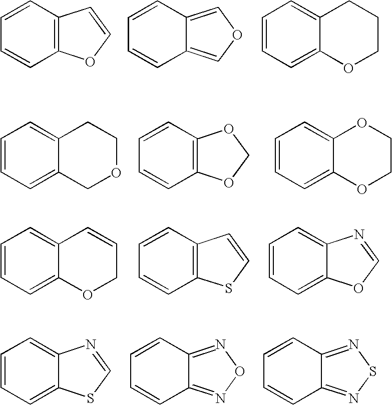

Alternatively, fused ring AB may form, by virtue of, for example, cyclisation of one or more substituents, a tricyclic ring system. This may arise because of intramolecular bonding, in particular hydrogen bonding.

The bicyclic system AB may of course be substituted by one or more substituents selected from R², R³ and —Y—R⁴, within the limitation that m is 0 to 4, and n is 0, 1 or 2. Thus, for example, when the options —CH═, —CH₂— and —NH— are mentioned in the context A₁ to A₁₀, the or each hydrogen atom attached to the carbon or nitrogen atoms may be substituted by any one of these substituents.

### R¹

R¹ is attached to ring B and is hydrogen or R¹¹ or a basic moiety.

Included, accordingly, are compounds in which R¹ is an R¹¹, e.g. halo.

In certain compounds, R¹ is a basic moiety, whether a basic moiety included in the options for R¹¹ or another one.

In one embodiment, R¹ comprises an amino group. Thus, R¹ may be an amino group, for example a substituted or unsubstituted amino group. Examples of substituted amino groups include N-alkylamino (e.g. N-methylamino), N,N-di-alkylamino, hydroxyalkylamino (e.g. 2-hydroxyethylamino or 2-hydroxypropylamino), alkoxyalkylamino (e.g. methoxyethylamino), phenylalkylamino, (e.g. benzylamino), N,N-di-alkylamino, N-phenylalkyl-N-alkylamino, N,N-dialkylphenylamino, alkanoylamino (e.g. acetylamino), benzoylamino, phenylalkoxycarbonylamino, carbamoylamino, aminocarbonylamino, aminoalkyloxyphenylamino, sulfamoylphenylamino and [N-(hydroxyalkyl)-carbamoyl]-phenylamino.

In a particular embodiment, R¹ is selected from:

(i) -G-NR^{c}R^{d}, especially —C(O)NR^{c}R^{d} or —C(O)NHR^{a};

### (ii) -G-NR^{a}OH;

(iii) -G-NR^{a}C(NR^{a})H, especially -G-NHC(NH)H;
(iv) -G-NR^{a}C(NR^{a})NR^{a}OH, especially -G-NHC(NH)NHOH;
(v) -G-NR^{a}C(NR^{a})NR^{a}CN, especially -G-NHC(NH)NHCN;
(vi) -G-NR^{a}C(NR^{a})NR^{a}C(O)R^{a}, especially -G-NHC(NH)NHC(O)R^{a};
(vii) -G-NR^{a}C(NR^{a})NR^{a}R^{b}, especially -G-NHC(NH)NHR^{a};
(viii) -G-C(NR^{a})NR^{a}R^{b}, especially -G-C(NH)NHR^{a};
(ix) -G-C(NR^{a})NR^{a}NR^{a}COR^{a}, especially -G-C(NH)NHNHC(O)R^{a};
(x) -G-C(NR^{a})NR^{a}C(NR^{a})NR^{a}R^{b}, especially -G-C(NH)NHC(NH)NH₂;
(xi) -G-C(NR^{a})NR^{a}COR^{a}, especially -G-C(NH)NHC(O)R^{a};
(xii) -G-NR^{a}C(O)R^{a}, especially -G-NHC(O)R^{a} (e.g. —C(O)NHC(O)R^{a});
(xiii) —OC(O)NR^{a}R^{b}, especially —OC(O)NHR^{a};
(xiv) —OC(O)NR^{a}C(O)R^{a}, especially —OC(O)NHC(O)R^{E};

### (xv) —C(O)ONR^{c}R^{d};

(xvi) -G-N(R¹)C(O)OR^{a}, especially —CON(R^{a})C(O)OR^{a};

(xvii) -G-N(C(O)OR^{a})C(NH₂)═NC(O)OR^{a}, especially —N(C(O)OR^{a})C(NH₂)═NC(O)OR^{a}; and

(xviii) -G-SC(═NH)NHR^{a};
wherein
G is a bond, —S(O)ᵢ, —C(O)— or —C(O)—(CH₂)ₚ—C(O)—, wherein p is 1, 2, 3 or 4;
R^{a} and R^{b} are each independently an inert organic moiety, typically containing no more than 20 atoms which are not hydrogen or halogen; and
R^{c} and R^{d} are each independently hydrogen or a moiety in which the atoms other than hydrogen and halogen are selected from the group consisting of C, N, O and S and number from 1 to 20 (especially 1, 2, 3, 4, 5, 6 or 7) and which contains at least one hydrocarbyl group which is unsubstituted or substituted by halogen and may be aliphatic or carbocyclic, and is for example selected from aryl, alkyl, alkylene, cycloalkyl, cycloalkylene, alkenyl, alkenylene, cycloalkenyl, cycloalkenylene, alkynyl and alkynylene (which may be substituted by halogen and of which alkyl, alkylene, cycloalkyl and aryl form a preferred class), and optionally 1, 2 or 3 heteroatoms selected from O, N and S;
or R^{c} and R^{d} together with the attached nitrogen atom form optionally substituted heterocyclyl, for example imidazolyl, oxazolyl, thiazolyl, benzoxazolinyl or thiazolinyl.

In one embodiment, R^{a} and R^{b} are each independently hydrogen or a moiety in which the non-hydrogen atoms are selected from the group consisting of C, N, O and S and number from 1 to 20 (especially 1, 2, 3, 4, 5, 6 or 7, for example methyl, ethyl, butyl, propyl) and which contains at least one hydrocarbyl group which may be aliphatic or carbocyclic. Thus, for example, R^{a} and R^{b} may each be independently selected from aryl, alkyl, alkylene, cycloalkyl, cycloalkylene, alkenyl, alkenylene, cycloalkenyl, cycloalkenylene, alkynyl and alkynylene, and optionally 1, 2 or 3 heteroatoms selected from O, N and S.

In a further embodiment, R^{a} and R^{b} are each independently hydrogen or C₁₋₆ alkyl (especially C₁, C₂, C₃ or C₄ alkyl), carbocyclyl, —C₁₋₆ alkyl-carbocyclyl, -carbocyclyl-C₁₋₆ alkyl, or carbocyclyl (e.g. phenyl or cyclohexyl) optionally substituted by up to three moieties selected from C₁₋₆ alkyl, C₁₋₆ alkoxy and halogen. Those R^{a} and R^{b} groups which contain one or more alkylic carbon atoms may be interrupted at an alkylic carbon by an —O— linkage.

In a further embodiment, R^{a} and R^{b} are each independently selected from hydrogen, C₁₋₆ alkyl (e.g. methyl or ethyl), phenyl and cyclohexyl. Usually, at least one or both of R^{a} and R^{b} is hydrogen in groups containing —NR^{a}R^{b}.

In a further embodiment, R^{c} and R^{d} are each independently selected from hydrogen; C₁₋₆ alkyl optionally substituted with one or more substituents selected from hydrogen, halogen, carboxyl, C₁₋₆ alkoxy C₁₋₆ alkoxycarbonyl; carbocyclyl (especially phenyl or cyclohexyl) optionally substituted with one or more substituents selected from C₁, C₂, C₃ or C₄ alkyl; C₁, C₂, C₃ or C₄ alkoxy; and halogen; and -alkyl-carbocyclyl, wherein the carbocyclyl part is, for example, phenyl or cyclohexyl, and is optionally substituted with one or more substituents selected from C₁, C₂, C₃ or C₄ alkyl; C₁, C₂, C₃ or C₄ alkoxy; and halogen.

In a further embodiment, R^{c} and R^{d} are taken together with the attached nitrogen atom form optionally substituted heterocyclyl, for example imidazolyl, oxazolyl, thiazolyl, benzoxazolinyl or thiazolinyl, and of which is optionally substituted.

In a particular embodiment, R¹ is a group of Formula (i):

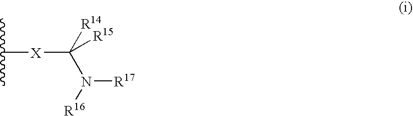

wherein
X is a bond, —NR³⁰— or —C(O)—;
R¹⁴, R¹⁵ and R³⁰ are each independently selected from R¹⁸, —OR¹⁸, —C(O)R¹⁸, —C(O)OR¹⁸, —OC(O)R¹⁸, —N(R¹⁸)R¹⁹, —C(O)N(R¹⁹)R²⁰, —S(O)ₗR¹⁸ and —C(R¹⁸)₃, e.g. are hydrogen, hydroxy or C₁₋₆ alkyl;
or R¹⁴ and R¹⁵ taken together form ═NR²⁰, ═O or ═S;
R¹⁶ and R¹⁷ are each independently selected from hydrogen, C₁₋₆ alkyl, —OR²¹ and —NR¹⁸R¹⁹;
R¹⁸ and R¹⁹ are each independently selected from hydrogen, R¹¹, hydrocarbyl optionally substituted with 1, 2, 3, 4 or 5 R¹¹; and heterocyclyl optionally substituted with 1, 2, 3, 4 or 5 R¹¹;
R²⁰ hydrogen, hydroxy, C₁₋₆ alkoxy or C₁₋₆ alkyl, e.g. is hydrogen or C₁₋₆ alkyl; and
R²¹ is hydrogen or R⁷.

In one embodiment of Formula (i), X is a bond or —N(R³⁰)—.

In another embodiment, X is a bond.

In one class of compounds, R¹⁴ and R¹⁵ together form NR²⁰ and R¹⁶, R¹⁷ and R²⁰ are each the same or different and selected from hydrogen, alkyl and hydroxy; for example they may be selected from hydrogen and hydroxy or from hydrogen and alkyl. Alkyl may have 1, 2, 3, 4, 5 or 6 carbon atoms.

In a further embodiment, R¹⁴ and R¹⁵ taken together form ═NR²⁰, wherein R²⁰ is usually hydrogen, alkyl or hydroxy, e.g. hydrogen or hydroxy. Accordingly, the invention includes compounds in which R¹ is:

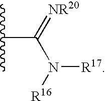

Included in the invention are compounds in which R¹⁶ and R¹⁷ are each independently selected from hydrogen, C₁₋₆ alkyl, C₁₋₆ alkoxy or hydroxy, e.g. hydrogen or C₁₋₆ alkyl.

In embodiments, R¹⁶ and/or R¹⁷ and/or R²⁰ are hydrogen.

In a particular embodiment, R¹ is a group of Formula (ii):

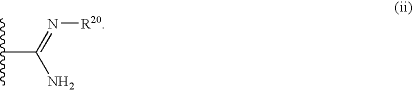

In a further embodiment, R¹ is —C(═NH)NH₂ or —C(═NOH)NH₂, particularly —C(═NH)NH₂ (amidino).

The invention includes a class of compounds in which R¹ is of formula (i) or (ii) above or is halogen, particularly F or Cl.

Particularly where R¹ is a basic group, for example of formula (i) or (ii) above, it may be in the form of a pharmaceutically acceptable salt of prodrug thereof, as in the case of other functional groups capable of being converted to a salt or prodrug form.

### R²

R² is an optional substitutent of ring B and is present when q is 1, 2, 3 or 4. R² is an organic or inorganic substituent and is often an R¹¹ atom or group. Typically, each R² is independently selected from halogen (e.g. fluorine, chlorine, bromine or iodine), hydroxy, trifluoromethyl, cyano, nitro, oxo, —OR², —C(O)R², —C(O)OR², —OC(O)R², —N(R²)R³, —C(O)N(R²)R³, —S(O)ₗR¹², —C(R²)₃ and R⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

In one embodiment, q is 0, 1 or 2.

In another embodiment, q is 0 or 1.

In a further embodiment, q is 0.

For the avoidance of doubt, where ring B is substituted with more than one R², each R² is independently selected from the range of substituents specified.

### R³

R³ is an optional substitutent of ring A and is present when m is 1, 2, 3 or 4. R³ is an organic or inorganic substituent and is often an R¹¹ atom or group. Typically, each R³ is independently selected from halogen (e.g. fluorine, chlorine, bromine or iodine), hydroxy, trifluoromethyl, cyano, nitro, oxo, —OR¹², —C(O)R¹², —C(O)OR², —OC(O)R², —N(R²)R¹³—C(O)N(R²)R³, —S(O)ₗR¹², —C(R²)₃ and R⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). However, please see under the heading “Summary of the Invention” for a more complete listing of R¹¹, R¹², R¹³ and R¹⁴ atoms or groups.

For the avoidance of doubt, where ring A is substituted with more than one R³, each R³ is independently selected from the range of substituents specified.

### R¹¹

R11 is as described under the heading “Summary of the Invention”, namely each R¹¹ is independently selected from halogen, hydroxy, trifluoromethyl, cyano, nitro, oxo, amidino, —B(OH)₂, ═NR¹², —OR², —SR², —C(O)R², —C(O)OR¹², —OC(O)R¹²—N(R²)R¹³—C(O)N(R²)R¹³, —OC(O)N(R¹²)R¹³, —S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹², —NR¹³S(O)ₗR¹², —NR¹³C(O)NR¹²R¹³, —C(R¹²)₃ and R¹⁴.

In one class of compounds, each R¹¹ is independently selected from halogen, hydroxy, trifluoromethyl, cyano, nitro, oxo, —B(OH)₂, ═NR², —OR², —C(O)R², —C(O)OR², —OC(O)R¹², —N(R²)R³, —C(O)N(R²)R³, —S(O)ₗR², —C(R¹²)₃ and R⁴. In another class of compounds, R¹¹ is as described in this paragraph except that R¹¹ as a substituent of R⁴ is as described in the previous paragraph.

Typically, each R¹¹ is independently selected from halogen (e.g. fluorine, chlorine, bromine or iodine), hydroxy, trifluoromethyl, cyano, nitro, oxo, —OR², —C(O)R², —C(O)OR², —OC(O)R¹², —N(R²)R³, —C(O)N(R²)R³, —S(O)ₗR², —C(R²)₃ and R⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

For the avoidance of doubt, where a group is substituted with more than one R¹¹, each R¹¹ is independently selected from the range of substituents specified. The same applies to compounds of the invention comprising more than one R¹¹ substituent; each R¹¹ is selected independently of any other R¹¹ substituent present in the compound.

### m & n

The index m defines the number of R³ substituents present, and is 0, 1, 2, 3 or 4.

The index n defines the number of —Y—R⁴ substituents present, and is 0, 1 or 2.

In a particular embodiment, the sum of m and n is 1, i.e. either m is 0 and n is 1, or m is 1 and n is 0.

In another embodiment, the sum of m and n is 2, i.e. m is 0 and n is 2; m is 1 and n is 1; or m is 2 and n is 0.

Included also are compounds in which n is 2 and m is 1.

### Y

Y is present when n is 1 or 2, and is a bond or a linker having 1 to 20 (e.g. 1 to 10) in-chain atoms (e.g. selected from C, N, O and S) and comprising, for example, one or more linkages selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C≡C—, carbocyclylene optionally substituted with 1, 2, 3, 4 or 5 R¹¹, and heterocyclylene optionally substituted with 1, 2, 3, 4 or 5 R¹¹. In embodiments, the one or more linkages may additionally be selected from —N(R⁶)—.

In one embodiment, n is 1 and Y is selected from:
a bond
-Y¹-;
-Y¹-Y²-Y³-;
-Y¹-Y²-Y³-Y⁴-;
-Y¹-Y²-Y³-Y⁴-Y⁵-;
-Y¹-Y²-Y³-Y⁴-Y⁵-Y⁶-;
-Y¹-Y²-Y³-Y⁴-Y⁵-Y⁶-Y⁷-;
-Y¹-Y²-Y³-Y⁴-Y⁵-Y⁶-Y⁷-Y⁸-;
-Y¹-Y²-Y³-Y⁴-Y⁵-Y⁶-Y⁷-Y⁸-Y⁹-; and
Y¹-Y²-Y³-Y⁴-Y⁵-Y⁶-Y⁷-Y⁸-Y⁹-Y¹⁰-;
   wherein Y¹, Y², Y³, Y⁴, Y⁵, Y⁶, Y⁷, Y⁸, Y⁹ and Y¹⁰ are each independently selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C═C—, carbocyclylene optionally substituted with 1, 2, 3, 4 or 5 R¹, and heterocyclylene optionally substituted with 1, 2, 3, 4 or 5 R¹¹. In some compounds, Y¹-Y¹⁰ may additionally be selected from —N(R⁶)—.

In another embodiment, n is 2 and each Y is independently selected from:
a bond
-Y¹-Y²-;
-Y¹-Y²-Y³-;
-Y¹-Y²-Y³-Y⁴-;
-Y¹-Y²-Y³-Y⁴-Y⁵-;
-Y¹-Y²-Y³-Y⁴-Y⁵-Y⁶-;
-Y¹-Y²-Y³-Y⁴-Y⁵-Y⁶-Y⁷-;
-Y¹-Y²-Y³-Y⁴-Y⁵-Y⁶-Y⁷-Y⁸-;
-Y¹-Y²-Y³-Y⁴-Y⁵-Y⁶-Y⁷-Y⁸-Y⁹-; and
-Y¹-Y²-Y³-Y⁴-Y⁵-Y⁶-Y⁷-Y⁸-Y⁹-Y¹⁰-;
   wherein Y¹, Y², Y³, Y⁴, Y¹, Y⁶, Y⁷, Y⁸, Y⁹ and Y¹⁰ are each independently selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C≡C— carbocyclylene optionally substituted with 1, 2, 3, 4 or 5 R¹¹, and heterocyclylene optionally substituted with 1, 2, 3, 4 or 5 R¹¹. In embodiments, Y¹-Y¹⁰ may additionally be selected from —N(R⁶)—.

Y¹ designates a moiety attached to ring AB, i.e. a linker comprising the structure -Y¹-Y²- is directly covalently bonded to ring AB through Y¹.

Of particular mention are compounds in which the or each Y is a linker and is attached to ring AB via a terminal —O— atom of Y. Thus, in the linkers specified above, Y¹ is especially —O—.

Of particular mention are compounds comprising at least one —Y—R⁴ substituent which is other than alkoxy (in particular methoxy).

Also of particular mention are compounds having Y groups which comprise a -Y¹-Y²- moiety other than —O—C(O)— or —S—C(O)—. Thus, certain compounds may comprise at least one —Y—R⁴ substituent other than —O—C(O)—R⁴ or —S—C(O)—R⁴. In certain compounds, a substituent of the formula —O—C(O)—R⁴ or —S—C(O)—R⁴ is absent.

Also of particular mention are compounds in which the or each Y is a linker and is attached to ring AB via carbocyclylene (e.g phenylene), more particularly —O-carbocyclylene, which is optionally substituted with 1, 2, 3, 4 or 5 R¹¹. Thus, in the linkers specified above, Y¹ and Y² are especially carbocyclylene (e.g. arylene) optionally substituted with 1, 2, 3, 4 or 5 R¹¹, and —O— respectively.

Examples of the linker Y¹ are described in Table 2:

TABLE 2

| No. | Y¹ |
|---|---|
| 1 | —O— |
| 2 | —N(R⁵)— |
| 3 | —C(O)— |
| 4 | —C(S)— |
| 5 | —S(O)ₗ— |
| 6 | —(CH₂)ₖ— |
| 7 | —C(R⁶)(R⁷)— |
| 8 | —C(R⁵)═C(R⁵)— |
| 9 | —C≡C— |
| 10 | carbocyclylene |
| 11 | heterocyclylene |

Further examples of Y¹ are described in Table 2:

TABLE 3

| No. | Y¹ |
|---|---|
| 1 | —O— |
| 2 | —NH— |
| 3 | —N(-alkyl)- |
| 4 | —N[—(CH₂)ₖ-aryl]- |
| 5 | —C(O)— |
| 6 | —C(S)— |
| 7 | —S— |
| 8 | —S(O)— |
| 9 | —S(O)₂— |
| 10 | —CH₂— |
| 11 | —(CH₂)₂— |
| 12 | —(CH₂)₃— |
| 13 | —CH(alkyl)- |
| 14 | —CH[—(CH₂)ₖ-aryl]- |
| 15 | —C(alkyl)₂- |
| 16 | —CH═CH— |
| 17 | —CH═C(alkyl)- |
| 18 | —C(alkyl)═CH— |
| 19 | —C≡C— |
| 20 | cycloalkylene |
| 21 | arylene |
| 22 | heterocycloalkylene |
| 23 | heteroarylene |

Further examples of Y¹ are described in Table 4:

TABLE 4

| No. | Y¹ |
|---|---|
| 1 | —O— |
| 2 | —NH— |
| 3 | —N(CH₃)— |
| 4 | —C(O)— |
| 5 | —C(S)— |
| 6 | —CH₂— |
| 7 | —(CH₂)₂— |
| 8 | —(CH₂)₃— |
| 9 | —CH═CH— |
| 10 | —C≡C— |
| 11 | phenylene |
| 12 | 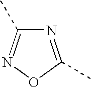 |

Examples of the linker -Y¹-Y²- are given in Table 5:

TABLE 5

| No. | Y¹ | Y² |
|---|---|---|
| 1 | —N(R⁵)— | —O— |
| 2 | —C(O)— | —O— |
| 5 | —(CH₂)ₖ— | —O— |
| 6 | —C(R⁶)(R⁷)— | —O— |
| 7 | carbocyclylene | —O— |
| 8 | heterocyclylene | —O— |
| 9 | —O— | —N(R⁵)— |
| 10 | —N(R⁵)— | —N(R⁵)— |
| 11 | —C(O)— | —N(R⁵)— |
| 13 | —S(O)ₗ— | —N(R⁵)— |
| 14 | —(CH₂)ₖ— | —N(R⁵)— |
| 15 | —C(R⁶)(R⁷)— | —N(R⁵)— |
| 16 | carbocyclylene | —N(R⁵)— |
| 17 | heterocyclylene | —N(R⁵)— |
| 18 | —O— | —C(O)— |
| 19 | —N(R⁵)— | —C(O)— |
| 21 | —(CH₂)ₖ— | —C(O)— |
| 22 | —C(R⁶)(R⁷)— | —C(O)— |
| 23 | carbocyclylene | —C(O)— |
| 24 | heterocyclylene | —C(O)— |
| 27 | —(CH₂)ₖ— | —C(S)— |
| 28 | —C(R⁶)(R⁷)— | —C(S)— |
| 29 | carbocyclylene | —C(S)— |
| 30 | heterocyclylene | —C(S)— |
| 32 | —N(R⁵)— | —S(O)ₗ— |
| 33 | —(CH₂)ₖ— | —S(O)ₗ— |
| 34 | —C(R⁶)(R⁷)— | —S(O)ₗ— |
| 35 | carbocyclylene | —S(O)ₗ— |
| 36 | heterocyclylene | —S(O)ₗ— |
| 37 | —O— | —(CH₂)ₖ— |
| 38 | —N(R⁵)— | —(CH₂)ₖ— |
| 39 | —C(O)— | —(CH₂)ₖ— |
| 40 | —C(S)— | —(CH₂)ₖ— |
| 41 | —S(O)ₗ— | —(CH₂)ₖ— |
| 43 | —C(R⁶)(R⁷)— | —(CH₂)ₖ— |
| 44 | —C(R⁵)═C(R⁵)— | —(CH₂)ₖ— |
| 45 | —C≡C— | —(CH₂)ₖ— |
| 46 | carbocyclylene | —(CH₂)ₖ— |
| 47 | heterocyclylene | —(CH₂)ₖ— |
| 48 | —O— | —C(R⁶)(R⁷)— |
| 49 | —N(R⁵)— | —C(R⁶)(R⁷)— |
| 50 | —C(O)— | —C(R⁶)(R⁷)— |
| 51 | —C(S)— | —C(R⁶)(R⁷)— |
| 52 | —S(O)ₗ— | —C(R⁶)(R⁷)— |
| 53 | —(CH₂)ₖ— | —C(R⁶)(R⁷)— |
| 54 | —C(R⁶)(R⁷)— | —C(R⁶)(R⁷)— |
| 55 | —C(R⁵)═C(R⁵)— | —C(R⁶)(R⁷)— |
| 56 | —C≡C— | —C(R⁶)(R⁷)— |
| 57 | carbocyclylene | —C(R⁶)(R⁷)— |
| 58 | heterocyclylene | —C(R⁶)(R⁷)— |
| 64 | —(CH₂)ₖ— | —C(R⁵)═C(R⁵)— |
| 65 | —C(R⁶)(R⁷)— | —C(R⁵)═C(R⁵)— |
| 66 | carbocyclylene | —C(R⁵)═C(R⁵)— |
| 67 | heterocyclylene | —C(R⁵)═C(R⁵)— |
| 68 | —(CH₂)ₖ— | —C≡C— |
| 69 | —C(R⁶)(R⁷)— | —C≡C— |
| 70 | carbocyclylene | —C≡C— |
| 71 | heterocyclylene | —C≡C— |
| 72 | —O— | carbocyclylene |
| 73 | —N(R⁵)— | carbocyclylene |
| 74 | —C(O)— | carbocyclylene |
| 75 | —C(S)— | carbocyclylene |
| 76 | —S(O)ₗ— | carbocyclylene |
| 77 | —(CH₂)ₖ— | carbocyclylene |
| 78 | —C(R⁶)(R⁷)— | carbocyclylene |
| 79 | carbocyclylene | carbocyclylene |
| 80 | heterocyclylene | carbocyclylene |
| 81 | —O— | heterocyclylene |
| 82 | —N(R⁵)— | heterocyclylene |
| 83 | —C(O)— | heterocyclylene |
| 84 | —C(S)— | heterocyclylene |
| 85 | —S(O)ₗ— | heterocyclylene |
| 86 | —(CH₂)ₖ— | heterocyclylene |
| 87 | —C(R⁶)(R⁷)— | heterocyclylene |
| 88 | carbocyclylene | heterocyclylene |
| 89 | heterocyclylene | heterocyclylene |

Particular examples of -Y¹-Y²- are given in Table 6:

TABLE 6

| No. | Y¹ | Y² |
|---|---|---|
| 1 | —O— | —N(R⁵)— |
| 2 | —O— | —C(O)— |
| 3 | —O— | —(CH₂⁾ₖ— |
| 4 | —O— | —C(R⁶)(R⁷)— |

Further particular examples of -Y¹-Y²- are given in Table 7:

TABLE 7

| No. | Y¹ | Y² |
|---|---|---|
| 1 | —O— | —NH— |
| 2 | —O— | —N(-alkyl)— |
| 3 | —O— | —C(O)— |
| 4 | —O— | —CH₂— |
| 5 | —O— | —(CH₂)₂— |
| 6 | —O— | —CH(phenyl)— |
| 7 | —O— | —CH(benzyl)— |

Examples of the linker -Y¹-Y²-Y³- are given in Table 8:

TABLE 8

| No. | Y¹ | Y² | Y³ |
|---|---|---|---|
| 1 | —C(O)— | —O— | —N(R⁵)— |
| 2 | carbocyclylene | —O— | —N(R⁵)— |
| 3 | heterocyclylene | —O— | —N(R⁵)— |
| 4 | —C(O)— | —N(R⁵)— | —N(R⁵)— |
| 5 | carbocyclylene | —N(R⁵)— | —N(R⁵)— |
| 6 | heterocyclylene | —N(R⁵)— | —N(R⁵)— |
| 7 | —O— | —C(O)— | —N(R⁵)— |
| 8 | —N(R⁵)— | —C(O)— | —N(R⁵)— |
| 9 | carbocyclylene | —C(O)— | —N(R⁵)— |
| 10 | heterocyclylene | —C(O)— | —N(R⁵)— |
| 11 | —N(R⁵)— | —S(O)ₗ— | —N(R⁵)— |
| 12 | carbocyclylene | —S(O)ₗ— | —N(R⁵)— |
| 13 | heterocyclylene | —S(O)ₗ— | —N(R⁵)— |
| 14 | —O— | —(CH₂)ₖ— | —N(R⁵)— |
| 15 | —N(R⁵)— | —(CH₂)ₖ— | —N(R⁵)— |
| 16 | —C(O)— | —(CH₂)ₖ— | —N(R⁵)— |
| 17 | carbocyclylene | —(CH₂)ₖ— | —N(R⁵)— |
| 18 | heterocyclylene | —(CH₂)ₖ— | —N(R⁵)— |
| 19 | —O— | —C(R⁶)(R⁷)— | —N(R⁵)— |
| 20 | —N(R⁵)— | —C(R⁶)(R⁷)— | —N(R⁵)— |
| 21 | —C(O)— | —C(R⁶)(R⁷)— | —N(R⁵)— |
| 22 | carbocyclylene | —C(R⁶)(R⁷)— | —N(R⁵)— |
| 23 | heterocyclylene | —C(R⁶)(R⁷)— | —N(R⁵)— |
| 24 | —O— | carbocyclylene | —N(R⁵)— |
| 25 | —N(R⁵)— | carbocyclylene | —N(R⁵)— |
| 26 | —C(O)— | carbocyclylene | —N(R⁵)— |
| 27 | carbocyclylene | carbocyclylene | —N(R⁵)— |
| 28 | heterocyclylene | carbocyclylene | —N(R⁵)— |
| 29 | —O— | heterocyclylene | —N(R⁵)— |
| 30 | —N(R⁵)— | heterocyclylene | —N(R⁵)— |
| 31 | —C(O)— | heterocyclylene | —N(R⁵)— |
| 32 | carbocyclylene | heterocyclylene | —N(R⁵)— |
| 33 | heterocyclylene | heterocyclylene | —N(R⁵)— |
| 34 | —N(R⁵)— | —O— | —C(O)— |
| 35 | —C(O)— | —O— | —C(O)— |
| 36 | carbocyclylene | —O— | —C(O)— |
| 37 | heterocyclylene | —O— | —C(O)— |
| 38 | —O— | —N(R⁵)— | —C(O)— |
| 39 | —N(R⁵)— | —N(R⁵)— | —C(O)— |
| 40 | —C(O)— | —N(R⁵)— | —C(O)— |
| 41 | carbocyclylene | —N(R⁵)— | —C(O)— |
| 42 | heterocyclylene | —N(R⁵)— | —C(O)— |
| 43 | —O— | —(CH₂)ₖ— | —C(O)— |
| 44 | —N(R⁵)— | —(CH₂)ₖ— | —C(O)— |
| 45 | —C(O)— | —(CH₂)ₖ— | —C(O)— |
| 46 | carbocyclylene | —(CH₂)ₖ— | —C(O)— |
| 47 | heterocyclylene | —(CH₂)ₖ— | —C(O)— |
| 48 | —O— | —C(R⁶)(R⁷)— | —C(O)— |
| 49 | —N(R⁵)— | —C(R⁶)(R⁷)— | —C(O)— |
| 50 | —C(O)— | —C(R⁶)(R⁷)— | —C(O)— |
| 51 | carbocyclylene | —C(R⁶)(R⁷)— | —C(O)— |
| 52 | heterocyclylene | —C(R⁶)(R⁷)— | —C(O)— |
| 53 | —O— | carbocyclylene | —C(O)— |
| 54 | —N(R⁵)— | carbocyclylene | —C(O)— |
| 55 | —C(O)— | carbocyclylene | —C(O)— |
| 56 | carbocyclylene | carbocyclylene | —C(O)— |
| 57 | heterocyclylene | carbocyclylene | —C(O)— |
| 58 | —O— | heterocyclylene | —C(O)— |
| 59 | —N(R⁵)— | heterocyclylene | —C(O)— |
| 60 | —C(O)— | heterocyclylene | —C(O)— |
| 61 | carbocyclylene | heterocyclylene | —C(O)— |
| 62 | heterocyclylene | heterocyclylene | —C(O)— |
| 63 | —O— | —(CH₂)ₖ— | —C(S)— |
| 64 | —N(R⁵)— | —(CH₂)ₖ— | —C(S)— |
| 65 | —C(O)— | —(CH₂)ₖ— | —C(S)— |
| 66 | carbocyclylene | —(CH₂)ₖ— | —C(S)— |
| 67 | heterocyclylene | —(CH₂)ₖ— | —C(S)— |
| 68 | —O— | —C(R⁶)(R⁷)— | —C(S)— |
| 69 | —N(R⁵)— | —C(R⁶)(R⁷)— | —C(S)— |
| 70 | —C(O)— | —C(R⁶)(R⁷)— | —C(S)— |
| 71 | carbocyclylene | —C(R⁶)(R⁷)— | —C(S)— |
| 72 | heterocyclylene | —C(R⁶)(R⁷)— | —C(S)— |
| 73 | —O— | carbocyclylene | —C(S)— |
| 74 | —N(R⁵)— | carbocyclylene | —C(S)— |
| 75 | —C(O)— | carbocyclylene | —C(S)— |
| 76 | carbocyclylene | carbocyclylene | —C(S)— |
| 77 | heterocyclylene | carbocyclylene | —C(S)— |
| 78 | —O— | heterocyclylene | —C(S)— |
| 79 | —N(R⁵)— | heterocyclylene | —C(S)— |
| 80 | —C(O)— | heterocyclylene | —C(S)— |
| 81 | carbocyclylene | heterocyclylene | —C(S)— |
| 82 | heterocyclylene | heterocyclylene | —C(S)— |
| 83 | —O— | —N(R⁵)— | —S(O)ₗ— |
| 84 | —N(R⁵)— | —N(R⁵)— | —S(O)ₗ— |
| 85 | —C(O)— | —N(R⁵)— | —S(O)ₗ— |
| 86 | carbocyclylene | —N(R⁵)— | —S(O)ₗ— |
| 87 | heterocyclylene | —N(R⁵)— | —S(O)ₗ— |
| 88 | —O— | —(CH₂)ₖ— | —S(O)ₗ— |
| 89 | —N(R⁵)— | —(CH₂)ₖ— | —S(O)ₗ— |
| 90 | —C(O)— | —(CH₂)ₖ— | —S(O)ₗ— |
| 91 | carbocyclylene | —(CH₂)ₖ— | —S(O)ₗ— |
| 92 | heterocyclylene | —(CH₂)ₖ— | —S(O)ₗ— |
| 93 | —O— | —C(R⁶)(R⁷)— | —S(O)ₗ— |
| 94 | —N(R⁵)— | —C(R⁶)(R⁷)— | —S(O)ₗ— |
| 95 | —C(O)— | —C(R⁶)(R⁷)— | —S(O)ₗ— |
| 96 | carbocyclylene | —C(R⁶)(R⁷)— | —S(O)ₗ— |
| 97 | heterocyclylene | —C(R⁶)(R⁷)— | —S(O)ₗ— |
| 98 | —O— | carbocyclylene | —S(O)ₗ— |
| 99 | —N(R⁵)— | carbocyclylene | —S(O)ₗ— |
| 100 | —C(O)— | carbocyclylene | —S(O)ₗ— |
| 101 | carbocyclylene | carbocyclylene | —S(O)ₗ— |
| 102 | heterocyclylene | carbocyclylene | —S(O)ₗ— |
| 103 | —O— | heterocyclylene | —S(O)ₗ— |
| 104 | —N(R⁵)— | heterocyclylene | —S(O)ₗ— |
| 105 | —C(O)— | heterocyclylene | —S(O)ₗ— |
| 106 | carbocyclylene | heterocyclylene | —S(O)ₗ— |
| 107 | heterocyclylene | heterocyclylene | —S(O)ₗ— |
| 108 | —N(R⁵)— | —O— | —(CH₂)ₖ— |
| 109 | —C(O)— | —O— | —(CH₂)ₖ— |
| 110 | carbocyclylene | —O— | —(CH₂)ₖ— |
| 111 | heterocyclylene | —O— | —(CH₂)ₖ— |
| 112 | —O— | —N(R⁵)— | —(CH₂)ₖ— |
| 113 | —N(R⁵)— | —N(R⁵)— | —(CH₂)ₖ— |
| 114 | —C(O)— | —N(R⁵)— | —(CH₂)ₖ— |
| 115 | carbocyclylene | —N(R⁵)— | —(CH₂)ₖ— |
| 116 | heterocyclylene | —N(R⁵)— | —(CH₂)ₖ— |
| 117 | —O— | —C(O)— | —(CH₂)ₖ— |
| 118 | —N(R⁵)— | —C(O)— | —(CH₂)ₖ— |
| 119 | carbocyclylene | —C(O)— | —(CH₂)ₖ— |
| 120 | heterocyclylene | —C(O)— | —(CH₂)ₖ— |
| 121 | carbocyclylene | —C(S)— | —(CH₂)ₖ— |
| 122 | heterocyclylene | —C(S)— | —(CH₂)ₖ— |
| 123 | —N(R⁵)— | —S(O)ₗ— | —(CH₂)ₖ— |
| 124 | carbocyclylene | —S(O)ₗ— | —(CH₂)ₖ— |
| 125 | heterocyclylene | —S(O)ₗ— | —(CH₂)ₖ— |
| 126 | —O— | —C(R⁶)(R⁷)— | —(CH₂)ₖ— |
| 127 | —N(R⁵)— | —C(R⁶)(R⁷)— | —(CH₂)ₖ— |
| 128 | —C(O)— | —C(R⁶)(R⁷)— | —(CH₂)ₖ— |
| 129 | carbocyclylene | —C(R⁶)(R⁷)— | —(CH₂)ₖ— |
| 130 | heterocyclylene | —C(R⁶)(R⁷)— | —(CH₂)ₖ— |
| 131 | carbocyclylene | —C(R⁵)═C(R⁵)— | —(CH₂)ₖ— |
| 132 | heterocyclylene | —C(R⁵)═C(R⁵)— | —(CH₂)ₖ— |
| 133 | —O— | carbocyclylene | —(CH₂)ₖ— |
| 134 | —N(R⁵)— | carbocyclylene | —(CH₂)ₖ— |
| 135 | —C(O)— | carbocyclylene | —(CH₂)ₖ— |
| 136 | carbocyclylene | carbocyclylene | —(CH₂)ₖ— |
| 137 | heterocyclylene | carbocyclylene | —(CH₂)ₖ— |
| 138 | —O— | heterocyclylene | —(CH₂)ₖ— |
| 139 | —N(R⁵)— | heterocyclylene | —(CH₂)ₖ— |
| 140 | —C(O)— | heterocyclylene | —(CH₂)ₖ— |
| 141 | carbocyclylene | heterocyclylene | —(CH₂)ₖ— |
| 142 | heterocyclylene | heterocyclylene | —(CH₂)ₖ— |
| 143 | —N(R⁵)— | —O— | —C(R⁶)(R⁷)— |
| 144 | —C(O)— | —O— | —C(R⁶)(R⁷)— |
| 145 | carbocyclylene | —O— | —C(R⁶)(R⁷)— |
| 146 | heterocyclylene | —O— | —C(R⁶)(R⁷)— |
| 147 | —O— | —N(R⁵)— | —C(R⁶)(R⁷)— |
| 148 | —N(R⁵)— | —N(R⁵)— | —C(R⁶)(R⁷)— |
| 149 | —C(O)— | —N(R⁵)— | —C(R⁶)(R⁷)— |
| 150 | carbocyclylene | —N(R⁵)— | —C(R⁶)(R⁷)— |
| 151 | heterocyclylene | —N(R⁵)— | —C(R⁶)(R⁷)— |
| 152 | —O— | —C(O)— | —C(R⁶)(R⁷)— |
| 153 | —N(R⁵)— | —C(O)— | —C(R⁶)(R⁷)— |
| 154 | carbocyclylene | —C(O)— | —C(R⁶)(R⁷)— |
| 155 | heterocyclylene | —C(O)— | —C(R⁶)(R⁷)— |
| 156 | carbocyclylene | —C(S)— | —C(R⁶)(R⁷)— |
| 157 | heterocyclylene | —C(S)— | —C(R⁶)(R⁷)— |
| 158 | —N(R⁵)— | —S(O)ₗ— | —C(R⁶)(R⁷)— |
| 159 | carbocyclylene | —S(O)ₗ— | —C(R⁶)(R⁷)— |
| 160 | heterocyclylene | —S(O)ₗ— | —C(R⁶)(R⁷)— |
| 161 | —O— | —(CH₂)ₖ— | —C(R⁶)(R⁷)— |
| 162 | —N(R⁵)— | —(CH₂)ₖ— | —C(R⁶)(R⁷)— |
| 163 | —C(O)— | —(CH₂)ₖ— | —C(R⁶)(R⁷)— |
| 164 | carbocyclylene | —(CH₂)ₖ— | —C(R⁶)(R⁷)— |
| 165 | heterocyclylene | —(CH₂)ₖ— | —C(R⁶)(R⁷)— |
| 166 | —O— | —C(R⁶)(R⁷)— | —C(R⁶)(R⁷)— |
| 167 | —N(R⁵)— | —C(R⁶)(R⁷)— | —C(R⁶)(R⁷)— |
| 168 | —C(O)— | —C(R⁶)(R⁷)— | —C(R⁶)(R⁷)— |
| 169 | carbocyclylene | —C(R⁶)(R⁷)— | —C(R⁶)(R⁷)— |
| 170 | heterocyclylene | —C(R⁶)(R⁷)— | —C(R⁶)(R⁷)— |
| 171 | carbocyclylene | —C(R⁵)═C(R⁵)— | —C(R⁶)(R⁷)— |
| 172 | heterocyclylene | —C(R⁵)═C(R⁵)— | —C(R⁶)(R⁷)— |
| 173 | —O— | carbocyclylene | —C(R⁶)(R⁷)— |
| 174 | —N(R⁵)— | carbocyclylene | —C(R⁶)(R⁷)— |
| 175 | —C(O)— | carbocyclylene | —C(R⁶)(R⁷)— |
| 176 | carbocyclylene | carbocyclylene | —C(R⁶)(R⁷)— |
| 177 | heterocyclylene | carbocyclylene | —C(R⁶)(R⁷)— |
| 178 | —O— | heterocyclylene | —C(R⁶)(R⁷)— |
| 179 | —N(R⁵)— | heterocyclylene | —C(R⁶)(R⁷)— |
| 180 | —C(O)— | heterocyclylene | —C(R⁶)(R⁷)— |
| 181 | carbocyclylene | heterocyclylene | —C(R⁶)(R⁷)— |
| 182 | heterocyclylene | heterocyclylene | —C(R⁶)(R⁷)— |
| 183 | —N(R⁵)— | —O— | carbocyclylene |
| 184 | —C(O)— | —O— | carbocyclylene |
| 185 | carbocyclylene | —O— | carbocyclylene |
| 186 | heterocyclylene | —O— | carbocyclylene |
| 187 | —O— | —N(R⁵)— | carbocyclylene |
| 188 | —N(R⁵)— | —N(R⁵)— | carbocyclylene |
| 189 | —C(O)— | —N(R⁵)— | carbocyclylene |
| 190 | carbocyclylene | —N(R⁵)— | carbocyclylene |
| 191 | heterocyclylene | —N(R⁵)— | carbocyclylene |
| 192 | —O— | —C(O)— | carbocyclylene |
| 193 | —N(R⁵)— | —C(O)— | carbocyclylene |
| 194 | carbocyclylene | —C(O)— | carbocyclylene |
| 195 | heterocyclylene | —C(O)— | carbocyclylene |
| 196 | carbocyclylene | —C(S)— | carbocyclylene |
| 197 | heterocyclylene | —C(S)— | carbocyclylene |
| 198 | —N(R⁵)— | —S(O)ₗ— | carbocyclylene |
| 199 | carbocyclylene | —S(O)ₗ— | carbocyclylene |
| 200 | heterocyclylene | —S(O)ₗ— | carbocyclylene |
| 201 | —O— | —(CH₂)ₖ— | carbocyclylene |
| 202 | —N(R⁵)— | —(CH₂)ₖ— | carbocyclylene |
| 203 | —C(O)— | —(CH₂)ₖ— | carbocyclylene |
| 204 | carbocyclylene | —(CH₂)ₖ— | carbocyclylene |
| 205 | heterocyclylene | —(CH₂)ₖ— | carbocyclylene |
| 206 | —O— | —C(R⁶)(R⁷)— | carbocyclylene |
| 207 | —N(R⁵)— | —C(R⁶)(R⁷)— | carbocyclylene |
| 208 | —C(O)— | —C(R⁶)(R⁷)— | carbocyclylene |
| 209 | carbocyclylene | —C(R⁶)(R⁷)— | carbocyclylene |
| 210 | heterocyclylene | —C(R⁶)(R⁷)— | carbocyclylene |
| 211 | carbocyclylene | —C(R⁵)═C(R⁵)— | carbocyclylene |
| 212 | heterocyclylene | —C(R⁵)═C(R⁵)— | carbocyclylene |
| 213 | —O— | carbocyclylene | carbocyclylene |
| 214 | —N(R⁵)— | carbocyclylene | carbocyclylene |
| 215 | —C(O)— | carbocyclylene | carbocyclylene |
| 216 | —O— | heterocyclylene | carbocyclylene |
| 217 | —N(R⁵)— | heterocyclylene | carbocyclylene |
| 218 | —C(O)— | heterocyclylene | carbocyclylene |
| 219 | —N(R⁵)— | —O— | heterocyclylene |
| 220 | —C(O)— | —O— | heterocyclylene |
| 221 | carbocyclylene | —O— | heterocyclylene |
| 222 | heterocyclylene | —O— | heterocyclylene |
| 223 | —O— | —N(R⁵)— | heterocyclylene |
| 224 | —N(R⁵)— | —N(R⁵)— | heterocyclylene |
| 225 | —C(O)— | —N(R⁵)— | heterocyclylene |
| 226 | carbocyclylene | —N(R⁵)— | heterocyclylene |
| 227 | heterocyclylene | —N(R⁵)— | heterocyclylene |
| 228 | —O— | —C(O)— | heterocyclylene |
| 229 | —N(R⁵)— | —C(O)— | heterocyclylene |
| 230 | carbocyclylene | —C(O)— | heterocyclylene |
| 231 | heterocyclylene | —C(O)— | heterocyclylene |
| 232 | carbocyclylene | —C(S)— | heterocyclylene |
| 233 | heterocyclylene | —C(S)— | heterocyclylene |
| 234 | —N(R⁵)— | —S(O)ₗ— | heterocyclylene |
| 235 | carbocyclylene | —S(O)ₗ— | heterocyclylene |
| 236 | heterocyclylene | —S(O)ₗ— | heterocyclylene |
| 237 | —O— | —(CH₂)ₖ— | heterocyclylene |
| 238 | —N(R⁵)— | —(CH₂)ₖ— | heterocyclylene |
| 239 | —C(O)— | —(CH₂)ₖ— | heterocyclylene |
| 240 | carbocyclylene | —(CH₂)ₖ— | heterocyclylene |
| 241 | heterocyclylene | —(CH₂)ₖ— | heterocyclylene |
| 242 | —O— | —C(R⁶)(R⁷)— | heterocyclylene |
| 243 | —N(R⁵)— | —C(R⁶)(R⁷)— | heterocyclylene |
| 244 | —C(O)— | —C(R⁶)(R⁷)— | heterocyclylene |
| 245 | carbocyclylene | —C(R⁶)(R⁷)— | heterocyclylene |
| 246 | heterocyclylene | —C(R⁶)(R⁷)— | heterocyclylene |
| 247 | carbocyclylene | —C(R⁵)═C(R⁵)— | heterocyclylene |
| 248 | heterocyclylene | —C(R⁵)═C(R⁵)— | heterocyclylene |
| 249 | —O— | carbocyclylene | heterocyclylene |
| 250 | —N(R⁵)— | carbocyclylene | heterocyclylene |
| 251 | —C(O)— | carbocyclylene | heterocyclylene |
| 252 | —O— | heterocyclylene | heterocyclylene |
| 253 | —N(R⁵)— | heterocyclylene | heterocyclylene |
| 254 | —C(O)— | heterocyclylene | heterocyclylene |

Particular examples of -Y¹-Y²-Y³- are given in Table 9:

TABLE 9

| No. | Y¹ | Y² | Y³ |
|---|---|---|---|
| 1 | —O— | —(CH₂)ₖ— | carbocyclylene |
| 2 | —O— | —(CH₂)ₖ— | heterocyclylene |
| 3 | —O— | —(CH₂)ₖ— | —N(R⁵)— |
| 4 | —O— | —C(R⁶)(R⁷)— | —(CH₂)ₖ— |
| 5 | —O— | —C(R⁶)(R⁷)— | carbocyclylene |
| 6 | —O— | —C(R⁶)(R⁷)— | heterocyclylene |
| 7 | —N(R⁵)— | —C(O)— | —(CH₂)ₖ— |
| 8 | —C(O)— | —O— | —(CH₂)ₖ— |
| 9 | —C(O)— | —N(R⁵)— | —(CH₂)ₖ— |
| 10 | —C(O)— | —N(R⁵)— | —C(R⁶)(R⁷)— |
| 11 | —C(O)— | —N(R⁵)— | —O— |
| 12 | carbocyclylene | —O— | —N(R⁵)— |
| 13 | carbocyclylene | —O— | —C(O)— |
| 14 | carbocyclylene | —O— | —(CH₂)ₖ— |
| 15 | carbocyclylene | —O— | —C(R⁶)(R⁷)— |
| 16 | carbocyclylene | —C(O)— | —N(R⁵)— |
| 17 | —C(R⁶)(R⁷)— | —O— | —C(O)— |

Further particular examples of -Y¹-Y²-Y³- are given in Table 10:

TABLE 10

| No. | Y¹ | Y² | Y³ |
|---|---|---|---|
| 1 | —O— | —C(R⁶)(R⁷)— | —C(O)— |
| 2 | —O— | —C(R⁶)(R⁷)— | phenylene |
| 3 | phenylene | —O— | —N(R⁵)— |
| 4 | phenylene | —O— | —C(O)— |
| 5 | phenylene | —O— | —(CH₂)ₖ— |
| 6 | phenylene | —O— | —C(R⁶)(R⁷)— |

Further particular examples of -Y¹-Y²-Y³- are given in Table 11:

TABLE 11

| No. | Y¹ | Y² | Y³ |
|---|---|---|---|
| 1 | —O— | —CH₂— | phenylene |
| 2 | —O— | —CH₂— | heterocyclylene |
| 3 | —O— | —(CH₂)₂— | NH |
| 4 | —O— | —(CH₂)₂— | —N(alkyl)- |
| 5 | —O— | —CH(phenyl)- | —CH₂— |
| 6 | —O— | —CH(phenyl)- | phenylene |
| 7 | —O— | —CH(phenyl)- | heterocyclylene |
| 8 | —NH— | —C(O)— | —CH₂— |
| 9 | —N(alkyl)- | —C(O)— | —CH₂— |
| 10 | —C(O)— | —O— | —CH₂— |
| 11 | —C(O)— | —NH— | —CH₂— |
| 12 | —C(O)— | —NH— | —O— |
| 13 | —C(O)— | —N(alkyl)- | —O— |
| 14 | phenylene | —O— | —NH— |
| 15 | phenylene | —O— | —N(alkyl)- |
| 16 | phenylene | —O— | —C(O)— |
| 17 | phenylene | —O— | —CH₂— |
| 18 | phenylene | —O— | —(CH₂)₂— |
| 19 | phenylene | —O— | —CH(phenyl)- |
| 20 | phenylene | —O— | —CH(benzyl)- |
| 21 | phenylene | —C(O)— | —NH— |
| 22 | —CH(phenyl)- | —O— | —C(O)— |

Examples of the linker -Y¹-Y²-Y³-Y⁴- are given in Table 12:

TABLE 12

| No. | Y¹ | Y² | Y³ | Y⁴ |
|---|---|---|---|---|
| 1 | —O— | —C(O)— | —N(R⁵)— | —O— |
| 2 | —O— | —(CH₂)ₖ— | —N(R⁵)— | —O— |
| 3 | —O— | —C(R⁶)(R⁷)— | —N(R⁵)— | —O— |
| 4 | —O— | carbocyclylene | —N(R⁵)— | —O— |
| 5 | —O— | heterocyclylene | —N(R⁵)— | —O— |
| 6 | —O— | —N(R⁵)— | —C(O)— | —O— |
| 7 | —O— | —(CH₂)ₖ— | —C(O)— | —O— |
| 8 | —O— | —C(R⁶)(R⁷)— | —C(O)— | —O— |
| 9 | —O— | carbocyclylene | —C(O)— | —O— |
| 10 | —O— | heterocyclylene | —C(O)— | —O— |
| 11 | —O— | —N(R⁵)— | —(CH₂)ₖ— | —O— |
| 12 | —O— | —C(O)— | —(CH₂)ₖ— | —O— |
| 13 | —O— | —C(R⁶)(R⁷)— | —(CH₂)ₖ— | —O— |
| 14 | —O— | carbocyclylene | —(CH₂)ₖ— | —O— |
| 15 | —O— | heterocyclylene | —(CH₂)ₖ— | —O— |
| 16 | —O— | —N(R⁵)— | —C(R⁶)(R⁷)— | —O— |
| 17 | —O— | —C(O)— | —C(R⁶)(R⁷)— | —O— |
| 18 | —O— | —(CH₂)ₖ— | —C(R⁶)(R⁷)— | —O— |
| 19 | —O— | —C(R⁶)(R⁷)— | —C(R⁶)(R⁷)— | —O— |
| 20 | —O— | carbocyclylene | —C(R⁶)(R⁷)— | —O— |
| 21 | —O— | heterocyclylene | —C(R⁶)(R⁷)— | —O— |
| 22 | —O— | —N(R⁵)— | carbocyclylene | —O— |
| 23 | —O— | —C(O)— | carbocyclylene | —O— |
| 24 | —O— | —(CH₂)ₖ— | carbocyclylene | —O— |
| 25 | —O— | —C(R⁶)(R⁷)— | carbocyclylene | —O— |
| 26 | —O— | carbocyclylene | carbocyclylene | —O— |
| 27 | —O— | heterocyclylene | carbocyclylene | —O— |
| 28 | —O— | —N(R⁵)— | heterocyclylene | —O— |
| 29 | —O— | —C(O)— | heterocyclylene | —O— |
| 30 | —O— | —(CH₂)ₖ— | heterocyclylene | —O— |
| 31 | —O— | —C(R⁶)(R⁷)— | heterocyclylene | —O— |
| 32 | —O— | carbocyclylene | heterocyclylene | —O— |
| 33 | —O— | heterocyclylene | heterocyclylene | —O— |
| 34 | —O— | —(CH₂)ₖ— | —O— | —N(R⁵)— |
| 35 | —O— | —C(R⁶)(R⁷)— | —O— | —N(R⁵)— |
| 36 | —O— | carbocyclylene | —O— | —N(R⁵)— |
| 37 | —O— | heterocyclylene | —O— | —N(R⁵)— |
| 38 | —O— | —C(O)— | —N(R⁵)— | —N(R⁵)— |
| 39 | —O— | —(CH₂)ₖ— | —N(R⁵)— | —N(R⁵)— |
| 40 | —O— | —C(R⁶)(R⁷)— | —N(R⁵)— | —N(R⁵)— |
| 41 | —O— | carbocyclylene | —N(R⁵)— | —N(R⁵)— |
| 42 | —O— | heterocyclylene | —N(R⁵)— | —N(R⁵)— |
| 43 | —O— | —N(R⁵)— | —C(O)— | —N(R⁵)— |
| 44 | —O— | —(CH₂)ₖ— | —C(O)— | —N(R⁵)— |
| 45 | —O— | —C(R⁶)(R⁷)— | —C(O)— | —N(R⁵)— |
| 46 | —O— | carbocyclylene | —C(O)— | —N(R⁵)— |
| 47 | —O— | heterocyclylene | —C(O)— | —N(R⁵)— |
| 48 | —O— | —N(R⁵)— | —S(O)ₗ— | —N(R⁵)— |
| 49 | —O— | —(CH₂)ₖ— | —S(O)ₗ— | —N(R⁵)— |
| 50 | —O— | —C(R⁶)(R⁷)— | —S(O)ₗ— | —N(R⁵)— |
| 51 | —O— | carbocyclylene | —S(O)ₗ— | —N(R⁵)— |
| 52 | —O— | heterocyclylene | —S(O)ₗ— | —N(R⁵)— |
| 53 | —O— | —N(R⁵)— | —(CH₂)ₖ— | —N(R⁵)— |
| 54 | —O— | —C(O)— | —(CH₂)ₖ— | —N(R⁵)— |
| 55 | —O— | —C(R⁶)(R⁷)— | —(CH₂)ₖ— | —N(R⁵)— |
| 56 | —O— | carbocyclylene | —(CH₂)ₖ— | —N(R⁵)— |
| 57 | —O— | heterocyclylene | —(CH₂)ₖ— | —N(R⁵)— |
| 58 | —O— | —N(R⁵)— | —C(R⁶)(R⁷)— | —N(R⁵)— |
| 59 | —O— | —C(O)— | —C(R⁶)(R⁷)— | —N(R⁵)— |
| 60 | —O— | —(CH₂)ₖ— | —C(R⁶)(R⁷)— | —N(R⁵)— |
| 61 | —O— | —C(R⁶)(R⁷)— | —C(R⁶)(R⁷)— | —N(R⁵)— |
| 62 | —O— | carbocyclylene | —C(R⁶)(R⁷)— | —N(R⁵)— |
| 63 | —O— | heterocyclylene | —C(R⁶)(R⁷)— | —N(R⁵)— |
| 64 | —O— | —N(R⁵)— | carbocyclylene | —N(R⁵)— |
| 65 | —O— | —C(O)— | carbocyclylene | —N(R⁵)— |
| 66 | —O— | —(CH₂)ₖ— | carbocyclylene | —N(R⁵)— |
| 67 | —O— | —C(R⁶)(R⁷)— | carbocyclylene | —N(R⁵)— |
| 68 | —O— | carbocyclylene | carbocyclylene | —N(R⁵)— |
| 69 | —O— | heterocyclylene | carbocyclylene | —N(R⁵)— |
| 70 | —O— | —N(R⁵)— | heterocyclylene | —N(R⁵)— |
| 71 | —O— | —C(O)— | heterocyclylene | —N(R⁵)— |
| 72 | —O— | —(CH₂)ₖ— | heterocyclylene | —N(R⁵)— |
| 73 | —O— | —C(R⁶)(R⁷)— | heterocyclylene | —N(R⁵)— |
| 74 | —O— | carbocyclylene | heterocyclylene | —N(R⁵)— |
| 75 | —O— | heterocyclylene | heterocyclylene | —N(R⁵)— |
| 76 | —O— | —(CH₂)ₖ— | —O— | —C(O)— |
| 77 | —O— | —C(R⁶)(R⁷)— | —O— | —C(O)— |
| 78 | —O— | carbocyclylene | —O— | —C(O)— |
| 79 | —O— | heterocyclylene | —O— | —C(O)— |
| 80 | —O— | —C(O)— | —N(R⁵)— | —C(O)— |
| 81 | —O— | —(CH₂)ₖ— | —N(R⁵)— | —C(O)— |
| 82 | —O— | —C(R⁶)(R⁷)— | —N(R⁵)— | —C(O)— |
| 83 | —O— | carbocyclylene | —N(R⁵)— | —C(O)— |
| 84 | —O— | heterocyclylene | —N(R⁵)— | —C(O)— |
| 85 | —O— | —N(R⁵)— | —(CH₂)ₖ— | —C(O)— |
| 86 | —O— | —C(O)— | —(CH₂)ₖ— | —C(O)— |
| 87 | —O— | —(CH₂)ₖ— | —(CH₂)ₖ— | —C(O)— |
| 88 | —O— | —C(R⁶)(R⁷)— | —(CH₂)ₖ— | —C(O)— |
| 89 | —O— | carbocyclylene | —(CH₂)ₖ— | —C(O)— |
| 90 | —O— | heterocyclylene | —(CH₂)ₖ— | —C(O)— |
| 91 | —O— | —N(R⁵)— | —C(R⁶)(R⁷)— | —C(O)— |
| 92 | —O— | —C(O)— | —C(R⁶)(R⁷)— | —C(O)— |
| 93 | —O— | —(CH₂)ₖ— | —C(R⁶)(R⁷)— | —C(O)— |
| 94 | —O— | —C(R⁶)(R⁷)— | —C(R⁶)(R⁷)— | —C(O)— |
| 95 | —O— | carbocyclylene | —C(R⁶)(R⁷)— | —C(O)— |
| 96 | —O— | heterocyclylene | —C(R⁶)(R⁷)— | —C(O)— |
| 97 | —O— | —N(R⁵)— | carbocyclylene | —C(O)— |
| 98 | —O— | —C(O)— | carbocyclylene | —C(O)— |
| 99 | —O— | —(CH₂)ₖ— | carbocyclylene | —C(O)— |
| 100 | —O— | —C(R⁶)(R⁷)— | carbocyclylene | —C(O)— |
| 101 | —O— | carbocyclylene | carbocyclylene | —C(O)— |
| 102 | —O— | heterocyclylene | carbocyclylene | —C(O)— |
| 103 | —O— | —N(R⁵)— | heterocyclylene | —C(O)— |
| 104 | —O— | —C(O)— | heterocyclylene | —C(O)— |
| 105 | —O— | —(CH₂)ₖ— | heterocyclylene | —C(O)— |
| 106 | —O— | —C(R⁶)(R⁷)— | heterocyclylene | —C(O)— |
| 107 | —O— | carbocyclylene | heterocyclylene | —C(O)— |
| 108 | —O— | heterocyclylene | heterocyclylene | —C(O)— |
| 109 | —O— | —(CH₂)ₖ— | —O— | —(CH₂)ₖ— |
| 110 | —O— | —C(R⁶)(R⁷)— | —O— | —(CH₂)ₖ— |
| 111 | —O— | carbocyclylene | —O— | —(CH₂)ₖ— |
| 112 | —O— | heterocyclylene | —O— | —(CH₂)ₖ— |
| 113 | —O— | —N(R⁵)— | —N(R⁵)— | —(CH₂)ₖ— |
| 114 | —O— | —C(O)— | —N(R⁵)— | —(CH₂)ₖ— |
| 115 | —O— | —(CH₂)ₖ— | —N(R⁵)— | —(CH₂)ₖ— |
| 116 | —O— | —C(R⁶)(R⁷)— | —N(R⁵)— | —(CH₂)ₖ— |
| 117 | —O— | carbocyclylene | —N(R⁵)— | —(CH₂)ₖ— |
| 118 | —O— | heterocyclylene | —N(R⁵)— | —(CH₂)ₖ— |
| 119 | —O— | —N(R⁵)— | —C(O)— | —(CH₂)ₖ— |
| 120 | —O— | —(CH₂)ₖ— | —C(O)— | —(CH₂)ₖ— |
| 121 | —O— | —C(R⁶)(R⁷)— | —C(O)— | —(CH₂)ₖ— |
| 122 | —O— | carbocyclylene | —C(O)— | —(CH₂)ₖ— |
| 123 | —O— | heterocyclylene | —C(O)— | —(CH₂)ₖ— |
| 124 | —O— | —(CH₂)ₖ— | —C(S)— | —(CH₂)ₖ— |
| 125 | —O— | —C(R⁶)(R⁷)— | —C(S)— | —(CH₂)ₖ— |
| 126 | —O— | carbocyclylene | —C(S)— | —(CH₂)ₖ— |
| 127 | —O— | heterocyclylene | —C(S)— | —(CH₂)ₖ— |
| 128 | —O— | —N(R⁵)— | —S(O)ₗ— | —(CH₂)ₖ— |
| 129 | —O— | —(CH₂)ₖ— | —S(O)ₗ— | —(CH₂)ₖ— |
| 130 | —O— | —C(R⁶)(R⁷)— | —S(O)ₗ— | —(CH₂)ₖ— |
| 131 | —O— | carbocyclylene | —S(O)ₗ— | —(CH₂)ₖ— |
| 132 | —O— | heterocyclylene | —S(O)ₗ— | —(CH₂)ₖ— |
| 133 | —O— | —N(R⁵)— | —C(R⁶)(R⁷)— | —(CH₂)ₖ— |
| 134 | —O— | —C(O)— | —C(R⁶)(R⁷)— | —(CH₂)ₖ— |
| 135 | —O— | —(CH₂)ₖ— | —C(R⁶)(R⁷)— | —(CH₂)ₖ— |
| 136 | —O— | —C(R⁶)(R⁷)— | —C(R⁶)(R⁷)— | —(CH₂)ₖ— |
| 137 | —O— | carbocyclylene | —C(R⁶)(R⁷)— | —(CH₂)ₖ— |
| 138 | —O— | heterocyclylene | —C(R⁶)(R⁷)— | —(CH₂)ₖ— |
| 139 | —O— | —N(R⁵)— | carbocyclylene | —(CH₂)ₖ— |
| 140 | —O— | —C(O)— | carbocyclylene | —(CH₂)ₖ— |
| 141 | —O— | —(CH₂)ₖ— | carbocyclylene | —(CH₂)ₖ— |
| 142 | —O— | —C(R⁶)(R⁷)— | carbocyclylene | —(CH₂)ₖ— |
| 143 | —O— | carbocyclylene | carbocyclylene | —(CH₂)ₖ— |
| 144 | —O— | heterocyclylene | carbocyclylene | —(CH₂)ₖ— |
| 145 | —O— | —N(R⁵)— | heterocyclylene | —(CH₂)ₖ— |
| 146 | —O— | —C(O)— | heterocyclylene | —(CH₂)ₖ— |
| 147 | —O— | —(CH₂)ₖ— | heterocyclylene | —(CH₂)ₖ— |
| 148 | —O— | —C(R⁶)(R⁷)— | heterocyclylene | —(CH₂)ₖ— |
| 149 | —O— | carbocyclylene | heterocyclylene | —(CH₂)ₖ— |
| 150 | —O— | heterocyclylene | heterocyclylene | —(CH₂)ₖ— |
| 151 | —O— | —(CH₂)ₖ— | —O— | —C(R⁶)(R⁷)— |
| 152 | —O— | —C(R⁶)(R⁷)— | —O— | —C(R⁶)(R⁷)— |
| 153 | —O— | carbocyclylene | —O— | —C(R⁶)(R⁷)— |
| 154 | —O— | heterocyclylene | —O— | —C(R⁶)(R⁷)— |
| 155 | —O— | —C(O)— | —N(R⁵)— | —C(R⁶)(R⁷)— |
| 156 | —O— | —(CH₂)ₖ— | —N(R⁵)— | —C(R⁶)(R⁷)— |
| 157 | —O— | —C(R⁶)(R⁷)— | —N(R⁵)— | —C(R⁶)(R⁷)— |
| 158 | —O— | carbocyclylene | —N(R⁵)— | —C(R⁶)(R⁷)— |
| 159 | —O— | heterocyclylene | —N(R⁵)— | —C(R⁶)(R⁷)— |
| 160 | —O— | —N(R⁵)— | —C(O)— | —C(R⁶)(R⁷)— |
| 161 | —O— | —(CH₂)ₖ— | —C(O)— | —C(R⁶)(R⁷)— |
| 162 | —O— | —C(R⁶)(R⁷)— | —C(O)— | —C(R⁶)(R⁷)— |
| 163 | —O— | carbocyclylene | —C(O)— | —C(R⁶)(R⁷)— |
| 164 | —O— | heterocyclylene | —C(O)— | —C(R⁶)(R⁷)— |
| 165 | —O— | —(CH₂)ₖ— | —C(S)— | —C(R⁶)(R⁷)— |
| 166 | —O— | —C(R⁶)(R⁷)— | —C(S)— | —C(R⁶)(R⁷)— |
| 167 | —O— | carbocyclylene | —C(S)— | —C(R⁶)(R⁷)— |
| 168 | —O— | heterocyclylene | —C(S)— | —C(R⁶)(R⁷)— |
| 169 | —O— | —N(R⁵)— | —S(O)ₗ— | —C(R⁶)(R⁷)— |
| 170 | —O— | —(CH₂)ₖ— | —S(O)ₗ— | —C(R⁶)(R⁷)— |
| 171 | —O— | —C(R⁶)(R⁷)— | —S(O)ₗ— | —C(R⁶)(R⁷)— |
| 172 | —O— | carbocyclylene | —S(O)ₗ— | —C(R⁶)(R⁷)— |
| 173 | —O— | heterocyclylene | —S(O)ₗ— | —C(R⁶)(R⁷)— |
| 174 | —O— | —N(R⁵)— | —(CH₂)ₖ— | —C(R⁶)(R⁷)— |
| 175 | —O— | —C(O)— | —(CH₂)ₖ— | —C(R⁶)(R⁷)— |
| 176 | —O— | —(CH₂)ₖ— | —(CH₂)ₖ— | —C(R⁶)(R⁷)— |
| 177 | —O— | —C(R⁶)(R⁷)— | —(CH₂)ₖ— | —C(R⁶)(R⁷)— |
| 178 | —O— | carbocyclylene | —(CH₂)ₖ— | —C(R⁶)(R⁷)— |
| 179 | —O— | heterocyclylene | —(CH₂)ₖ— | —C(R⁶)(R⁷)— |
| 180 | —O— | —N(R⁵)— | —C(R⁶)(R⁷)— | —C(R⁶)(R⁷)— |
| 181 | —O— | —C(O)— | —C(R⁶)(R⁷)— | —C(R⁶)(R⁷)— |
| 182 | —O— | —(CH₂)ₖ— | —C(R⁶)(R⁷)— | —C(R⁶)(R⁷)— |
| 183 | —O— | —C(R⁶)(R⁷)— | —C(R⁶)(R⁷)— | —C(R⁶)(R⁷)— |
| 184 | —O— | carbocyclylene | —C(R⁶)(R⁷)— | —C(R⁶)(R⁷)— |
| 185 | —O— | heterocyclylene | —C(R⁶)(R⁷)— | —C(R⁶)(R⁷)— |
| 186 | —O— | —N(R⁵)— | carbocyclylene | —C(R⁶)(R⁷)— |
| 187 | —O— | —C(O)— | carbocyclylene | —C(R⁶)(R⁷)— |
| 188 | —O— | —(CH₂)ₖ— | carbocyclylene | —C(R⁶)(R⁷)— |
| 189 | —O— | —C(R⁶)(R⁷)— | carbocyclylene | —C(R⁶)(R⁷)— |
| 190 | —O— | carbocyclylene | carbocyclylene | —C(R⁶)(R⁷)— |
| 191 | —O— | heterocyclylene | carbocyclylene | —C(R⁶)(R⁷)— |
| 192 | —O— | —N(R⁵)— | heterocyclylene | —C(R⁶)(R⁷)— |
| 193 | —O— | —C(O)— | heterocyclylene | —C(R⁶)(R⁷)— |
| 194 | —O— | —(CH₂)ₖ— | heterocyclylene | —C(R⁶)(R⁷)— |
| 195 | —O— | —C(R⁶)(R⁷)— | heterocyclylene | —C(R⁶)(R⁷)— |
| 196 | —O— | carbocyclylene | heterocyclylene | —C(R⁶)(R⁷)— |
| 197 | —O— | heterocyclylene | heterocyclylene | —C(R⁶)(R⁷)— |
| 198 | —O— | —N(R⁵)— | —(CH₂)ₖ— | —C(S)— |
| 199 | —O— | —C(O)— | —(CH₂)ₖ— | —C(S)— |
| 200 | —O— | —(CH₂)ₖ— | —(CH₂)ₖ— | —C(S)— |
| 201 | —O— | —C(R⁶)(R⁷)— | —(CH₂)ₖ— | —C(S)— |
| 202 | —O— | carbocyclylene | —(CH₂)ₖ— | —C(S)— |
| 203 | —O— | heterocyclylene | —(CH₂)ₖ— | —C(S)— |
| 204 | —O— | —N(R⁵)— | —C(R⁶)(R⁷)— | —C(S)— |
| 205 | —O— | —C(O)— | —C(R⁶)(R⁷)— | —C(S)— |
| 206 | —O— | —(CH₂)ₖ— | —C(R⁶)(R⁷)— | —C(S)— |
| 207 | —O— | —C(R⁶)(R⁷)— | —C(R⁶)(R⁷)— | —C(S)— |
| 208 | —O— | carbocyclylene | —C(R⁶)(R⁷)— | —C(S)— |
| 209 | —O— | heterocyclylene | —C(R⁶)(R⁷)— | —C(S)— |
| 210 | —O— | —N(R⁵)— | carbocyclylene | —C(S)— |
| 211 | —O— | —C(O)— | carbocyclylene | —C(S)— |
| 212 | —O— | —(CH₂)ₖ— | carbocyclylene | —C(S)— |
| 213 | —O— | —C(R⁶)(R⁷)— | carbocyclylene | —C(S)— |
| 214 | —O— | carbocyclylene | carbocyclylene | —C(S)— |
| 215 | —O— | heterocyclylene | carbocyclylene | —C(S)— |
| 216 | —O— | —O— | heterocyclylene | —C(S)— |
| 217 | —O— | —N(R⁵)— | heterocyclylene | —C(S)— |
| 218 | —O— | —C(O)— | heterocyclylene | —C(S)— |
| 219 | —O— | —(CH₂)ₖ— | heterocyclylene | —C(S)— |
| 220 | —O— | —C(R⁶)(R⁷)— | heterocyclylene | —C(S)— |
| 221 | —O— | carbocyclylene | heterocyclylene | —C(S)— |
| 222 | —O— | heterocyclylene | heterocyclylene | —C(S)— |
| 223 | —O— | —C(O)— | —N(R⁵)— | —S(O)ₗ— |
| 224 | —O— | —(CH₂)ₖ— | —N(R⁵)— | —S(O)ₗ— |
| 225 | —O— | —C(R⁶)(R⁷)— | —N(R⁵)— | —S(O)ₗ— |
| 226 | —O— | carbocyclylene | —N(R⁵)— | —S(O)ₗ— |
| 227 | —O— | heterocyclylene | —N(R⁵)— | —S(O)ₗ— |
| 228 | —O— | —N(R⁵)— | —(CH₂)ₖ— | —S(O)ₗ— |
| 229 | —O— | —C(O)— | —(CH₂)ₖ— | —S(O)ₗ— |
| 230 | —O— | —(CH₂)ₖ— | —(CH₂)ₖ— | —S(O)ₗ— |
| 231 | —O— | —C(R⁶)(R⁷)— | —(CH₂)ₖ— | —S(O)ₗ— |
| 232 | —O— | carbocyclylene | —(CH₂)ₖ— | —S(O)ₗ— |
| 233 | —O— | heterocyclylene | —(CH₂)ₖ— | —S(O)ₗ— |
| 234 | —O— | —N(R⁵)— | —C(R⁶)(R⁷)— | —S(O)ₗ— |
| 235 | —O— | —C(O)— | —C(R⁶)(R⁷)— | —S(O)ₗ— |
| 236 | —O— | —(CH₂)ₖ— | —C(R⁶)(R⁷)— | —S(O)ₗ— |
| 237 | —O— | —C(R⁶)(R⁷)— | —C(R⁶)(R⁷)— | —S(O)ₗ— |
| 238 | —O— | carbocyclylene | —C(R⁶)(R⁷)— | —S(O)ₗ— |
| 239 | —O— | heterocyclylene | —C(R⁶)(R⁷)— | —S(O)ₗ— |
| 240 | —O— | —O— | carbocyclylene | —S(O)ₗ— |
| 241 | —O— | —N(R⁵)— | carbocyclylene | —S(O)ₗ— |
| 242 | —O— | —C(O)— | carbocyclylene | —S(O)ₗ— |
| 243 | —O— | —(CH₂)ₖ— | carbocyclylene | —S(O)ₗ— |
| 244 | —O— | —C(R⁶)(R⁷)— | carbocyclylene | —S(O)ₗ— |
| 245 | —O— | carbocyclylene | carbocyclylene | —S(O)ₗ— |
| 246 | —O— | heterocyclylene | carbocyclylene | —S(O)ₗ— |
| 247 | —O— | —N(R⁵)— | heterocyclylene | —S(O)ₗ— |
| 248 | —O— | —C(O)— | heterocyclylene | —S(O)ₗ— |
| 249 | —O— | —(CH₂)ₖ— | heterocyclylene | —S(O)ₗ— |
| 250 | —O— | —C(R⁶)(R⁷)— | heterocyclylene | —S(O)ₗ— |
| 251 | —O— | carbocyclylene | heterocyclylene | —S(O)ₗ— |
| 252 | —O— | heterocyclylene | heterocyclylene | —S(O)ₗ— |
| 253 | —O— | —(CH₂)ₖ— | —O— | carbocyclylene |
| 254 | —O— | —C(R⁶)(R⁷)— | —O— | carbocyclylene |
| 255 | —O— | carbocyclylene | —O— | carbocyclylene |
| 256 | —O— | heterocyclylene | —O— | carbocyclylene |
| 257 | —O— | —N(R⁵)— | —N(R⁵)— | carbocyclylene |
| 258 | —O— | —C(O)— | —N(R⁵)— | carbocyclylene |
| 259 | —O— | —(CH₂)ₖ— | —N(R⁵)— | carbocyclylene |
| 260 | —O— | —C(R⁶)(R⁷)— | —N(R⁵)— | carbocyclylene |
| 261 | —O— | carbocyclylene | —N(R⁵)— | carbocyclylene |
| 262 | —O— | heterocyclylene | —N(R⁵)— | carbocyclylene |
| 263 | —O— | —N(R⁵)— | —C(O)— | carbocyclylene |
| 264 | —O— | —(CH₂)ₖ— | —C(O)— | carbocyclylene |
| 265 | —O— | —C(R⁶)(R⁷)— | —C(O)— | carbocyclylene |
| 266 | —O— | carbocyclylene | —C(O)— | carbocyclylene |
| 267 | —O— | heterocyclylene | —C(O)— | carbocyclylene |
| 268 | —O— | —(CH₂)ₖ— | —C(S)— | carbocyclylene |
| 269 | —O— | —C(R⁶)(R⁷)— | —C(S)— | carbocyclylene |
| 270 | —O— | carbocyclylene | —C(S)— | carbocyclylene |
| 271 | —O— | heterocyclylene | —C(S)— | carbocyclylene |
| 272 | —O— | —N(R⁵)— | —S(O)ₗ— | carbocyclylene |
| 273 | —O— | —(CH₂)ₖ— | —S(O)ₗ— | carbocyclylene |
| 274 | —O— | —C(R⁶)(R⁷)— | —S(O)ₗ— | carbocyclylene |
| 275 | —O— | carbocyclylene | —S(O)ₗ— | carbocyclylene |
| 276 | —O— | heterocyclylene | —S(O)ₗ— | carbocyclylene |
| 277 | —O— | —N(R⁵)— | —(CH₂)ₖ— | carbocyclylene |
| 278 | —O— | —C(O)— | —(CH₂)ₖ— | carbocyclylene |
| 279 | —O— | —C(R⁶)(R⁷)— | —(CH₂)ₖ— | carbocyclylene |
| 280 | —O— | carbocyclylene | —(CH₂)ₖ— | carbocyclylene |
| 281 | —O— | heterocyclylene | —(CH₂)ₖ— | carbocyclylene |
| 282 | —O— | —N(R⁵)— | —C(R⁶)(R⁷)— | carbocyclylene |
| 283 | —O— | —C(O)— | —C(R⁶)(R⁷)— | carbocyclylene |
| 284 | —O— | —(CH₂)ₖ— | —C(R⁶)(R⁷)— | carbocyclylene |
| 285 | —O— | —C(R⁶)(R⁷)— | —C(R⁶)(R⁷)— | carbocyclylene |
| 286 | —O— | carbocyclylene | —C(R⁶)(R⁷)— | carbocyclylene |
| 287 | —O— | heterocyclylene | —C(R⁶)(R⁷)— | carbocyclylene |
| 288 | —O— | —N(R⁵)— | carbocyclylene | carbocyclylene |
| 289 | —O— | —C(O)— | carbocyclylene | carbocyclylene |
| 290 | —O— | —(CH₂)ₖ— | carbocyclylene | carbocyclylene |
| 291 | —O— | —C(R⁶)(R⁷)— | carbocyclylene | carbocyclylene |
| 292 | —O— | —N(R⁵)— | heterocyclylene | carbocyclylene |
| 293 | —O— | —C(O)— | heterocyclylene | carbocyclylene |
| 294 | —O— | —(CH₂)ₖ— | heterocyclylene | carbocyclylene |
| 295 | —O— | —C(R⁶)(R⁷)— | heterocyclylene | carbocyclylene |
| 296 | —O— | —(CH₂)ₖ— | —O— | heterocyclylene |
| 297 | —O— | —C(R⁶)(R⁷)— | —O— | heterocyclylene |
| 298 | —O— | carbocyclylene | —O— | heterocyclylene |
| 299 | —O— | heterocyclylene | —O— | heterocyclylene |
| 300 | —O— | —C(O)— | —N(R⁵)— | heterocyclylene |
| 301 | —O— | —(CH₂)ₖ— | —N(R⁵)— | heterocyclylene |
| 302 | —O— | —C(R⁶)(R⁷)— | —N(R⁵)— | heterocyclylene |
| 303 | —O— | carbocyclylene | —N(R⁵)— | heterocyclylene |
| 304 | —O— | heterocyclylene | —N(R⁵)— | heterocyclylene |
| 305 | —O— | —N(R⁵)— | —C(O)— | heterocyclylene |
| 306 | —O— | —(CH₂)ₖ— | —C(O)— | heterocyclylene |
| 307 | —O— | —C(R⁶)(R⁷)— | —C(O)— | heterocyclylene |
| 308 | —O— | carbocyclylene | —C(O)— | heterocyclylene |
| 309 | —O— | heterocyclylene | —C(O)— | heterocyclylene |
| 310 | —O— | —(CH₂)ₖ— | —C(S)— | heterocyclylene |
| 311 | —O— | —C(R⁶)(R⁷)— | —C(S)— | heterocyclylene |
| 312 | —O— | carbocyclylene | —C(S)— | heterocyclylene |
| 313 | —O— | heterocyclylene | —C(S)— | heterocyclylene |
| 314 | —O— | —N(R⁵)— | —S(O)ₗ— | heterocyclylene |
| 315 | —O— | —C(O)— | —S(O)ₗ— | heterocyclylene |
| 316 | —O— | —C(R⁶)(R⁷)— | —S(O)ₗ— | heterocyclylene |
| 317 | —O— | carbocyclylene | —S(O)ₗ— | heterocyclylene |
| 318 | —O— | heterocyclylene | —S(O)ₗ— | heterocyclylene |
| 319 | —O— | —N(R⁵)— | —(CH₂)ₖ— | heterocyclylene |
| 320 | —O— | —C(O)— | —(CH₂)ₖ— | heterocyclylene |
| 321 | —O— | —(CH₂)ₖ— | —(CH₂)ₖ— | heterocyclylene |
| 322 | —O— | —C(R⁶)(R⁷)— | —(CH₂)ₖ— | heterocyclylene |
| 323 | —O— | carbocyclylene | —(CH₂)ₖ— | heterocyclylene |
| 324 | —O— | heterocyclylene | —(CH₂)ₖ— | heterocyclylene |
| 325 | —O— | —N(R⁵)— | —C(R⁶)(R⁷)— | heterocyclylene |
| 326 | —O— | —C(O)— | —C(R⁶)(R⁷)— | heterocyclylene |
| 327 | —O— | —(CH₂)ₖ— | —C(R⁶)(R⁷)— | heterocyclylene |
| 328 | —O— | —C(R⁶)(R⁷)— | —C(R⁶)(R⁷)— | heterocyclylene |
| 329 | —O— | carbocyclylene | —C(R⁶)(R⁷)— | heterocyclylene |
| 330 | —O— | heterocyclylene | —C(R⁶)(R⁷)— | heterocyclylene |
| 331 | —O— | —N(R⁵)— | carbocyclylene | heterocyclylene |
| 332 | —O— | —C(O)— | carbocyclylene | heterocyclylene |
| 333 | —O— | —(CH₂)ₖ— | carbocyclylene | heterocyclylene |
| 334 | —O— | —C(R⁶)(R⁷)— | carbocyclylene | heterocyclylene |
| 335 | —O— | —N(R⁵)— | heterocyclylene | heterocyclylene |
| 336 | —O— | —C(O)— | heterocyclylene | heterocyclylene |
| 337 | —O— | —(CH₂)ₖ— | heterocyclylene | heterocyclylene |
| 338 | —O— | —C(R⁶)(R⁷)— | heterocyclylene | heterocyclylene |
| 339 | carbocyclylene | —O— | —(CH₂)ₖ— | —O— |
| 340 | carbocyclylene | —O— | —C(R⁶)(R⁷)— | —O— |
| 341 | carbocyclylene | —O— | carbocyclylene | —O— |
| 342 | carbocyclylene | —O— | heterocyclylene | —O— |
| 343 | carbocyclylene | —O— | —C(O)— | —N(R⁵)— |
| 344 | carbocyclylene | —O— | —(CH₂)ₖ— | —N(R⁵)— |
| 345 | carbocyclylene | —O— | —C(R⁶)(R⁷)— | —N(R⁵)— |
| 346 | carbocyclylene | —O— | carbocyclylene | —N(R⁵)— |
| 347 | carbocyclylene | —O— | heterocyclylene | —N(R⁵)— |
| 348 | carbocyclylene | —O— | —N(R⁵)— | —C(O)— |
| 349 | carbocyclylene | —O— | —(CH₂)ₖ— | —C(O)— |
| 350 | carbocyclylene | —O— | —C(R⁶)(R⁷)— | —C(O)— |
| 351 | carbocyclylene | —O— | carbocyclylene | —C(O)— |
| 352 | carbocyclylene | —O— | heterocyclylene | —C(O)— |
| 353 | carbocyclylene | —O— | —(CH₂)ₖ— | —C(S)— |
| 354 | carbocyclylene | —O— | —C(R⁶)(R⁷)— | —C(S)— |
| 355 | carbocyclylene | —O— | carbocyclylene | —C(S)— |
| 356 | carbocyclylene | —O— | heterocyclylene | —C(S)— |
| 357 | carbocyclylene | —O— | —N(R⁵)— | —S(O)ₗ— |
| 358 | carbocyclylene | —O— | —(CH₂)ₖ— | —S(O)ₗ— |
| 359 | carbocyclylene | —O— | —C(R⁶)(R⁷)— | —S(O)ₗ— |
| 360 | carbocyclylene | —O— | carbocyclylene | —S(O)ₗ— |
| 361 | carbocyclylene | —O— | heterocyclylene | —S(O)ₗ— |
| 362 | carbocyclylene | —O— | —N(R⁵)— | —(CH₂)ₖ— |
| 363 | carbocyclylene | —O— | —C(O)— | —(CH₂)ₖ— |
| 364 | carbocyclylene | —O— | —C(R⁶)(R⁷)— | —(CH₂)ₖ— |
| 365 | carbocyclylene | —O— | carbocyclylene | —(CH₂)ₖ— |
| 366 | carbocyclylene | —O— | heterocyclylene | —(CH₂)ₖ— |
| 367 | carbocyclylene | —O— | —N(R⁵)— | —C(R⁶)(R⁷)— |
| 368 | carbocyclylene | —O— | —C(O)— | —C(R⁶)(R⁷)— |
| 369 | carbocyclylene | —O— | —(CH₂)ₖ— | —C(R⁶)(R⁷)— |
| 370 | carbocyclylene | —O— | —C(R⁶)(R⁷)— | —C(R⁶)(R⁷)— |
| 371 | carbocyclylene | —O— | carbocyclylene | —C(R⁶)(R⁷)— |
| 372 | carbocyclylene | —O— | heterocyclylene | —C(R⁶)(R⁷)— |
| 373 | carbocyclylene | —O— | —N(R⁵)— | carbocyclylene |
| 374 | carbocyclylene | —O— | —C(O)— | carbocyclylene |
| 375 | carbocyclylene | —O— | —(CH₂)ₖ— | carbocyclylene |
| 376 | carbocyclylene | —O— | —C(R⁶)(R⁷)— | carbocyclylene |
| 377 | carbocyclylene | —O— | carbocyclylene | carbocyclylene |
| 378 | carbocyclylene | —O— | heterocyclylene | carbocyclylene |
| 379 | carbocyclylene | —O— | —N(R⁵)— | heterocyclylene |
| 380 | carbocyclylene | —O— | —C(O)— | heterocyclylene |
| 381 | carbocyclylene | —O— | —(CH₂)ₖ— | heterocyclylene |
| 382 | carbocyclylene | —O— | —C(R⁶)(R⁷)— | heterocyclylene |
| 383 | carbocyclylene | —O— | carbocyclylene | heterocyclylene |
| 384 | carbocyclylene | —O— | heterocyclylene | heterocyclylene |

Particular examples of -Y¹-Y²-Y³-Y⁴- are given in Table 13:

TABLE 13

| No. | Y¹ | Y² | Y³ | Y⁴ |
|---|---|---|---|---|
| 1 | —O— | —(CH₂)ₖ— | —C(O)— | —O— |
| 2 | —O— | —C(R⁶)(R⁷)— | —C(O)— | —O— |
| 3 | —O— | —(CH₂)ₖ— | —C(O)— | —N(R⁵)— |
| 4 | —O— | —C(R⁶)(R⁷)— | —C(O)— | —N(R⁵)— |
| 5 | —O— | —C(H₂)ₖ— | —O— | —C(O)— |
| 6 | —O— | —C(R⁶)(R⁷)— | —O— | —C(O)— |
| 7 | carbocyclylene | —O— | —C(H₂)ₖ— | —C(O)— |
| 8 | carbocyclylene | —O— | —C(R⁶)(R⁷)— | —C(O)— |
| 9 | carbocyclylene | —O— | —C(H₂)ₖ— | carbocyclylene |
| 10 | carbocyclylene | —O— | —C(R⁶)(R⁷)— | carbocyclylene |

Further particular examples of -Y¹-Y²-Y³-Y⁴- are given in Table 14:

TABLE 14

| No. | Y¹ | Y² | Y³ | Y⁴ |
|---|---|---|---|---|
| 1 | —O— | —CH₂— | —C(O)— | —O— |
| 2 | —O— | —C(R⁶)(R⁷)— | —C(O)— | —O— |
| 3 | —O— | —CH₂— | —C(O)— | —N(R⁵)— |
| 4 | —O— | —C(R⁶)(R⁷)— | —C(O)— | —N(R⁵)— |
| 5 | —O— | —C(H₂)₂— | —O— | —C(O)— |
| 6 | phenylene | —O— | —C(H₂)ₖ— | —C(O)— |
| 7 | phenylene | —O— | —C(R⁶)(R⁷)— | —C(O)— |
| 8 | phenylene | —O— | —C(H₂)ₖ— | phenylene |
| 9 | phenylene | —O— | —C(R⁶)(R⁷)— | phenylene |

Examples of the linker -Y¹-Y²-Y³-Y⁴-Y⁵- are given in Table 15:

TABLE 15

| No. | Y¹ | Y² | Y³ | Y⁴ | Y⁵ |
|---|---|---|---|---|---|
| 1 | —O— | —(CH₂)ₖ— | —C(O)— | —O— | —(CH₂)ₖ— |
| 2 | —O— | —(CH₂)ₖ— | —C(R⁶)(R⁷)— | —O— | —(CH₂)ₖ— |
| 3 | —O— | —(CH₂)ₖ— | carbocyclylene | —O— | —(CH₂)ₖ— |
| 4 | —O— | —(CH₂)ₖ— | heterocyclylene | —O— | —(CH₂)ₖ— |
| 5 | —O— | —C(R⁶)(R⁷)— | —C(O)— | —O— | —(CH₂)ₖ— |
| 6 | —O— | —C(R⁶)(R⁷)— | —(CH₂)ₖ— | —O— | —(CH₂)ₖ— |
| 7 | —O— | —C(R⁶)(R⁷)— | —C(R⁶)(R⁷)— | —O— | —(CH₂)ₖ— |
| 8 | —O— | —C(R⁶)(R⁷)— | carbocyclylene | —O— | —(CH₂)ₖ— |
| 9 | —O— | —C(R⁶)(R⁷)— | heterocyclylene | —O— | —(CH₂)ₖ— |
| 10 | —O— | —(CH₂)ₖ— | —C(O)— | —N(R⁵)— | —(CH₂)ₖ— |
| 11 | —O— | —(CH₂)ₖ— | —C(R⁶)(R⁷)— | —N(R⁵)— | —(CH₂)ₖ— |
| 12 | —O— | —(CH₂)ₖ— | carbocyclylene | —N(R⁵)— | —(CH₂)ₖ— |
| 13 | —O— | —(CH₂)ₖ— | heterocyclylene | —N(R⁵)— | —(CH₂)ₖ— |
| 14 | —O— | —C(R⁶)(R⁷)— | —C(O)— | —N(R⁵)— | —(CH₂)ₖ— |
| 15 | —O— | —C(R⁶)(R⁷)— | —(CH₂)ₖ— | —N(R⁵)— | —(CH₂)ₖ— |
| 16 | —O— | —C(R⁶)(R⁷)— | —C(R⁶)(R⁷)— | —N(R⁵)— | —(CH₂)ₖ— |
| 17 | —O— | —C(R⁶)(R⁷)— | carbocyclylene | —N(R⁵)— | —(CH₂)ₖ— |
| 18 | —O— | —C(R⁶)(R⁷)— | heterocyclylene | —N(R⁵)— | —(CH₂)ₖ— |
| 19 | —O— | —(CH₂)ₖ— | —C(O)— | —C(O)— | —(CH₂)ₖ— |
| 20 | —O— | —(CH₂)ₖ— | —C(R⁶)(R⁷)— | —C(O)— | —(CH₂)ₖ— |
| 21 | —O— | —(CH₂)ₖ— | carbocyclylene | —C(O)— | —(CH₂)ₖ— |
| 22 | —O— | —(CH₂)ₖ— | heterocyclylene | —C(O)— | —(CH₂)ₖ— |
| 23 | —O— | —C(R⁶)(R⁷)— | —C(O)— | —C(O)— | —(CH₂)ₖ— |
| 24 | —O— | —C(R⁶)(R⁷)— | —(CH₂)ₖ— | —C(O)— | —(CH₂)ₖ— |
| 25 | —O— | —C(R⁶)(R⁷)— | —C(R⁶)(R⁷)— | —C(O)— | —(CH₂)ₖ— |
| 26 | —O— | —C(R⁶)(R⁷)— | carbocyclylene | —C(O)— | —(CH₂)ₖ— |
| 27 | —O— | —C(R⁶)(R⁷)— | heterocyclylene | —C(O)— | —(CH₂)ₖ— |
| 28 | —O— | —(CH₂)ₖ— | —C(O)— | —O— | —(CH₂)ₖ— |
| 29 | —O— | —(CH₂)ₖ— | —C(R⁶)(R⁷)— | —O— | —(CH₂)ₖ— |
| 30 | —O— | —(CH₂)ₖ— | carbocyclylene | —O— | —(CH₂)ₖ— |
| 31 | —O— | —(CH₂)ₖ— | heterocyclylene | —O— | —(CH₂)ₖ— |
| 32 | —O— | —C(R⁶)(R⁷)— | —C(O)— | —O— | —(CH₂)ₖ— |
| 33 | —O— | —C(R⁶)(R⁷)— | —(CH₂)ₖ— | —O— | —(CH₂)ₖ— |
| 34 | —O— | —C(R⁶)(R⁷)— | —C(R⁶)(R⁷)— | —O— | —(CH₂)ₖ— |
| 35 | —O— | —C(R⁶)(R⁷)— | carbocyclylene | —O— | —(CH₂)ₖ— |
| 36 | —O— | —C(R⁶)(R⁷)— | heterocyclylene | —O— | —(CH₂)ₖ— |
| 37 | —O— | —(CH₂)ₖ— | —C(O)— | —N(R⁵)— | —S(O)ₗ— |
| 38 | —O— | —(CH₂)ₖ— | —C(R⁶)(R⁷)— | —N(R⁵)— | —S(O)ₗ— |
| 39 | —O— | —(CH₂)ₖ— | carbocyclylene | —N(R⁵)— | —S(O)ₗ— |
| 40 | —O— | —(CH₂)ₖ— | heterocyclylene | —N(R⁵)— | —S(O)ₗ— |
| 41 | —O— | —C(R⁶)(R⁷)— | —C(O)— | —N(R⁵)— | —S(O)ₗ— |
| 42 | —O— | —C(R⁶)(R⁷)— | —(CH₂)ₖ— | —N(R⁵)— | —S(O)ₗ— |
| 43 | —O— | —C(R⁶)(R⁷)— | —C(R⁶)(R⁷)— | —N(R⁵)— | —S(O)ₗ— |
| 44 | —O— | —C(R⁶)(R⁷)— | carbocyclylene | —N(R⁵)— | —S(O)ₗ— |
| 45 | —O— | —C(R⁶)(R⁷)— | heterocyclylene | —N(R⁵)— | —S(O)ₗ— |
| 46 | —O— | —(CH₂)k | —O— | —C(O)— | —O— |
| 47 | —O— | —(CH₂)k | —O— | —C(O)— | —N(R⁵)— |
| 48 | —O— | —(CH₂)ₖ— | —C(R⁶)(R⁷)— | —C(O)— | —(CH₂)ₖ— |
| 49 | —O— | —(CH₂)ₖ— | carbocyclylene | —C(O)— | —(CH₂)ₖ— |
| 50 | —O— | —(CH₂)ₖ— | heterocyclylene | —C(O)— | —(CH₂)ₖ— |
| 51 | —O— | —C(R⁶)(R⁷)— | —C(O)— | —C(O)— | —(CH₂)ₖ— |
| 52 | —O— | —C(R⁶)(R⁷)— | —(CH₂)ₖ— | —C(O)— | —(CH₂)ₖ— |
| 53 | —O— | —C(R⁶)(R⁷)— | —C(R⁶)(R⁷)— | —C(O)— | —(CH₂)ₖ— |
| 54 | —O— | —C(R⁶)(R⁷)— | carbocyclylene | —C(O)— | —(CH₂)ₖ— |
| 55 | —O— | —C(R⁶)(R⁷)— | heterocyclylene | —C(O)— | —(CH₂)ₖ— |
| 56 | —O— | —(CH₂)ₖ— | —C(O)— | —(CH₂)ₖ— | —S(O)ₗ— |
| 57 | —O— | —(CH₂)ₖ— | —C(R⁶)(R⁷)— | —(CH₂)ₖ— | —S(O)ₗ— |
| 58 | —O— | —(CH₂)ₖ— | carbocyclylene | —(CH₂)ₖ— | —S(O)ₗ— |
| 59 | —O— | —(CH₂)ₖ— | heterocyclylene | —(CH₂)ₖ— | —S(O)ₗ— |
| 60 | —O— | —C(R⁶)(R⁷)— | —C(O)— | —(CH₂)ₖ— | —S(O)ₗ— |
| 61 | —O— | —C(R⁶)(R⁷)— | —(CH₂)ₖ— | —(CH₂)ₖ— | —S(O)ₗ— |
| 62 | —O— | —C(R⁶)(R⁷)— | —C(R⁶)(R⁷)— | —(CH₂)ₖ— | —S(O)ₗ— |
| 63 | —O— | —C(R⁶)(R⁷)— | carbocyclylene | —(CH₂)ₖ— | —S(O)ₗ— |
| 64 | —O— | —C(R⁶)(R⁷)— | heterocyclylene | —(CH₂)ₖ— | —S(O)ₗ— |
| 65 | carbocyclylene | —O— | —(CH₂)ₖ— | —C(O)— | —O— |
| 66 | carbocyclylene | —O— | —C(R⁶)(R⁷)— | —C(O)— | —N(R⁵)— |

Particular examples of -Y¹-Y²-Y³-Y⁴-Y⁵- are given in Table 16:

TABLE 16

| No. | Y¹ | Y² | Y³ | Y⁴ | Y⁵ |
|---|---|---|---|---|---|
| 1 | —O— | —(CH₂)₂— | —O— | —C(O)— | —O— |
| 2 | —O— | —(CH₂)₂— | —O— | —C(O)— | —N(R⁵)— |
| 3 | —O— | —C(R⁶)(R⁷)— | —CH₂— | —O— | —C(O)— |
| 4 | —O— | —C(R⁶)(R⁷)— | —CH₂— | —O— | —C(O)— |
| 5 | phenylene | —O— | —(CH₂)ₖ— | —C(O)— | —O— |
| 6 | phenylene | —O— | —C(R⁶)(R⁷)— | —C(O)— | —N(R⁵)— |

Examples of -Y¹-Y²-Y³-Y⁴-Y⁵-Y⁶ are given in Table 17:

TABLE 17

| No. | Y¹ | Y² | Y³ | Y⁴ | Y⁵ | Y⁶ |
|---|---|---|---|---|---|---|
| 1 | —O— | —C(R⁶)(R⁷)— | —(CH₂)ₖ— | —O— | —C(O)— | —O— |
| 2 | —O— | —C(R⁶)(R⁷)— | —(CH₂)ₖ— | —O— | —C(O)— | —N(R⁵)— |
| 3 | —O— | —(CH₂)ₖ— | —C(O)— | —N(R⁵)— | heterocyclylene | —(CH₂)ₖ— |
| 4 | —O— | —(CH₂)ₖ— | —C(O)— | —N(R⁵)— | —(CH₂)ₖ— | —N(R⁵)— |
| 5 | —O— | —(CH₂)ₖ— | heterocyclylene | —C(O)— | —O— | —(CH₂)ₖ— |
| 6 | —O— | —(CH₂)ₖ— | heterocyclylene | —C(O)— | —(CH₂)ₖ— | carbocyclylene |
| 7 | carbocyclylene | —O— | —(CH₂)ₖ— | —C(O)— | —O— | —(CH₂)ₖ— |
| 8 | carbocyclylene | —O— | —C(R⁶)(R⁷)— | —C(O)— | —O— | —(CH₂)ₖ— |

Particular examples of -Y¹-Y²-Y³-Y⁴-Y⁵-Y⁶- are given in Table 18:

TABLE 18

| No. | Y¹ | Y² | Y³ | Y⁴ | Y⁵ | Y⁶ |
|---|---|---|---|---|---|---|
| 1 | —O— | —C(R⁶)(R⁷)— | —CH₂— | —O— | —C(O)— | —O— |
| 2 | —O— | —C(R⁶)(R⁷)— | —CH₂— | —O— | —C(O)— | —N(R⁵)— |

Further particular examples of -Y¹-Y²-Y³-Y⁴-Y⁵-Y⁶- are given in Table 19:

TABLE 19

| | | | | | | |
|---|---|---|---|---|---|---|
| No. | Y¹ | Y² | Y³ | Y⁴ | Y⁵ | Y⁶ |

| | | | | | | |
|---|---|---|---|---|---|---|
| 1 | —O— | —CH(phenyl)- | —CH₂— | —O— | —C(O)— | —O— |
| 2 | —O— | —CH(phenyl)- | —CH₂— | —O— | —C(O)— | —NH— |

Examples of -Y¹-Y²-Y³-Y⁴-Y⁵-Y⁶-Y⁷- are given in Table 20:

TABLE 20

| No. | Y¹ | Y² | Y³ | Y⁴ | Y⁵ | Y⁶ | Y⁷ |
|---|---|---|---|---|---|---|---|
| 1 | —O— | —C(R⁶)(R⁷)— | —(CH₂)ₖ— | —O— | —C(O)— | —O— | —(CH₂)ₖ— |
| 2 | —O— | —C(R⁶)(R⁷)— | —(CH₂)ₖ— | —O— | —C(O)— | —N(R⁵)— | —(CH₂)ₖ— |
| 3 | —O— | —(CH₂)ₖ— | —C(R⁶)(R⁷)— | —O— | —(CH₂)ₖ— | —C(O)— | —O— |
| 4 | —O— | —(CH₂)ₖ— | —C(R⁶)(R⁷)— | —O— | —C(O)— | —N(R⁵)— | —(CH₂)ₖ— |

Particular examples of -Y¹-Y²-Y³-Y⁴-Y⁵-Y⁶-Y⁷- are given in Table 21:

TABLE 21

| No. | Y¹ | Y² | Y³ | Y⁴ | Y⁵ | Y⁶ | Y⁷ |
|---|---|---|---|---|---|---|---|
| 1 | —O— | —CH(R⁷)— | —CH₂— | —O— | —C(O)— | —O— | —CH₂— |
| 2 | —O— | —CH(R⁷)— | —CH₂— | —O— | —C(O)— | —NH— | —CH₂— |
| 3 | —O— | —CH(phenyl)- | —CH₂— | —O— | —C(O)— | —O— | —CH₂— |
| 4 | —O— | —CH(phenyl)- | —CH₂— | —O— | —C(O)— | —NH— | —CH₂— |

With regard to Tables 2 to 21, where —(CH₂)ₖ— is mentioned it is often —CH₂— or —(CH₂)₂—.

Where —N(R⁵)— is mentioned, it is often —NH—, —N(CH₃)— or —N(benzyl)-.

Where —S(O)ₗ— is mentioned, it may be —S—, —S(O)— or —S(O)₂—.

Where —C(R⁶)(R⁷)— is mentioned, R⁶ is usually selected from hydrogen, C₁₋₆ alkyl or —C(O)O—C₁₋₆ alkyl; and R⁷ is usually C₁₋₆ alkyl, —(CH₂)ⱼ-carbocyclyl or —(CH₂)ⱼ-heterocyclyl. In particular, R⁶ may be hydrogen or C₁₋₆ alkyl optionally substituted with 1, 2, 3, 4 or 5 R¹¹; and R⁷ may be C₁₋₆ alkyl, —(CH₂)ₖ-cycloalkyl, —(CH₂)ⱼ-aryl or —(CH₂)ⱼ-heterocyclyl, any of which is optionally substituted with 1, 2, 3, 4 or 5 R¹¹. Index j is often 0 or 1, e.g. 0. Thus, in particular in the relevant examples given in Tables 2 to 21, —C(R⁶)(R⁷)— may be —CH(CH₃)—, —C(CH₃)₂—, —CH(phenyl)- or —C(CH₃)(phenyl)-, wherein the methyl or phenyl parts are optionally substituted with 1, 2, 3, 4 or 5 R¹¹.

Where carbocyclylene is mentioned, it may be substituted with 1, 2, 3, 4 or 5 R¹¹. Carbocyclylene is usually cycloalkylene (e.g. cyclopropylene, cyclobutylene, cyclopentylene or cyclohexylene) or arylene (e.g. phenylene or naphthylene), either of which is optionally substituted with 1, 2, 3, 4 or 5 R¹¹.

Where heterocyclylene is mentioned, it may be substituted with 1, 2, 3, 4 or 5 R¹¹. Heterocyclylene may be selected from oxiranylene, azirinylene, 1,2-oxathiolanylene, imidazolylene, thienylene, furylene, tetrahydrofurylene, pyranylene, thiopyranylene, thianthrenylene, isobenzofuranylene, benzofuranylene, chromenylene, 2H-pyrrolylene, pyrrolylene, pyrrolinylene, pyrrolidinylene, imidazolylene, imidazolidinylene, benzimidazolylene, pyrazolylene, pyrazinylene, pyrazolidinylene, pyranyol, thiazolylene, isothiazolylene, dithiazolylene, oxazolylene, isoxazolylene, pyridylene, pyrazinylene, pyrimidinylene, piperidylene, especially piperidin-1-ylene, piperazinylene, especially piperazin-1-ylene, pyridazinylene, morpholinylene, especially morpholino, thiomorpholinylene, especially thiomorpholino, indolizinylene, isoindolylene, 3H-indolylene, indolylene, benzimidazolylene, cumarylene, indazolylene, triazolylene, tetrazolylene, purinylene, 4H-quinolizinylene, isoquinolylene, quinolylene, tetrahydroquinolylene, tetrahydroisoquinolylene, decahydroquinolylene, octahydroisoquinolylene, benzofuranylene, dibenzofuranylene, benzothiophenylene, dibenzothiophenylene, phthalazinylene, naphthyridinylene, quinoxalylene, quinazolinylene, quinazolinylene, cinnolinylene, pteridinylene, carbazolylene, β-carbolinylene, phenanthridinylene, acridinylene, perimidinylene, phenanthrolinylene, furazanylene, phenazinylene, phenothiazinylene, phenoxazinylene, chromenylene, isochromanylene and chromanylene, any of which is optionally substituted with 1, 2, 3, 4 or 5 R¹¹.

Where alkyl, phenyl, benzyl and phenylene are mentioned, they may be substituted with 1, 2, 3, 4 or 5 R¹¹.

Included in the invention are linkers which comprise the structure:

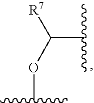

linked to ring AB through the terminal oxygen. R⁷ is as previously described, e.g. C₁₋₆ alkyl, —(CH₂)ₖ-cycloalkyl, —(CH₂)ₖ-cycloalkenyl, —(CH₂)ⱼ-aryl or —(CH₂)ⱼ-heterocyclyl, any of which is optionally substituted with 1, 2, 3, 4 or 5 R¹¹, e.g. halo. In particular embodiments, R⁷ is a 5- or 6-membered ring, particularly carbocyclic ring, or such a ring substituted by 1, 2, 3, 4 or 5 R¹¹, e.g. halo; in either case, the carbocyclic ring may be phenyl but in other embodiments is a wholly or partially saturated analogue thereof. The invention includes compounds which have a linker of said structure and in which n is 1. The invention further includes compounds which have exactly one linker of said structure.

Particular linkers comprise or consist of, e.g. consist of, the structure:

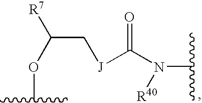

linked to ring AB through the terminal oxygen. J is O, S, CH₂ or NR⁴⁰, where R⁴⁰ is selected from H, hydroxy, C₁₈ aliphatic (e.g. alkyl having 1, 2, 3 or 4 carbon atoms) optionally substituted by halogen (e.g. methyl or halomethyl, as an example of the latter of which trifluoromethyl may be mentioned). In embodiments, R⁴⁰ is H. J is in particular O and in many compounds J is O and R⁴⁰ is H. R⁷ is as described in the preceding paragraph. The invention includes compounds which have a linker of said structure and in which n is 1. The invention further includes compounds which have exactly one linker of said structure.

### R⁴

R⁴ is present when n is 1 or 2 and is hydrogen, except when Y is a bond; or is hydrocarbyl or heterocyclyl, either of which is optionally substituted with 1, 2, 3, 4 or 5 R¹¹. Where n is 2, the R⁴ moieties may be the same or different.

In one embodiment, the or each R⁴ is independently selected from hydrogen (except when Y is a bond); C₁₋₆ alkyl optionally substituted with 1, 2, 3, 4 or 5 R¹¹; carbocyclyl (e.g. cycloalkyl or aryl) optionally substituted with 1, 2, 3, 4 or 5 R¹¹; and heterocyclyl (e.g. heterocycloalkyl or heteroaryl) optionally substituted with 1, 2, 3, 4 or 5 R¹¹.

R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazole, quinolyl, isoquinolyl, benzoxazole, benzofurazanyl, piperidinyl, pyrrolidinyl or 1,4-benzodioxanyl), any of which is optionally substituted with at least one organic or inorganic substituent, e.g. 1, 2, 3, 4 or 5 R¹¹.

Exemplary carbocycles include those shown in Table 22 below, any of which may be substituted. In embodiments and without limitation, the carbocycles are substituted with 1, 2, 3, 4 or 5 R¹¹.

TABLE 22

| No | R⁴ |
|---|---|
| 1 | 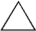 |
| 2 | 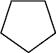 |
| 3 | 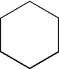 |
| 4 | 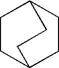 |
| 5 | 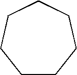 |
| 6 | 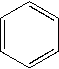 |
| 7 | 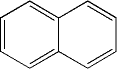 |
| 8 | 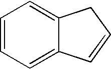 |
| 9 | 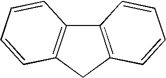 |

Also to be mentioned are wholly or partially saturated analogues of the unsaturated carbocycles in Table 22. Structures 1-4 and 6-9 of Table 22 form one embodiment of the invention.

Exemplary heterocycles include those shown in Table 23 below, any of which may be substituted. In embodiments and without limitation, the carbocycles are substituted with 1, 2, 3, 4 or 5 R¹¹.

TABLE 23

| No | R⁴ |
|---|---|
| 1 | 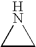 |
| 2 | 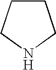 |
| 3 | 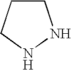 |
| 4 | 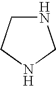 |
| 5 | 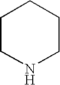 |
| 6 | 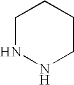 |
| | 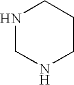 |
| 8 | 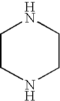 |
| 9 | 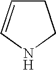 |
| 10 | 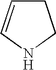 |
| 11 | 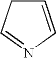 |
| 12 | 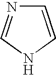 |
| 13 | 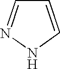 |
| 14 | 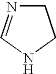 |
| 15 | 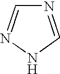 |
| 16 | 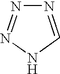 |
| 17 | 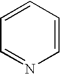 |
| 18 | 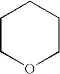 |
| 19 | 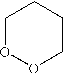 |
| 20 | 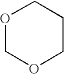 |
| 21 | 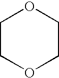 |
| 22 | 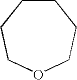 |
| 23 | 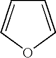 |
| 24 | 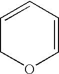 |
| 25 | 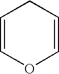 |
| 26 | 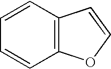 |
| 27 | 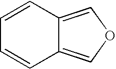 |
| 28 | 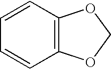 |
| 29 | 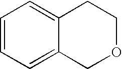 |
| 30 | 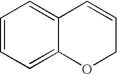 |
| 31 | 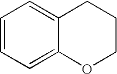 |
| 32 | 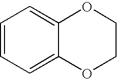 |
| 33 | 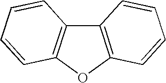 |
| 34 | 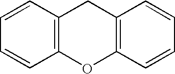 |
| 35 | 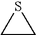 |
| 36 | 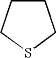 |
| 37 | 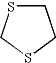 |
| 38 | 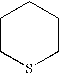 |
| 39 | 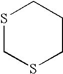 |
| 40 | 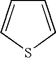 |
| 41 | 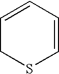 |
| 42 | 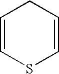 |
| 43 | 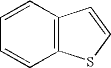 |
| 44 | 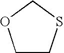 |
| 45 | 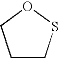 |
| 46 | 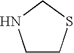 |
| 47 | 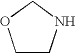 |
| 48 | 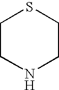 |
| 49 | 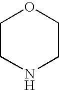 |
| 50 | 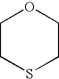 |
| 51 | 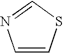 |
| 52 | 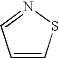 |
| 53 | 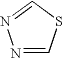 |
| 54 | 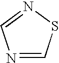 |
| 55 | 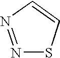 |
| 56 | 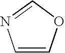 |
| 57 | 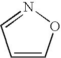 |
| 58 | 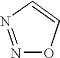 |
| 59 | 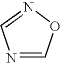 |
| 60 | 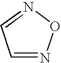 |
| 61 | 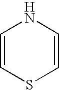 |
| 62 | 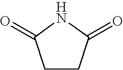 |
| 63 | 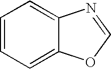 |
| 64 | 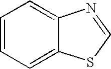 |
| 65 | 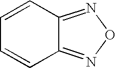 |
| 66 | 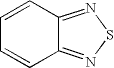 |
| 67 | 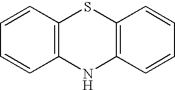 |
| 68 | 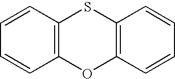 |
| 69 | 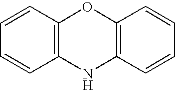 |

When R⁴ is carbocyclyl or heterocyclyl, it may in principle be bound to Y (or, when Y is a bond, Ring A) at any available position on the ring. Thus, for example, when R⁴ is thiophenyl it may be attached to Y or Ring A at any of the 2-, 3-, 4- or 5-positions.

In some compounds, when R⁴ is substituted by 1, 2, 3, 4 or 5 R¹¹, the or each R¹¹ is independently selected from halogen (e.g. fluorine, chlorine, bromine or iodine), hydroxy, trifluoromethyl, cyano, nitro, oxo, —OR², —C(O)R², —C(O)OR², —OC(O)R², —N(R²)R¹³, —C(O)N(R²)R³, —S(O)ₗR², —S(O)ₗN(R²)R³, —C(R²)₃ and R⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl), —(CH₂)ⱼ-heterocyclyl where heterocyclyl is a saturated or unsaturated heterocyclic ring (for example heteroaryl, e.g. pyridinyl or thiophenyl, or heterocycloalkyl, e.g. piperazinyl, piperadinyl, pyrrolidinyl or morpholinyl), any of which is optionally substituted with 1, 2, 3, 4 or 5 substituents independently selected from halogen (e.g. fluorine or chlorine), hydroxy, C₁₋₆ alkoxy, amino, mono- or di-alkylamino, C₁₋₆ haloalkyl (e.g. trifluoromethyl) or C₁₋₆ alkyl (e.g. methyl, ethyl, propyl or butyl), and of these substituents may particularly be mentioned halogen (e.g. fluorine or chlorine), hydroxy, or C₁₋₆ alkyl (e.g. methyl, ethyl, propyl or butyl). R¹⁴ is in some compounds selected from C₁-C₆ alkyl (e.g. methyl, ethyl, propyl or butyl), —(CH₂)ᵢ—O—(CH₂)ⱼ-heteroalkyl, —(CH₂)ᵢ—O—(CH₂)ⱼ-aryl (e.g. phenyl or benzyl) and —(CH₂)ᵢ—O—(CH₂)ⱼ-heterocyclyl (e.g. piperazinyl, pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). In particular, R¹⁴ is often selected from C₁-C₆ alkyl (e.g. methyl, ethyl, propyl or butyl), —(CH₂)ⱼ-aryl (e.g. phenyl or benzyl) and —(CH₂)ⱼ-heteroaryl (e.g. piperazinyl, pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

In a class of compounds comprising individual exemplary compounds presented later in this specification, R¹¹ as a substituent of R⁴, especially when R⁴ is carbocyclyl or heterocyclyl, is C₁-C₆ alkyl (e.g. methyl); F; Cl; trifluoromethyl; cyano; nitro; hydroxy; hydroxy(C₁-C₆)alkyl; —(CH₂)ⱼ—NR^{y}R^{z} where R^{y} and R^{z} are independently selected from H, C₁-C₆ alkyl and, less frequently, —OH (e.g. both are H or both are methyl); C₁-C₆ alkoxy (e.g. methoxy); C₁-C₆ alkylthio; alkoxyalkyl having from 2 to 8 carbon atoms (e.g. methoxymethyl, methoxyethyl, ethoxymethyl, ethoxyethyl); alkylcarbonyl of which the alkyl part (i) is unsubstituted or is substituted by 1, 2, 3, 4 or 5 halogens or by hydroxy and (ii) has from 1 to 6 carbon atoms (e.g. acetyl); —(CH₂)ⱼ—N(R^{w})C(O)alkyl where R^{w} is H or C₁-C₆ alkyl; —(O)ᵤ—(CH₂)ⱼ-heterocyclyl where u is 0 or 1 and heterocyclyl is a 5- or 6-membered saturated or unsaturated heterocycle, e.g. piperazinyl, piperidinyl, morpholinyl, pyridinyl; —(O)ᵤ—(CH₂)ⱼ-carbocyclyl where u is 0 or 1 and carbocyclyl is a 5- or 6-membered saturated or unsaturated carbocycle, e.g. phenyl or cyclohexyl; —C(O)—(CH₂)ⱼ-carbocyclyl or —C(O)—(CH₂)ⱼ-heterocyclyl where and heterocyclyl are as earlier described in this paragraph; —(CH₂)ⱼ—S(O)₂NH-carbocyclyl or —(CH₂)ⱼ—S(O)₂NH-heterocyclyl where g, carbocyclyl and heterocyclyl are as earlier described in this paragraph; —(CH₂)ⱼ—S(O)₂NR^{y}R^{z}; —(CH₂)ⱼ—S(O)₂NHCOOH; —S(O)₂NHCOOalkyl where alkyl has from 1 to 6 carbon atoms (e.g. methyl); —(CH₂)ⱼ—S(O)₂-alkyl where alkyl has from 1 to 6 carbon atoms (e.g. methyl); —(CH₂)ⱼ—NR^{y}S(O)₂-alkyl where alkyl has from 1 to 6 carbon atoms (e.g. methyl); —NR^{y}C(O)-alkyl where alkyl has from 1 to 6 carbon atoms (e.g. methyl); —(CH₂)ⱼ—OC(O)NR^{y}R^{z}; —(CH₂)ⱼ—NR^{w}C(O)NR^{y}R^{z}; and —(CH₂)ⱼ—C(O)NR^{y}R^{z}. The value of j in the groups mentioned in this paragraph is 0, 1, 2, 3, 4, 5 or 6 and is 0 in some embodiments; in other embodiments it is 1 or 2, e.g. 1. The carbocyclyl and heterocyclyl groups mentioned in this paragraph as comprised within an R¹¹ group may be unsubstituted or substituted by 1, 2, 3, 4 or 5 cycle-free R¹¹ moieties, e.g. by one or two such moieties; often no more than one such substituent contains more than four multivalent atoms.

Typically, when R⁴ is carbocyclyl or heterocyclyl, it has 0, 1, 2, 3 or 4 substituents; often no more than one such substituent contains more than four multivalent atoms. Thus in one embodiment, all substituent(s) (if there are any) have 0, 1, 2, 3 or 4 multivalent atoms (e.g. 0-3). In another embodiment, there is a single substituent which has more than 4 multivalent atoms whilst any other substituents have 0, 1, 2, 3 or 4 multivalent atoms; in a sub-class there is a single substituent which has more than 3 multivalent atoms whilst any other substituents have 0, 1, 2 or 3.

Compounds in which n is 0

In one class of compounds, n is 0.

Embodiments of Formulae (I) to (VII) in which n is 0 include those shown below, and pharmaceutically acceptable salts or prodrugs thereof:

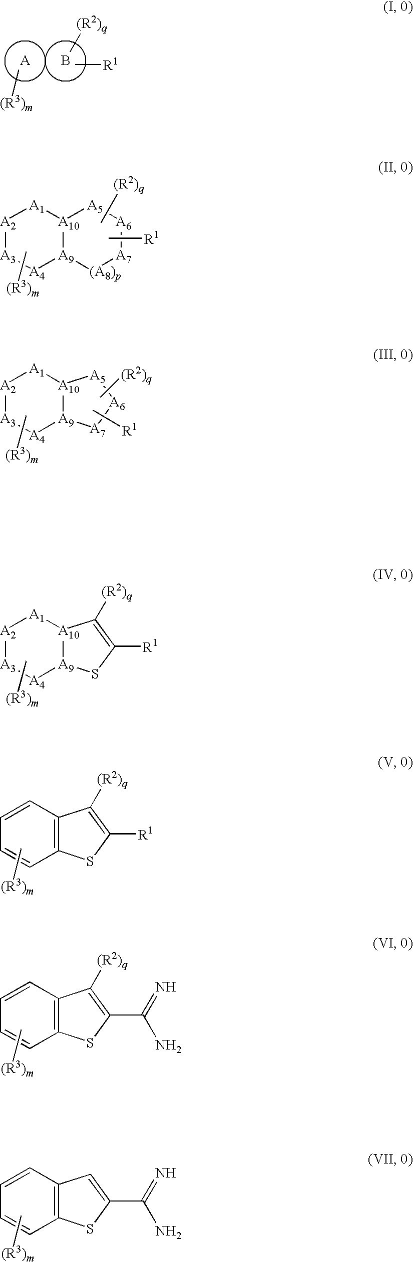

In the above embodiments, R³ is usually 1 or 2, and R³ is typically selected from halogen (e.g. fluorine, chlorine, bromine or iodine), hydroxy, trifluoromethyl, cyano, nitro, oxo, —OR¹², —C(O)R¹², —C(O)OR¹², —OC(O)R¹², —N(R¹²)R¹³—C(O)N(R¹²)R¹³, —S(O)R¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 1

In another class of compounds, n is 1.

Embodiments of Formulae (I) to (VII) in which n is 1 include those shown below, and pharmaceutically acceptable salts or prodrugs thereof:

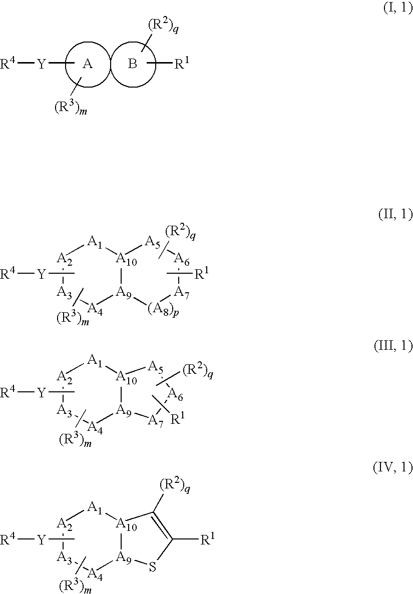

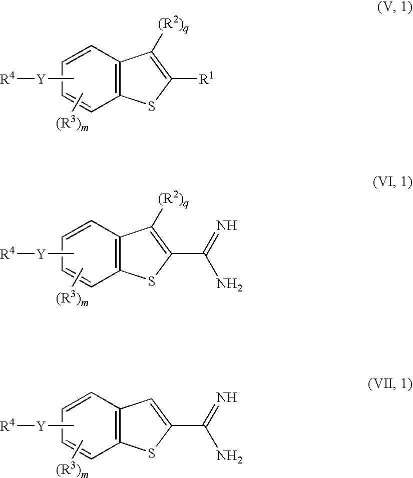

Particular embodiments of Formula (V, 1) include those shown below, and pharmaceutically acceptable salts or prodrugs thereof:

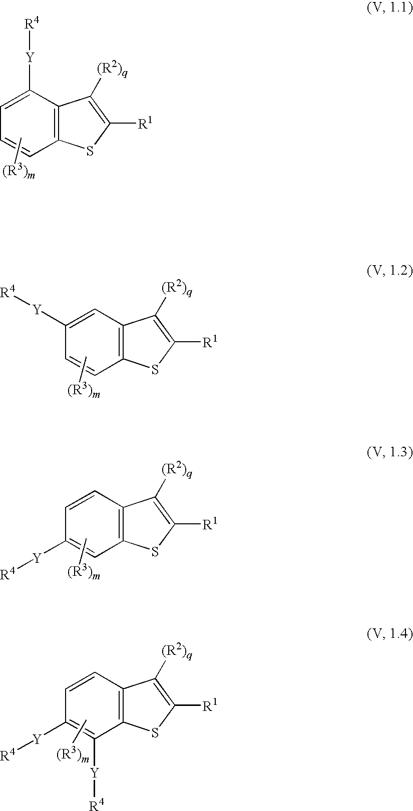

Particular embodiments of Formula (VI, 1) include those shown below, and pharmaceutically acceptable salts or prodrugs thereof:

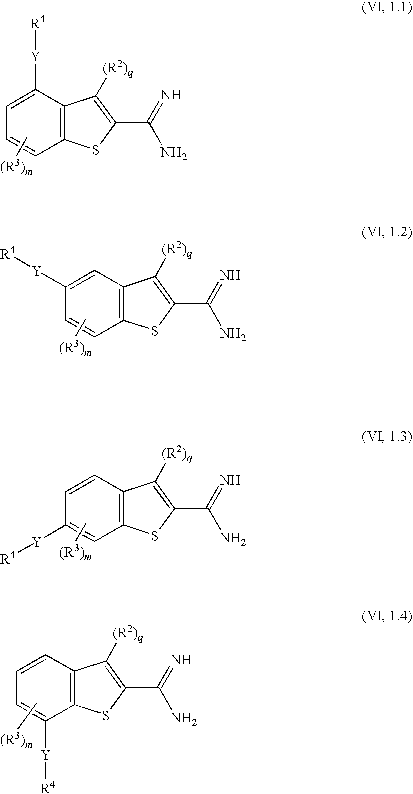

Particular embodiments of Formula (VII, 1) include those shown below, and pharmaceutically acceptable salts or prodrugs thereof:

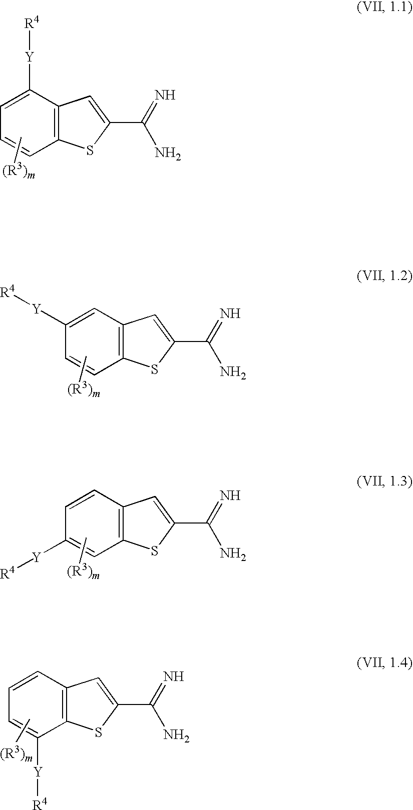

In the above embodiments, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazole, quinolyl, isoquinolyl, benzoxazole, benzofurazanyl, piperidinyl, pyrrolidinyl or 1,4-benzodioxanyl), any of which is optionally substituted with 1, 2, 3, 4 or 5 R¹¹.

Also, m is usually 0 or 1, and R³ is typically selected from halogen (e.g. fluorine, chlorine, bromine or iodine), hydroxy, trifluoromethyl, cyano, nitro, oxo, —OR², —C(O)R², —C(O)OR¹², —OC(O)R², —N(R²)R³, —C(O)N(R²)R³, —S(O)ₗR², —C(R²)₃ and R⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 1 and Y is a bond

In one embodiment, n is 1 and Y is a bond.

Embodiments of Formulae (I) to (VII) in which n is 1 and Y is a bond include those shown below, and pharmaceutically acceptable salts or prodrugs thereof:

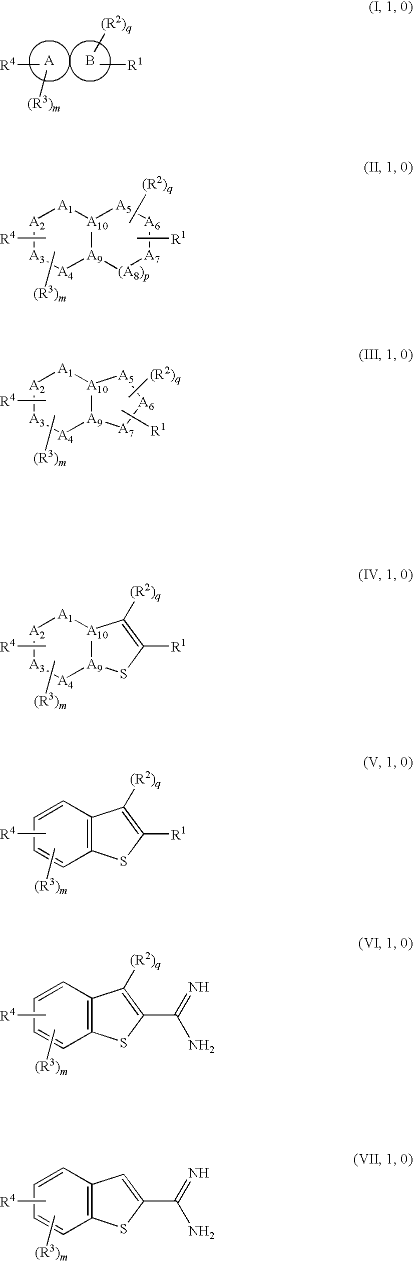

Particular embodiments of Formula (V, 1, 0) include those shown below, and pharmaceutically acceptable salts or prodrugs thereof:

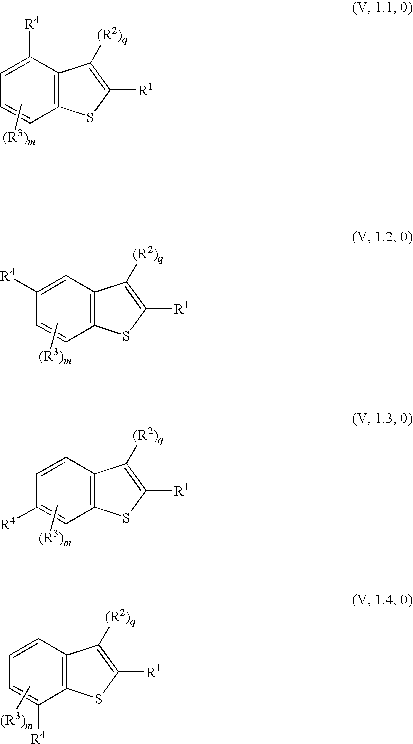

Particular embodiments of Formula (VI, 1, 0) include those shown below, and pharmaceutically acceptable salts or prodrugs thereof:

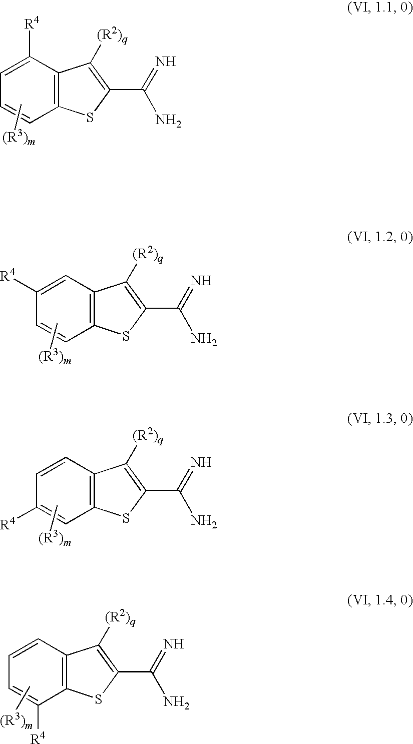

Particular embodiments of Formula (VII, 1, 0) include those shown below, and pharmaceutically acceptable salts or prodrugs thereof:

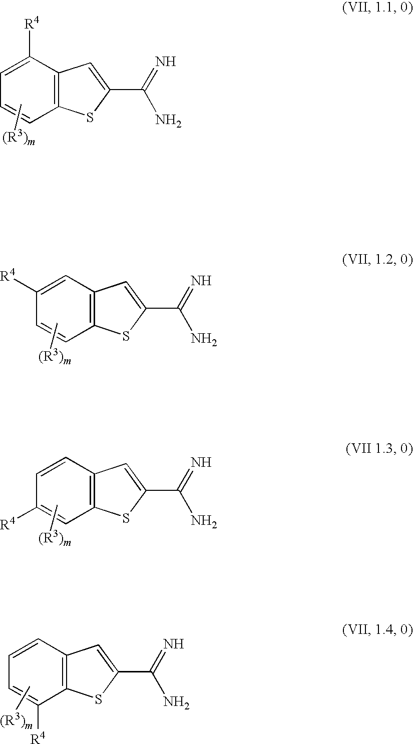

In the above embodiments, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

A particular embodiment of Formula (VII, 1, 0) is a compound of Formula (VIII):

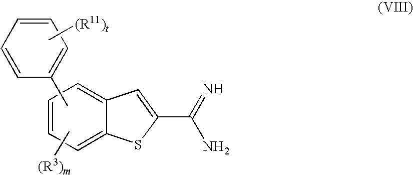

wherein t is 0, 1, 2, 3, 4 or 5;
or a pharmaceutically acceptable salt of prodrug thereof.

Particular embodiments of Formula (VIII) include those shown below, and pharmaceutically acceptable salts or prodrugs thereof:

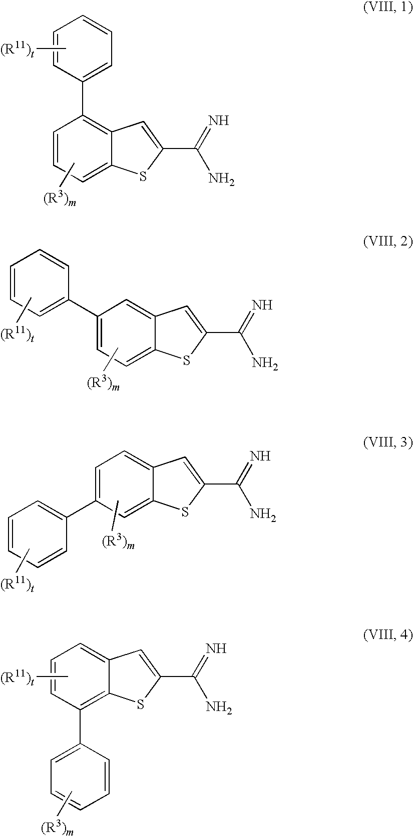

With regard to Formula (VIII), t is usually 0, 1 or 2, and R¹¹ is typically independently selected from halogen (e.g. fluorine, chlorine, bromine or iodine), hydroxy, trifluoromethyl, cyano, nitro, oxo, —R¹², —C(O)R¹², —C(O)R¹², —OC(O)R¹², N(R¹²)R¹³, —C(O)N(R¹²)R¹³, —S(O)ₗR¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

With regard to the various embodiments described in this section, m is usually 0 or 1, and R³ is typically selected from halogen (e.g. fluorine, chlorine, bromine or iodine), hydroxy, trifluoromethyl, cyano, nitro, oxo, —OR¹², —C(O)R¹², —C(O)OR¹², —OC(O)R¹², —N(R¹²)R¹³, —C(O)N(R¹²)R¹³, —S(O)ₗR¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 1 and Y is Y¹

In another embodiment, n is 1 and Y is Y¹.

Embodiments of Formulae (I) to (VII) in which n is 1 and Y is Y¹ include those shown below, and pharmaceutically acceptable salts or prodrugs thereof:

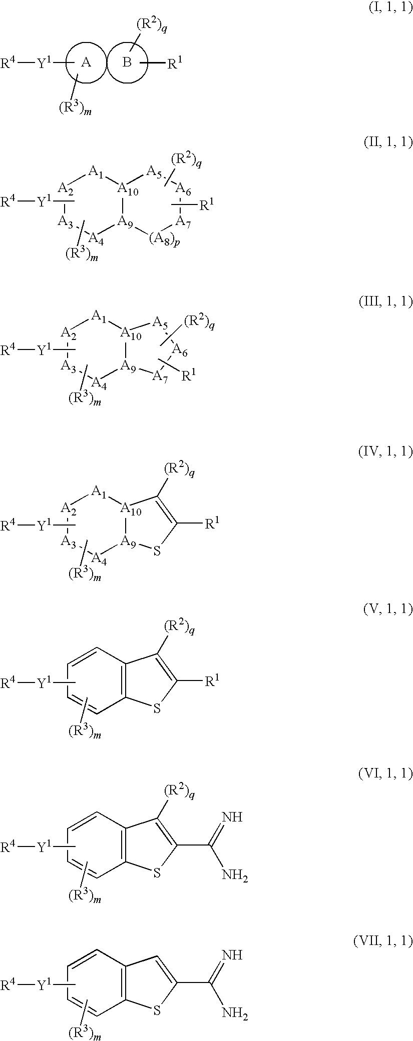

Particular embodiments of Formula (V, 1, 1) include those shown below, and pharmaceutically acceptable salts or prodrugs thereof:

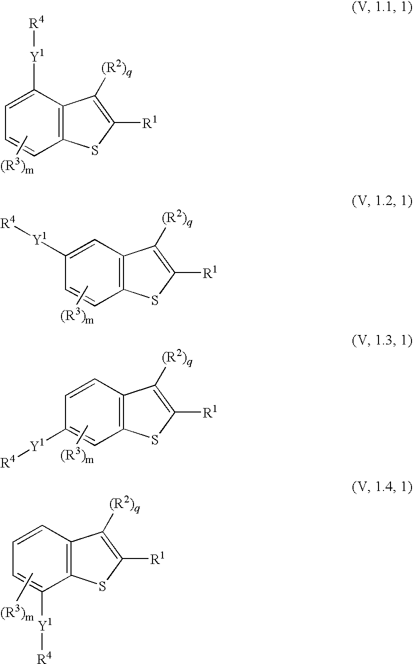

Particular embodiments of Formula (VI, 1, 1) include those shown below, and pharmaceutically acceptable salts or prodrugs thereof:

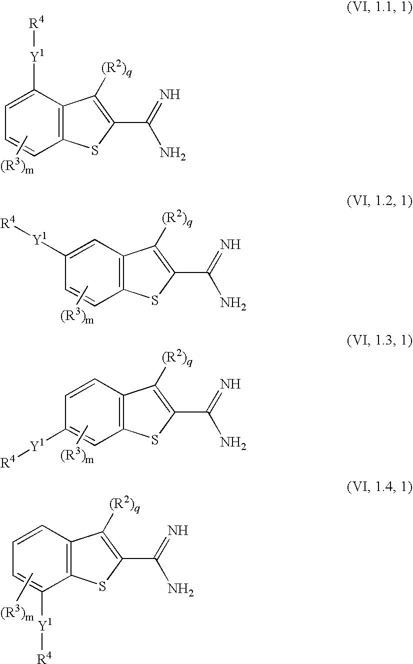

Particular embodiments of Formula (VII, 1, 1) include those shown below, and pharmaceutically acceptable salts or prodrugs thereof:

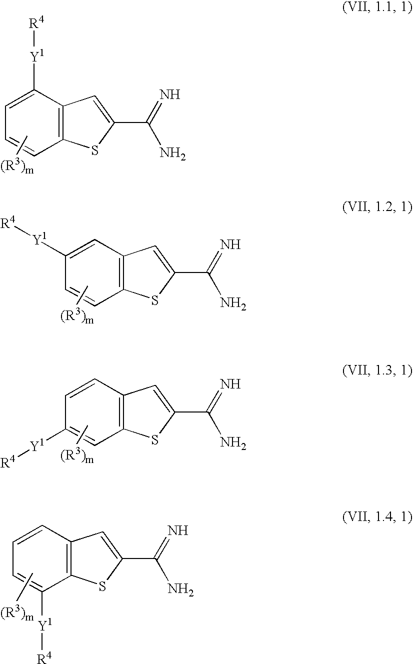

Y¹ may be as defined in any of Tables 2, 3 and 4, and is, in particular, —O— or —C≡C—.

A particular embodiment of Formula (VII, 1, 1) is a compound of Formula (IX):

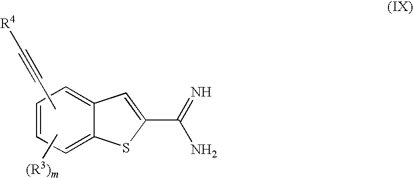

or a pharmaceutically acceptable salt of prodrug thereof.

Embodiments of Formula (IX) include those shown below, and pharmaceutically acceptable salts and prodrugs thereof:

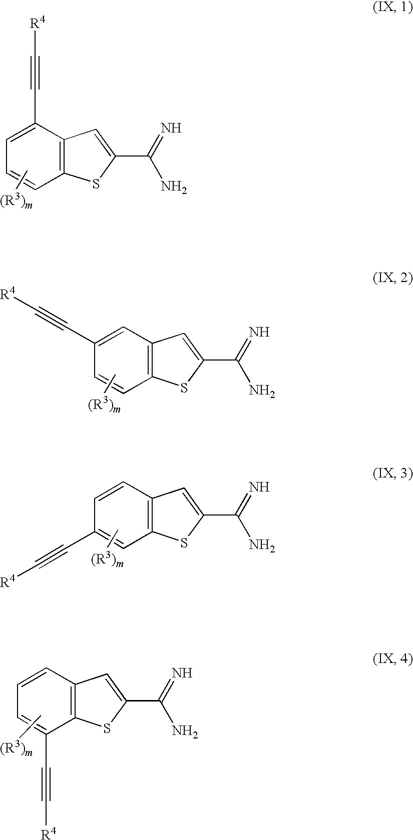

Another particular embodiment of Formula (VII, 1, 1) is a compound of Formula (X):

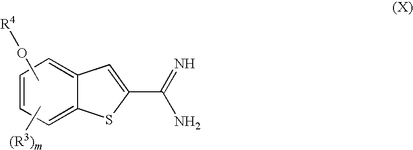

or a pharmaceutically acceptable salt of prodrug thereof.

Embodiments of Formula (X) include those shown below, and pharmaceutically acceptable salts and prodrugs thereof:

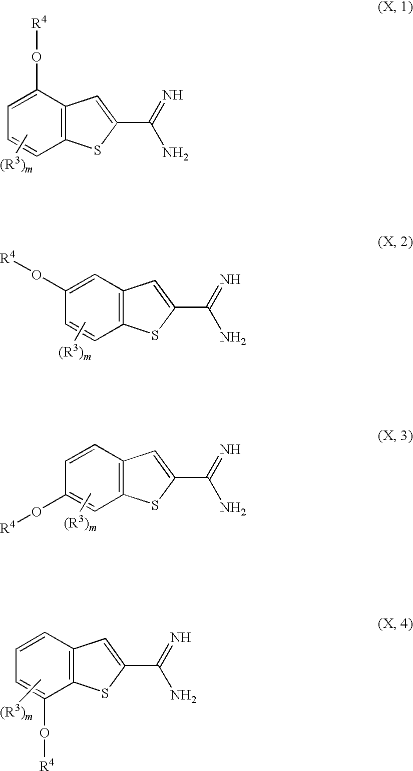

In one embodiment of Formula (X), R⁴ is other than methyl.

With regard to the various embodiments described in this section, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

Also, m is usually 0 or 1, and R³ is typically independently selected from halogen (e.g. fluorine, chlorine, bromine or iodine), hydroxy, trifluoromethyl, cyano, nitro, oxo, —OR¹², —C(O)R¹², —C(O)OR¹², —OC(O)R¹², —N(R¹²)R¹³, —C(O)N(R¹²)R¹³, —S(O)ₗR¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 1 and Y is -Y¹-Y²-.

In another embodiment, n is 1 and Y is -Y¹-Y².

Embodiments of Formulae (I) to (VII) in which n is 1 and Y is -Y¹-Y² include those shown below, and pharmaceutically acceptable salts or prodrugs thereof:

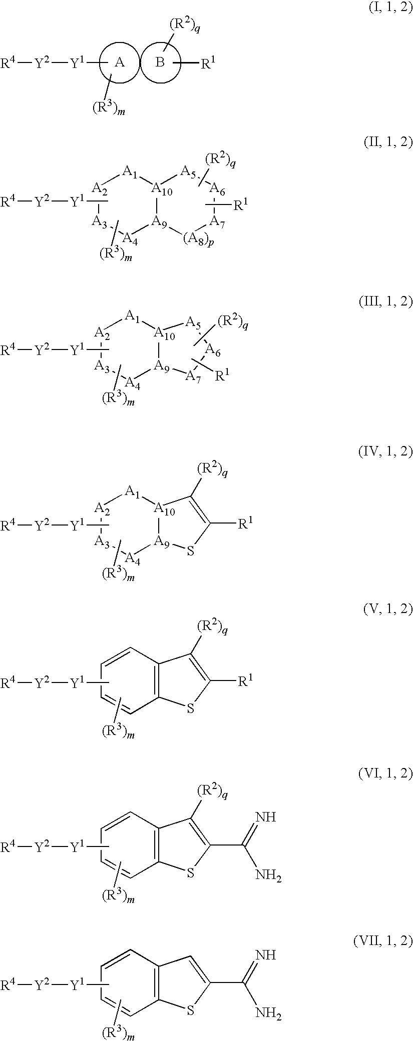

Particular embodiments of Formula (V, 1, 2) include those shown below, and pharmaceutically acceptable salts or prodrugs thereof:

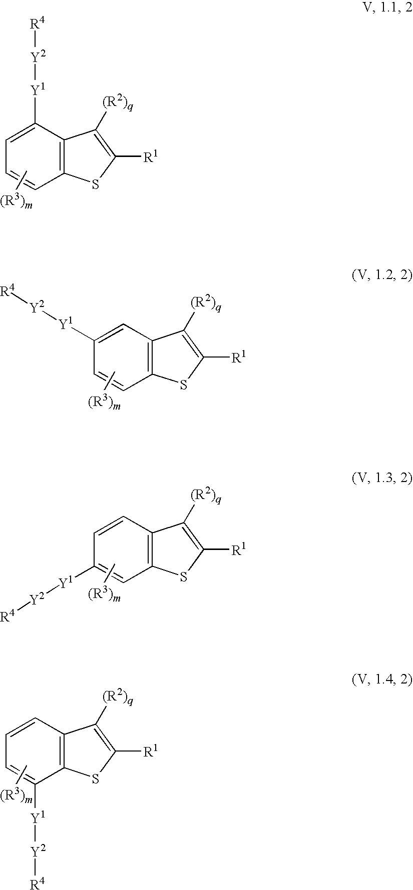

Particular embodiments of Formula (VI, 1, 2) include those shown below, and pharmaceutically acceptable salts or prodrugs thereof:

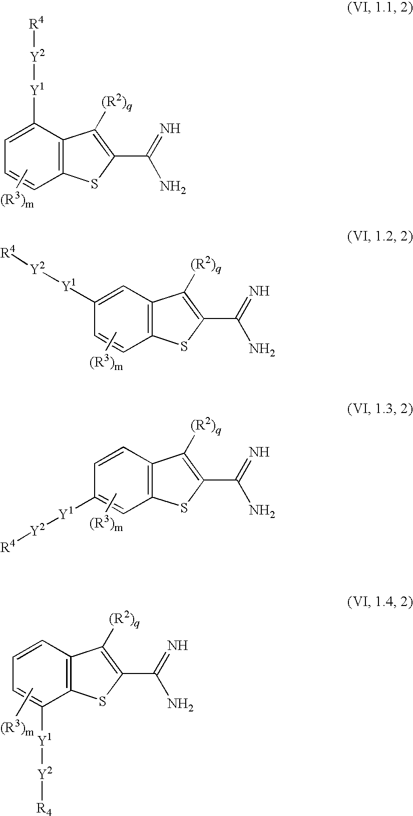

Particular embodiments of Formula (VII, 1, 2) include those shown below, and pharmaceutically acceptable salts or prodrugs thereof:

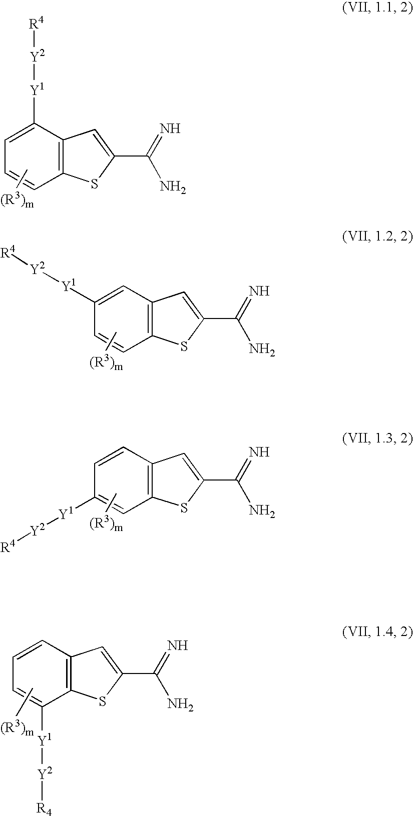

In the above embodiments, -Y¹-Y²- may be as defined in any of Tables 5, 6 and 7, and is in particular —O—CH₂— or —O—CH(R⁷)—. In a particular class of compounds, -Y¹-Y²- is other than —O—C(O)— or —S—C(O)—.

A particular embodiment of Formula (VII, 1, 2) is a compound of Formula (XI):

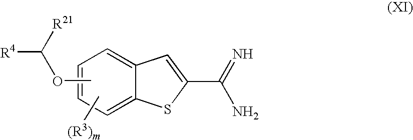

wherein R²¹ is hydrogen or R⁷;
or a pharmaceutically acceptable salt of prodrug thereof.

Embodiments of Formula (XI) include those shown below, and pharmaceutically acceptable salts and prodrugs thereof:

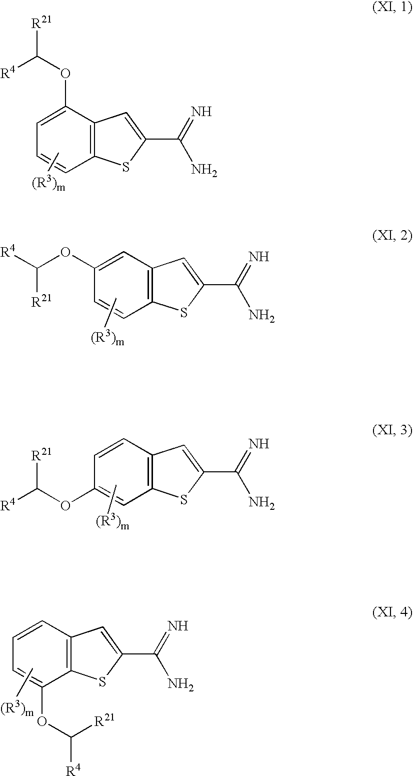

With regard to Formula (XI), R²¹ is typically hydrogen or is selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹. More usually, R²¹ is hydrogen, or is C₁₋₆ alkyl (e.g. methyl or ethyl) or phenyl, either of which is optionally substituted with 1, 2, 3, 4 or 5 R¹¹.

With regard to the various embodiments described in this section, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

Also, m is usually 0 or 1, and R³ is typically independently selected from halogen (e.g. fluorine, chlorine, bromine or iodine), hydroxy, trifluoromethyl, cyano, nitro, oxo, —OR¹², —C(O)R¹², —C(O)OR¹², —OC(O)R¹²—N(R¹²)R¹³, —C(O)N(R¹²)R¹³, —S(O)ₗR¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 1 and y is -Y¹-Y²-Y³-.

In another embodiment, n is 1 and Y is -Y¹-Y²-Y³-.

Embodiments of Formulae (I) to (VII) in which n is 1 and Y is -Y¹-Y²-Y³- include those shown below, and pharmaceutically acceptable salts or prodrugs thereof:

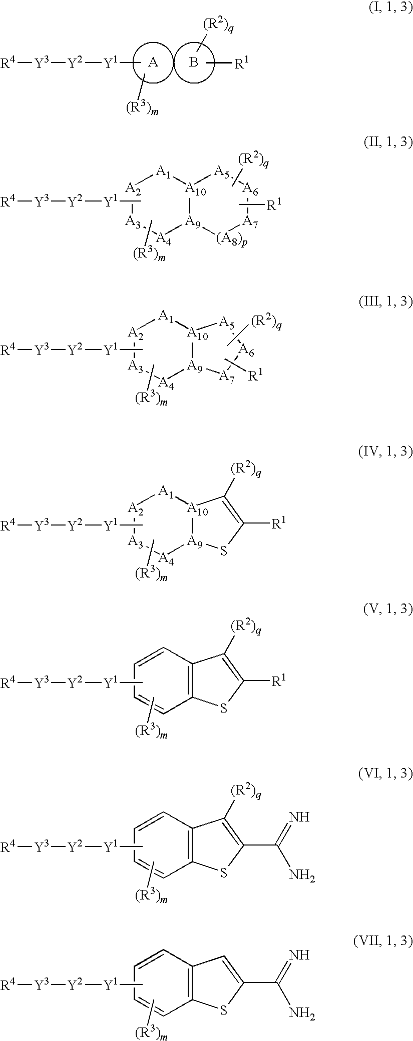

Particular embodiments of Formula (V, 1, 3) include those shown below, and pharmaceutically acceptable salts or prodrugs thereof:

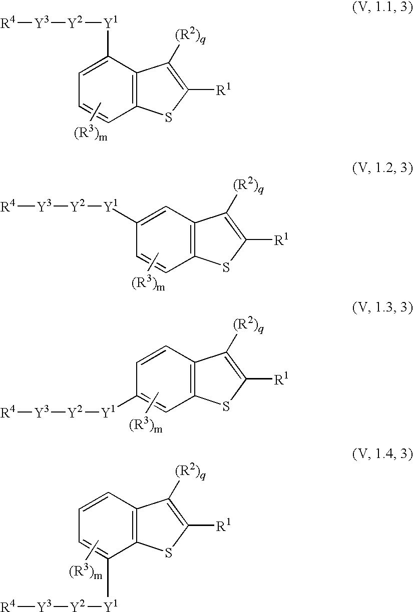

Particular embodiments of Formula (VI, 1, 3) include those shown below, and pharmaceutically acceptable salts or prodrugs thereof:

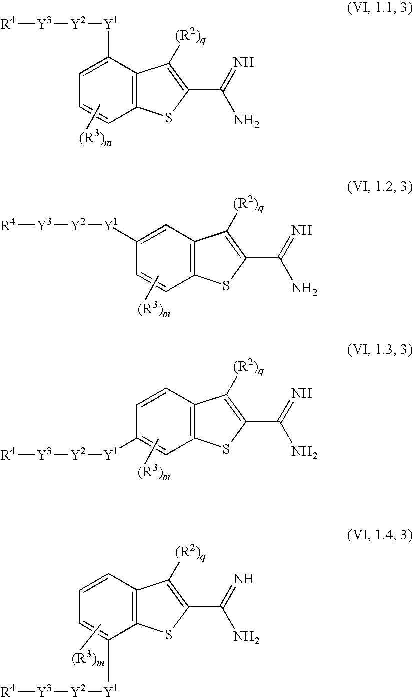

Particular embodiments of Formula (VII, 1, 3) include those shown below, and pharmaceutically acceptable salts or prodrugs thereof:

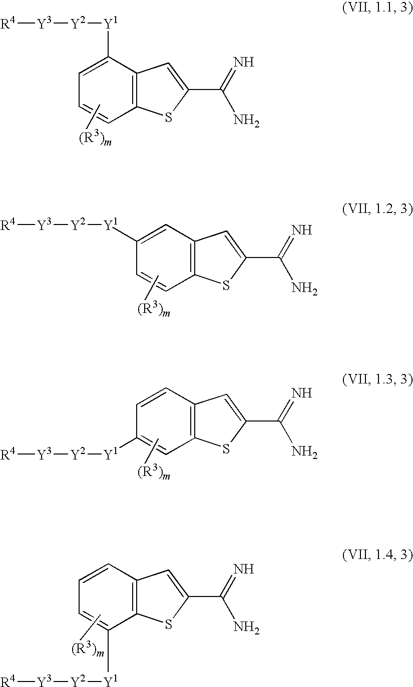

In the above embodiments, -Y¹-Y²-Y³- may be as defined in any of Tables 8, 9, 10 and 11.

A particular embodiment of Formula (VII, 1, 3) is a compound of Formula (XII):

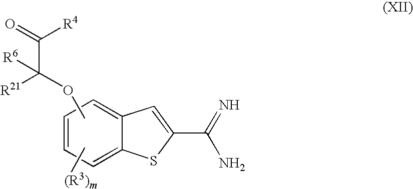

wherein R²¹ is hydrogen or R⁷;
or a pharmaceutically acceptable salt or prodrug thereof.

Embodiments of Formula (XII) include the following, and pharmaceutically acceptable salts and prodrugs thereof:

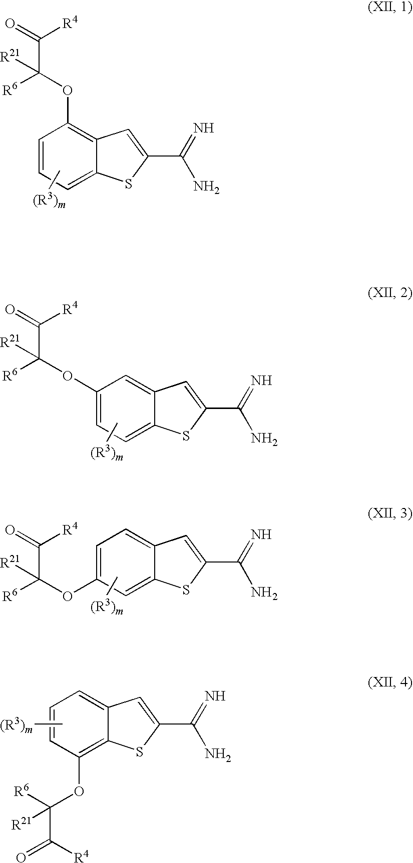

With regard to Formula (XII), R⁶ is usually hydrogen and R²¹ is typically hydrogen or is selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹. More usually, R⁶ is hydrogen and R²¹ is hydrogen, or is C₁₋₆ alkyl (e.g. methyl or ethyl) or phenyl, either of which is optionally substituted with 1, 2, 3, 4 or 5 R¹¹.

With regard to the various embodiments described in this section, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

Also, m is usually 0 or 1, and R³ is typically independently selected from halogen (e.g. fluorine, chlorine, bromine or iodine), hydroxy, trifluoromethyl, cyano, nitro, oxo, —OR¹², —C(O)R¹², —C(O)OR¹², —OC(O)R¹², —N(R¹²)R¹³, —C(O)N(R¹²)R¹³, —S(O)ₗR¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 1 and Y is -Y¹-Y²-Y³-Y⁴-

In another embodiment, n is 1 and Y is -Y¹-Y²-Y³-Y⁴-.

Embodiments of Formulae (I) to (VII) in which n is 1 and Y is -Y¹-Y²-Y³-Y⁴- include those shown below, and pharmaceutically acceptable salts or prodrugs thereof:

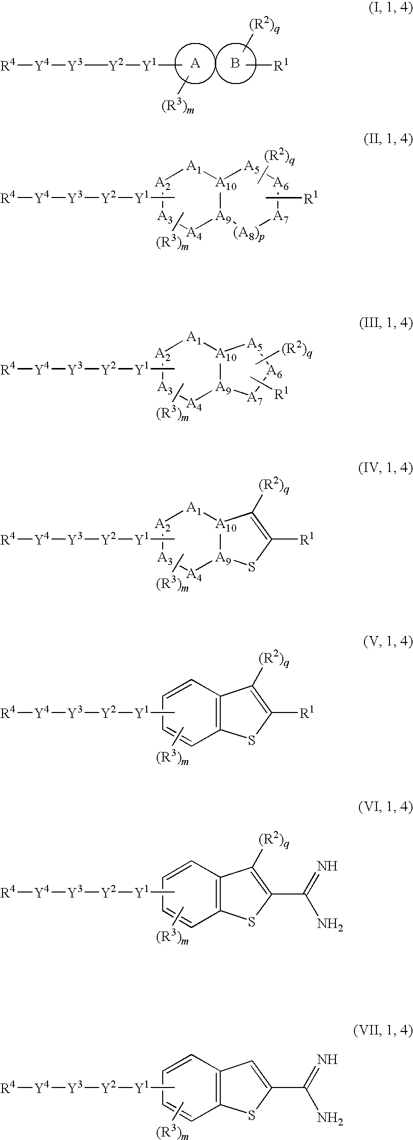

Particular embodiments of Formula (V, 1, 4) include those shown below, and pharmaceutically acceptable salts or prodrugs thereof:

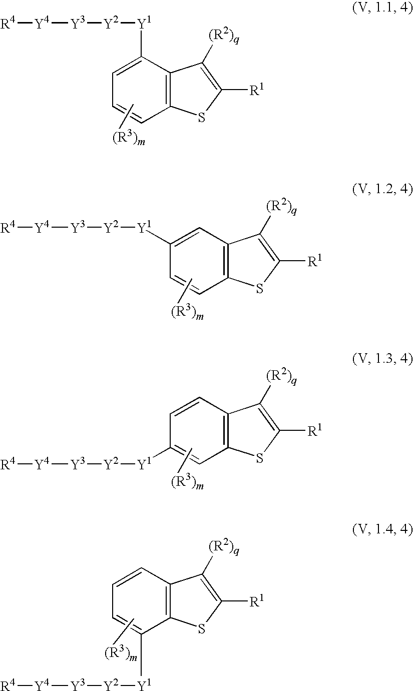

Particular embodiments of Formula (VI, 1, 4) include those shown below, and pharmaceutically acceptable salts or prodrugs thereof:

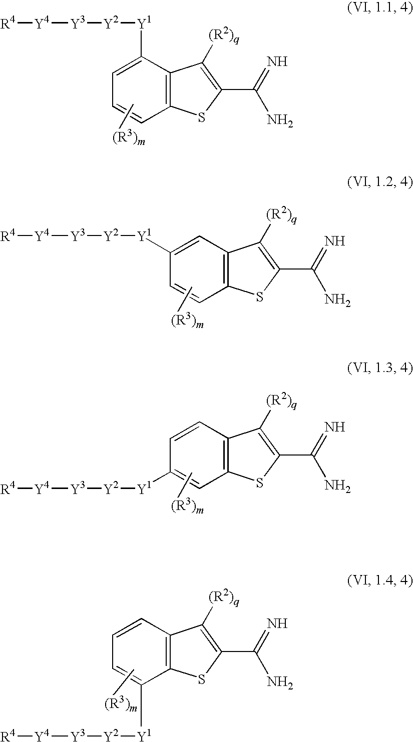

Particular embodiments of Formula (VII, 1, 4) include those shown below, and pharmaceutically acceptable salts or prodrugs thereof:

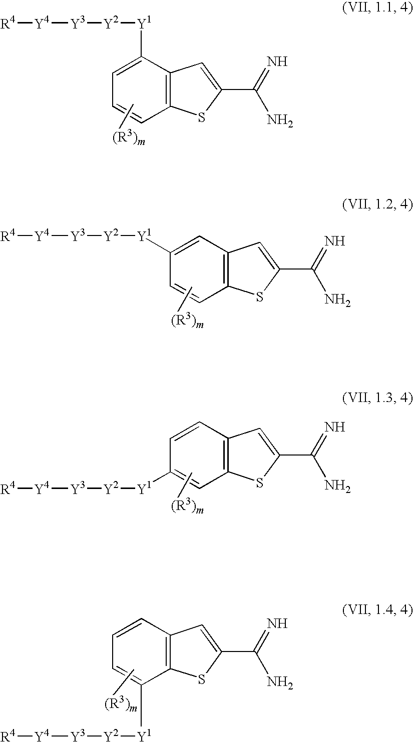

In the above embodiments, -Y¹-Y²-Y³-Y⁴- may be as defined in any of Tables 12, 13 and 14.

A particular embodiment of Formula (VII, 1, 4) is a compound of Formula (XIII):

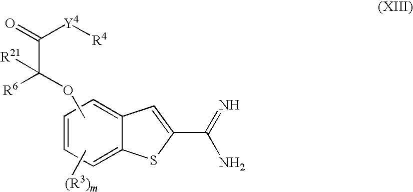

wherein R²¹ is hydrogen or R⁷;
or a pharmaceutically acceptable salt or prodrug thereof.

Embodiments of Formula (XIII) include the following, and pharmaceutically acceptable salts and prodrugs thereof:

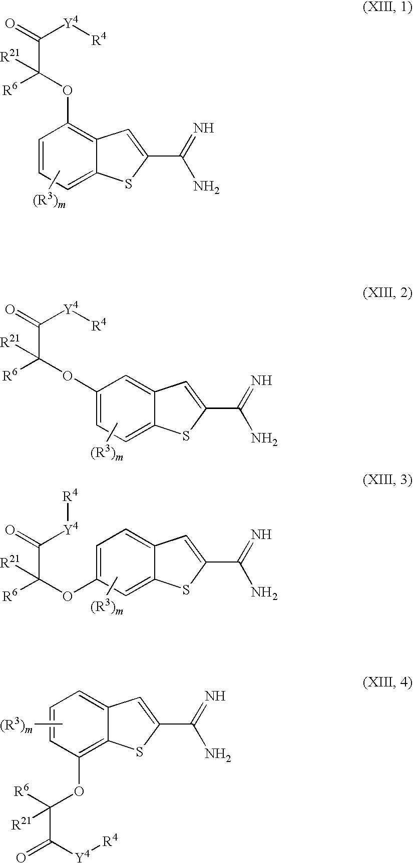

With regard to Formula (XIII), Y⁴ is often —O—, —N(R⁵)— (e.g. —NH—) or —CH₂—. R⁶ is usually hydrogen and R²¹ is typically hydrogen or is selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹. More usually, R⁶ is hydrogen and R²¹ is hydrogen, or is C₁₋₆ alkyl (e.g. methyl or ethyl) or phenyl, either of which is optionally substituted with 1, 2, 3, 4 or 5 R¹¹.

With regard to the various embodiments described in this section, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

Also, m is usually 0 or 1, and R³ is typically independently selected from halogen (e.g. fluorine, chlorine, bromine or iodine), hydroxy, trifluoromethyl, cyano, nitro, oxo, —OR¹², —C(O)R¹², —C(O)OR¹², —OC(O)R¹², —N(R¹²)R¹³, —C(O)N(R¹²)R¹³, —S(O)ₗR¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 1 and Y is -Y¹-Y¹-Y³-Y⁴-Y⁵-

In another embodiment, n is 1 and Y is -Y¹-Y²-Y³-Y⁴-Y⁵-.

Embodiments of Formulae (I) to (VII) in which n is 1 and Y is -Y¹-Y²-Y³-Y⁴-Y⁵- include those shown below, and pharmaceutically acceptable salts or prodrugs thereof:

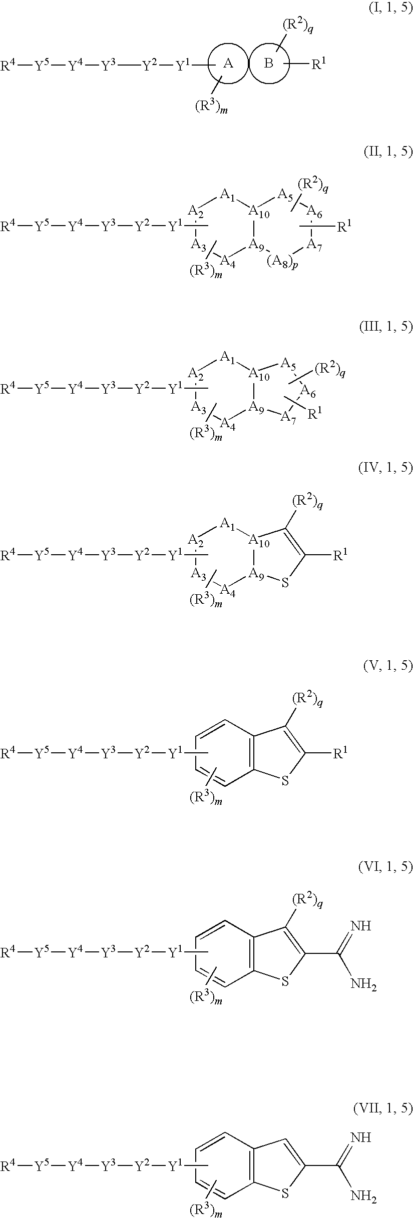

Particular embodiments of Formula (V, 1, 5) include those shown below, and pharmaceutically acceptable salts or prodrugs thereof:

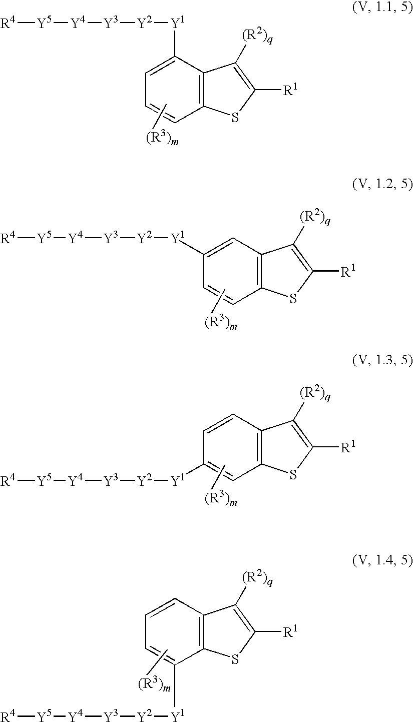

Particular embodiments of Formula (VI, 1, 5) include those shown below, and pharmaceutically acceptable salts or prodrugs thereof:

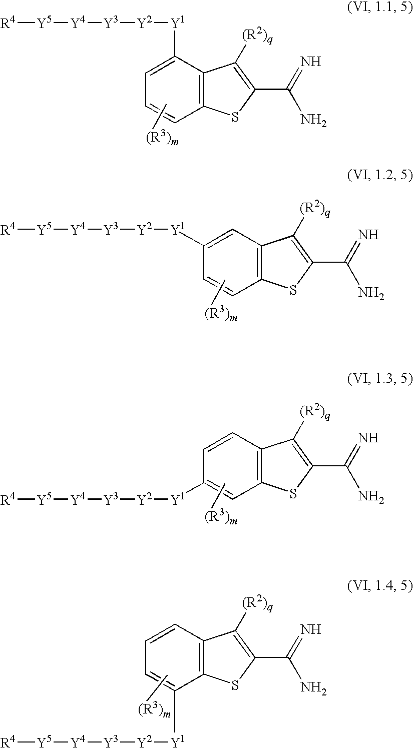

Particular embodiments of Formula (VII, 1, 5) include those shown below, and pharmaceutically acceptable salts or prodrugs thereof:

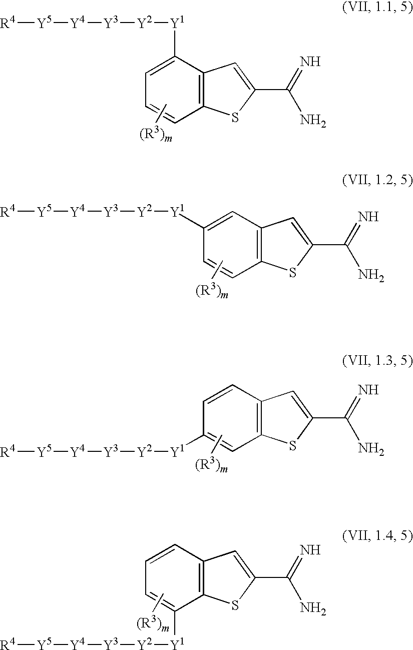

In the above embodiments, -Y¹-Y²-Y³-Y⁴-Y⁵- may be as defined in Table 15 or Table 16.

A particular embodiment of Formula (VII, 1, 5) is a compound of Formula (XIV):

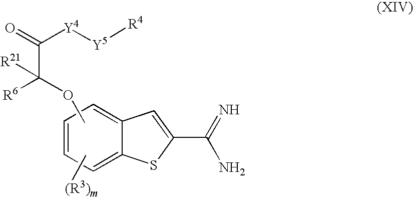

wherein R²¹ is hydrogen or R⁷;
or a pharmaceutically acceptable salt or prodrug thereof.

Embodiments of Formula (XIV) include the following, and pharmaceutically acceptable salts and prodrugs thereof:

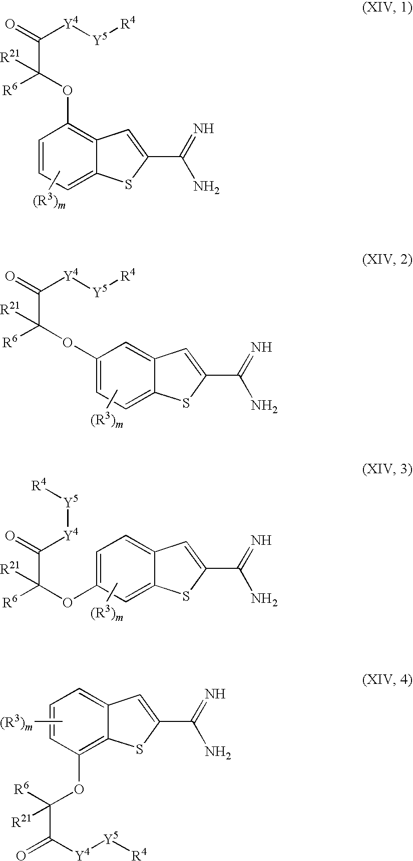

In compounds of Formula (XIV), Y⁴ is often —O—, —N(R⁵)— (e.g. —NH—) or —CH₂—; and Y⁵ is typically —CH₂—, carbocyclylene or heterocyclylene, any of which is optionally substituted with 1, 2, 3, 4 or 5 R¹¹.

A further embodiment of Formula (VII, 1, 5) is a compound of Formula (XV):

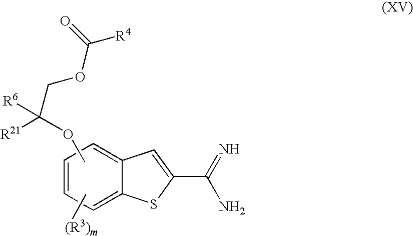

wherein R²¹ is hydrogen or R⁷;
or a pharmaceutically acceptable salt or prodrug thereof.

Embodiments of Formula (XV) include the following, and pharmaceutically acceptable salts and prodrugs thereof:

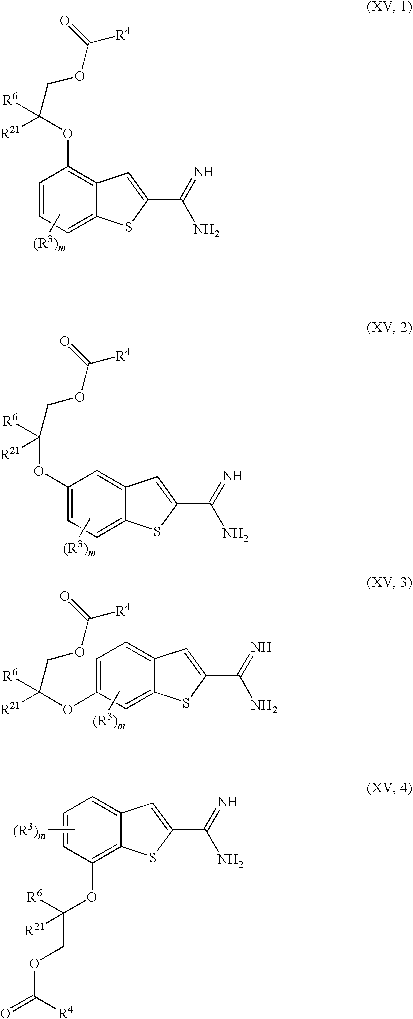

With regard to Formulae (XIV) and (XV), R⁶ is usually hydrogen and R²¹ is typically hydrogen or is selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹. More usually, R⁶ is hydrogen and R²¹ is hydrogen, or is C₁₋₆ alkyl (e.g. methyl or ethyl) or phenyl, either of which is optionally substituted with 1, 2, 3, 4 or 5 R¹¹.

With regard to the various embodiments described in this section, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

Also, m is usually 0 or 1, and R³ is typically independently selected from halogen (e.g. fluorine, chlorine, bromine or iodine), hydroxy, trifluoromethyl, cyano, nitro, oxo, —OR¹², —C(O)R¹², —C(O)OR¹², —OC(O)R¹², —N(R¹²)R¹³, —C(O)N(R¹²)R¹³, —S(O)ₗR¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 1 and Y is -Y¹-Y²-Y³-Y⁴-Y⁵-Y⁶-

In another embodiment, n is 1 and Y is -Y¹-Y²-Y³-Y⁴-Y⁵-Y⁶.

Embodiments of Formulae (I) to (VII) in which n is 1 and Y is -Y¹-Y²-Y³-Y⁴-Y⁵-Y⁶- include those shown below, and pharmaceutically acceptable salts or prodrugs thereof:

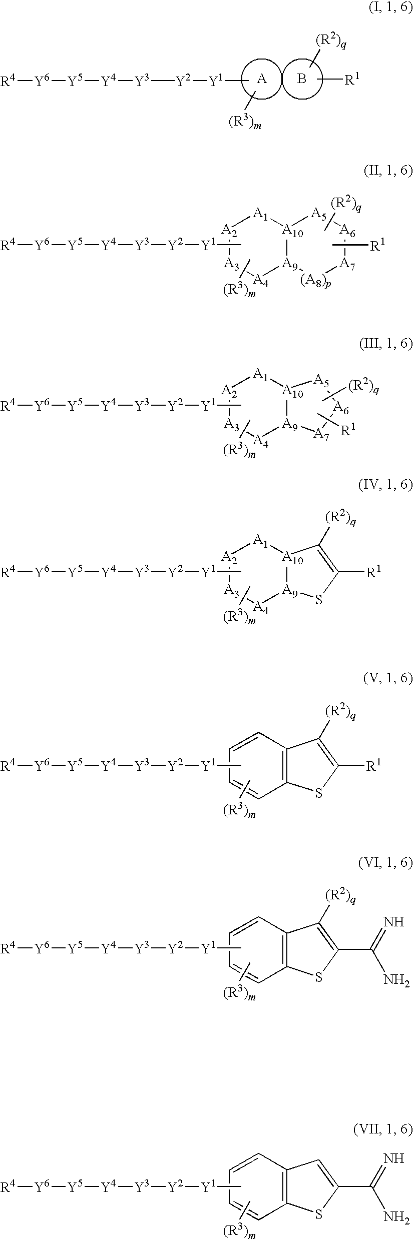

Particular embodiments of Formula (V, 1, 6) include those shown below, and pharmaceutically acceptable salts or prodrugs thereof:

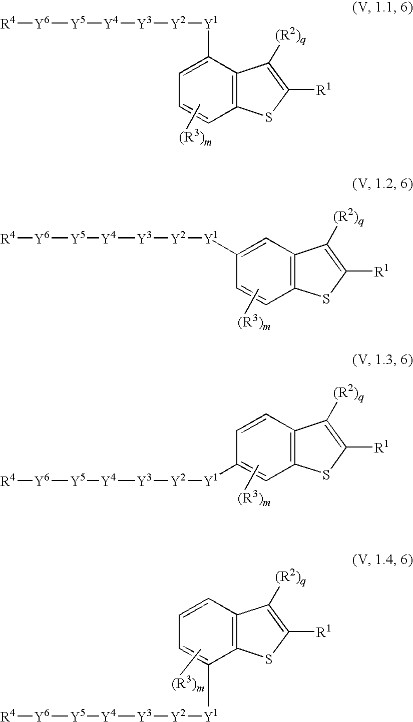

Particular embodiments of Formula (VI, 1, 6) include those shown below, and pharmaceutically acceptable salts or prodrugs thereof:

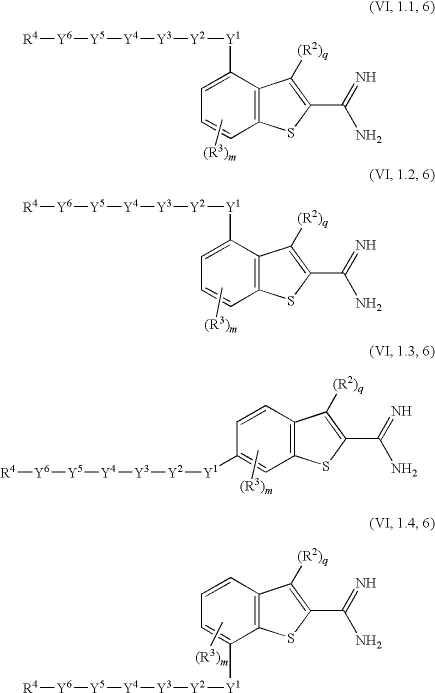

Particular embodiments of Formula (VII, 1, 6) include those shown below, and pharmaceutically acceptable salts or prodrugs thereof:

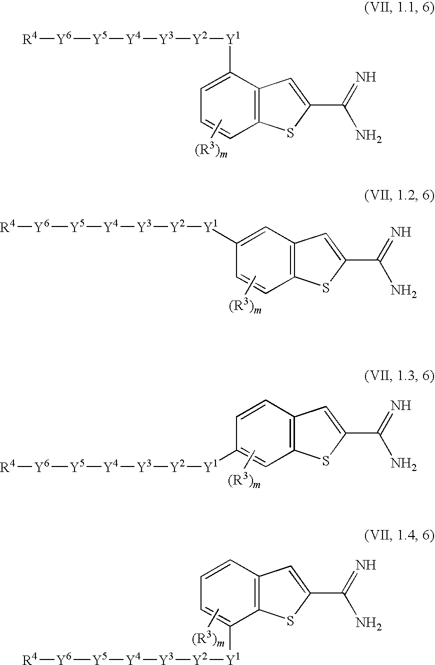

In the above embodiments, -Y¹-Y²-Y³-Y⁴-Y⁵-Y⁶- may be as defined in any of Tables 17, 18 and 19.

A particular embodiment of Formula (VII, 1, 6) is a compound of Formula (XVI):

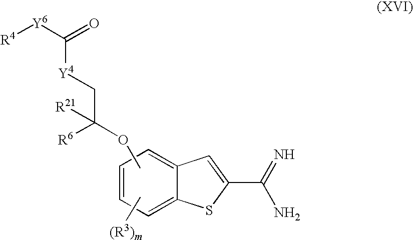

wherein R²¹ is hydrogen or R⁷;
or a pharmaceutically acceptable salt or prodrug thereof.

Embodiments of Formula (XVII) include the following, and pharmaceutically acceptable salts and prodrugs thereof:

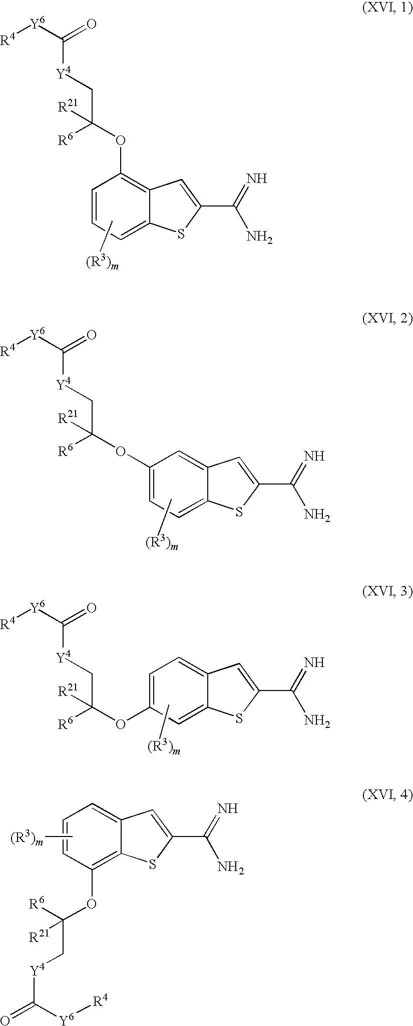

With regard to Formula (XVI), Y⁴ and Y⁶ are typically each independently —O— or —N(R⁵)— (e.g. —NH)—. R⁶ is usually hydrogen and R²¹ is typically hydrogen or is selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹. More usually, R⁶ is hydrogen and R²¹ is hydrogen, or is C₁₋₆ alkyl (e.g. methyl or ethyl) or phenyl, either of which is optionally substituted with 1, 2, 3, 4 or 5 R¹¹.

With regard to the various embodiments described in this section, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

Also, m is usually 0 or 1, and R³ is typically independently selected from halogen (e.g. fluorine, chlorine, bromine or iodine), hydroxy, trifluoromethyl, cyano, nitro, oxo, —OR¹², —C(O)R¹², —C(O)OR¹², —OC(O)R¹², —N(R¹²)R¹³, —C(O)N(R¹²)R¹³, —S(O)ₗR¹², —C(R¹²)₃ and R¹⁴. In this case, R¹² and R¹³ are usually each independently hydrogen or selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R¹⁴ is often selected from C₁₋₆ alkyl (e.g. methyl, ethyl, propyl or butyl), —(CH₂)ⱼ-aryl (e.g. phenyl or benzyl) and —(CH₂)ⱼ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

Compounds in which n is 2

In another class of compounds, n is 2.

Embodiments of Formulae (I) to (VII) in which n is 2 include those shown below, and pharmaceutically acceptable salts or prodrugs thereof:

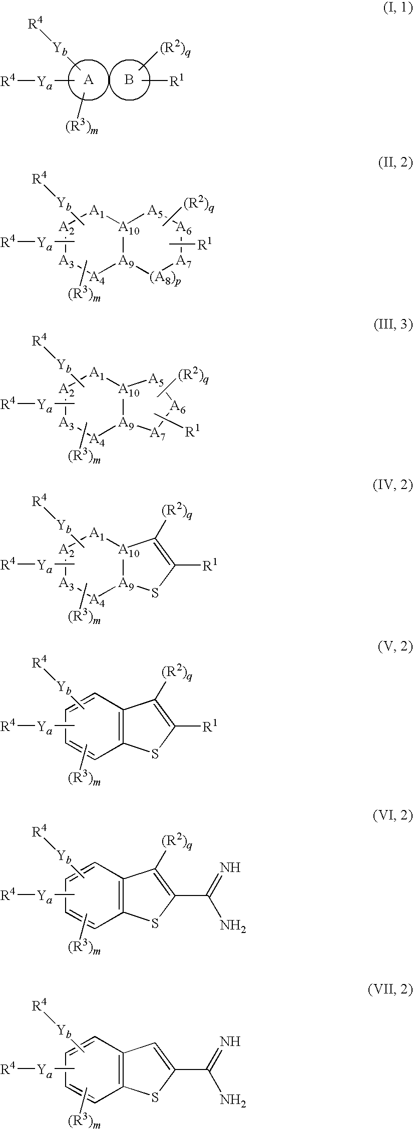

[The labels “a” and “b” are used to distinguish between each Y]

Particular embodiments of Formula (V, 2) include those shown below, and pharmaceutically acceptable salts or prodrugs thereof:

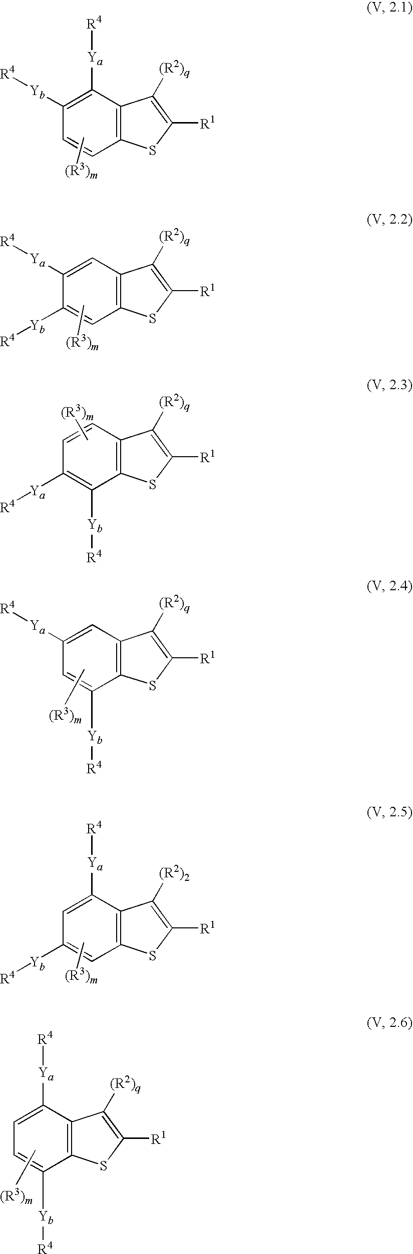

Particular embodiments of Formula (VI, 2) include those shown below, and pharmaceutically acceptable salts or prodrugs thereof:

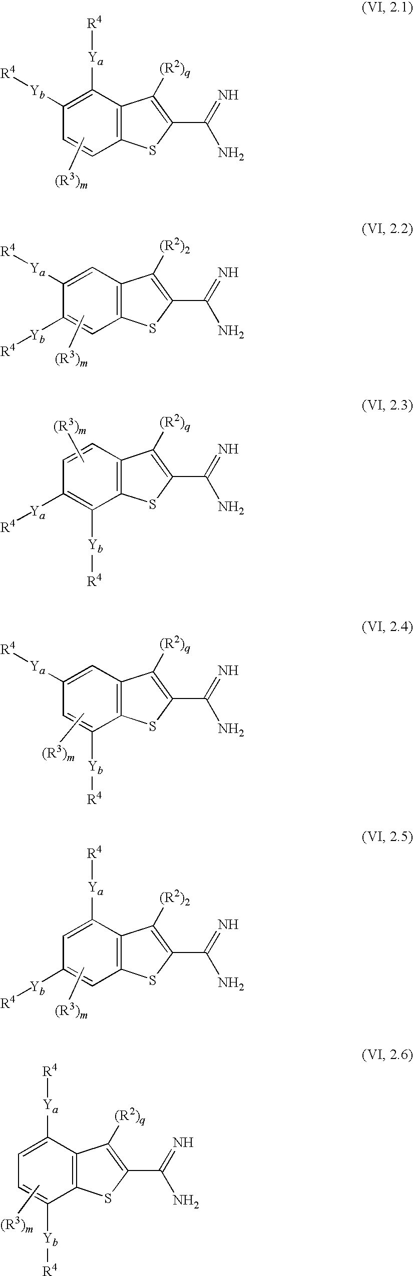

Particular embodiments of Formula (VII, 1) include those shown below, and pharmaceutically acceptable salts or prodrugs thereof:

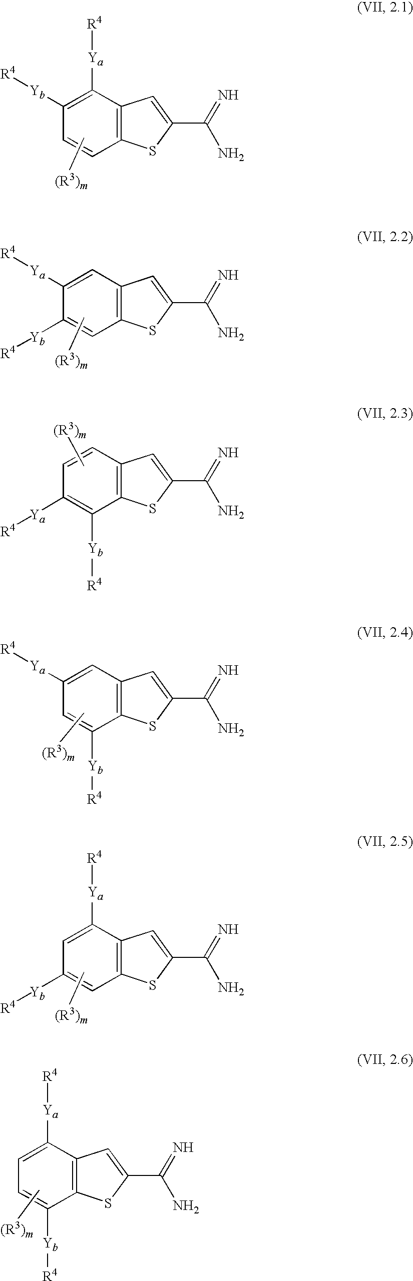

Of particular mention are compounds of Formulae (V, 2.5), (VI, 2.5) and (VII, 2.5), in which —Y, —R⁴ and -Y_{b}-R⁴ may be the same or different.

In the above embodiments, Yₐ and Y_{b} may each be independently a bond or a linker, in particular a linker selected from:
-Y¹-;
-Y¹-Y²-;
-Y¹-Y²-Y³-;
-Y¹-Y²-Y³-Y⁴;
-Y¹-Y²-Y³-Y⁴-Y⁵-;
-Y¹-Y²-Y³-Y⁴- Y⁵-Y⁶-;
-Y¹-Y²-Y³-Y⁴- Y⁵-Y⁶-Y⁷-;
-Y¹-Y²-Y³-Y⁴-Y⁵-Y⁶-Y⁷-Y⁸-;
-Y¹-Y²-Y³-Y⁴-Y⁵-Y⁶-Y⁷-Y⁸-Y⁹-; and
-Y¹-Y²-Y³-Y⁴-Y⁵-Y⁶-Y⁷-Y⁸-Y⁹-Y¹⁰-,

In particular, Yₐ and Y_{b} may each be independently selected from any of the options defined in Tables 2 to 21.

With regard to the various embodiments described in this section, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

Compounds in which n is 1 and Y is —O—C(R²²)(R²³)—Y′-

Also of mention are compounds of the following formula:

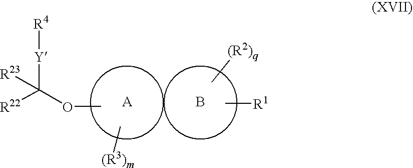

wherein
Y′ is a bond or a linker having 1 to 18 (e.g. 1 to 10) in-chain atoms (e.g. selected from C, N, O and S) and comprising, for example, one or more linkages selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C≡C—, carbocyclylene optionally substituted with 1, 2, 3, 4 or 5 R¹¹, and heterocyclylene optionally substituted with 1, 2, 3, 4 or 5 R¹¹;
R²² is carbocyclyl or heterocyclyl, either of which is optionally substituted with 1, 2, 3, 4 or 5 R¹¹; and
R²³ is independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃;
   or a pharmaceutically acceptable salt or prodrug thereof.

The invention therefore includes compounds of the following formulae:

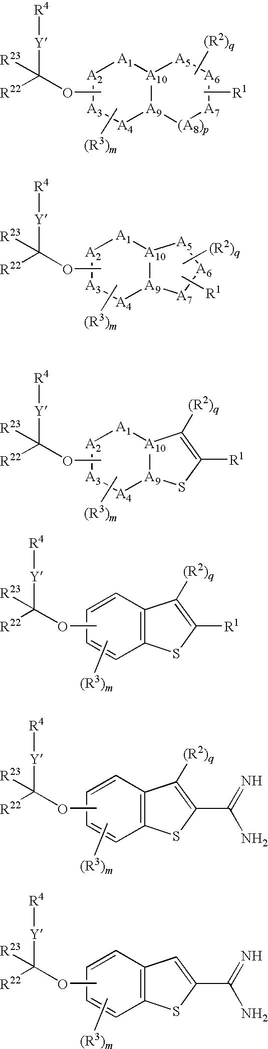

or, in each case, a pharmaceutically acceptable salt or prodrug thereof.

In particular, the invention includes compounds of the following formulae:

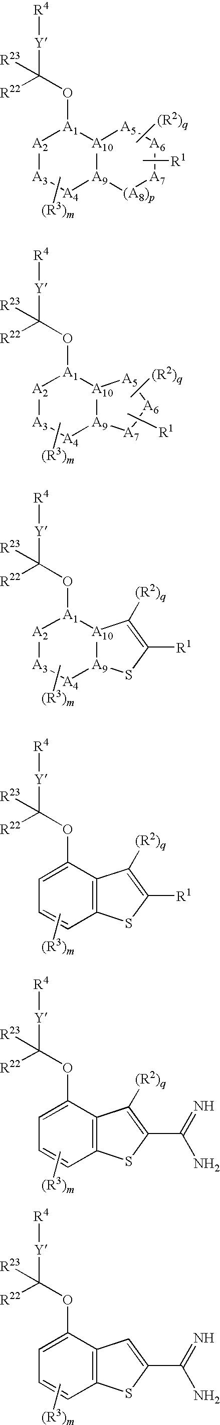

or, in each case, a pharmaceutically acceptable salt or prodrug thereof.

Of particular mention is a compound of the following formula:

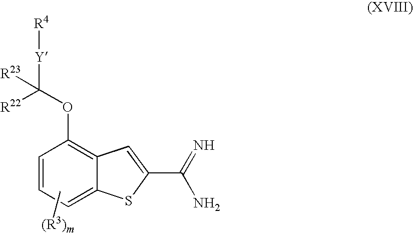

or a pharmaceutically acceptable salt or prodrug thereof.

With regard to said compounds, R²² is carbocyclyl or heterocyclyl, either of which is optionally substituted with 1, 2, 3, 4 or 5 R¹¹. In certain compounds, R²² is carbocyclyl, for example, selected from cycloalkyl (e.g. cyclopropyl or cyclohexyl) and aryl (e.g. phenyl or naphthyl), either of which is optionally substituted with 1, 2, 3, 4 or 5 R¹¹. In other compounds, R²² is heterocyclyl, for example, selected from morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl, any of which is optionally substituted with 1, 2, 3, 4 or 5 R¹¹. Often, R²² is monocyclic carbocyclyl or monocyclic heterocyclyl, e.g. containing 3, 4, 5, 6 or 7 ring atoms. In particular compounds, R²² is selected from phenyl, cyclopropyl or pyridinyl, any of which is optionally substituted with 1, 2, 3, 4 or 5 R¹¹. Of particular mention are compounds in which R²² is unsubstituted phenyl or phenyl optionally substituted with 1, 2 or 3 substituents independently selected from hydroxy, C₁₋₆ alkyl (e.g. C₁, C₂, C₃ or C₄ alkyl) and C₁₋₆ alkoxy (e.g. C₁, C₂, C₃ or C₄ alkoxy) and up to 5 halogens (e.g. fluorine or chlorine), wherein alkyl and the alkyl part of alkoxy are unsubstituted or are substituted by halogen, e.g. F or Cl, for example by 1, 2, 3, 4 or 5 halogens.

In certain compounds, R²³ is hydrogen or C₁₋₆ alkyl (e.g. C₁, C₂, C₃ or C₄ alkyl) optionally substituted with 1, 2, 3, 4 or 5 R¹¹. R²³ is often hydrogen.

Of mention are compounds in which R²² is selected from phenyl, cyclopropyl and pyridinyl, any of which is optionally substituted with 1, 2, 3, 4 or 5 R¹¹; and R²³ is hydrogen or C₁₋₆ alkyl (e.g. C₁, C₂, C₃ or C₄ alkyl) optionally substituted with 1, 2, 3, 4 or 5 R¹¹. Of particular mention are compounds in which R²² is phenyl optionally substituted with 1, 2, 3, 4 or 5 R¹¹, and R²³ is hydrogen.

In a further embodiment, the invention provides a compound of the following formula:

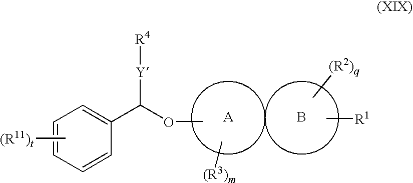

wherein t is 0, 1, 2, 3, 4 or 5;
   or a pharmaceutically acceptable salt or prodrug thereof. In a class of compounds, R¹¹ is halogen, e.g. F or Cl; in some compounds, R¹¹ is additionally selected from C₁₋₄ alkyl or C₁₋₄ alkoxy, optionally substituted in each case by up to 5 halogens.

Thus, the invention includes compounds of the following formulae:

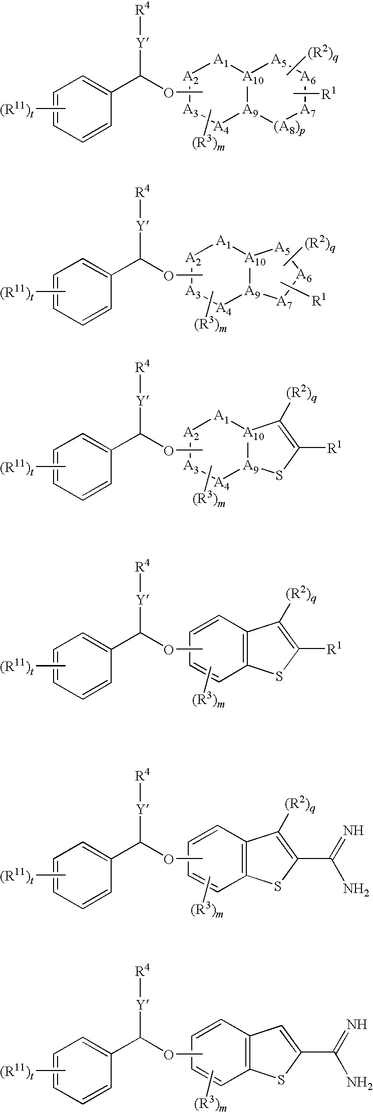

or, in each case, a pharmaceutically acceptable salt or prodrug thereof.

In particular, the invention includes compounds of the following formulae:

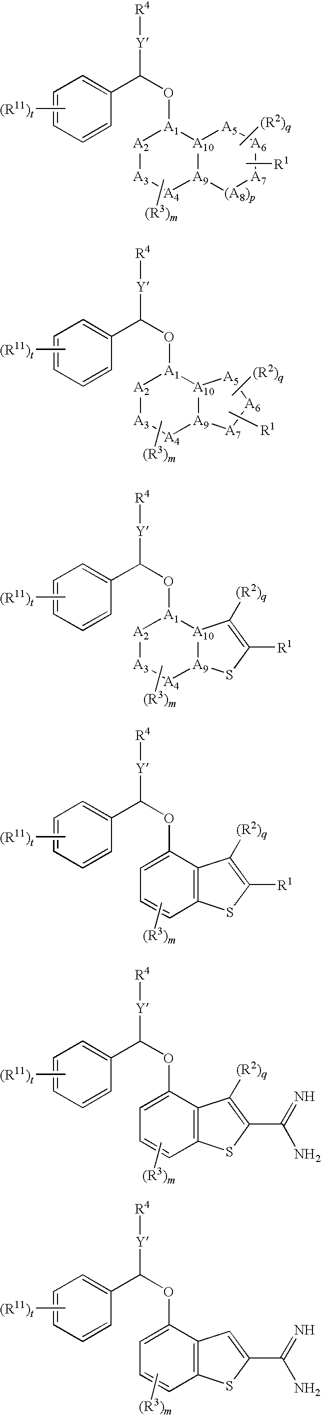

or, in each case, a pharmaceutically acceptable salt or prodrug thereof.

Of particular mention is a compound of the following formula:

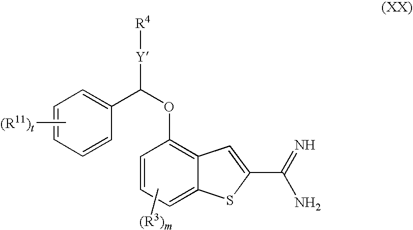

or a pharmaceutically acceptable salt or prodrug thereof.

With regard to the above compounds, Y′ may, for example, contain at least one linkage selected from —O—, —N(R⁵)—, —C(O)—, —S(O)ₗ—, —(CH₂)ₖ— and —C(R⁶)(R⁷)—. In embodiments, Y contains at least two or said linkages, for example two, three or four of said linkages. Of mention are compounds in which Y′ comprises at least one —C(O)— linkage. Also of mention are compounds in which Y′ comprises at least one heterocyclylene linkage. Of further mention are compounds in which Y′ comprises two, three, four or five in-chain atoms.

In certain compounds, -Y′-R⁴ is a group of the following formula:

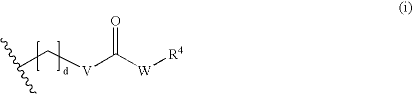

wherein
V and W are each independently selected from —O—, —N(R⁵)—, —(CH₂)ₖ— and —C(R⁶)(R⁷)—; and
d is 1, 2 or 3.

Exemplary -Y′-R⁴ groups include the following:

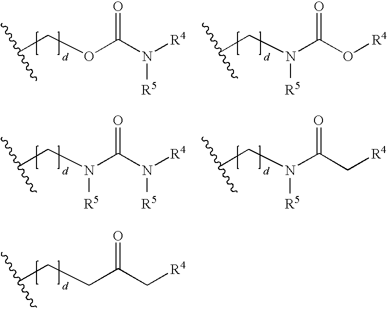

In certain compounds, d is 1 or 2. Of mention are compounds in which d is 1.

With regard to the various formulae described in this section, R⁴ may, in particular, be selected from C₁₋₆ alkyl (e.g. C₁, C₂, C₃ or C₄ alkyl), cycloalkyl (e.g. cyclopropyl or cyclohexyl), aryl (e.g. phenyl or naphthyl) and heterocyclyl (e.g. morpholinyl, pyridinyl, piperazinyl, benzothiophenyl, thiophenyl, pyrimidinyl, isoxazolyl, furazanyl, furanyl, benzothiazolyl, thiazolyl, pyrrolyl, triazolyl, thiadiazolyl, pyrazolyl, pyrazinyl, benzimidazolyl, quinolyl, isoquinolyl, benzoxazolyl, benzofurazanyl, piperidinyl, pyrrolidinyl or 1,4-benzodioxanyl), any of which is optionally substituted with 1, 2, 3, 4 or 5 R¹¹.

Of mention are compounds in which R⁴ is carbocyclyl or heterocyclyl, either of which is optionally substituted with 1, 2, 3, 4 or 5 R¹¹.

Also of mention are compounds in which R⁴ comprises a basic moiety, for example a basic nitrogen atom. Said basic moiety may be present in, for example, a carbocyclyl or heterocyclyl group, or in an R¹¹ substituent. In certain compounds, R⁴ is carbocyclyl or heterocyclyl, either of which is substituted with 1, 2, 3, 4 or 5 R¹¹, wherein at least one R¹¹ comprises a basic moiety. Said R¹¹ may, for example, comprise a basic nitrogen atom. In other compounds, R⁴ is heterocyclyl optionally substituted with 1, 2, 3, 4 or 5 R¹¹, wherein said heterocyclyl group comprises at least one basic nitrogen atom.

In certain compounds, R⁴ is selected from phenyl, pyrrolyl, imidazolyl, pyrazolyl, triazole and pyridinyl, any of which is optionally substituted with 1, 2, 3, 4 or 5 R¹¹.

With regard to compounds of said formulae, m is usually 0 or 1. Of mention are compounds in which m is 0. Also of mention are compounds in which m is 1 and R³ is selected from halogen (e.g. fluorine, chlorine, bromine or iodine), hydroxy, trifluoromethyl, cyano and nitro. Particular compounds include those in which m is 0 and those in which m is 1 and R³ is fluorine.

### Particular Benzothiophene Compounds

The symbols used in this section of the specification are as defined in the section.

In another aspect, the present invention provides benzothiophene, e.g. amidinobenzothiophene, Factor IXa inhibitors which comprise a substituent at one or both of the 4- and 6-positions, wherein the or each substituent comprises a fragment independently selected from any of Formulae (i) to (vi):

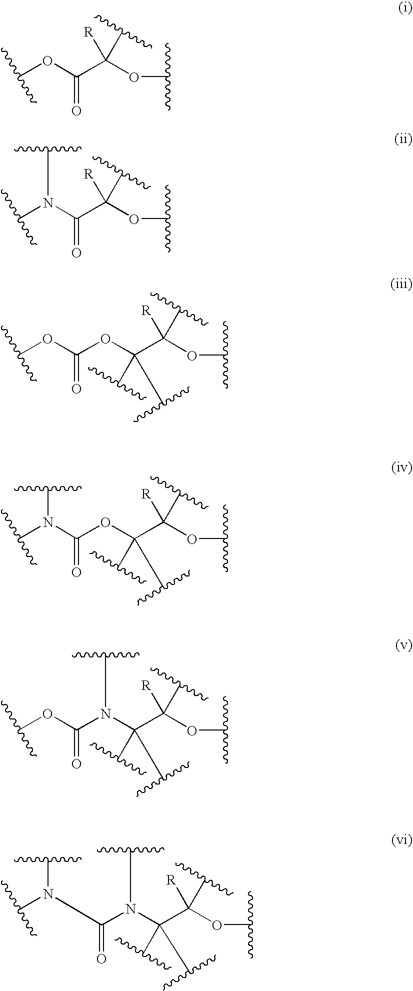

wherein
R is a moiety comprising an optionally substituted carbocyclic or heterocyclic group; and
the oxygen atom on the right hand side of the fragment as drawn is bound directly to the 4- or 6-carbon atom of the benzothiophene ring;
   or a pharmaceutically acceptable salt or prodrug thereof.

In a particular embodiment, the compounds are 2-amidinobenzothiophene compounds.

### Benzothiophene Ring

The 2-, 4- and 6-positions of benzothiophene are indicated below:

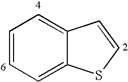

The benzothiophene ring may comprise one or more other substituents in addition to those mentioned above. The identity and number of any other substituents is not critical to this aspect of the invention. What is critical to this aspect is that the compound, a Factor IXa inhibitor, has the described substituent at one or both of the 4- and 6-positions. In embodiments, the thiophene part of benzothiophene is unsubstituted except for a 2-substituent selected from halo and Formula (A):

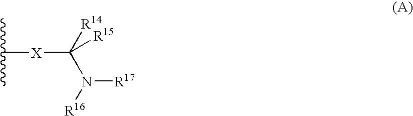

wherein
X is a bond, —NR³⁰— or —C(O)—;
R¹⁴, R¹⁵ and R³⁰ are each independently selected from R¹⁸, —OR¹⁸, —C(O)R¹⁸, —C(O)OR¹⁸, —OC(O)R¹⁸, —N(R¹⁸)R¹⁹, —C(O)N(R¹⁹)R²⁰—S(O)ₗR¹⁸ and —C(R¹⁸)₃, e.g. are hydrogen, hydroxy or C₁₋₆ alkyl;
or R¹⁴ and R¹⁵ taken together form ═NR²⁰, ═O or ═S;
R¹⁶ and R¹⁷ are each independently selected from hydrogen, C₁₋₆ alkyl, —OR²¹ and —NR¹⁸R¹⁹;
R¹⁸ and R¹⁹ are each independently selected from hydrogen, R¹¹, hydrocarbyl optionally substituted with 1, 2, 3, 4 or 5 R¹¹; and heterocyclyl optionally substituted with 1, 2, 3, 4 or 5 R¹¹;
R²⁰ hydrogen, hydroxy, C₁₋₆ alkoxy or C₁₋₆ alkyl, e.g. is hydrogen or C₁₋₆ alkyl; and
R²¹ is hydrogen or R⁷.

In one embodiment of Formula (A), X is a bond or —N(R³⁰)—.

In another embodiment, X is a bond.

In one class of compounds, R¹⁴ and R¹⁵ together form NR²⁰ and R¹⁶, R¹⁷ and R²⁰ are each the same or different and selected from hydrogen, alkyl and hydroxy; for example they may be selected from hydrogen and hydroxy or from hydrogen and alkyl. Alkyl may have 1, 2, 3, 4, 5 or 6 carbon atoms.

In a further embodiment, R¹⁴ and R¹⁵ taken together form ═NR²⁰, wherein R²⁰ is usually hydrogen, alkyl or hydroxy, e.g. hydrogen or hydroxy. Accordingly, the invention includes compounds in which R¹ is of Formula (B):

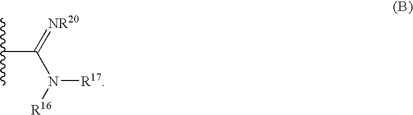

Included in the invention are compounds in which R¹⁶ and R¹⁷ are each independently selected from hydrogen, C₁₋₆ alkyl, C₁₋₆ alkoxy or hydroxy, e.g. hydrogen or C₁₋₆ alkyl.

In embodiments, R¹⁶ and/or R¹⁷ and/or R²⁰ are hydrogen.

In a particular embodiment, the 2-substituent is a group of Formula (C):

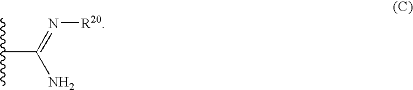

In a further embodiment, R¹ is —C(═NH)NH₂ or —C(═NOH)NH₂, particularly —C(═NH)NH₂ (amidino).

The 2-substituent is in particular amidino. Where the thiophene part is additionally substituted, it may be substituted with halogen or moieties having 1 or 2 plural valent atoms, for example, F, Cl, methyl, methoxy, ethyl and trifluoroethyl.

The benzene part of the benzothiophene ring may be substituted with 1, 2 or 3 substituents selected from (i) halogen; (ii) moieties having from 1 to 30 plural valent atoms (e.g. 1 to 20, for example 1 to 10, exemplary moieties having 1, 2, 3 or 4 plural valent atoms), typically selected from C, N, O and S as well as monovalent atoms selected from H and halogen, e.g. selected from hydrogen, F, Cl and Br, for example hydrogen, F and Cl.

In particular the benzene part may be substituted with from 1 to 5, e.g. 1, 2 or 3, R^{a}, wherein:

each R^{a} is independently selected from halogen, hydroxy, trifluoromethyl, cyano, nitro, oxo, amidino, —B(OH)₂, ═NR^{b}, OR^{b}, —SR^{b}, —C(O)R^{b}, —C(O)OR^{b}, —OC(O)R^{b}, N(R^{b})R^{c}, —C(O)N(R^{b})R^{c}, OC(O)N(R^{b})R^{c}, —S(O)ₗR^{b}, —S(O)ₗNR^{b}R^{c}, —S(O)ₗNR^{b}C(O)R^{c}, —S(O)ₗNR^{c}C(O)OR^{b}, —NR^{c}C(O)R^{b}, NR^{c}C(O)OR^{b}, —NR^{c}S(O)ₗR^{b}, —NR^{c}C(O)NR^{b}R^{c}, —C(R^{b})₃ and R^{d};
R^{b} and R^{c} are the same or different and are each hydrogen or are selected from C₁₋₆ acyclic aliphatic groups and particularly C₁₋₆ alkyl, carbocyclyl optionally substituted by a C₁₋₆ acyclic aliphatic group and bonded to the remainder of the molecule either directly or through a C₁₋₆ acyclic aliphatic group (particularly —(CH₂)ⱼ-carbocyclyl or —(CH₂)ⱼ-carbocyclyl(C₁-C₆)alkyl), and heterocyclyl optionally substituted by a C₁₋₆ acyclic aliphatic group and bonded to the remainder of the molecule either directly or through a C₁₋₆ acyclic aliphatic group (particularly —(CH₂)ⱼ-heterocyclyl or —(CH₂)ⱼ-heterocyclyl(C₁-C₆)alkyl), any of which is optionally substituted with 1, 2, 3, 4 or 5 substituents independently selected from halogen, hydroxy, trifluoromethyl, cyano, nitro, oxo, amidino, —B(OH)₂, ═NR^{u}, —OR^{v}, —SR^{v}, —C(O)R^{v}, —C(O)OR^{v}, —OC(O)R^{v}, —N(R^{u})R^{v}, C(O)N(R^{u})R^{v}, —OC(O)N(R^{u})R^{v}, —S(O)ₗR^{v}, —S(O)ₗNR^{u}R^{v}, —S(O)ₗNR^{u}C(O)R^{v}, —S(O)ₗNR^{u}C(O)OR^{v}, —NR^{u}C(O)R^{v}, —NR^{u}C(O)OR^{v}, —NR^{u}S(O)ₗR^{v}, —NR^{u}C(O)NR^{v}R^{u}, —C(R^{v})₃, and C₁₋₆ alkyl optionally substituted by 1, 2, 3, 4 or 5 halogens, where R^{u} is H, OH or C₁₋₆ alkyl optionally substituted by up to 5 halogens and R^{v} is H or C₁₋₆ alkyl optionally substituted by up to 5 halogens, e.g. R^{b} and R^{c} are the same or different and are each hydrogen or are selected from C₁₋₆ alkyl, —(CH₂)ⱼ-carbocyclyl and —(CH₂)ⱼ-heterocyclyl, any of which is optionally substituted with 1, 2, 3, 4 or 5 substituents independently selected from halogen, hydroxy and C₁₋₆ alkyl;
R^{c} additionally may be hydroxy or C₁₋₆ alkoxy;
R^{d} is selected from C₁₋₆ acyclic aliphatic groups and particularly C₁₋₆ alkyl, C₁₋₆ acyclic aliphatic-oxy and particularly C₁₋₆ alkoxy, —(CH₂)ᵢ—O—(CH₂)ⱼ-carbocyclyl, —(CH₂)ᵢ—O—(CH₂)ⱼ-heterocyclyl, —(CH₂)ᵢ—O—(CH₂)ⱼ-carbocyclyl(C₁-C₆)alkyl and —(CH₂)ᵢ—O—(CH₂)ⱼ-heterocyclyl(C₁-C₆)alkyl any of which is optionally substituted with 1, 2, 3, 4 or 5 substituents independently selected from halogen, hydroxy, trifluoromethyl, cyano, nitro, oxo, amidino, —B(OH)₂, ═NR^{b}, —OR^{b}, —SR^{b}, —C(O)R^{b}, —C(O)OR^{b}, —OC(O)R^{b}, —N(R^{b})R^{c}, —C(O)N(R^{b})R^{c}, —OC(O)N(R^{b})R^{c}, —S(O)ₗR^{b}, —S(O)ₗNR^{b}R^{c}, —S(O)ₗNR^{b}C(O)R^{c}, —S(O)ₗNR^{c}C(O)OR^{b}, —NR^{c}C(O)R^{b}, —NR^{c}C(O)OR^{b}, —NR^{c}S(O)ₗR^{b}, —NR^{c}C(O)NR^{b}R^{c}, and C(R^{b})₃;
wherein:
i and j are the same or different and are 0, 1, 2, 3, 4, 5 or 6.

In an embodiment:
each R¹ is independently selected from each halogen, hydroxy, trifluoromethyl, cyano, nitro, oxo, amidino, —B(OH)₂, ═NR^{b}, OR^{b}, —C(O)R^{b}, —C(O)OR^{b}, —OC(O)R^{b}, —N(R^{b})R^{c}, —C(O)N(R^{b})R^{c}, —S(O)ₗR^{b}, —C(R^{b})₃ and R^{d};
R^{b} and R^{c} are each independently hydrogen or selected from C₁₋₆ alkyl, —(CH₂)ₖ-carbocyclyl and —(CH₂)ₖ-heterocyclyl, any of which is optionally substituted with 1, 2, 3, 4 or 5 substituents independently selected from halogen, hydroxy and C₁₋₆ alkyl;
R^{d} is selected from C₁₋₆ alkyl, C₁₋₆ alkoxy, —(CH₂)ₖ-carbocyclyl and —(CH₂)ₖ-heterocyclyl, any of which is optionally substituted with 1, 2, 3, 4 or 5 substituents independently selected from halogen, hydroxy, C₁₋₆ alkyl and C₁₋₆ alkoxy;
k is 0, 1, 2, 3, 4, 5 or 6; and
l is 0, 1 or 2.

Usually, the or each R^{a} is halogen (e.g. fluorine or chlorine) or is an inert organic group, for example C₁₋₆ alkyl (e.g. methyl or ethyl) or C₁₋₆ alkoxy (e.g. methoxy or ethoxy), either of which is optionally substituted by halogen (e.g. fluorine or chlorine, as in the case of trifluoromethyl). As mentioned above, where an alkyl or alkyl-substituted group is mentioned, alkyl typically has 1, 2, 3 or 4 carbon atoms.

In some compounds of the invention, the benzene part is substituted once or twice, having a first substituent which comprises a fragment as defined herein; and an optional second substituent which is R^{a} (which in turn is usually halogen, e.g. fluorine or chlorine).

In other compounds, the benzothiophene ring is substituted at both of the 4- and 6-positions by fragments, which may be the same of different, of any of Formulae (i) to (vi). In one class of compounds, the 4-fragment is of the same formula as the 6-fragment. Thus, for example, the 4- and 6-substituents may be identical, or may be different but share a common formula. In another class, the 4-fragment is of a different formula to the 6-fragment.

Included are compounds substituted at the 4-position by a fragment of Formula (ii), (iii), (iv), (v) or (vi); at the 6-position by a fragment of Formula (i); or at the 4-position by a fragment of Formula (ii), (iii), (iv), (v) or (vi) and at the 6-position by a fragment of Formula (i). Of particular mention are compounds substituted at the 4-position by a fragment of Formula (ii); at the 6-position by a fragment of Formula (i); or at the 4-position by a fragment of Formula (ii) and at the 6-position by a fragment of Formula (i).

### Fragment (i)

A compound of the invention may comprise, at the 4- and/or 6-position, a substituent comprising a fragment of the Formula (i):

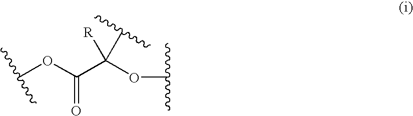

wherein R is as previously defined.

As previously stated, and as applies to each of fragments (i) to (vi), the oxygen atom on the right hand side of the fragment as drawn is bound directly to the 4- or 6-carbon atom of the benzothiophene ring.

In one class of compounds, a substituent comprising a fragment of Formula (i) is bound at the 4-position of the benzothiophene ring.

In another class of compounds, a substituent comprising a fragment of Formula (i) is bound at the 6-position of the benzothiophene ring.

The fragment of Formula (i) may be selected from one of the following fragments, particularly when the substituent is present at the 6-position:

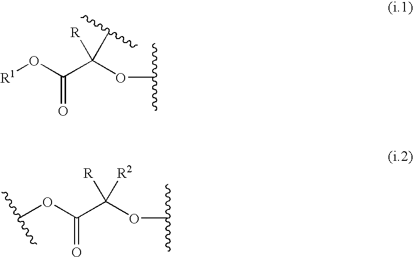

wherein
R¹ is hydrogen or is selected from C₁₋₆ alkyl, —(CH₂)ₖ-carbocyclyl and —(CH₂)ₖ-heterocyclyl, any of which is optionally substituted with 1, 2, 3, 4 or 5 R^{a};
R² is selected from hydrogen and C₁₋₆ alkyl optionally substituted with 1, 2, 3, 4 or 5 R^{a}; and
k is 0, 1, 2, 3, 4, 5 or 6.

Thus, a substituent comprising fragment (i) may be of the following Formula:

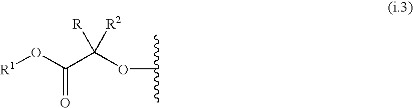

In one class of compounds, R¹ is hydrogen, or C₁₋₆ alkyl optionally substituted with 1, 2, 3, 4 or 5 R^{a}. In particular, R¹ may be hydrogen, methyl or ethyl.

In another class of compounds, R² is hydrogen or C₁, C₂, C₃ or C₄ alkyl optionally substituted with 1, 2, 3, 4 or 5 R^{a}. In particular, R² may be hydrogen or methyl.

In a further class of compounds, R comprises (e.g. is) aryl or heteroaryl, either of which is optionally substituted with 1, 2, 3, 4 or 5 R^{a}. Aryl and heteroaryl are often monocyclic, e.g. having 5 or 6 ring members, being, for example, phenyl or thiophenyl. Sometimes, aryl and heteroaryl are polycyclic, e.g. bicyclic, having, for example, 8, 9 or 10 ring members. In particular, R may be phenyl or thiophenyl (also called thienyl), either of which is optionally substituted with 1, 2, 3, 4 or 5 R^{a}. Where R is thiophenyl, it may be thiophen-2-yl.

Of particular mention are fragments of the following Formulae:

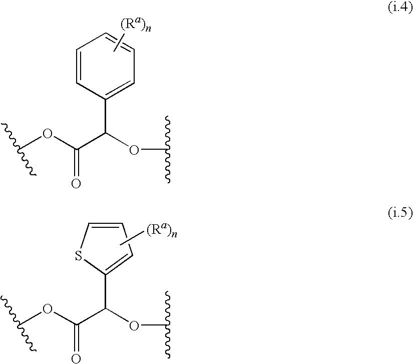

wherein n is 0, 1, 2, 3, 4 or 5.

Also of particular mention are substituents of the following Formulae:

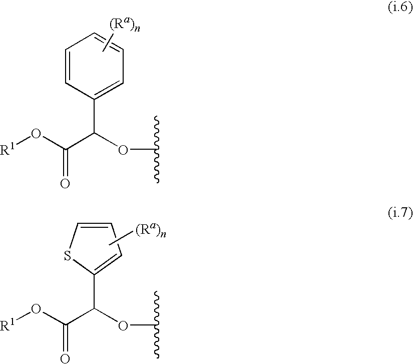

In Formulae (i.4), (i.5), (i.6) and (i.7), n may, in particular, be 0, 1, or 2. Where present, the or each R^{a} may be, for example, independently selected from halogen (e.g. fluorine or chlorine), C₁₋₆ alkoxy (e.g. methoxy or ethoxy) and alkoxycarbonyl (e.g. methoxycarbonyl or ethoxycarbonyl). More usually, n is 0.

In Formulae (i.6) and (i.7), R¹ is typically hydrogen or C₁, C₂, C₃ or C₄ alkyl optionally substituted with 1, 2, 3, 4 or 5 R^{a}. In particular, R¹ may be hydrogen, methyl or ethyl.

In a particular class of compounds, a fragment or substituent of any of Formulae (i.1) to (i.7) is present at the 6-position of the benzothiophene ring.

### Fragment (ii)

A compound of the invention may comprise, at the 4- and/or 6-position, a substituent comprising a fragment of the Formula (ii):

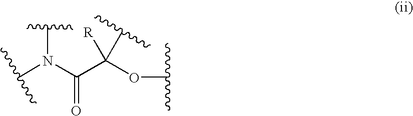

R is as previously defined.

In one class of compounds, a substituent comprising a fragment of Formula (ii) is bound at the 4-position of the benzothiophene ring.

In another class of compounds, a substituent comprising a fragment of Formula (ii) is bound at the 6-position of the benzothiophene ring.

The fragment of Formula (ii) may be selected from one of the following fragments, particularly when the substituent is present at the 4-position:

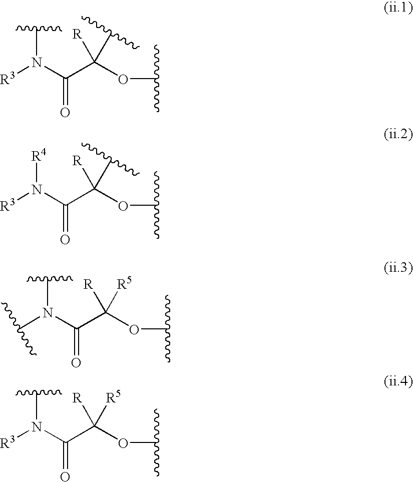

wherein
R³ and R⁴ are each independently hydrogen or selected from C₁₋₆ alkyl, —(CH₂)ₖ-carbocyclyl and —(CH₂)ₖ-heterocyclyl, any of which is optionally substituted with 1, 2, 3, 4 or 5 R^{a};
or R³ and R⁴ together with the nitrogen atom to which they are attached form heterocyclyl optionally substituted with 1, 2, 3, 4 or 5 R^{a};
R⁵ is selected from hydrogen and C₁₋₆ alkyl optionally substituted with 1, 2, 3, 4 or 5 R^{a}; and
k is 0, 1, 2, 3, 4, 5 or 6.

Thus, a substituent comprising fragment (ii) may be of the following Formula:

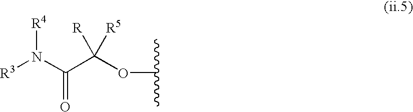

In one class compounds, R³ is hydrogen or is selected from C₁₋₆ alkyl, —(CH₂)ₖ-aryl and —(CH₂)ₖ-heterocyclyl, any of which is optionally substituted with 1, 2, 3, 4 or 5 R^{a}. In particular, R³ may be selected from cycloalkyl (e.g. cyclohexyl), aryl (e.g phenyl), —CH₂-aryl (e.g. benzyl), —CH₂CH₂-aryl, heterocyclyl, —CH₂-heterocyclyl and —CH₂CH₂-heterocyclyl, any of which is optionally substituted with 1, 2, 3, 4 or 5 R^{a}.

In a further class of compounds, R⁴ is hydrogen, or C₁₋₆ alkyl optionally substituted with 1, 2, 3, 4 or 5 R^{a}. In particular, R⁴ may hydrogen, methyl or ethyl.

In a further class of compounds, R³ and R⁴ taken together with the nitrogen atom to which they are attached form heterocycloalkyl (e.g. pyrrolidinyl or piperidinyl) optionally substituted with 1, 2, 3, 4 or 5 R^{a}.

In a further class of compounds, R⁵ is hydrogen or C₁, C₂, C₃ or C₄ alkyl optionally substituted with 1, 2, 3, 4 or 5 R^{a}. In particular, R⁵ may be hydrogen or methyl.

In a further class of compounds, R comprises (e.g. is) aryl or heteroaryl, either of which is optionally substituted with 1, 2, 3, 4 or 5 R^{a}. Aryl and heteroaryl are often monocyclic, e.g. having 5 or 6 ring members, being, for example, phenyl or thiophenyl. Sometimes, aryl and heteroaryl are polycyclic, e.g. bicyclic, having, for example, 8, 9 or 10 ring members. In particular, R may be phenyl optionally substituted with 1, 2, 3, 4 or 5 R^{a}.

Of particular mention are fragments of the following Formula:

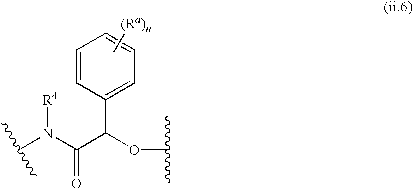

Of particular mention are substituents of the following Formula:

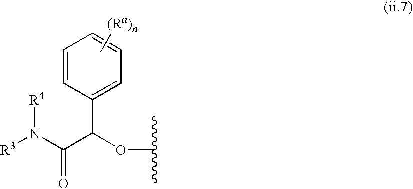

In Formulae (ii.6) and (ii.7), R⁴ is usually hydrogen or C₁₋₆ alkyl (e.g. methyl) and n may, in particular, be 0, 1, or 2. Where present, the or each R^{a} may be, for example, independently selected from halogen (e.g. fluorine or chlorine), C₁₋₆ alkoxy (e.g. methoxy or ethoxy) and C₁₋₆ alkoxycarbonyl (e.g. methoxycarbonyl or ethoxycarbonyl). More usually, n is 0.

In Formula (ii.7), R³ is typically —(CH₂)ₖ-carbocyclyl or —(CH₂)ₖ-heterocyclyl, either of which is optionally substituted with 1, 2, 3, 4 or 5 R^{a}. In particular, R³ may be selected from cycloalkyl (e.g. cyclohexyl), aryl (e.g phenyl), —CH₂-aryl (e.g. benzyl), —CH₂CH₂-aryl, heterocyclyl, —CH₂-heterocyclyl and —CH₂CH₂-heterocyclyl, any of which is optionally substituted with 1, 2, 3, 4 or 5 R^{a}.

In a particular class of compounds, a fragment or substituent of any of Formulae (ii.1) to (ii.7) is present at the 4-position of the benzothiophene ring.

Fragment (iii)

A compound of the invention may comprise, at the 4- and/or 6-position, a substituent comprising a fragment of the Formula (iii):

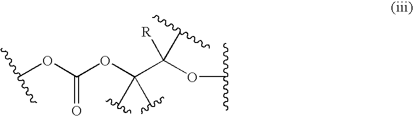

wherein R is as previously defined.

In one class of compounds, a substituent comprising a fragment of Formula (iii) is bound at the 4-position of the benzothiophene ring.

In another class of compounds, a substituent comprising a fragment of Formula (iii) is bound at the 6-position of the benzothiophene ring.

The fragment of Formula (iii) may be selected from one of the following fragments, particularly when the substituent is present at the 4-position:

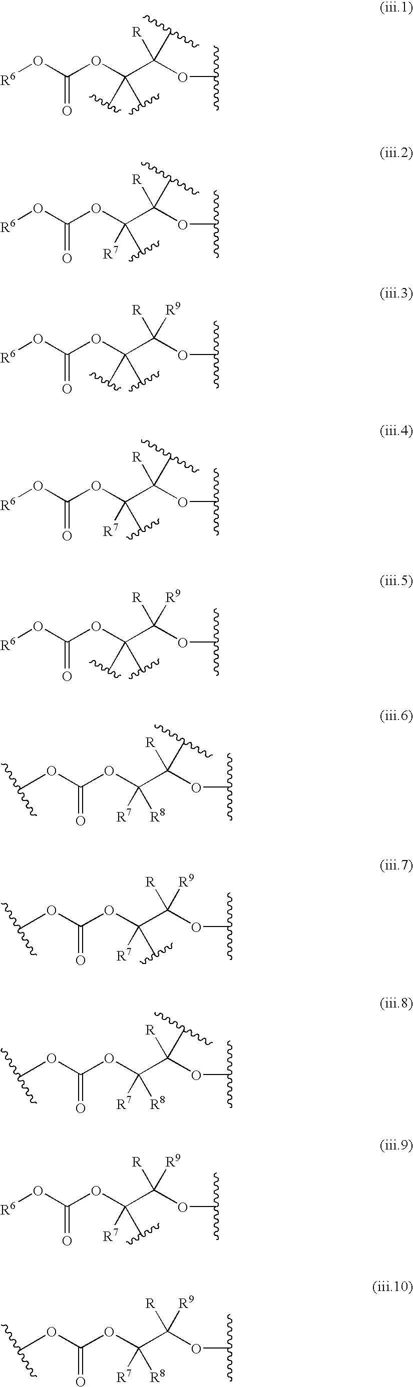

wherein
R⁶ is hydrogen or is selected from C₁₋₆ alkyl, —(CH₂)ₖ-carbocyclyl and —(CH₂)ₖ-heterocyclyl, any of which is optionally substituted with 1, 2, 3, 4 or 5 R^{a};
R⁷, R⁸ and R⁹ are each independently selected from hydrogen and C₁₋₆ alkyl optionally substituted with 1, 2, 3, 4 or 5 R^{a}; and
k is 0, 1, 2, 3, 4, 5 or 6.

Thus, a substituent comprising fragment (iii) may be of the following Formula:

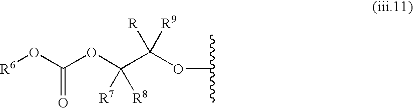

In one class of compounds, R⁶ is hydrogen, or C₁₋₆ alkyl optionally substituted with 1, 2, 3, 4 or 5 R^{a}. In particular, R⁶ may be hydrogen, methyl or ethyl.

In another class of compounds, R⁷, R⁸ and R⁹ are each independently hydrogen or C₁, C₂, C₃ or C₄ alkyl optionally substituted with 1, 2, 3, 4 or 5 R^{a}. In particular, R⁷, R⁸ and R⁹ may be each independently hydrogen or methyl.

In a further class of compounds, R comprises (e.g. is) aryl or heteroaryl, either of which is optionally substituted with 1, 2, 3, 4 or 5 R^{a}. Aryl and heteroaryl are often monocyclic, e.g. having 5 or 6 ring members, being, for example, phenyl or thiophenyl. Sometimes, aryl and heteroaryl are polycyclic, e.g. bicyclic, having, for example, 8, 9 or 10 ring members. In particular, R may be phenyl optionally substituted with 1, 2, 3, 4 or 5 R^{a}.

Of particular mention are fragments of the following Formula:

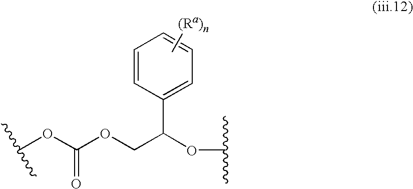

wherein n is 0, 1, 2, 3, 4 or 5.

Also of particular mention are substituents of the following Formula:

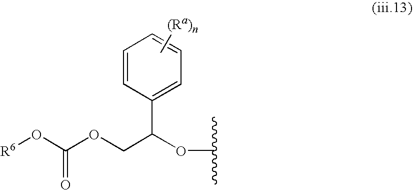

In Formulae (iii.12) and (iii.13), n may, in particular, be 0, 1, or 2. Where present, the or each R^{a} may be, for example, independently selected from halogen (e.g. fluorine or chlorine), C₁₋₆ alkoxy (e.g. methoxy or ethoxy) and alkoxycarbonyl (e.g. methoxycarbonyl or ethoxycarbonyl). More usually, n is 0.

In Formula (iii.13), R⁶ is typically hydrogen or C₁, C₂, C₃ or C₄ alkyl optionally substituted with 1, 2, 3, 4 or 5 R^{a}. In particular, R¹ may hydrogen, methyl or ethyl.

In a particular class of compounds, a fragment or substituent of any of Formulae (iii.1) to (iii.13) is present at the 4-position of the benzothiophene ring.

### Fragment (iv)

A compound of the invention may comprise, at the 4- and/or 6-position, a substituent comprising a fragment of the Formula (iv):

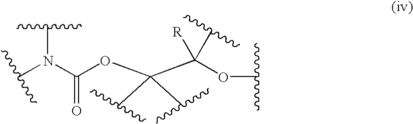

wherein R is as previously defined.

In one class of compounds, a substituent comprising a fragment of Formula (iv) is bound at the 4-position of the benzothiophene ring.

In another class of compounds, a substituent comprising a fragment of Formula (iv) is bound at the 6-position of the benzothiophene ring.

The fragment of Formula (iv) may be selected from one of the following fragments, particularly when the substituent is present at the 4-position:

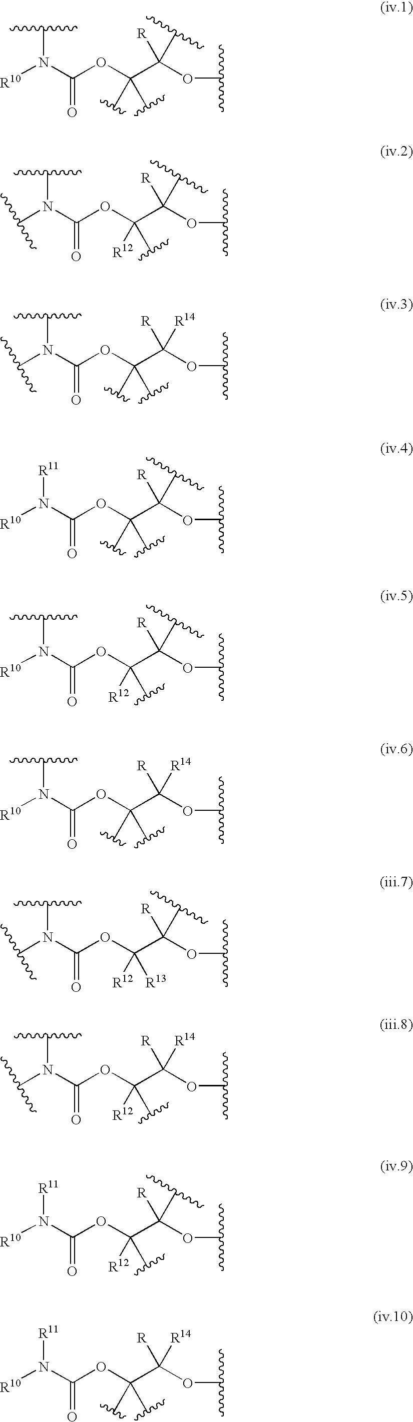

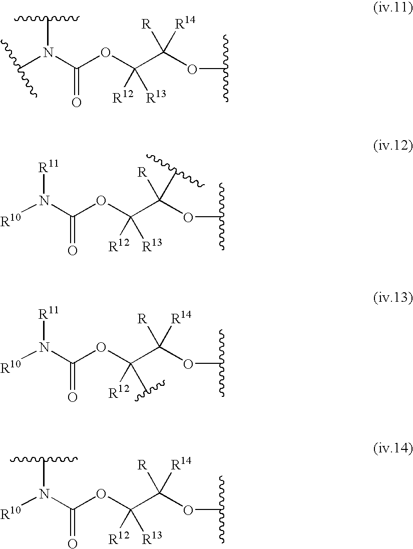

wherein
R¹⁰ and R¹¹ are each independently hydrogen or selected from C₁₋₆ alkyl, —(CH₂)ₖ-carbocyclyl and —(CH₂)ₖ-heterocyclyl, any of which is optionally substituted with 1, 2, 3, 4 or 5 R^{a};
or R¹⁰ and R¹¹ together with the nitrogen atom to which they are attached form heterocyclyl optionally substituted with 1, 2, 3, 4 or 5 R^{a};
R¹², R¹³ and R¹⁴ are each independently selected from hydrogen and C₁₋₆ alkyl optionally substituted with 1, 2, 3, 4 or 5 R^{a}; and
k is 0, 1, 2, 3, 4, 5 or 6.

Thus, a substituent comprising fragment (iv) may be of the following Formula:

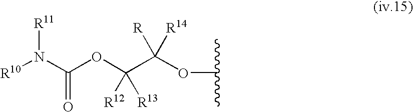

In one class compounds, R¹⁰ is hydrogen or is selected from C₁₋₆ alkyl, —(CH₂)ₖ-aryl and —(CH₂)ₖ-heterocyclyl, any of which is optionally substituted with 1, 2, 3, 4 or 5 R^{a}. In particular, R¹⁰ may be selected from cycloalkyl (e.g. cyclohexyl), aryl (e.g phenyl), —CH₂-aryl (e.g. benzyl), —CH₂CH₂-aryl, heterocyclyl, —CH₂-heterocyclyl and —CH₂CH₂-heterocyclyl, any of which is optionally substituted with 1, 2, 3, 4 or 5 R^{a}.

In a further class of compounds, R¹¹ is hydrogen or C₁₋₆ alkyl optionally substituted with 1, 2, 3, 4 or 5 R^{a}. In particular, R¹¹ may hydrogen, methyl or ethyl.

In a further class of compounds, R¹⁰ and R¹¹ taken together with the nitrogen atom to which they are attached form heterocycloalkyl (e.g. pyrrolidinyl or piperidinyl) optionally substituted with 1, 2, 3, 4 or 5 R^{a}.

In another class of compounds, R¹², R¹³ and R¹⁴ are each independently hydrogen or C₁, C₂, C₃ or C₄ alkyl optionally substituted with 1, 2, 3, 4 or 5 R^{a}. In particular, R¹², R¹³ and R¹⁴ may each be hydrogen or methyl.

In a further class of compounds, R comprises (e.g. is) aryl or heteroaryl, either of which is optionally substituted with 1, 2, 3, 4 or 5 R^{a}. Aryl and heteroaryl are often monocyclic, e.g. having 5 or 6 ring members, being, for example, phenyl or thiophenyl. Sometimes, aryl and heteroaryl are polycyclic, e.g. bicyclic, having, for example, 8, 9 or 10 ring members. In particular, R may be phenyl optionally substituted with 1, 2, 3, 4 or 5 R^{a}.

Of particular mention are fragments of the following formula:

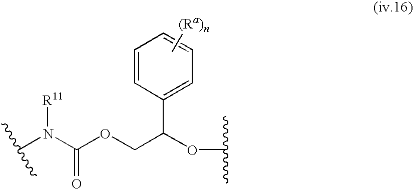

Also of particular mention are substituents of the Formula (iv.13):

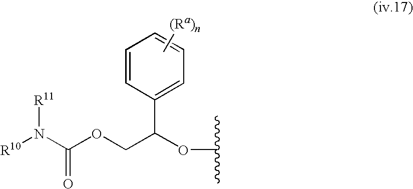

In Formulae (iv.6) and (iv.17), R¹¹ is usually hydrogen or C₁₋₆ alkyl (e.g. methyl) and n may, in particular, be 0, 1, or 2. Where present, the or each R^{a} may be, for example, independently selected from halogen (e.g. fluorine or chlorine), C₁₋₆ alkoxy (e.g. methoxy or ethoxy) and alkoxycarbonyl (e.g. methoxycarbonyl or ethoxycarbonyl). More usually, n is 0.

In Formula (iv.17), R³ is typically —(CH₂)ₖ-carbocyclyl or —(CH₂)ₖ-heterocyclyl, either of which is optionally substituted with 1, 2, 3, 4 or 5 RF. In particular, R³ may be selected from aryl (e.g phenyl), —CH₂-aryl (e.g. benzyl), —CH₂CH₂-aryl, heterocyclyl, —CH₂-heterocyclyl and —CH₂CH₂-heterocyclyl, any of which is optionally substituted with 1, 2, 3, 4 or 5 R^{a}.

In a particular class of compounds, a fragment or substituent of any of Formulae (iv.1) to (iv.17) is present at the 4-position of the benzothiophene ring.

### Fragment (v)

A compound of the invention may comprise, at the 4- and/or 6-position, a substituent comprising a fragment of the Formula (v):

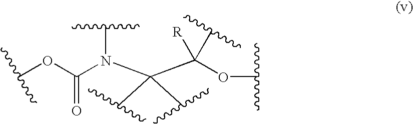

R is as previously defined.

In one class of compounds, a substituent comprising a fragment of Formula (v) is bound at the 4-position of the benzothiophene ring.

In another class of compounds, a substituent comprising a fragment of Formula (v) is bound at the 6-position of the benzothiophene ring.

The fragment of Formula (v) may be selected from one of the following fragments, particularly when the substituent is present at the 4-position:

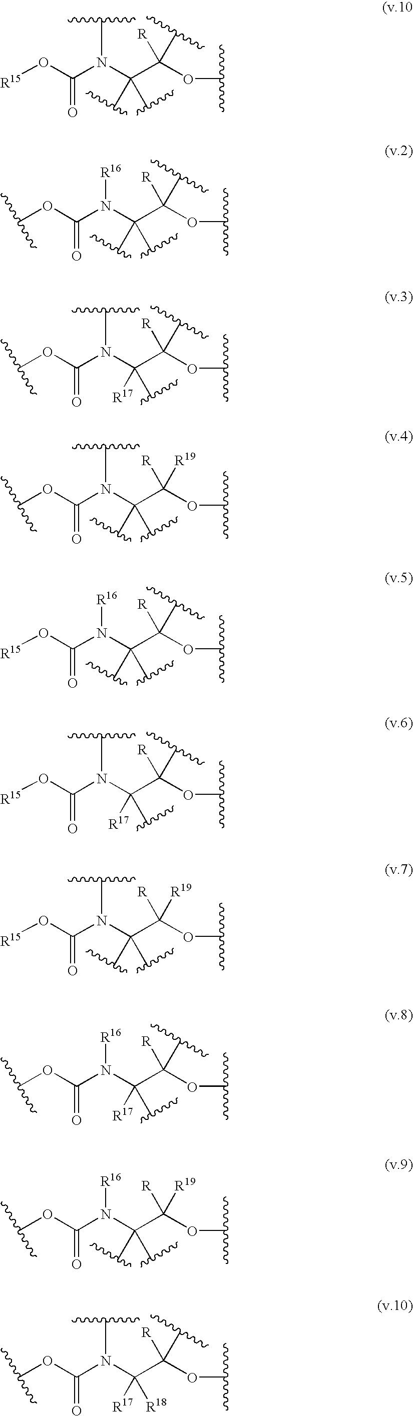

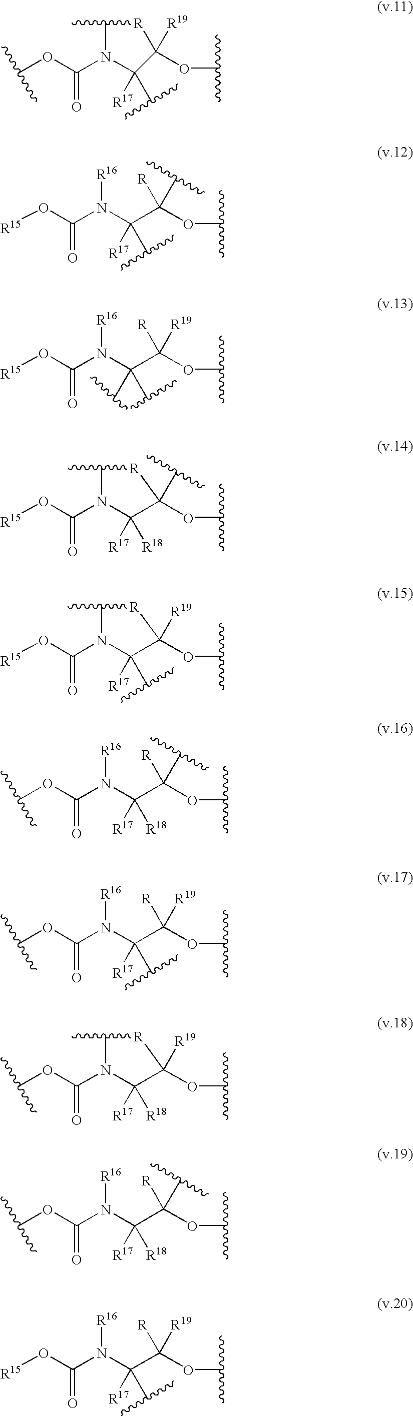

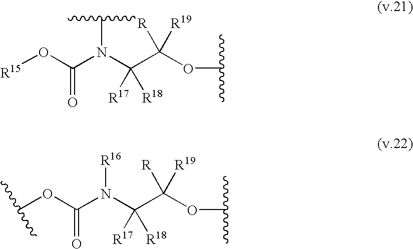

wherein
R¹⁵ is hydrogen or is selected from C₁₋₆ alkyl, —(CH₂)ₖ-carbocyclyl and —(CH₂)ₖ-heterocyclyl, any of which is optionally substituted with 1, 2, 3, 4 or 5 R^{a};
R¹⁶, R¹⁷, R¹⁸ and R¹⁹ are each independently selected from hydrogen and C₁₋₆ alkyl optionally substituted with 1, 2, 3, 4 or 5 R^{a}; and
k is 0, 1, 2, 3, 4, 5 or 6.

Thus, the substituent comprising fragment (v) may be of the following Formula:

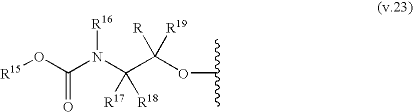

In one class of compounds, R¹⁵ is hydrogen, or C₁₋₆ alkyl optionally substituted with 1, 2, 3, 4 or 5 R^{a}. In particular, R¹⁵ may be hydrogen, methyl or ethyl.

In another class of compounds, R¹⁶, R¹⁷, R¹⁸ and R¹⁹ are each independently hydrogen or C₁, C₂, C₃ or C₄ alkyl optionally substituted with 1, 2, 3, 4 or 5 R^{a}. In particular, R¹⁶, R¹⁷, R¹⁸ and R¹⁹ may each be hydrogen or methyl.

In a further class of compounds, R comprises (e.g. is) aryl or heteroaryl, either of which is optionally substituted with 1, 2, 3, 4 or 5 R^{a}. Aryl and heteroaryl are often monocyclic, e.g. having 5 or 6 ring members, being, for example, phenyl or thiophenyl. Sometimes, aryl and heteroaryl are polycyclic, e.g. bicyclic, having, for example, 8, 9 or 10 ring members. In particular, R may be phenyl optionally substituted with 1, 2, 3, 4 or 5 R^{a}.

Of particular mention are fragments of the following Formula:

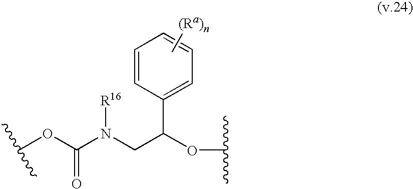

wherein n is 0, 1, 2, 3, 4 or 5.

Also of particular mention are substituents of the following Formula:

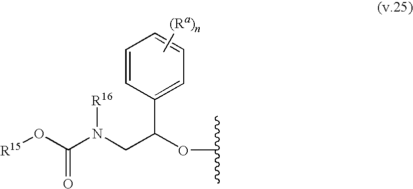

In Formulae (v.24) and (v.25), R¹⁶ is usually hydrogen or C₁₋₆ alkyl (e.g. methyl) and n may, in particular, be 0, 1, or 2. Where present, the or each R^{a} may be, for example, independently selected from halogen (e.g. fluorine or chlorine), C₁₋₆ alkoxy (e.g. methoxy or ethoxy) and alkoxycarbonyl (e.g. methoxycarbonyl or ethoxycarbonyl). More usually, n is 0.

In Formula (v.25), R¹⁵ is typically hydrogen or C₁, C₂, C₃ or C₄ alkyl optionally substituted with 1, 2, 3, 4 or 5 R^{a}. In particular, R¹⁵ may be hydrogen, methyl or ethyl.

In a particular class of compounds, a fragment or substituent of any of Formulae (v.1) to (v.25) is present at the 4-position of the benzothiophene ring.

### Fragment (vi)

A compound of the invention may comprise, at the 4- and/or 6-position, a substituent comprising a fragment of the Formula (vi):

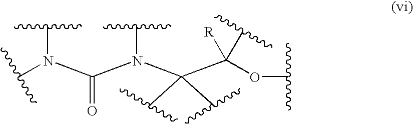

wherein R is as previously defined.

In one class of compounds, a substituent comprising a fragment of Formula (vi) is bound at the 4-position of the benzothiophene ring.

In another class of compounds, a substituent comprising a fragment of Formula (vi) is bound at the 6-position of the benzothiophene ring.

The fragment of Formula (vi) may be selected from one of the following fragments, particularly when the substituent is present at the 4-position:

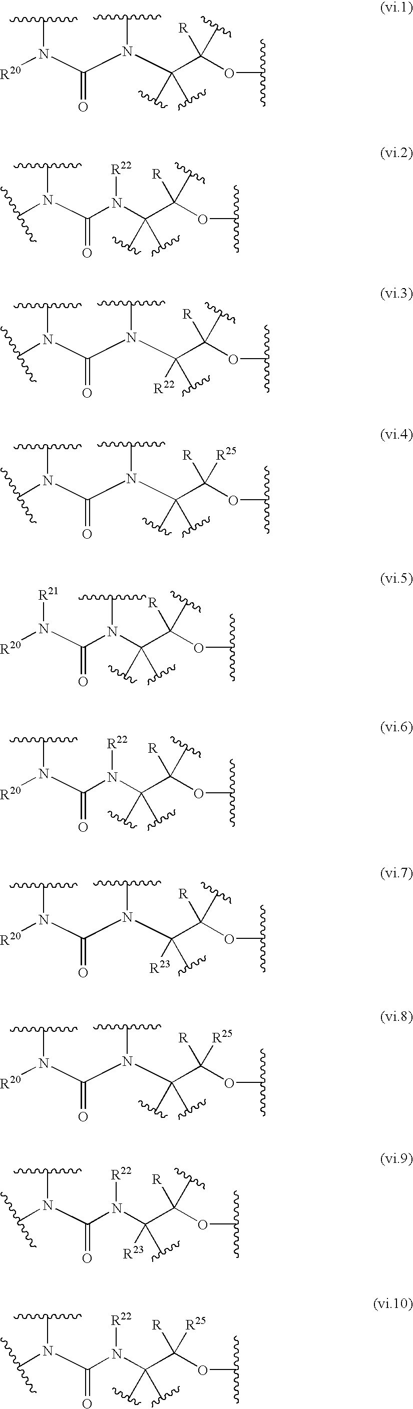

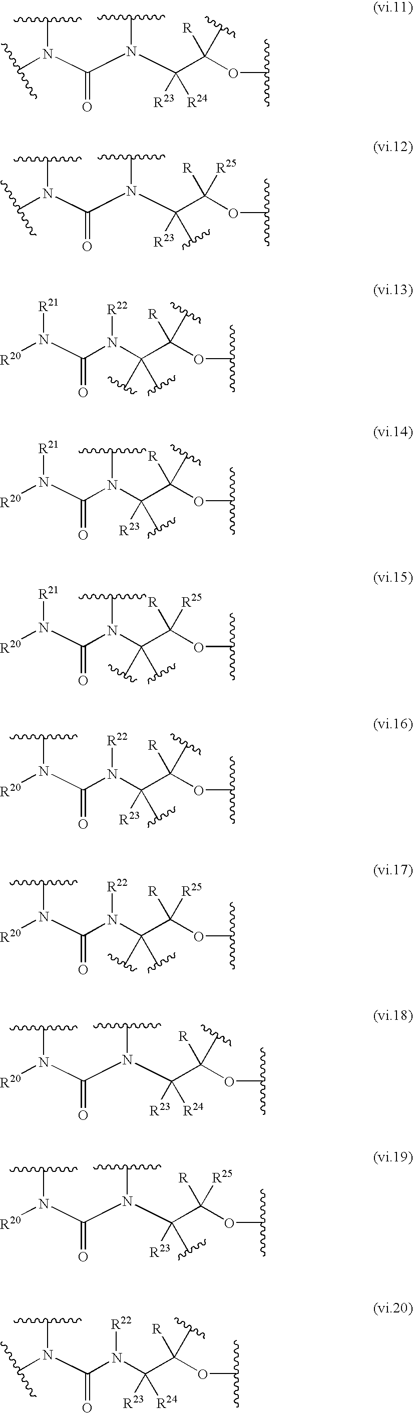

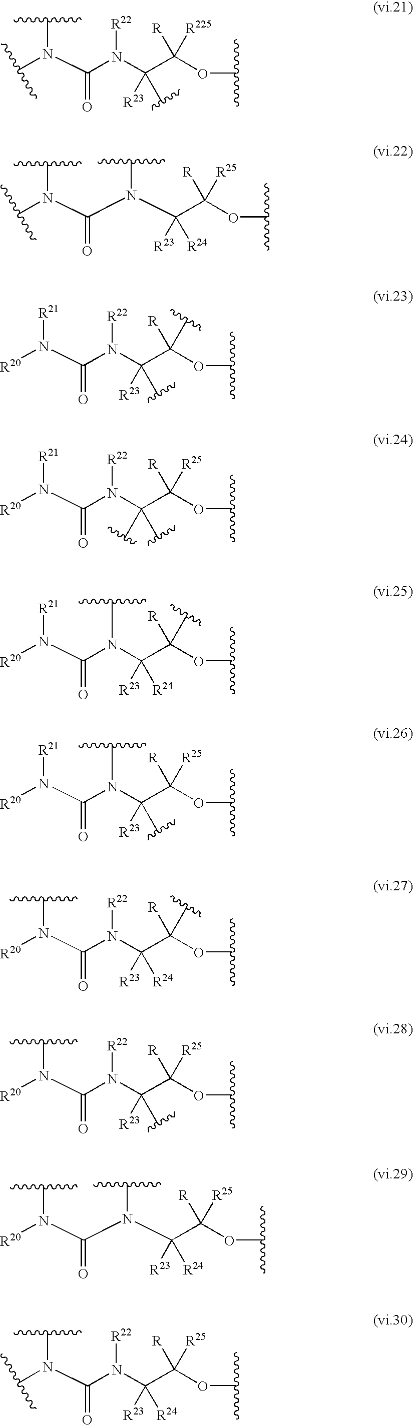

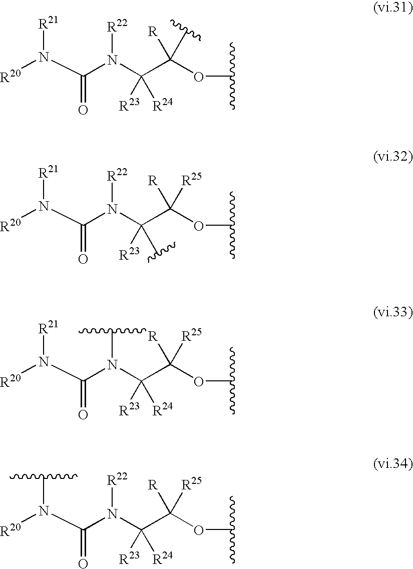

wherein
R²⁰ and R²¹ are each independently hydrogen or selected from C₁₋₆ alkyl, —(CH₂)ₖ-carbocyclyl and —(CH₂)ₖ-heterocyclyl, any of which is optionally substituted with 1, 2, 3, 4 or 5 R^{a};
or R²⁰ and R²¹ together with the nitrogen atom to which they are attached form heterocyclyl optionally substituted with 1, 2, 3, 4 or 5 R^{a};
R²², R²³, R²⁴ and R²⁵ are each independently selected from hydrogen and C₁₋₆ alkyl optionally substituted with 1, 2, 3, 4 or 5 R^{a}; and
k is 0, 1, 2, 3, 4, 5 or 6.

Thus, a substituent comprising fragment (vi) may be of the following Formula:

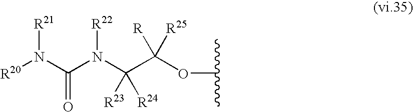

In one class compounds, R²⁰ is hydrogen or is selected from C₁₋₆ alkyl, —(CH₂)ₖ-aryl and —(CH₂)ₖ-heterocyclyl, any of which is optionally substituted with 1, 2, 3, 4 or 5 R^{a}. In particular, R²⁰ may be selected from cycloalkyl (e.g. cyclohexyl), aryl (e.g phenyl), —CH₂-aryl (e.g. benzyl), —CH₂CH₂-aryl, heterocyclyl, —CH₂-heterocyclyl and —CH₂CH₂-heterocyclyl, any of which is optionally substituted with 1, 2, 3, 4 or 5 R^{a}.

In a further class of compounds, R²¹ is hydrogen, or C₁₋₆ alkyl optionally substituted with 1, 2, 3, 4 or 5 R^{a}. In particular, R¹¹ may hydrogen, methyl or ethyl.

In a further class of compounds, R²⁰ and R²¹ taken together with the nitrogen atom to which they are attached form heterocycloalkyl (e.g. pyrrolidinyl or piperidinyl) optionally substituted with 1, 2, 3, 4 or 5 R^{a}.

In another class of compounds, R²², R²³, R²⁴ and R²⁵ are each independently hydrogen or C₁, C₂, C₃ or C₄ alkyl optionally substituted with 1, 2, 3, 4 or 5 R^{a}. In particular, R²², R²³, R²⁴ and R²⁵ may be each independently hydrogen or methyl.

In a further class of compounds, R comprises (e.g. is) aryl or heteroaryl, either of which is optionally substituted with 1, 2, 3, 4 or 5 R^{a}. Aryl and heteroaryl are often monocyclic, e.g. having 5 or 6 ring members, being, for example, phenyl or thiophenyl. Sometimes, aryl and heteroaryl are polycyclic, e.g. bicyclic, having, for example, 8, 9 or 10 ring members. In particular, R may be phenyl optionally substituted with 1, 2, 3, 4 or 5 R^{a}.

Of particular mention are fragments of the Formula (vi.36):

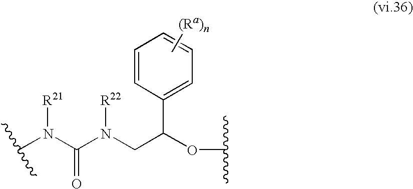

Also of particular mention are substituents of the Formula (vi.37):

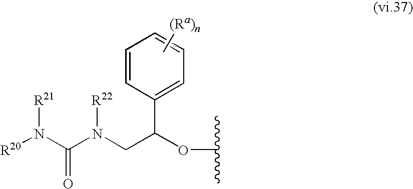

In Formulae (vi.36) and (vi.37), R²⁰ and R²¹ may be each independently hydrogen or C₁₋₆ alkyl (e.g. methyl), and n may, in particular, be 0, 1, or 2. Where present, the or each R^{a} may be, for example, independently selected from halogen (e.g. fluorine or chlorine), C₁₋₆ alkoxy (e.g. methoxy or ethoxy) and alkoxycarbonyl (e.g methoxycarbonyl or ethoxycarbonyl). More usually, n is 0.

In Formula (vi.37), R¹⁹ is typically —(CH₂)ₖ-carbocyclyl or —(CH₂)ₖ-heterocyclyl, either of which is optionally substituted with 1, 2, 3, 4 or 5 R^{a}. In particular, R¹⁹ may be selected from aryl (e.g phenyl), —CH₂-aryl (e.g. benzyl), —CH₂CH₂-aryl, heterocyclyl, —CH₂-heterocyclyl and —CH₂CH₂-heterocyclyl, any of which is optionally substituted with 1, 2, 3, 4 or 5 R^{a}.

In a particular class of compounds, a fragment or substituent of any of Formulae (vi.1) to (vi.37) is present at the 4-position of the benzothiophene ring.

### R

R comprises (e.g. is) a carbocyclic or heterocyclic group, either of which is optionally substituted.

In one class of compounds, R is a moiety comprising an optionally substituted carbocyclic or heterocyclic group linked to the remainder of the fragment via a linker, L, having 1, 2, 3, 4 or 5 in-chain atoms, e.g. 1 or 2 atoms. Exemplary linkers include alkylene and alkylene substituted by 1, 2, 3, 4 or 5 R^{a}, wherein each R^{a} is typically hydroxy or halogen (e.g. fluorine or chlorine). Particular linkers are methylene and ethylene.

In another class of compounds, R is an optionally substituted carbocyclic or heterocyclic group. The group may be unsubstituted, or substituted with 1, 2, 3, 4 or 5 R^{a}.

When R comprises (in particular, is) an optionally substituted carbocyclic group, the carbocycle may be a saturated (e.g. cycloalkyl) or unsaturated (e.g. aryl) ring moiety having 3, 4, 5, 6, 7, 8, 9, 10, 11, 12, 13, 14, 15 or 16 carbon ring atoms. In particular, R may comprise a 3- to 10-membered non-aromatic ring or ring system and, in particular, a 5- or 6-membered non-aromatic ring, which may be fully or partially saturated. Exemplary carbocyclyl groups include cyclopropyl, cyclobutyl, cyclopentyl, cyclohexyl, norbornyl, bicyclo[2.2.2]octyl, phenyl, naphthyl, fluorenyl, azulenyl, indenyl, anthryl and the like, any of which is optionally substituted with 1, 2, 3, 4 or 5 R^{a}. In a particular embodiment, R is phenyl optionally substituted with 1, 2, 3, 4 or 5 R^{a}. In a particular class of compounds, R is unsubstituted, an exemplary group being phenyl.

When R comprises (in particular, is) an optionally substituted heterocyclic group, the heterocycle may be a saturated (e.g. heterocycloalkyl) or unsaturated (e.g. heteroaryl) heterocyclic ring moiety having from 3, 4, 5, 6, 7, 8, 9, 10, 11, 12, 13, 14, 15 or 16 ring atoms, at least one of which is selected from nitrogen, oxygen, phosphorus, silicon and sulphur. In particular, R may comprise a 3- to 10-membered non-aromatic ring or ring system and more particularly a 5- or 6-membered ring, which may be fully or partially saturated. Exemplary heterocyclyl groups include oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, β-carbolinyl, phenanthridinyl, acridinyl, perimidinyl, phenanthrolinyl, furazanyl, phenazinyl, phenothiazinyl, phenoxazinyl, chromenyl, isochromanyl, chromanyl and the like, any of which is optionally substituted with 1, 2, 3, 4 or 5 R^{a}. In a particular embodiment, R is thiophenyl (e.g. thiophen-2-yl or thiophen-3-yl) optionally substituted with 1, 2, 3, 4 or 5 R^{a}. In another particular embodiment, R is thiophenyl.

### R^{a}

R^{a} may be as previously described. In embodiments, each R^{a} is independently selected from halogen, hydroxy, trifluoromethyl, cyano, nitro, oxo, amidino, —B(OH)₂, ═NR^{b}, —OR^{b}, —C(O)R^{b}, C(O)OR^{b}, OC(O)R^{b}, —N(R^{b})R^{c}, —C(O)N(R^{b})R^{c}, —S(O)ₗR^{b}, —C(R^{b})₃ and R^{d}; wherein R^{b} and R^{c} are each independently hydrogen or selected from C₁₋₆ alkyl, —(CH₂)ₖ-carbocyclyl and —(CH₂)ₖ-heterocyclyl, any of which is optionally substituted with 1, 2, 3, 4 or 5 substituents independently selected from halogen, hydroxy and C₁₋₆ alkyl; R^{d} is selected from C₁₋₆ alkyl, C₁₋₆ alkoxy, —(CH₂)ₖ-carbocyclyl and —(CH₂)ₖ-heterocyclyl, any of which is optionally substituted with 1, 2, 3, 4 or 5 substituents independently selected from halogen, hydroxy, C₁₋₆ alkyl and C₁₋₆ alkoxy; k is 0, 1, 2, 3, 4, 5 or 6; and l is 0, 1 or 2.

For example, the or each R^{a} may be halogen (e.g. fluorine or chlorine) or an inert organic group, for example C₁₋₆ alkyl (e.g. methyl or ethyl), C₁₋₆ alkoxy (e.g. methoxy or ethoxy), either of which is optionally substituted by halogen (e.g. fluorine or chlorine). As mentioned above, where an alkyl or alkyl-substituted group is mentioned, alkyl typically has 1, 2, 3 or 4 carbon atoms.

R^{a} may be selected from (i) halogen; (ii) moieties having from 1 to 30 plural valent atoms (e.g. 1 to 20, for example 1 to 10, in particular 1, 2, 3 or 4, plural valent atoms), selected from C, N, O and S as well as monovalent atoms selected from hydrogen and halogen, e.g. selected from hydrogen, F, Cl and Br, for example hydrogen, F and Cl.

R^{a} may be a hydrogen bond acceptor, examples of such groups include halogen (e.g. fluorine), hydroxy, tertiary amino groups and carbonyl groups. Alternatively, R^{a} may be a hydrogen bond donor, examples of such groups including hydroxy and primary and secondary amine groups.

In certain compounds, each R^{a} is independently selected from halogen (e.g. fluorine, chlorine, bromine or iodine), hydroxy, trifluoromethyl, cyano, nitro, oxo, —OR^{b}, —C(O)R^{b}, —C(O)OR^{b}, —OC(O)R^{b}, —N(R^{b})R^{c}, —C(O)N(R^{b})R^{c}, —S(O)ₗR^{b}, —C(R^{b})₃ and R^{d}. In this case, R^{b} and R^{c} are usually each independently hydrogen or selected from C₁₋₆ alkyl (e.g. methyl, ethyl, propyl or butyl), —(CH₂)ₖ-aryl (e.g. phenyl or benzyl) and —(CH₂)ₖ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl). R^{d} is usually selected from C₁₋₆ alkyl (e.g. methyl, ethyl, propyl or butyl), —(CH₂)ₖ-aryl (e.g. phenyl or benzyl) and —(CH₂)ₖ-heteroaryl (e.g. pyridinyl or thiophenyl), any of which is optionally substituted with 1, 2, 3, 4 or 5 substituents independently selected from halogen (e.g. fluorine or chlorine), hydroxy or C₁₋₆ alkyl (e.g. methyl, ethyl, propyl or butyl).

For the avoidance of doubt, where a group is substituted with more than one R^{a}, each R¹ is independently selected from the range of substituents specified. The same applies to compounds of the invention comprising more than one R^{a} substituent; each R^{a} is selected independently of any other R^{a} substituent present in the compound.

This is the end of the section “Particular Benzothiophene Compounds”.

### Exemplary Compounds

Examples of compounds of the invention include those shown below. It will of course be appreciated that where a salt is shown, this is merely an illustrative example and non-limiting and other salts are contemplated, as are the free acids and bases of the salts. Each compound may, therefore, be in the form of the free compound, a salt, or a prodrug. Where a nitrogen atom forming only two bonds is shown, this represents NH.

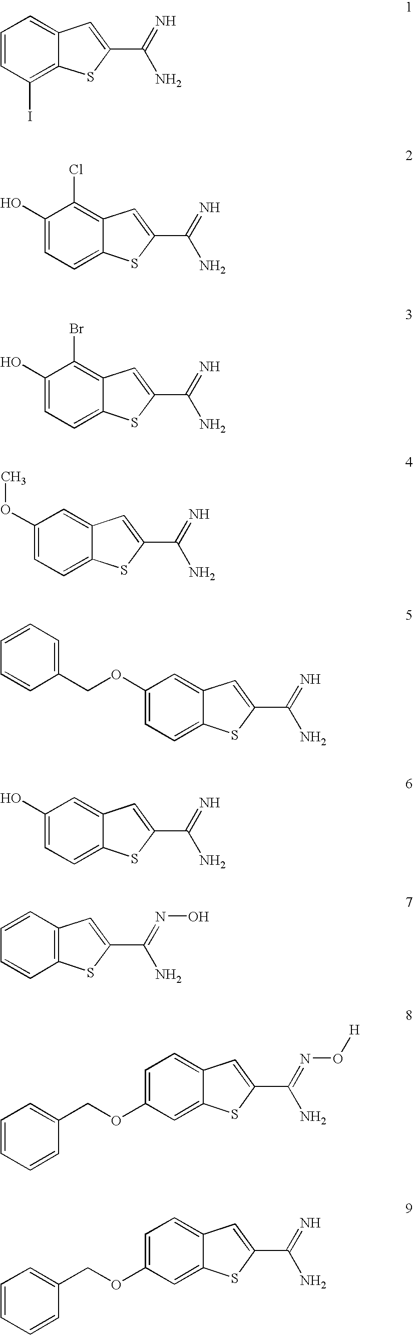

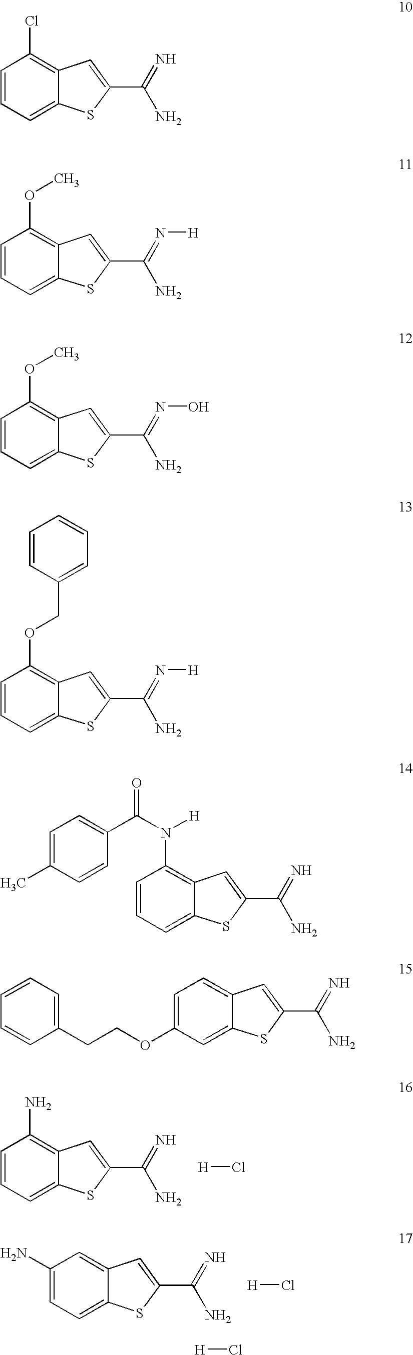

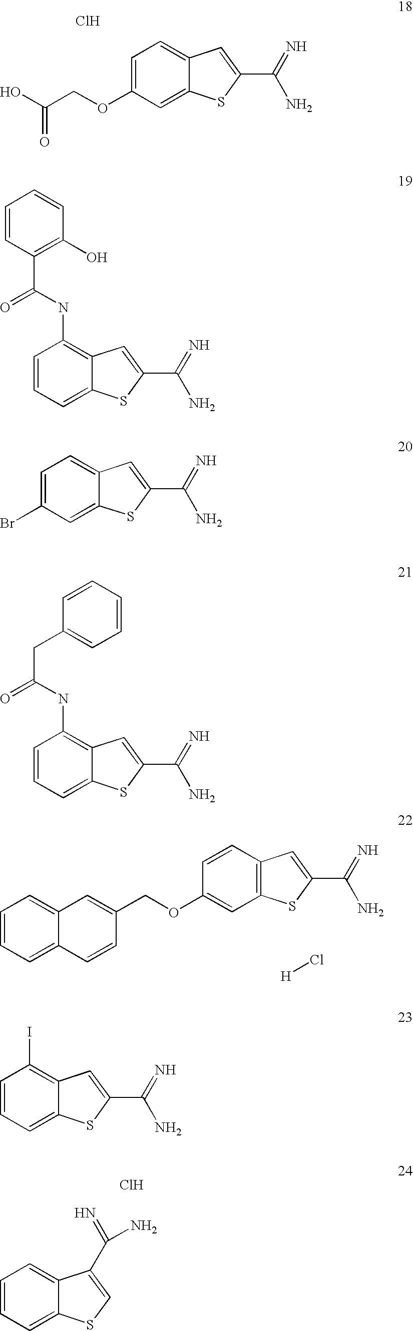

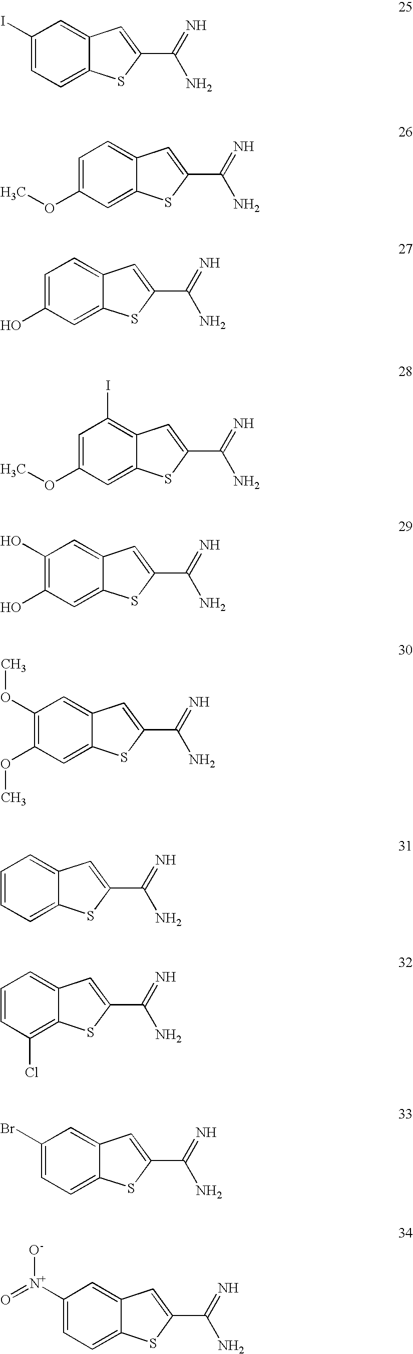

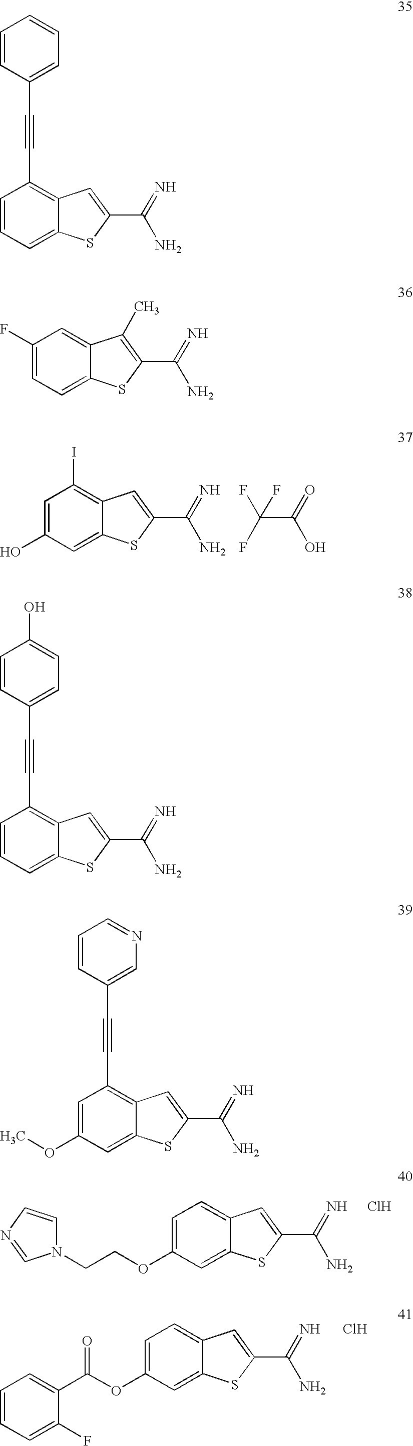

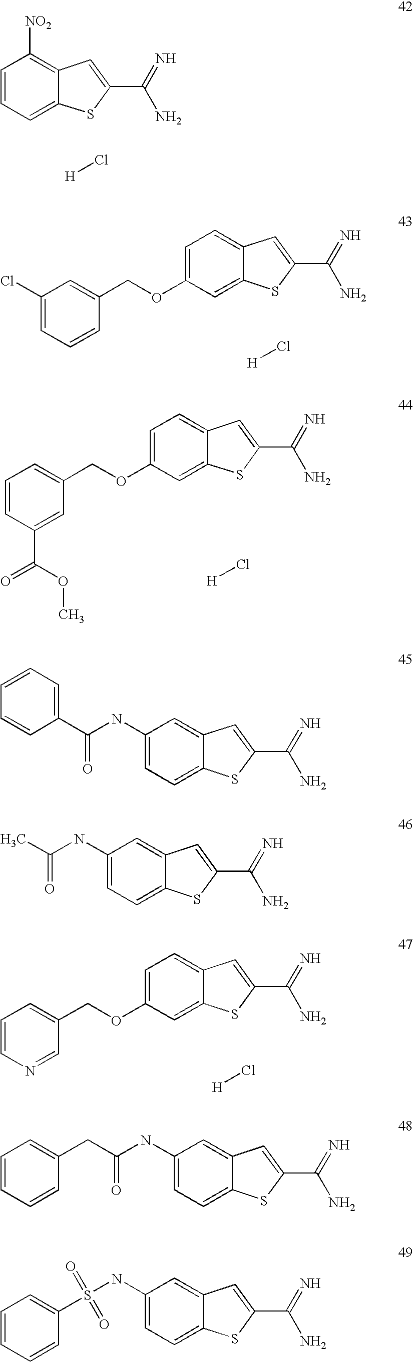

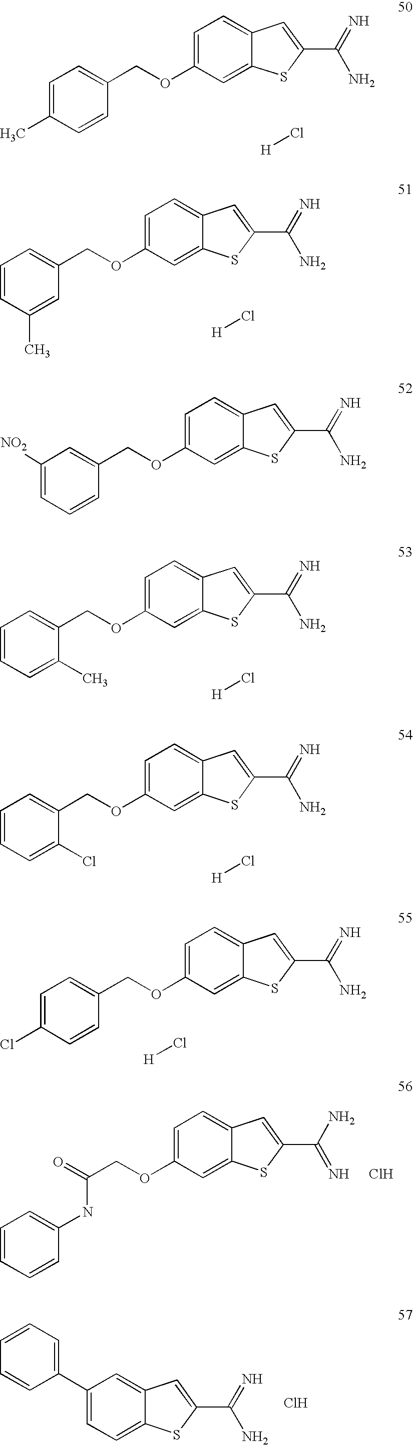

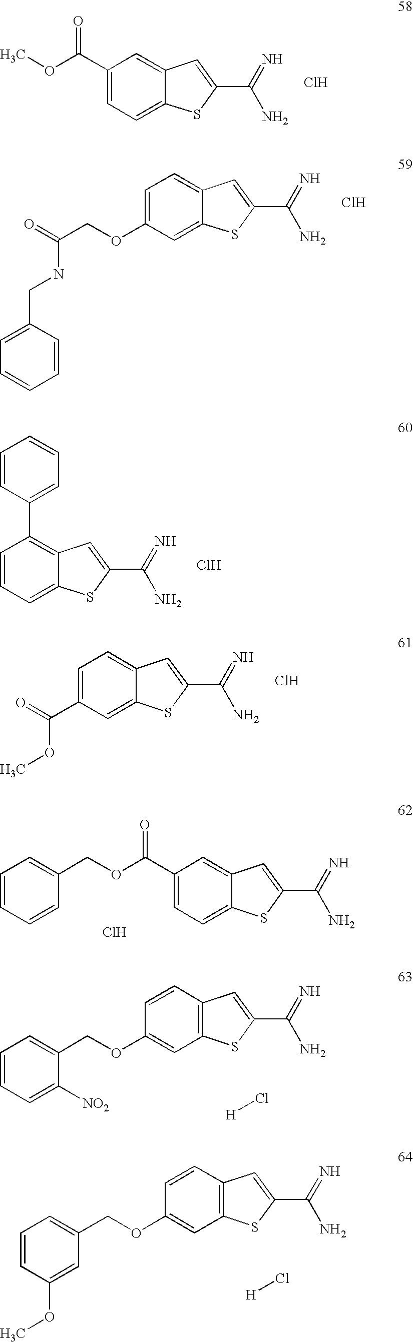

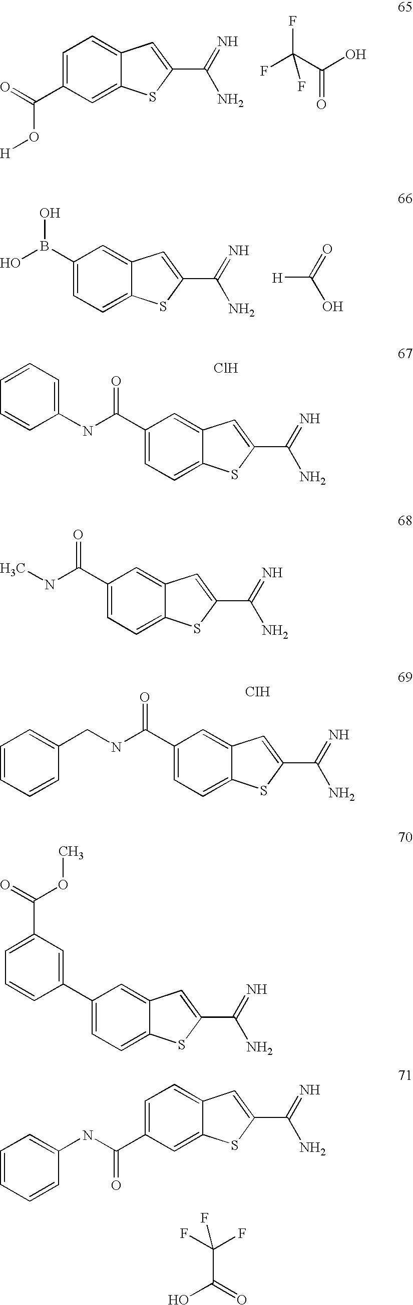

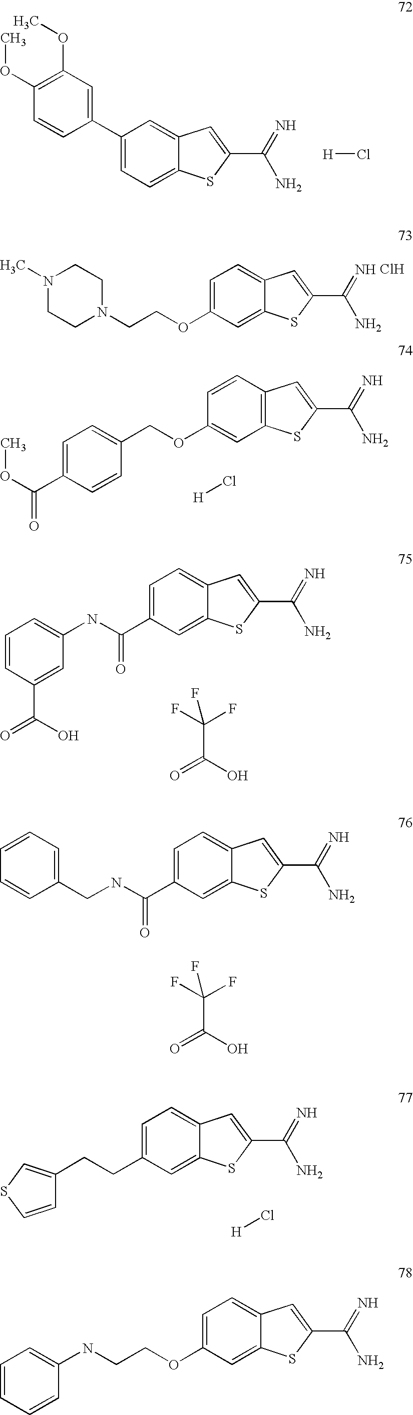

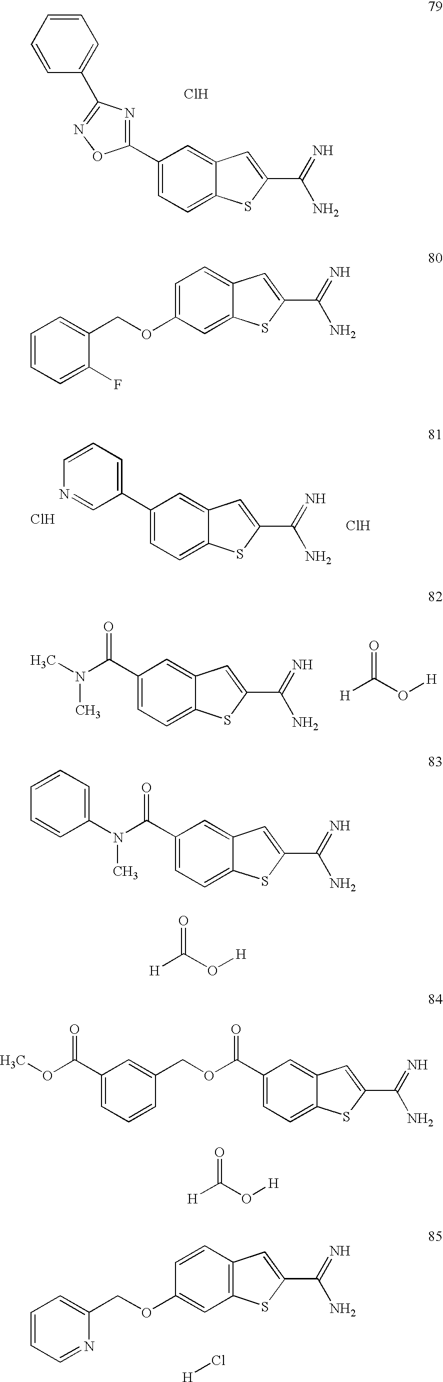

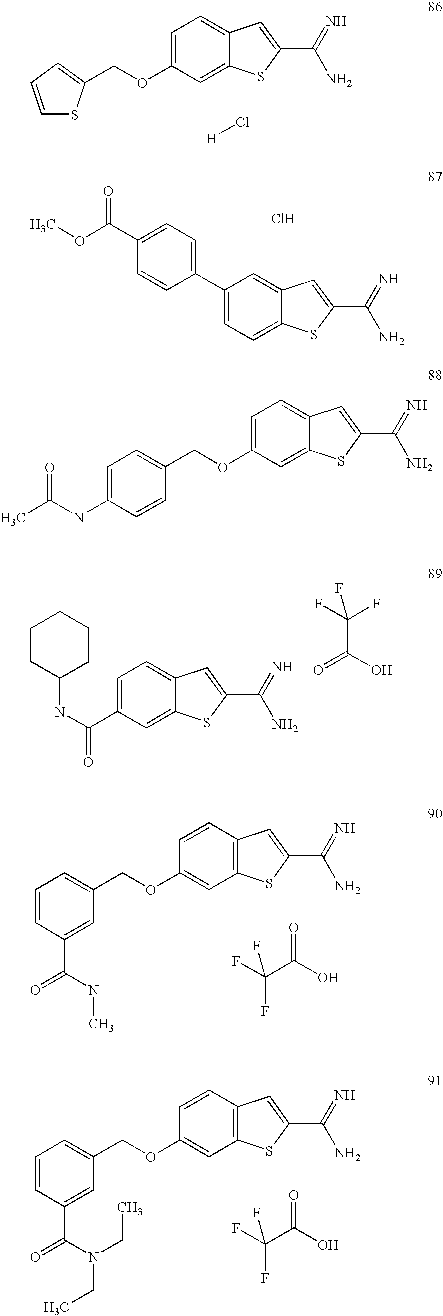

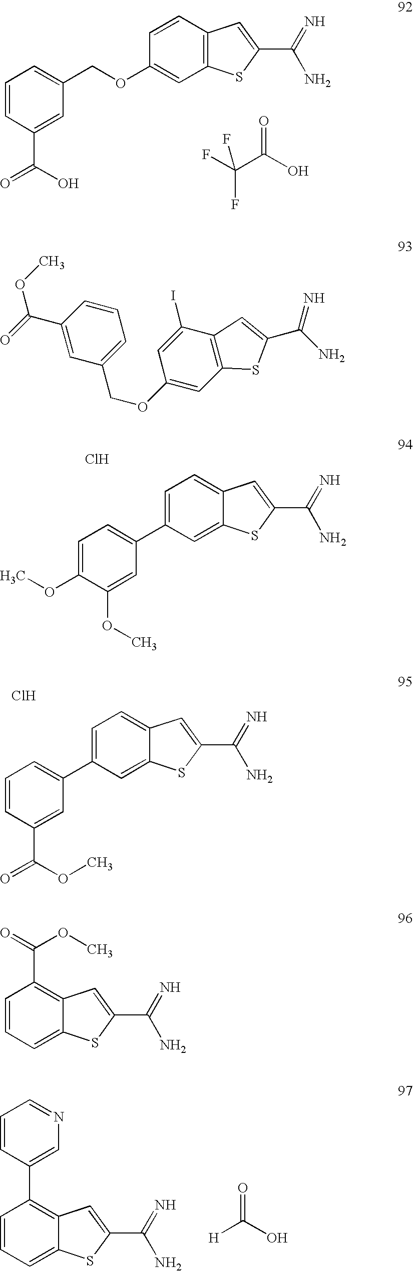

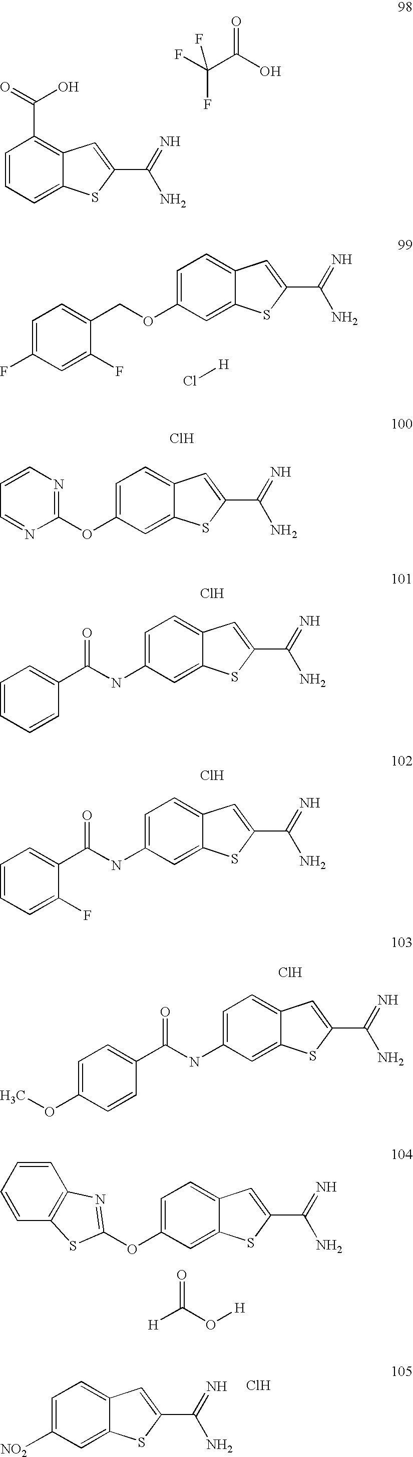

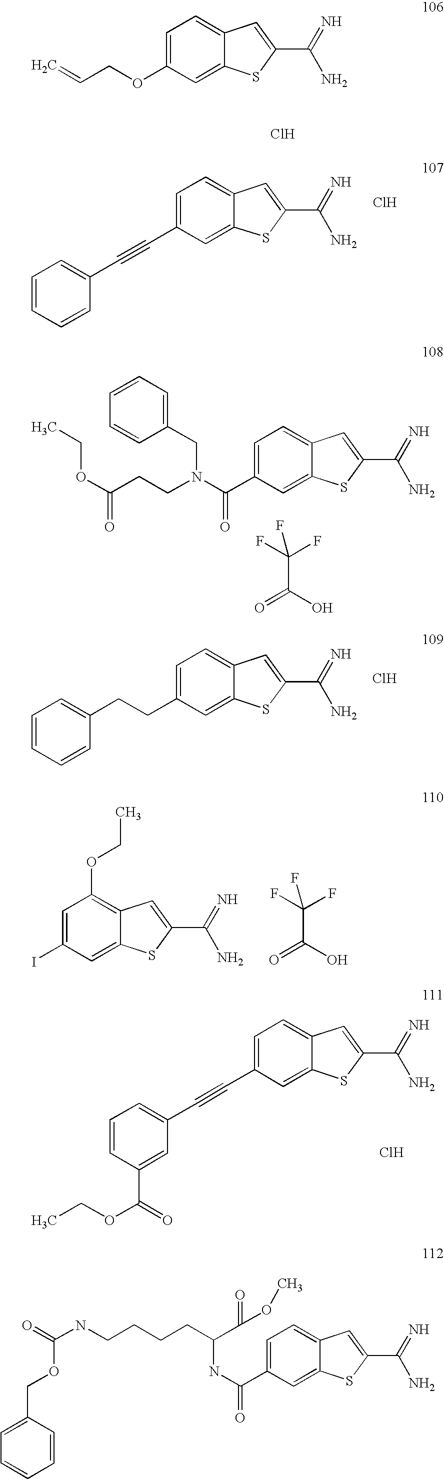

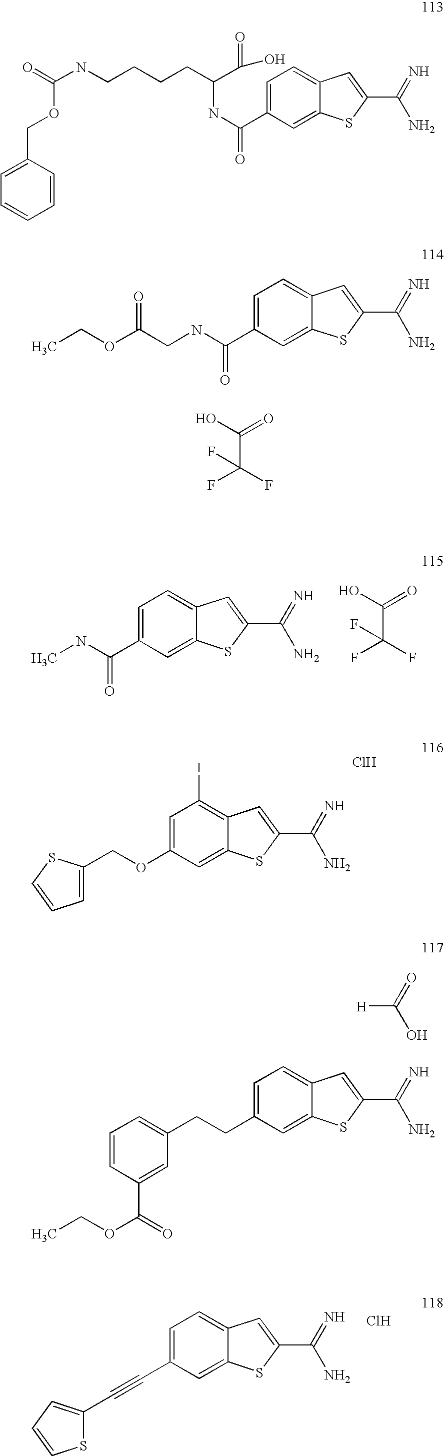

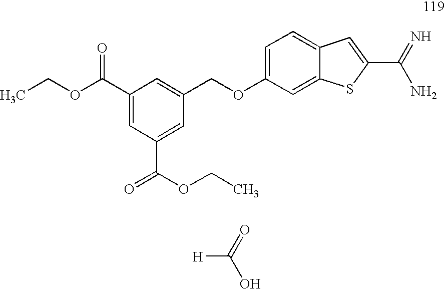

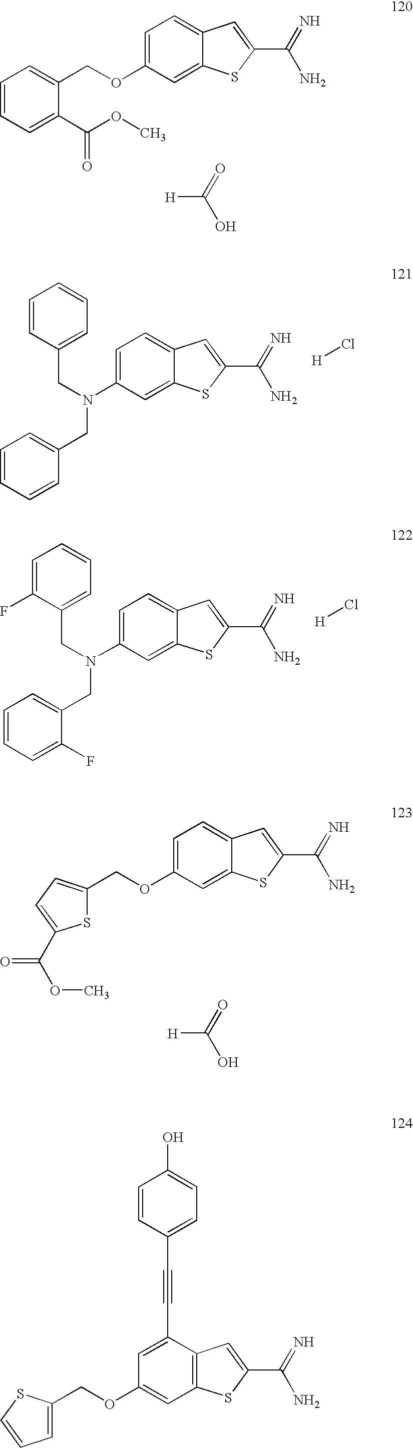

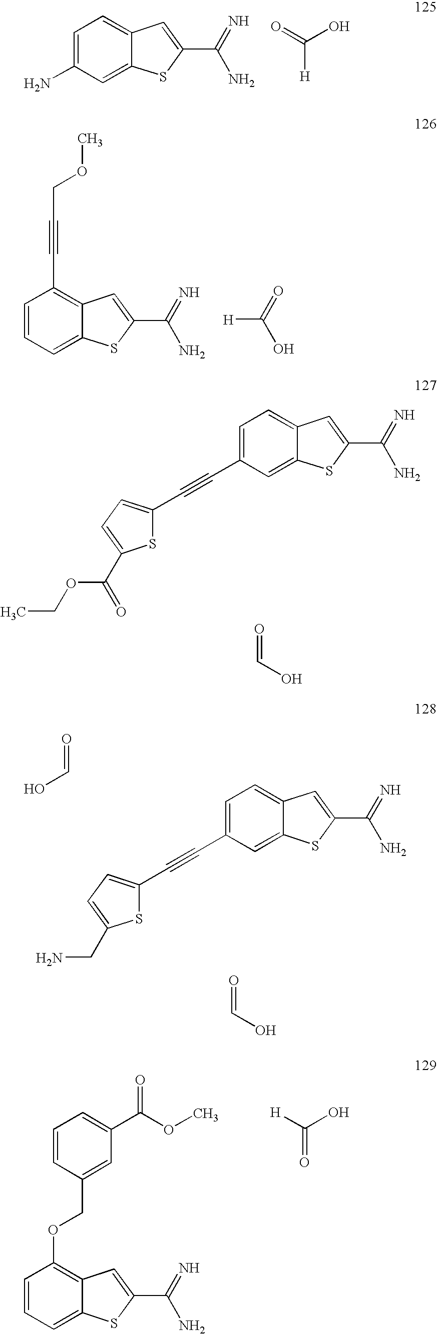

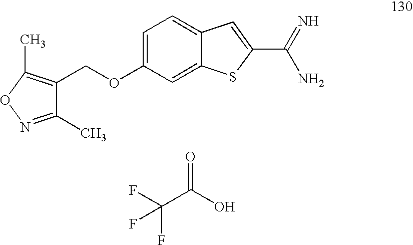

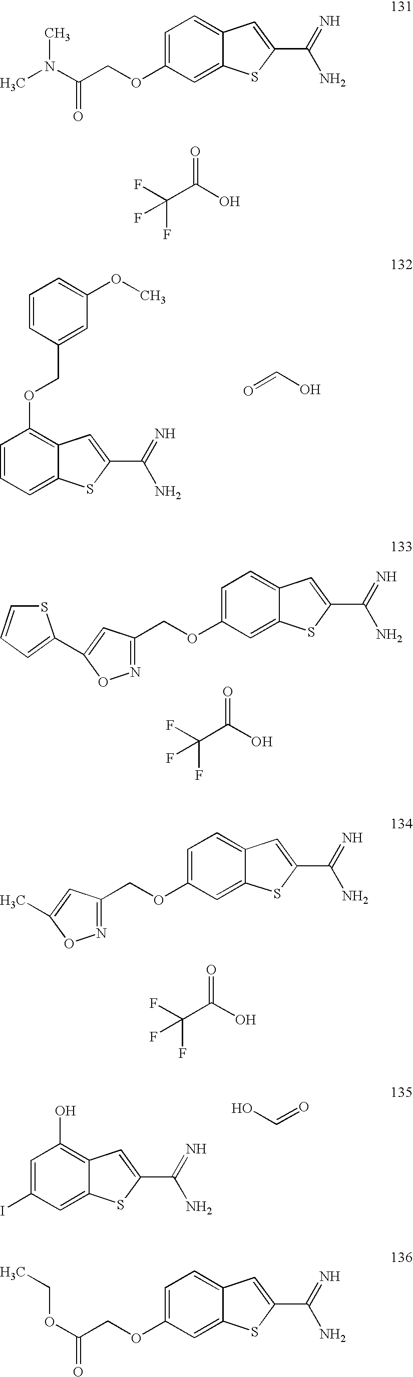

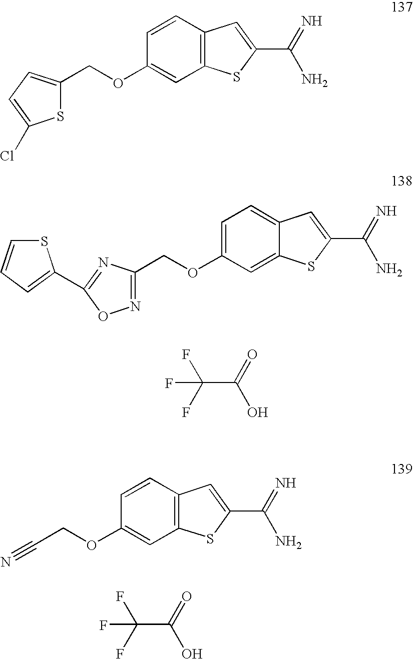

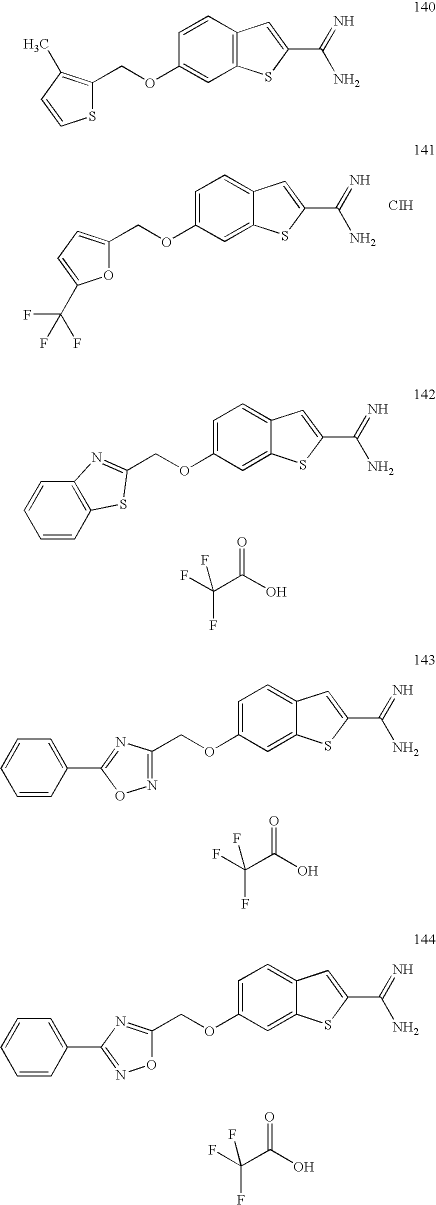

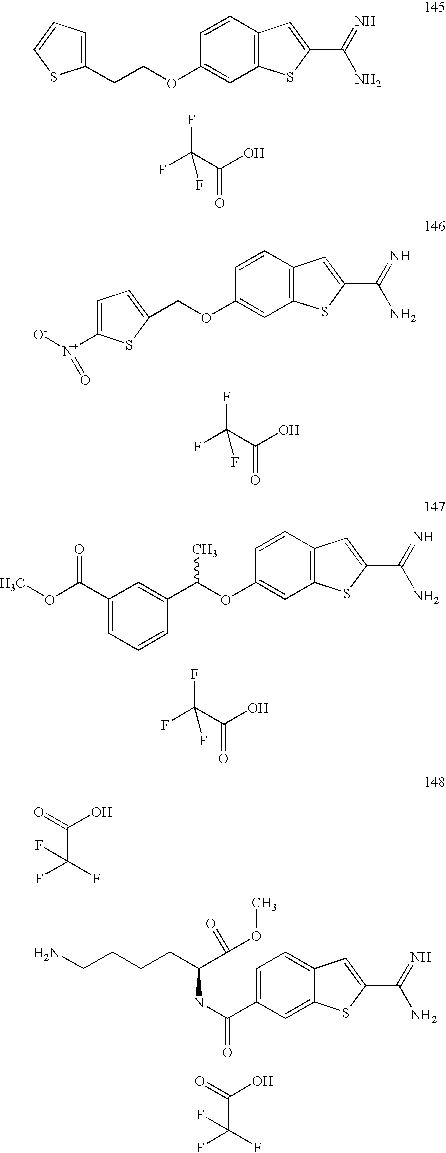

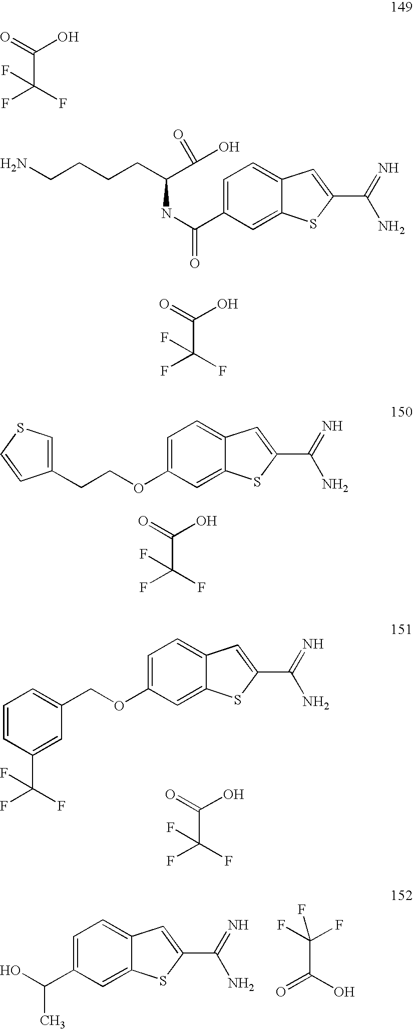

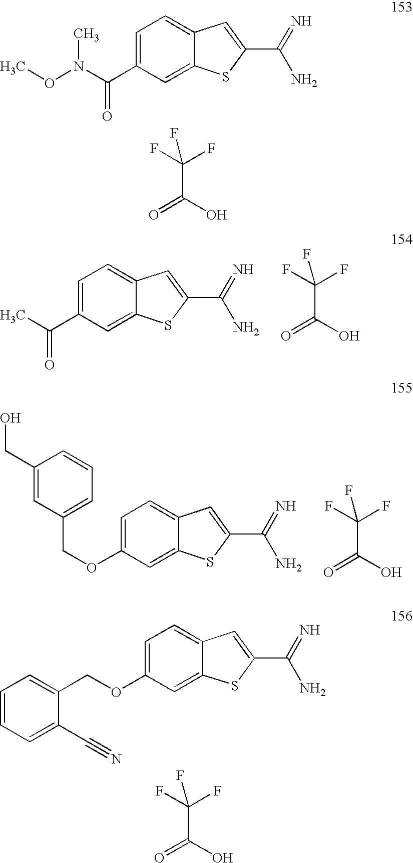

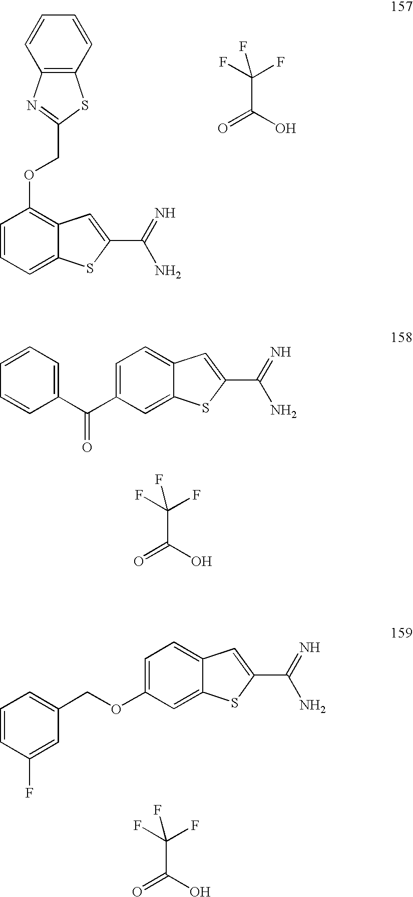

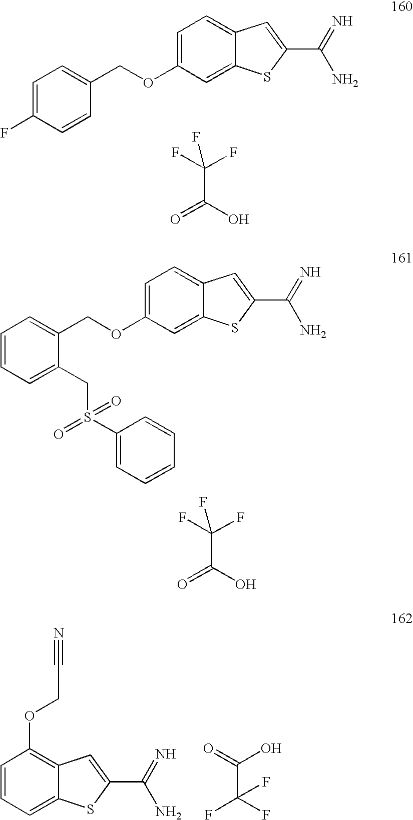

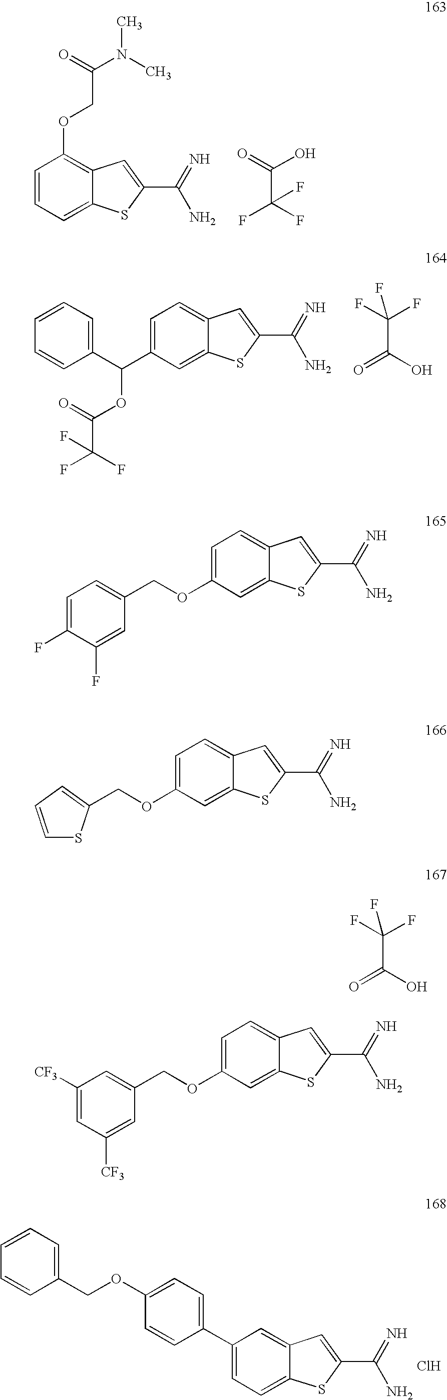

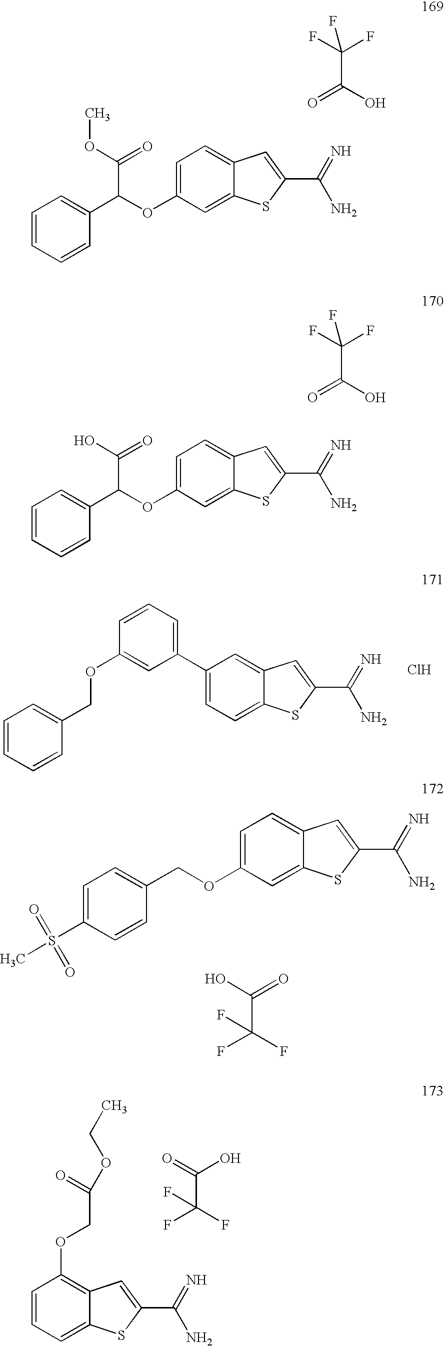

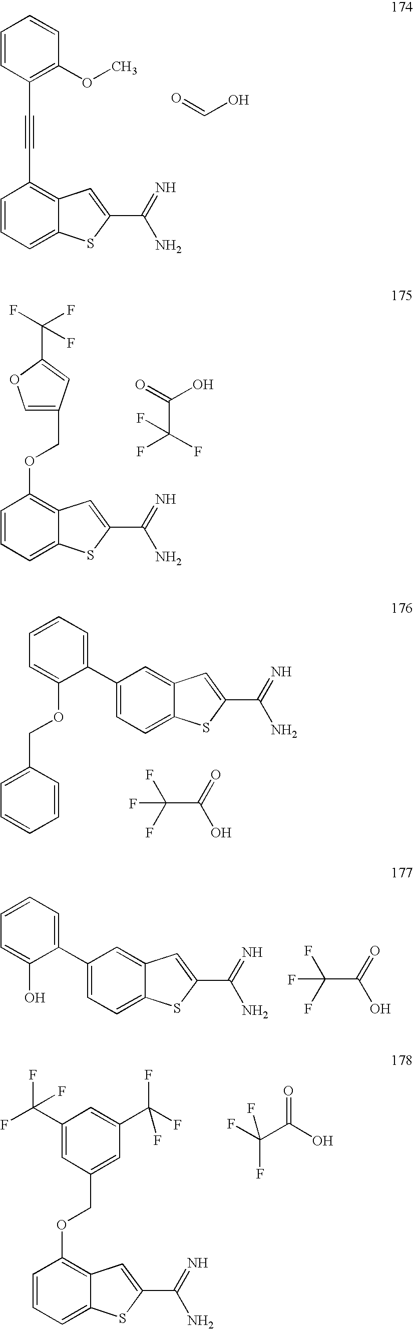

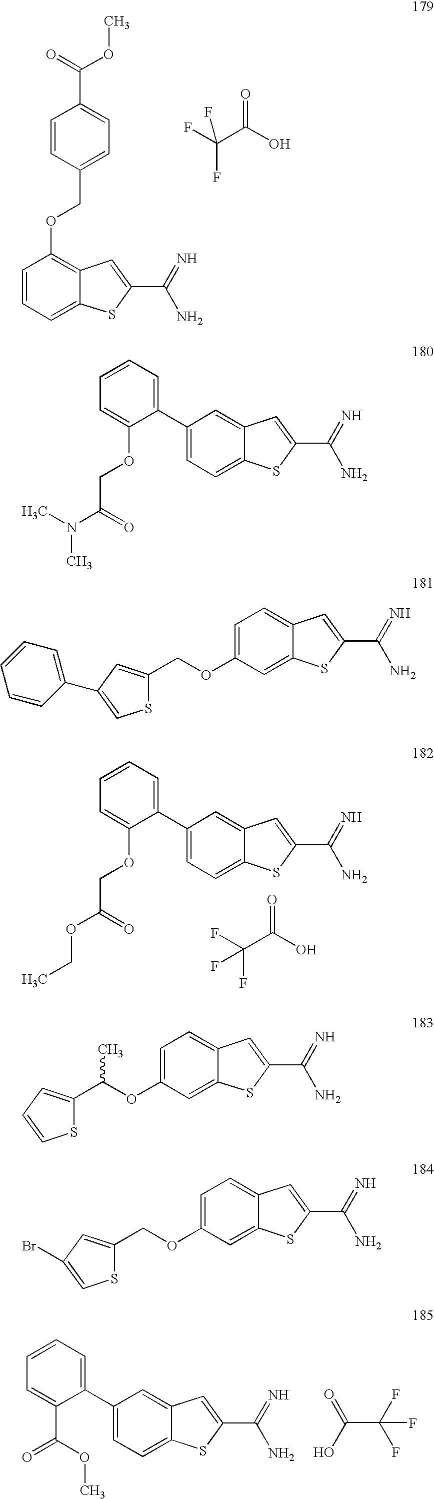

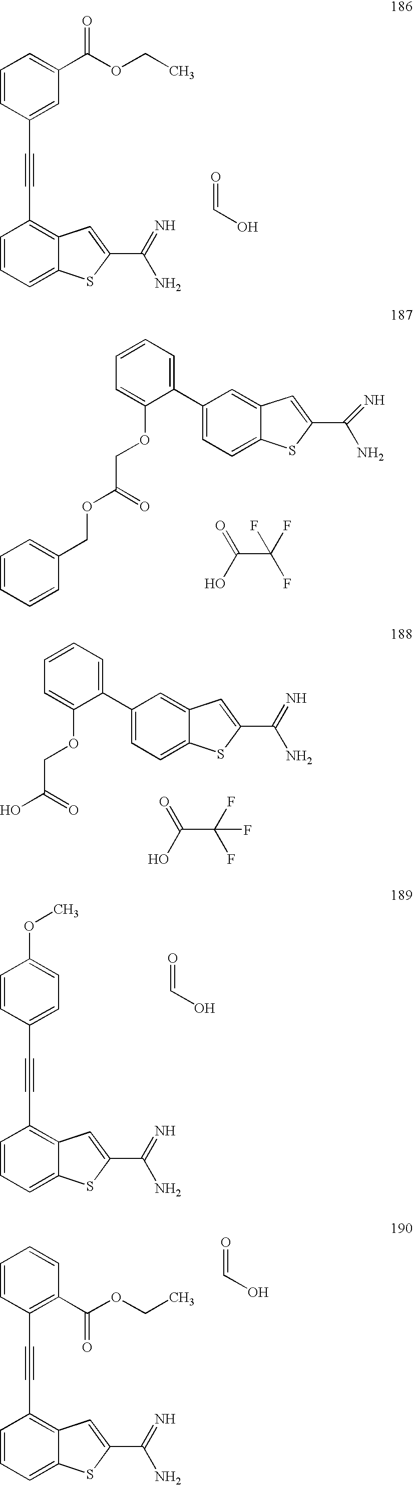

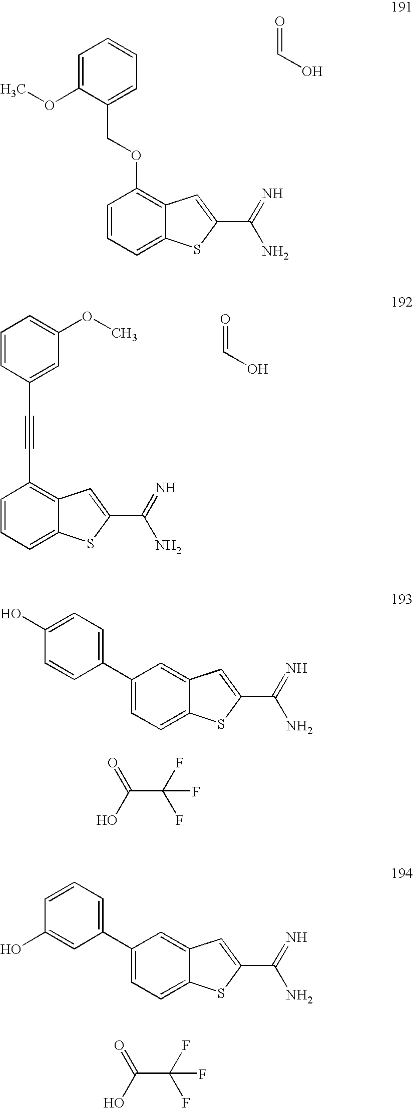

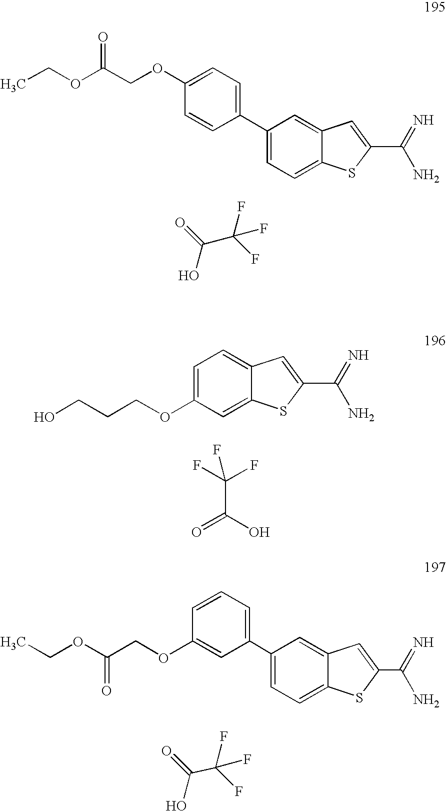

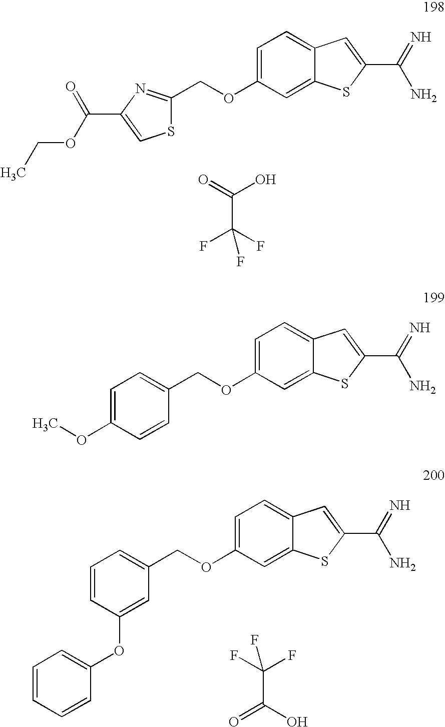

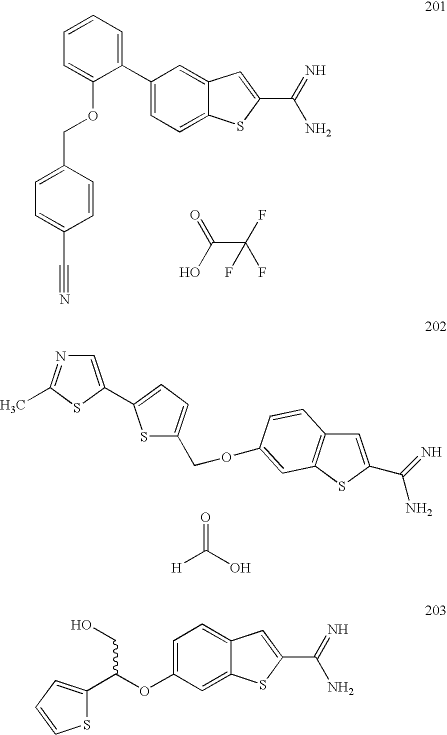

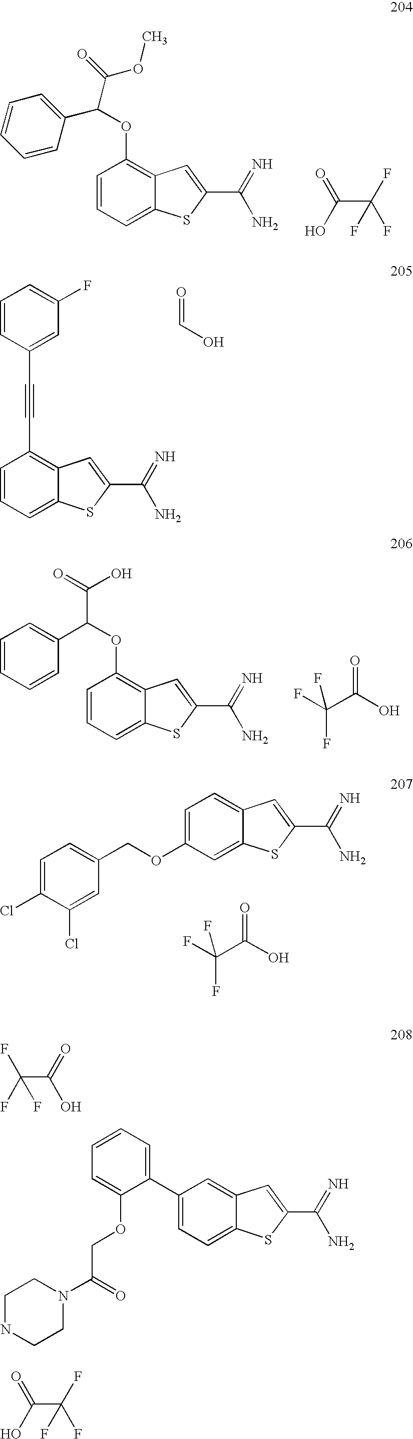

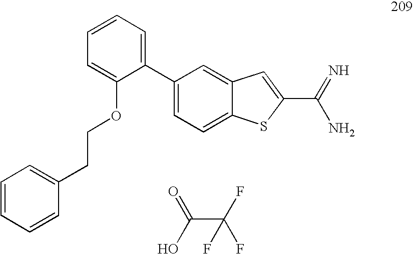

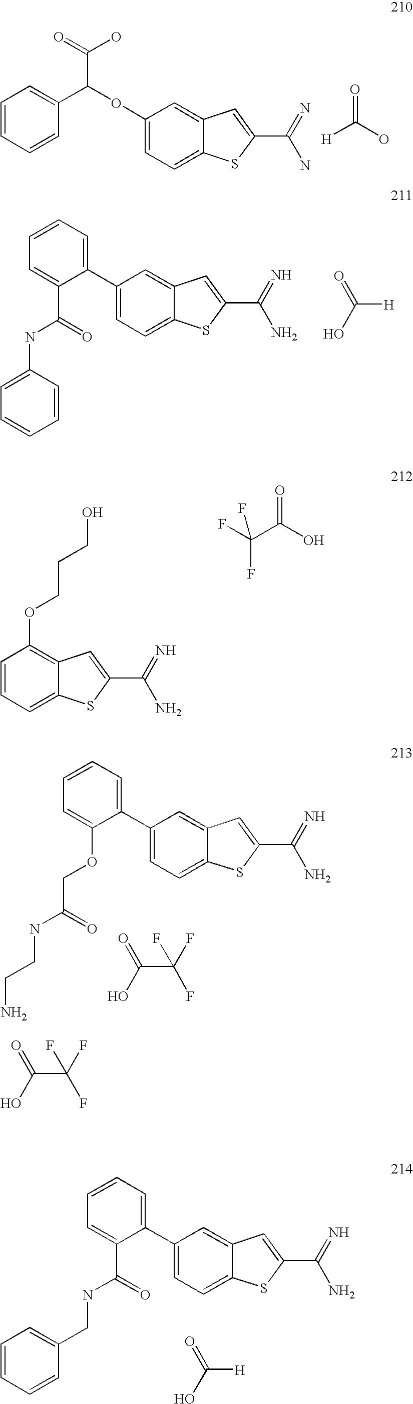

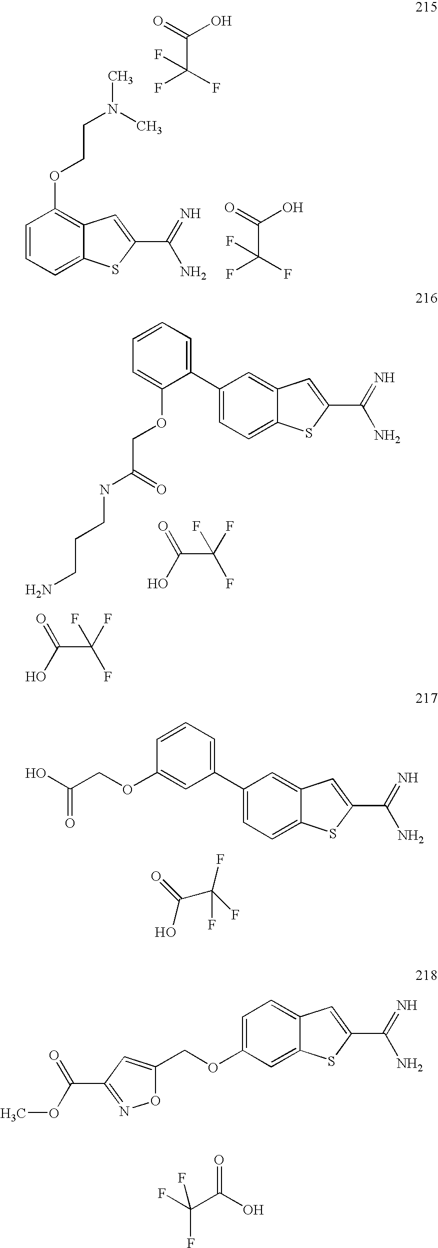

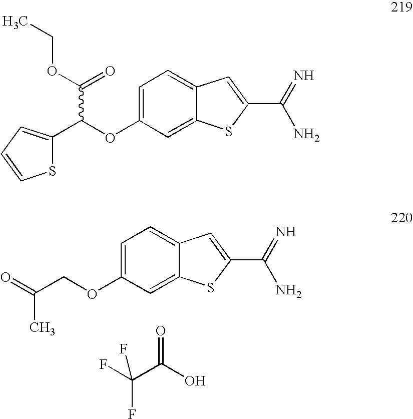

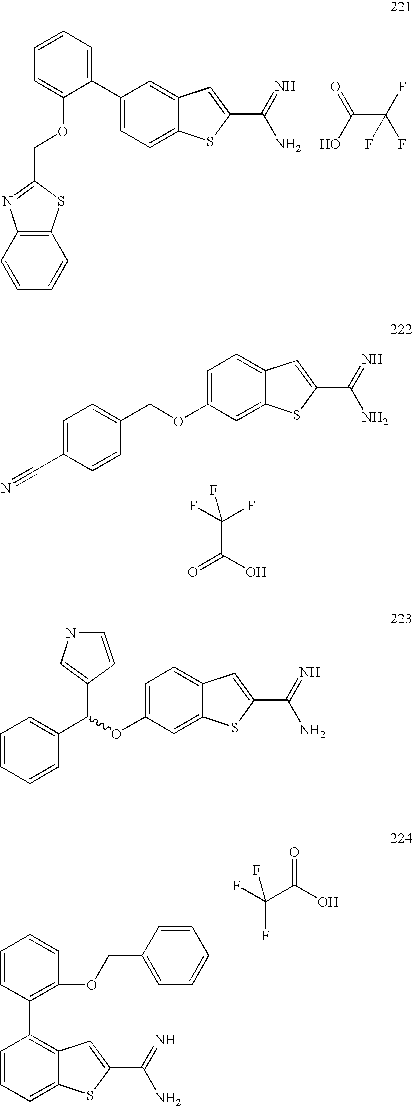

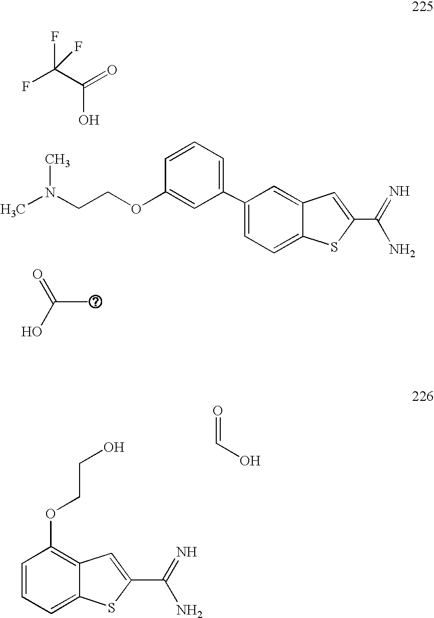

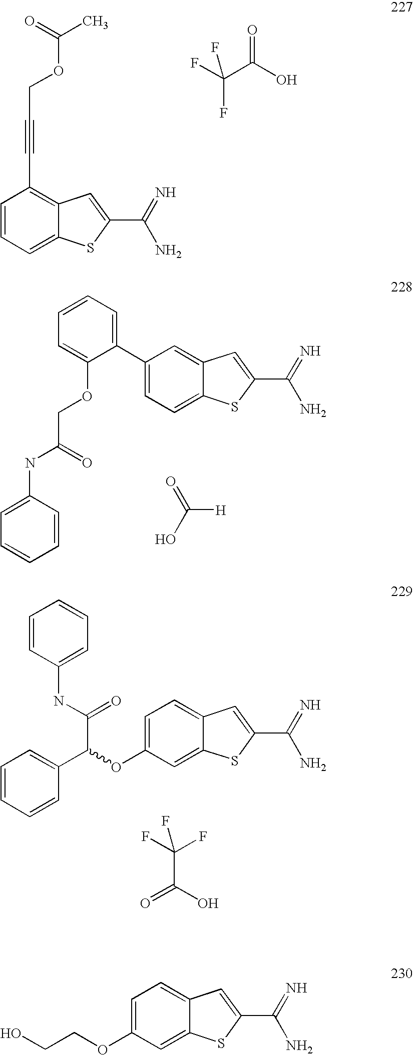

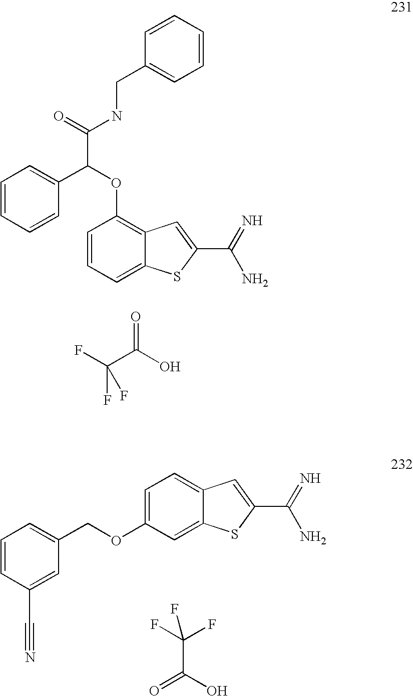

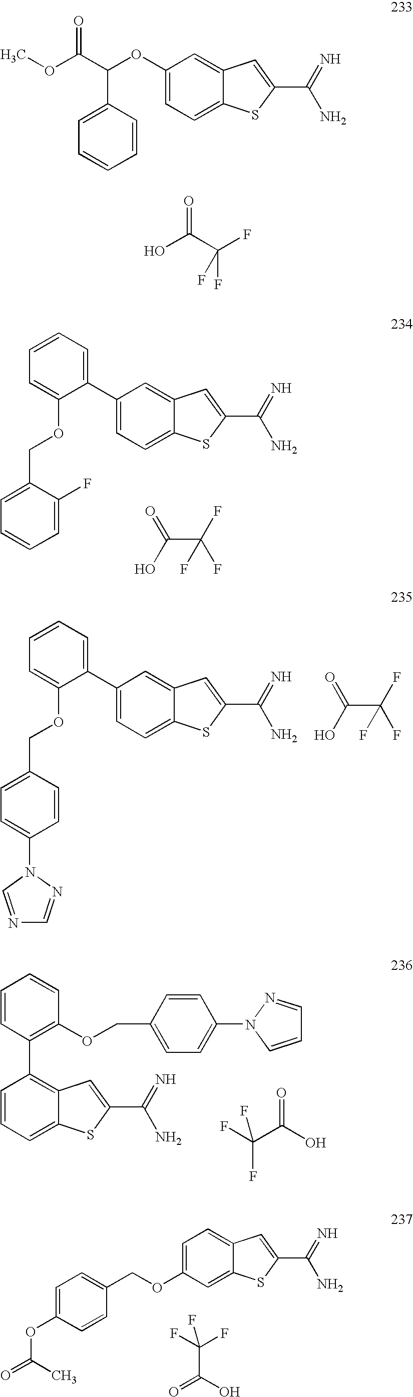

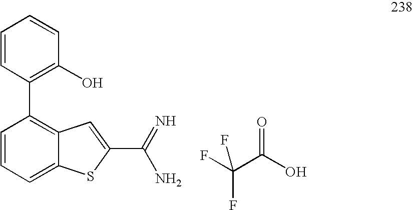

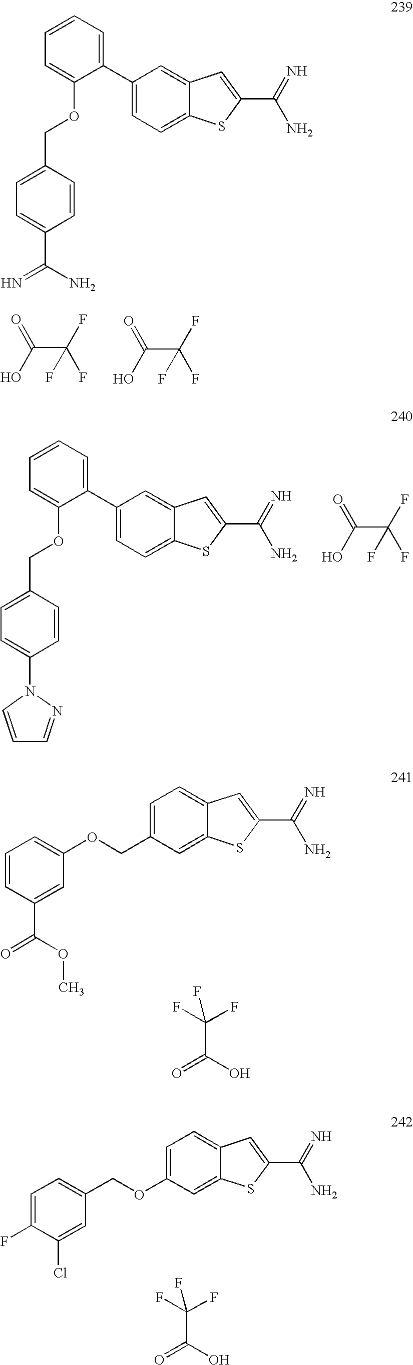

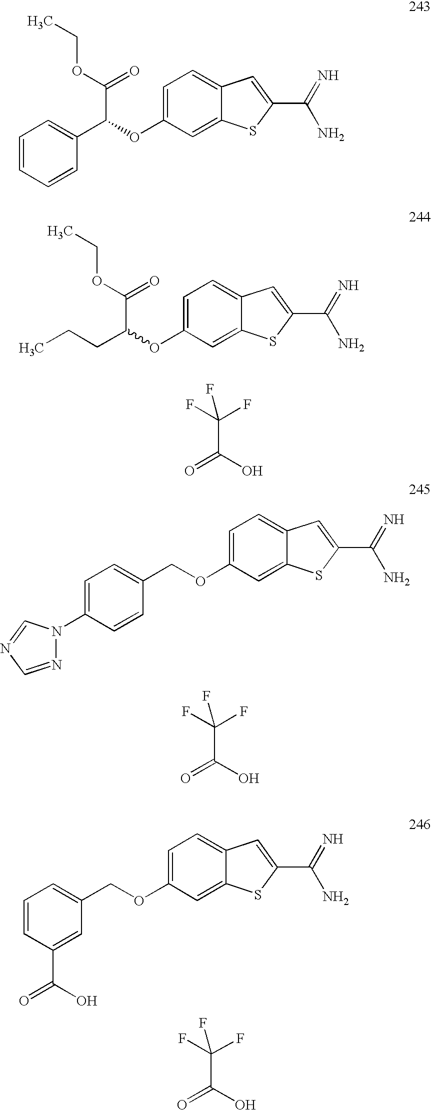

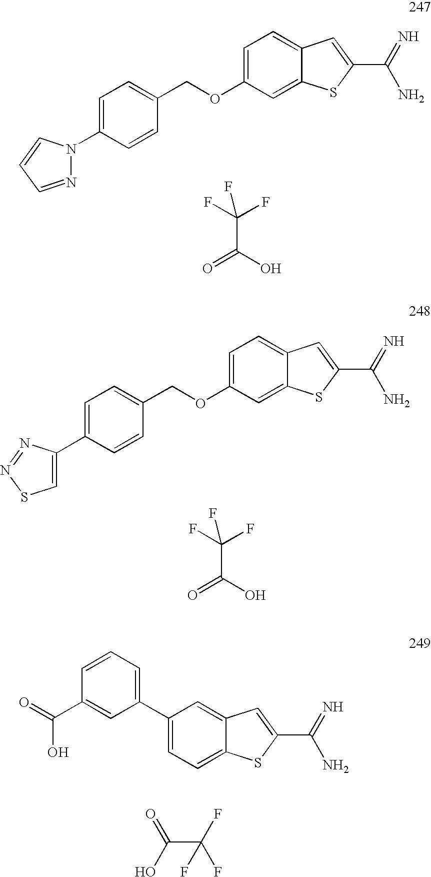

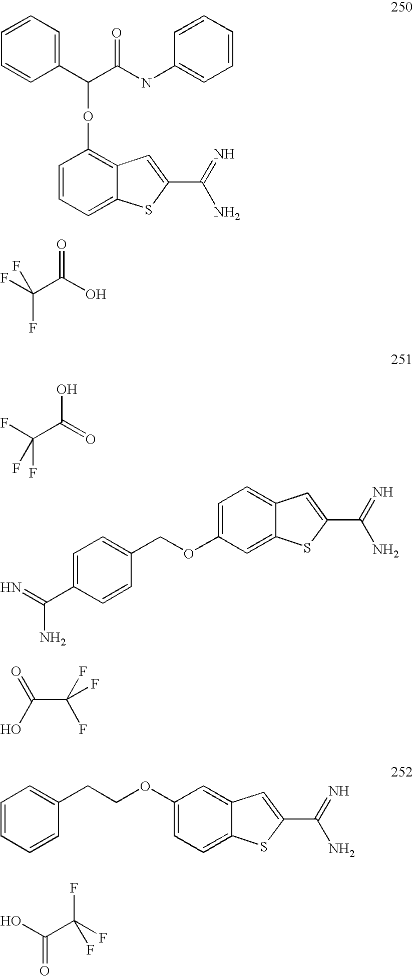

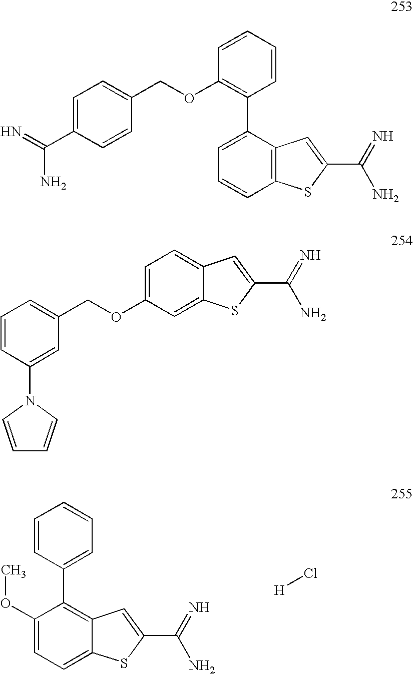

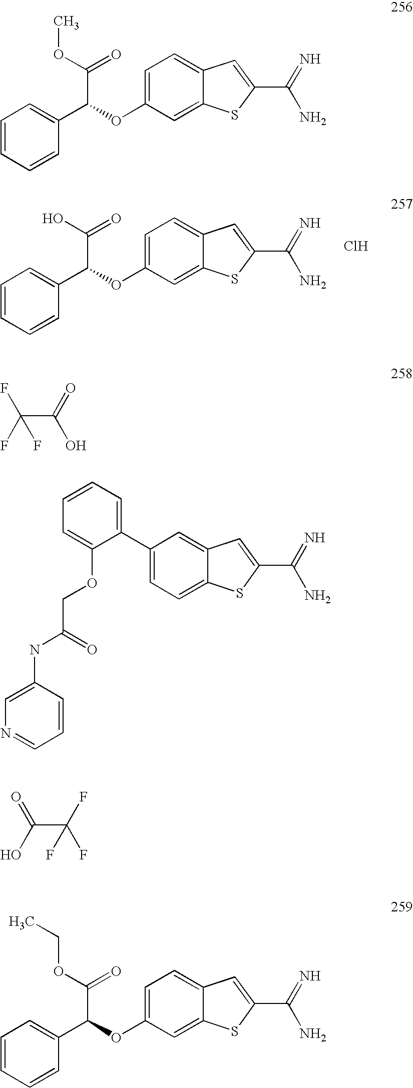

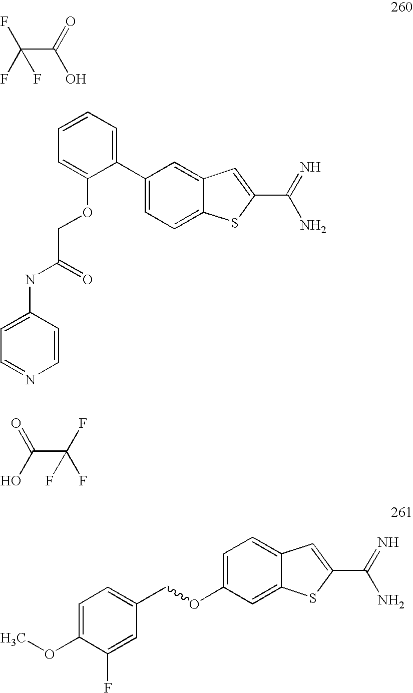

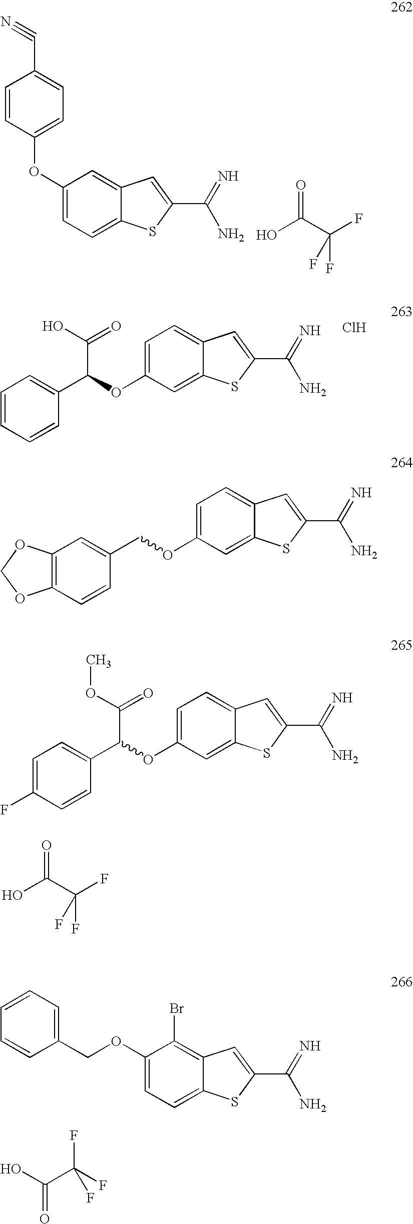

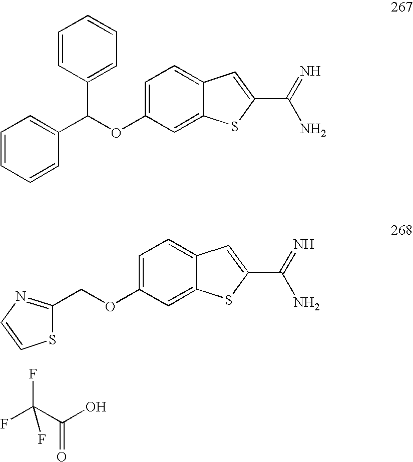

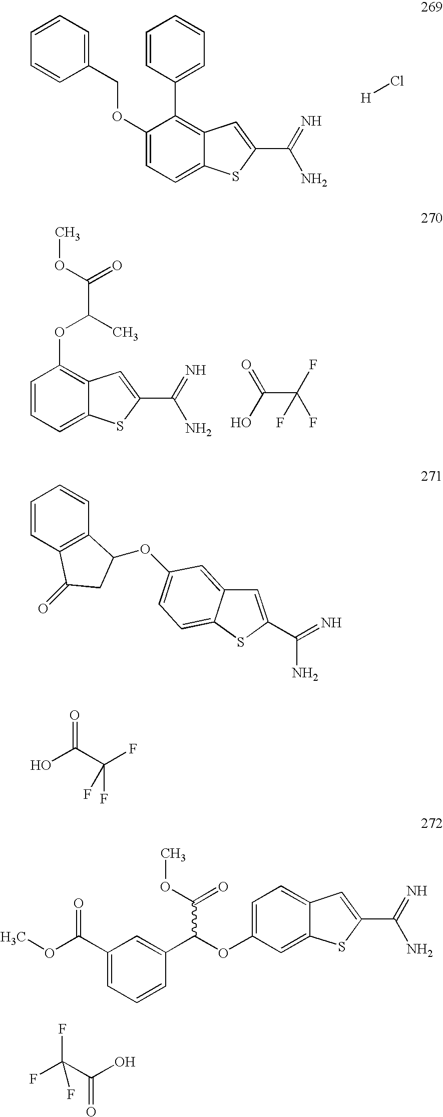

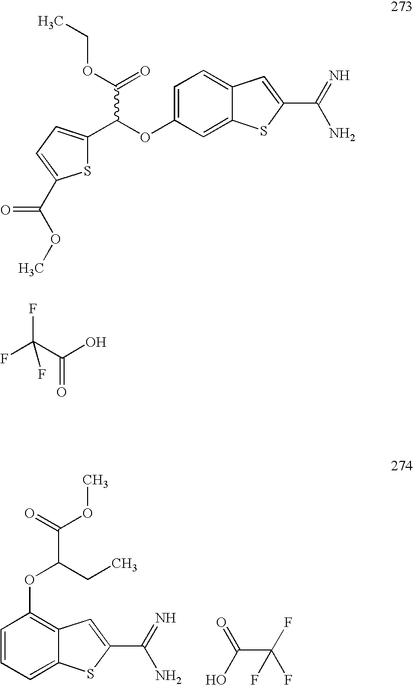

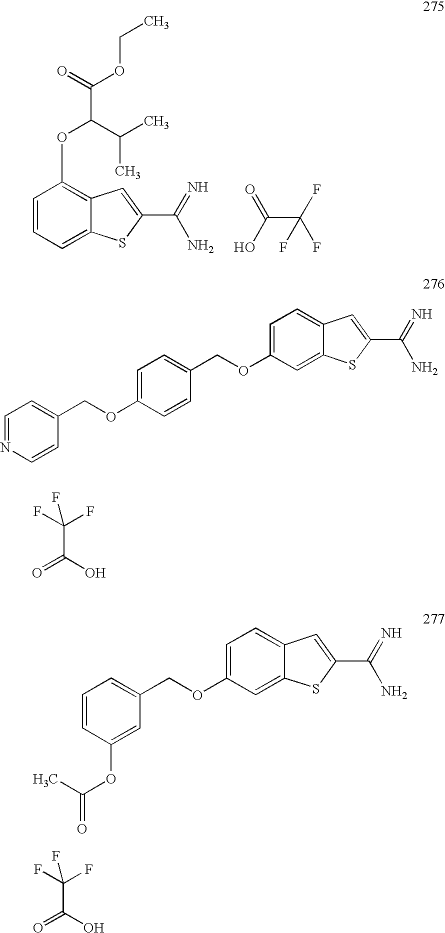

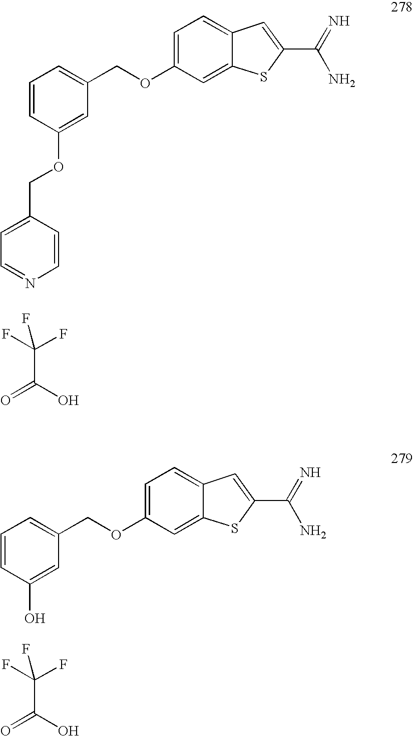

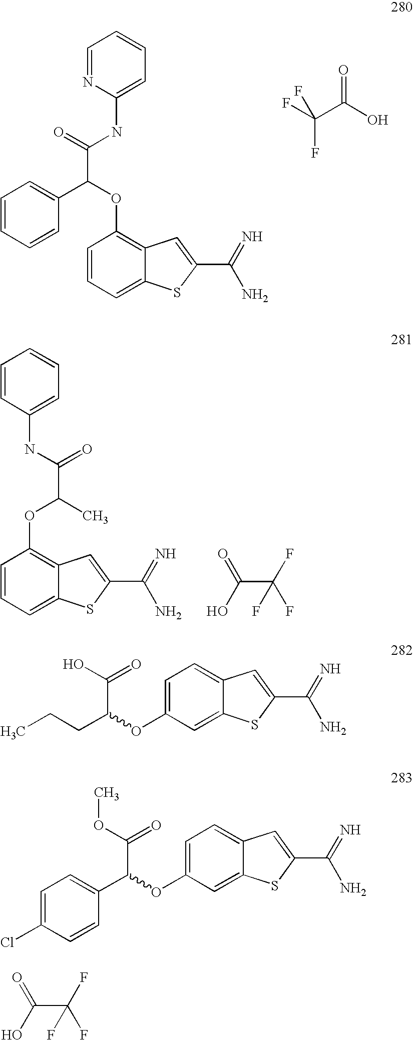

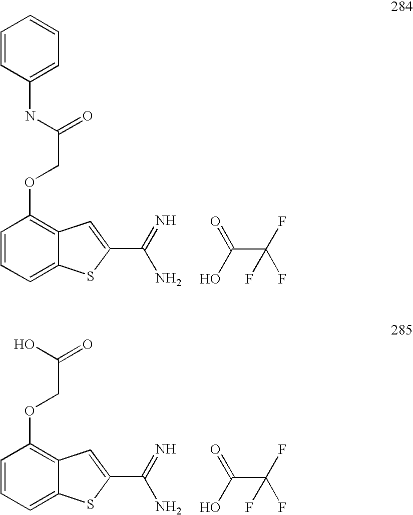

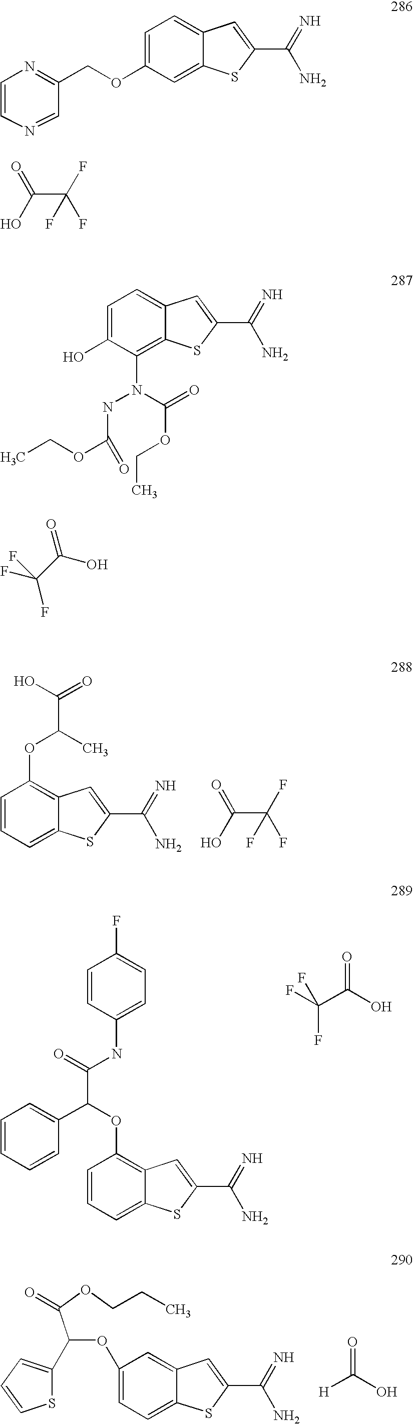

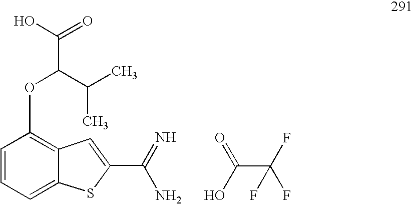

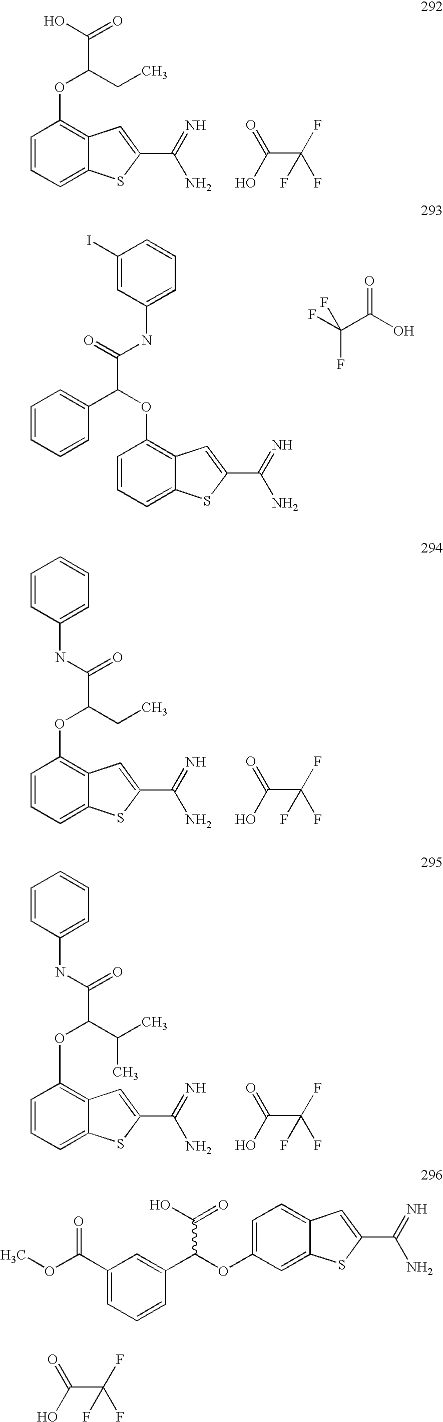

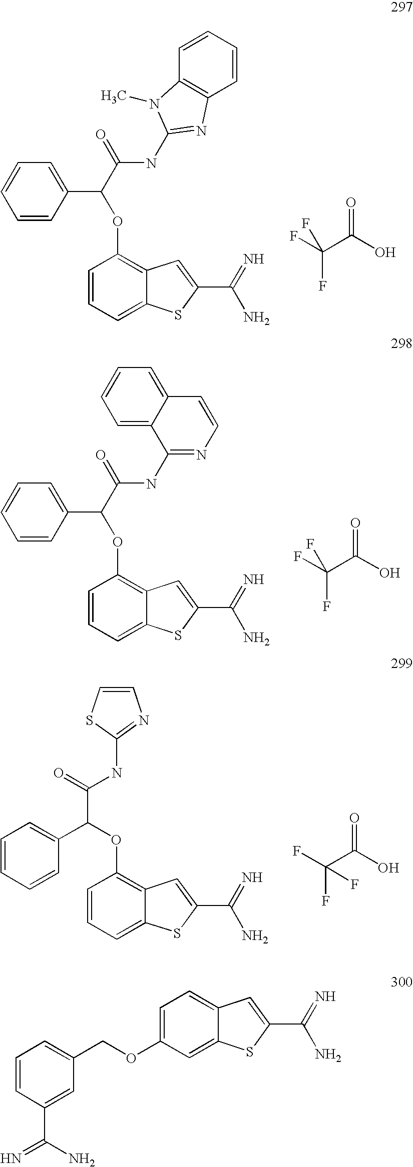

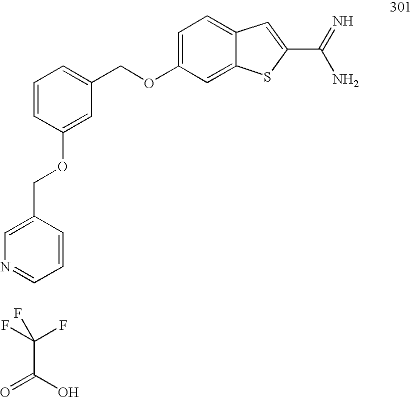

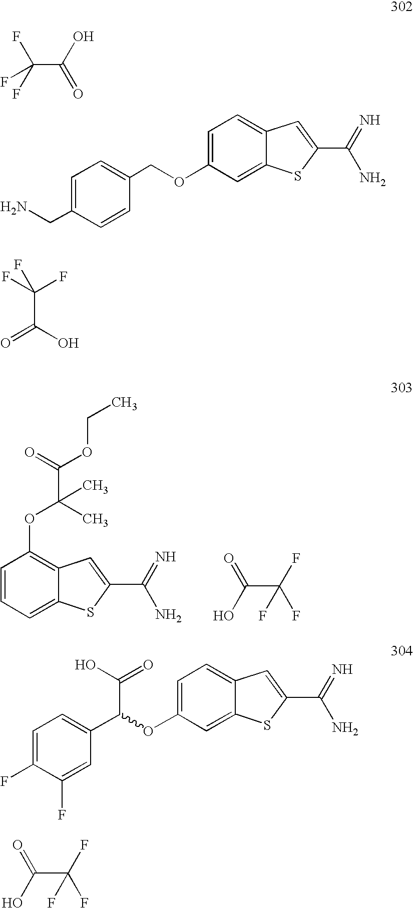

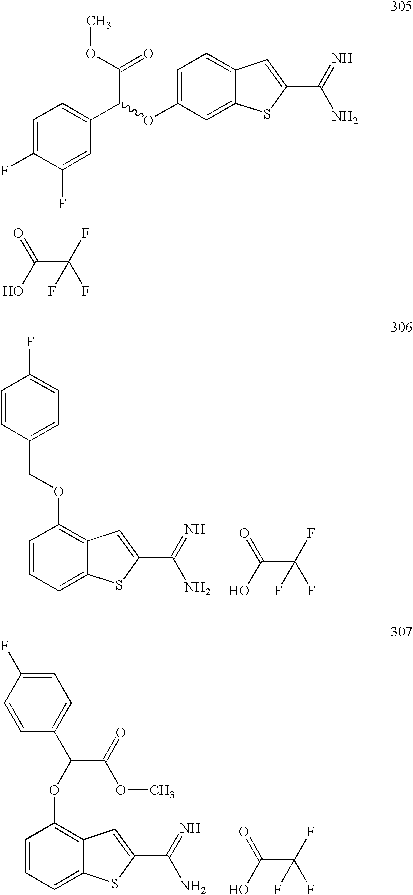

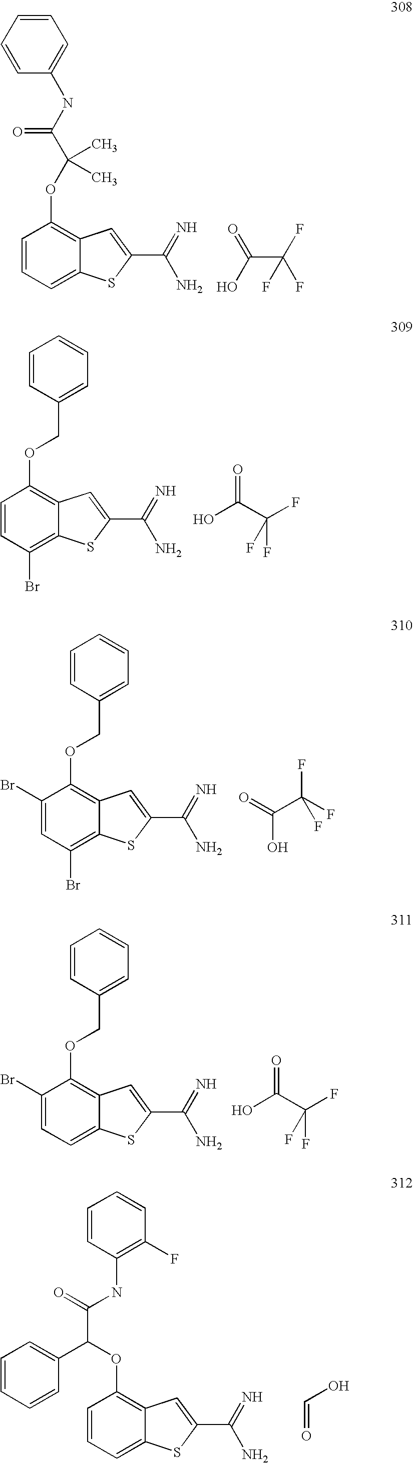

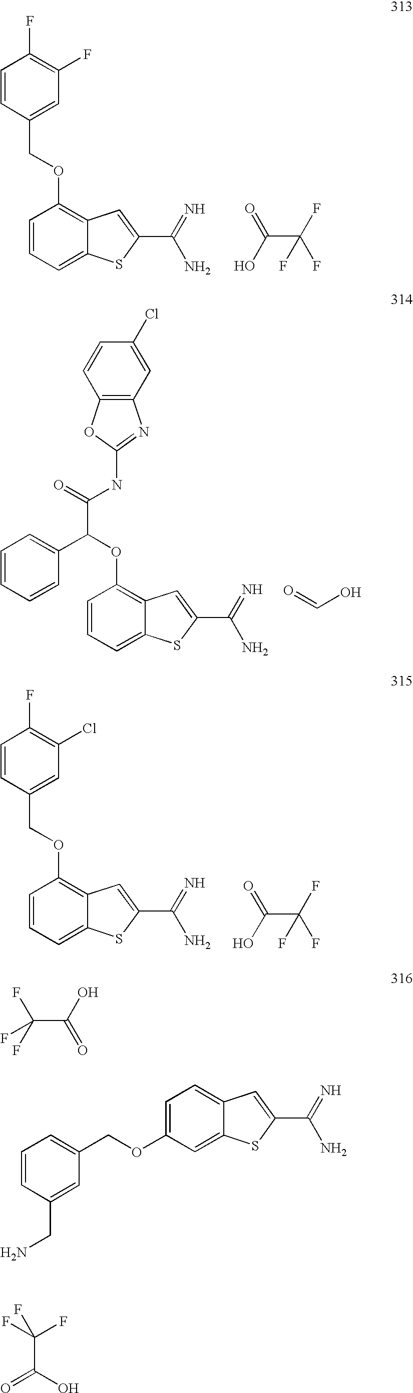

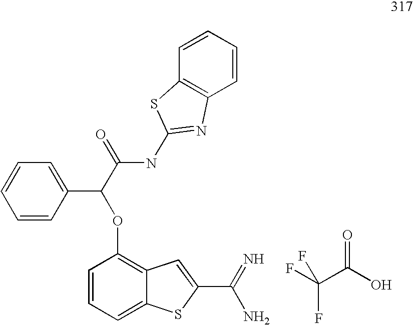

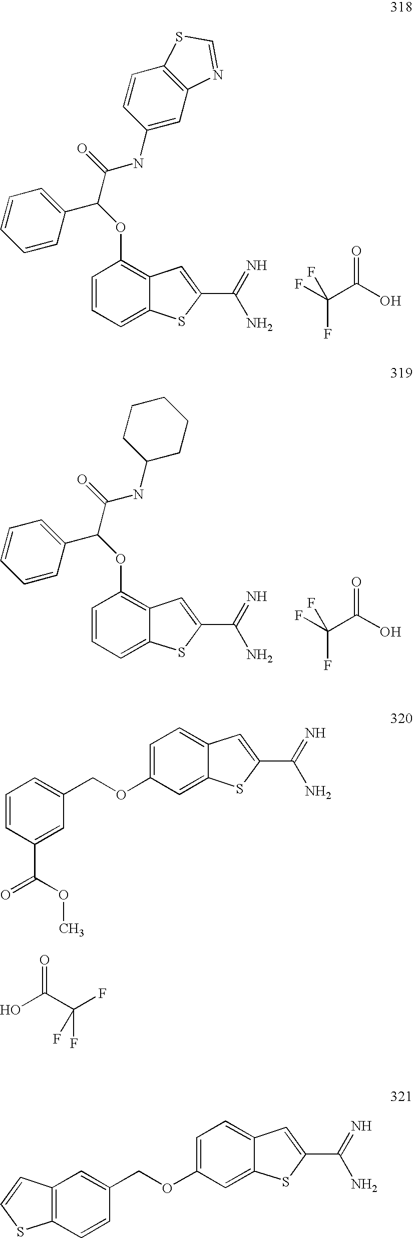

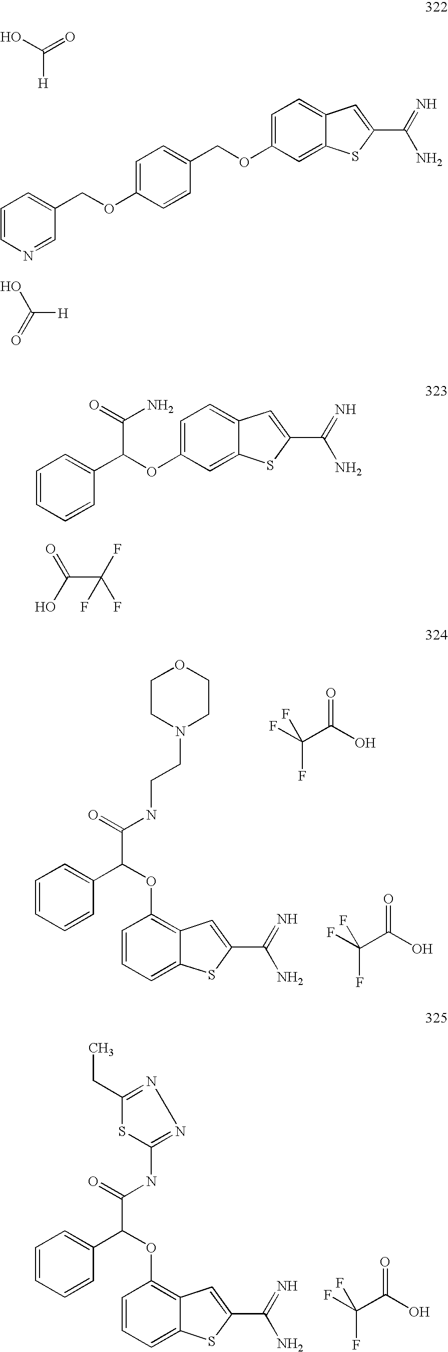

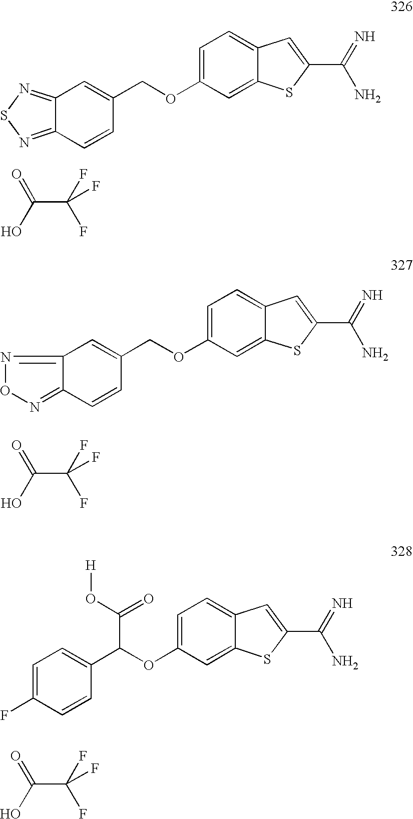

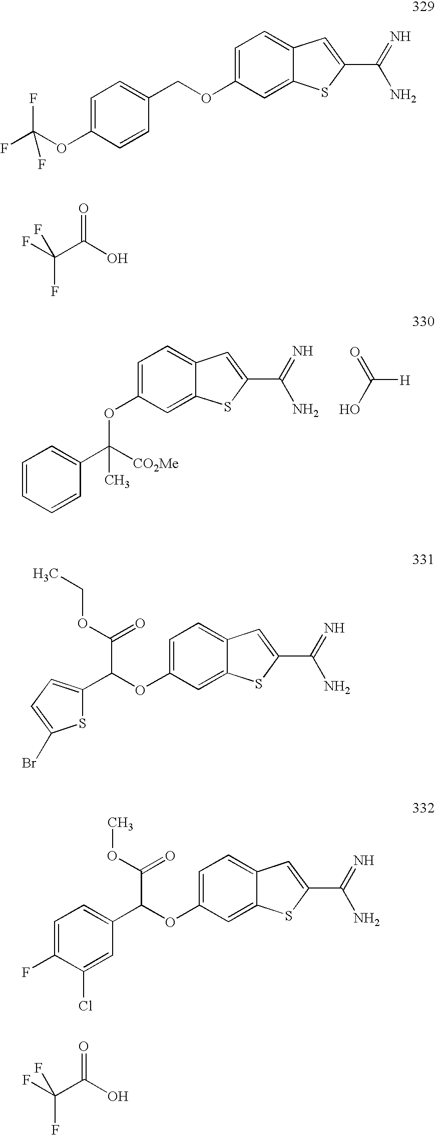

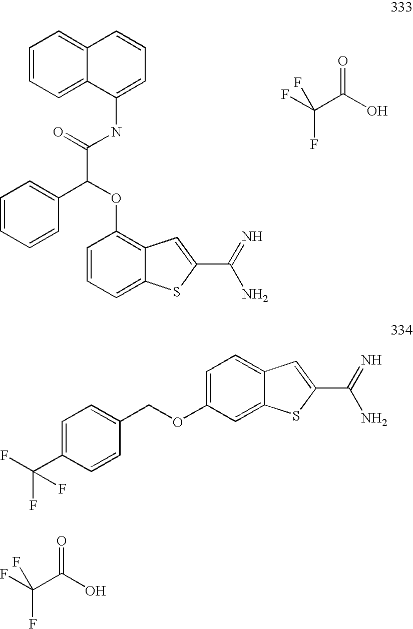

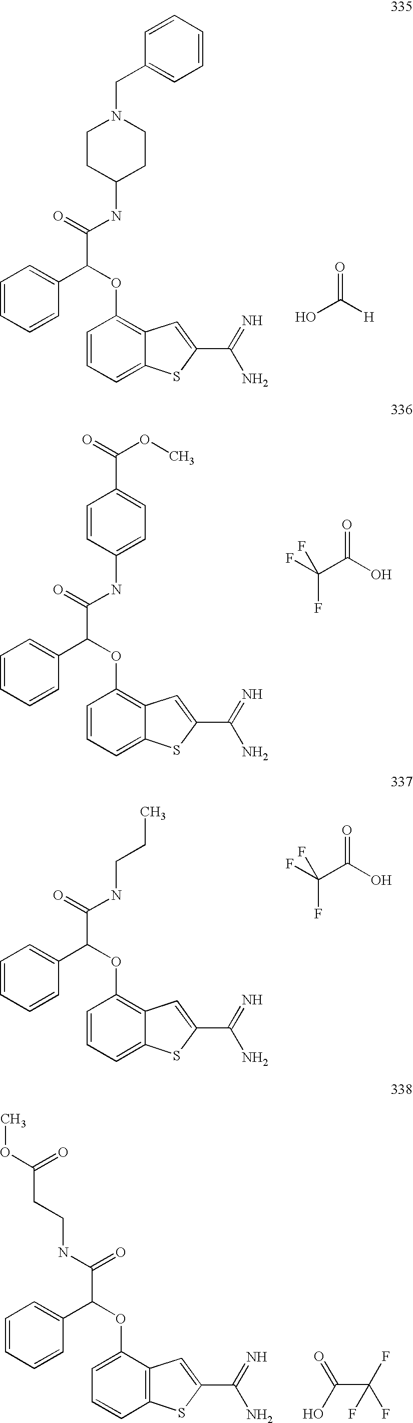

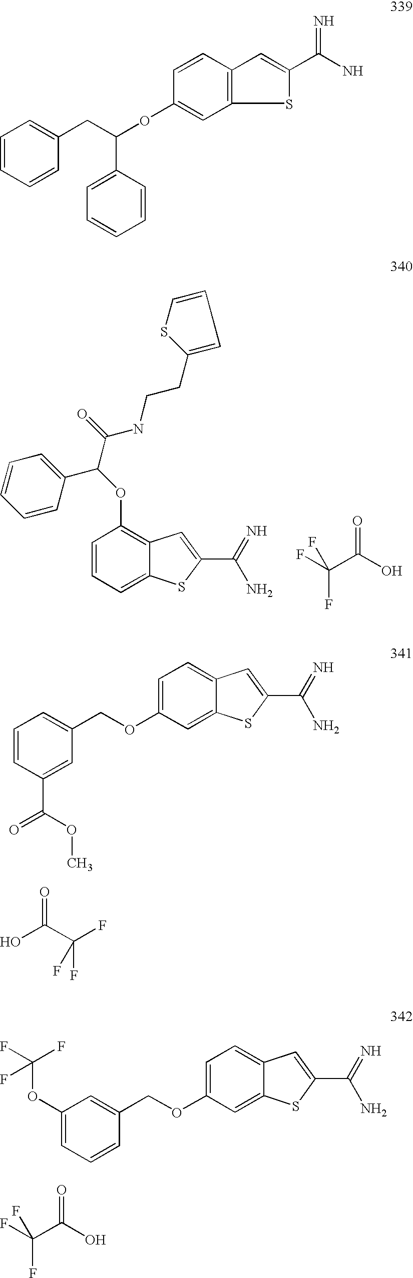

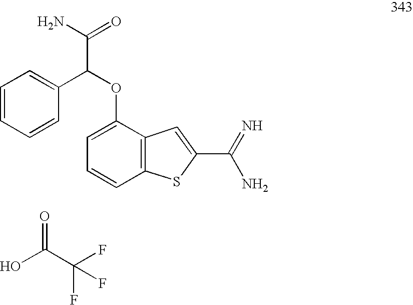

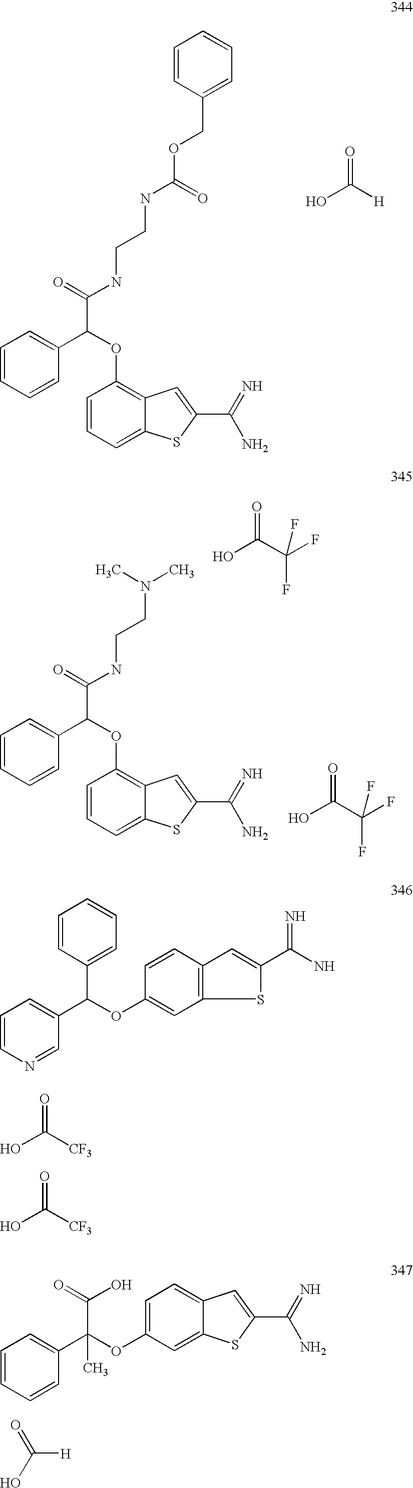

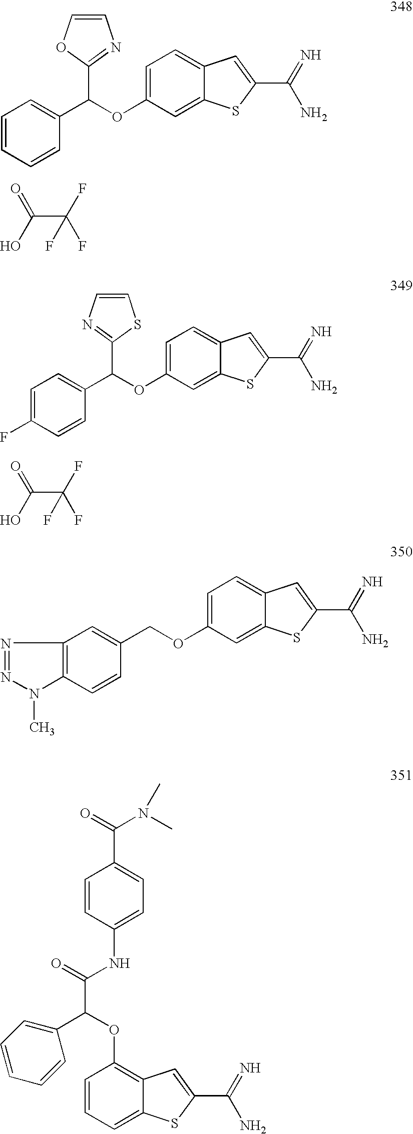

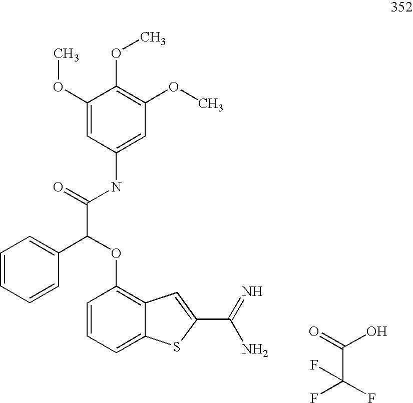

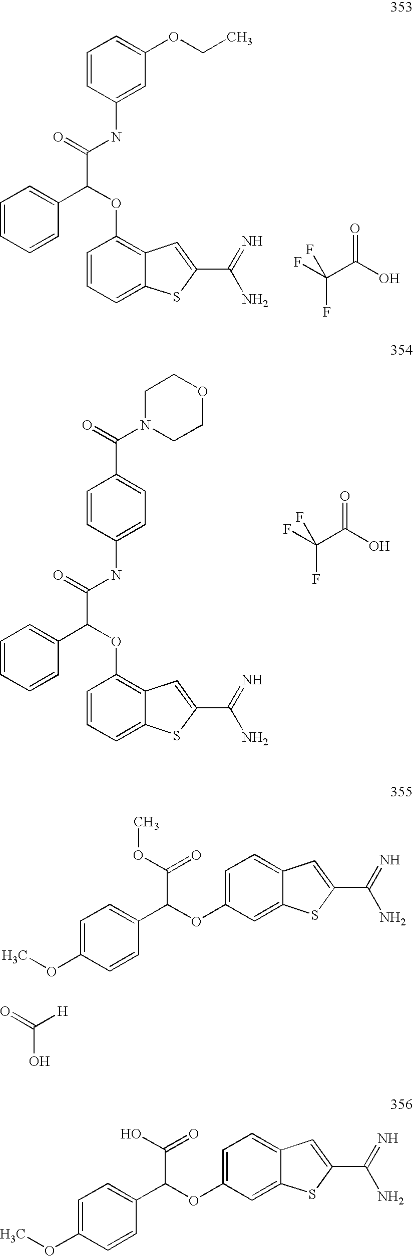

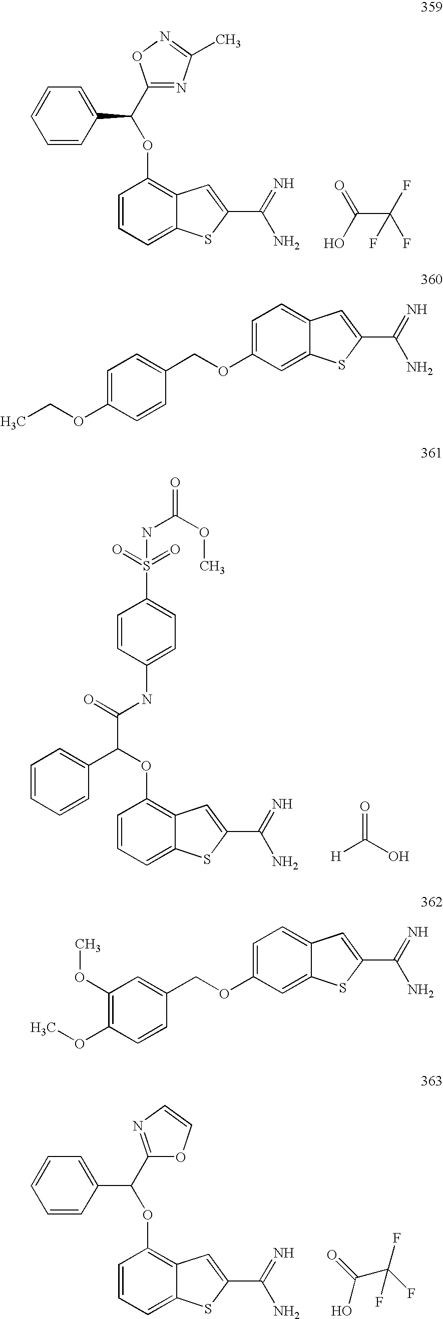

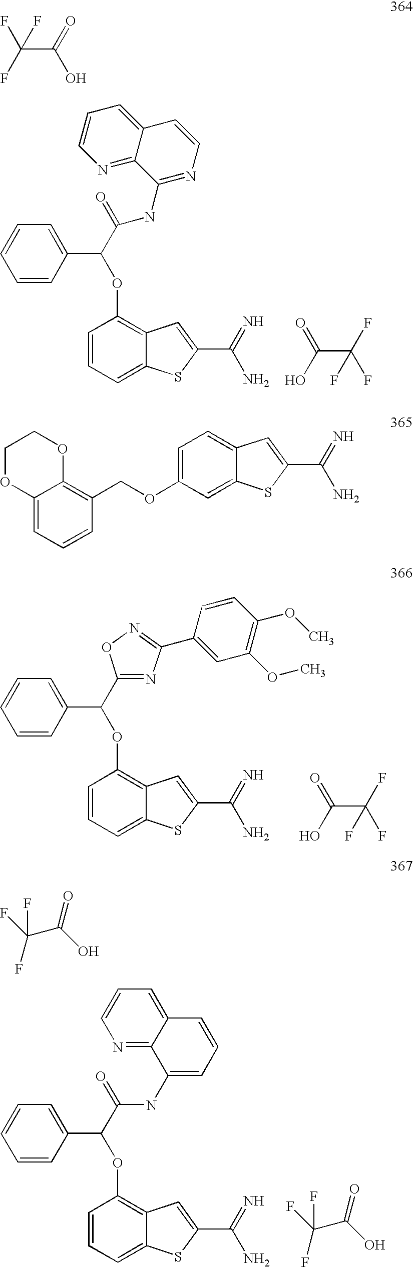

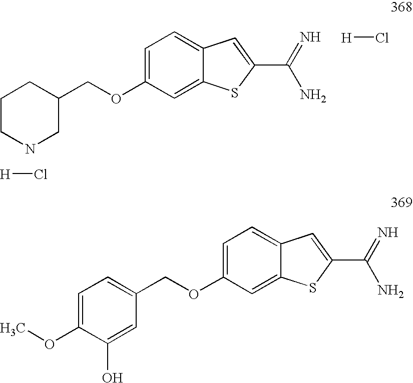

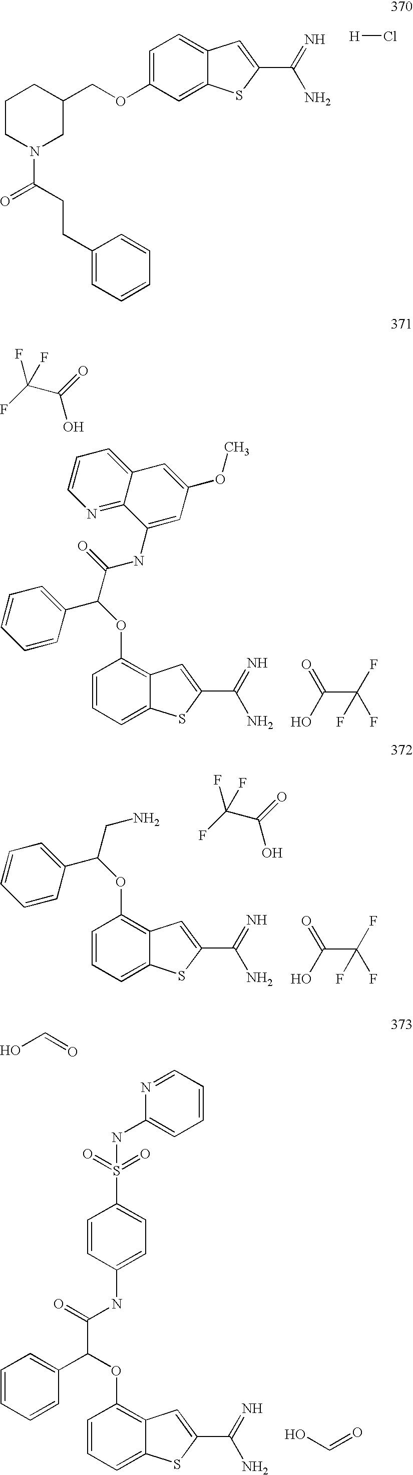

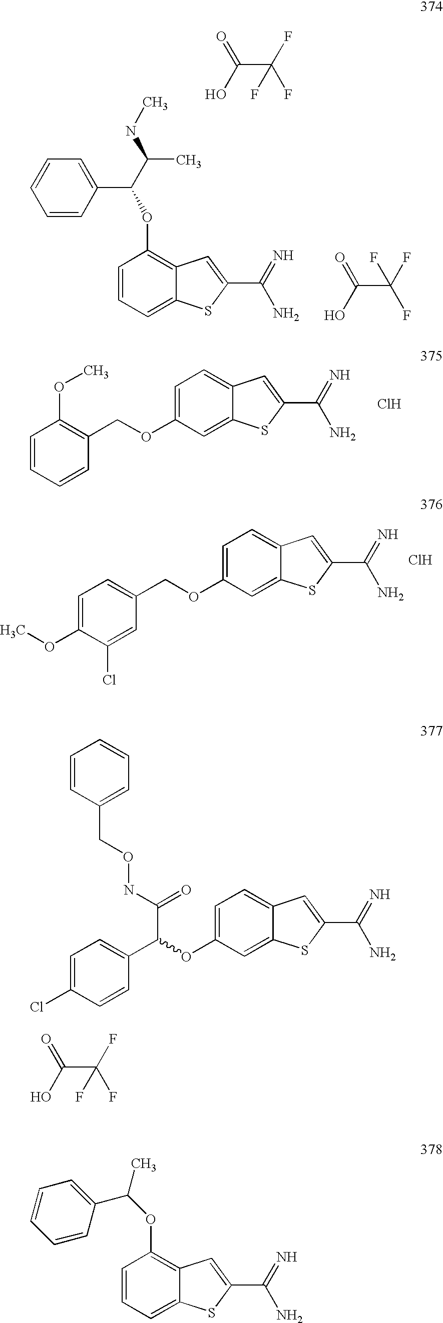

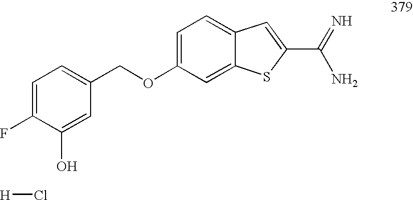

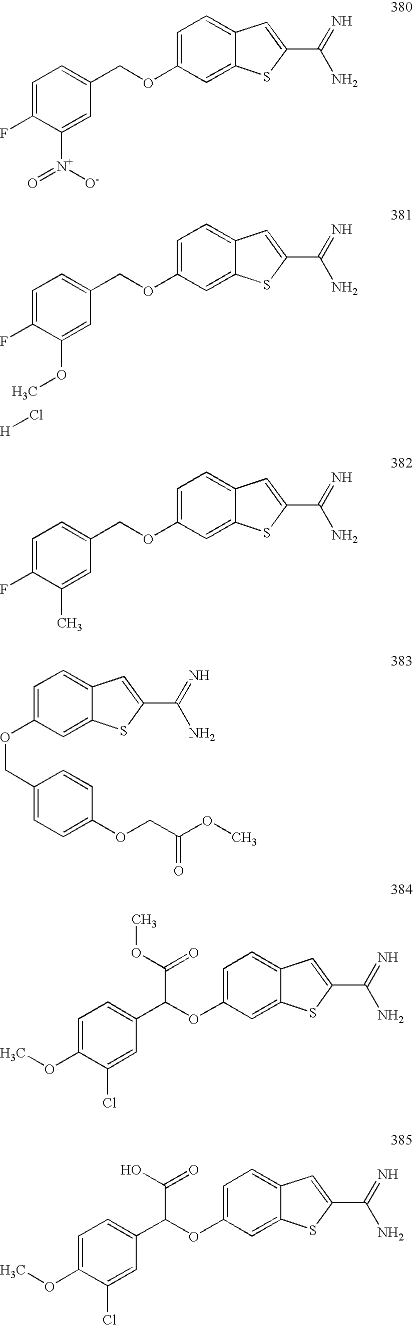

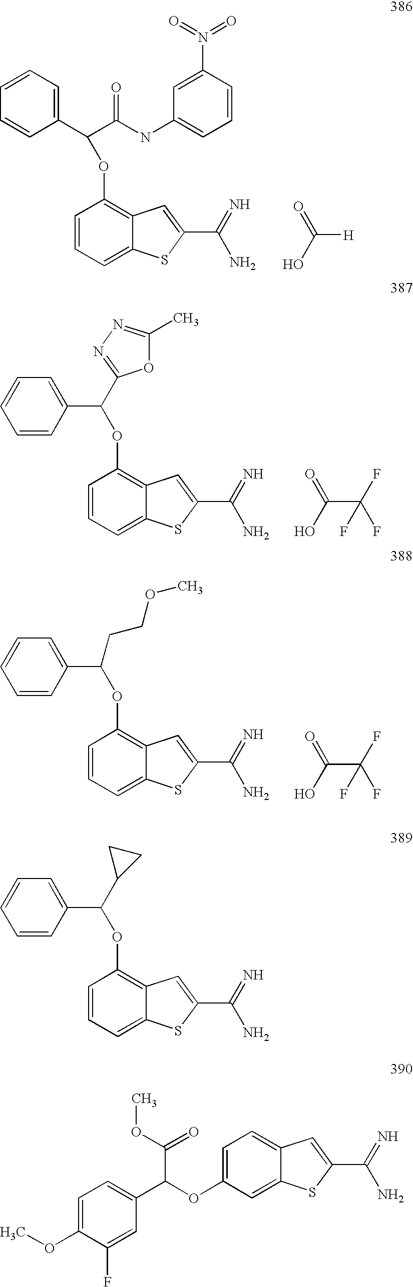

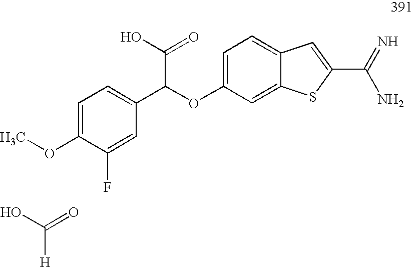

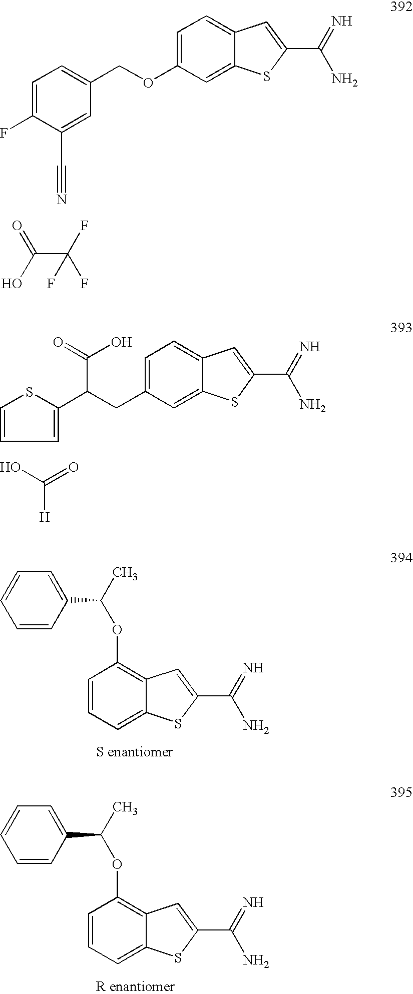

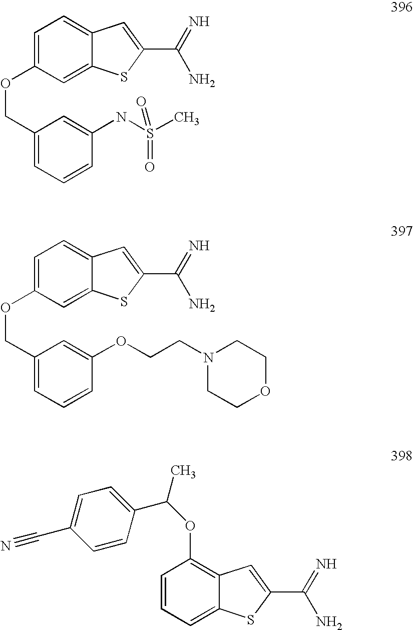

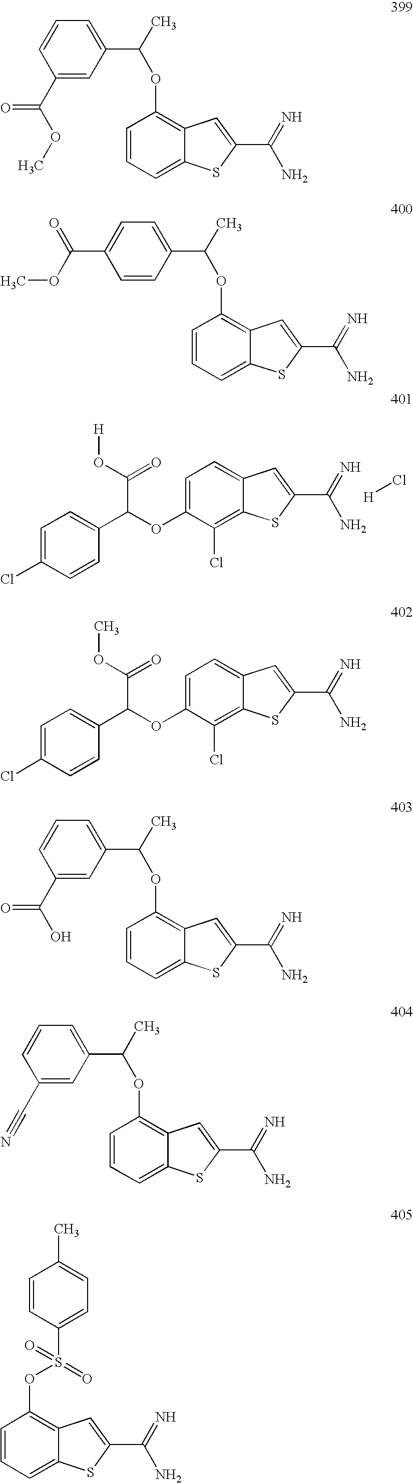

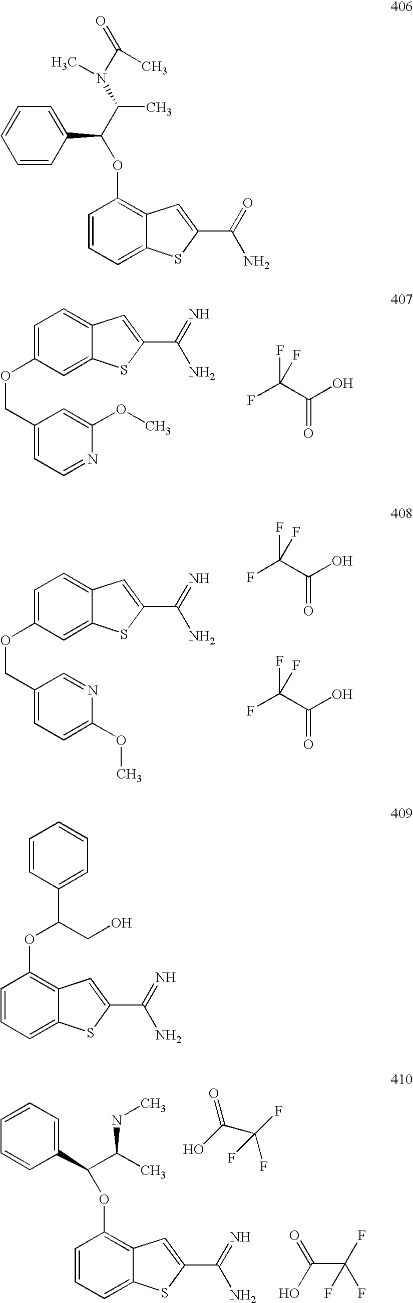

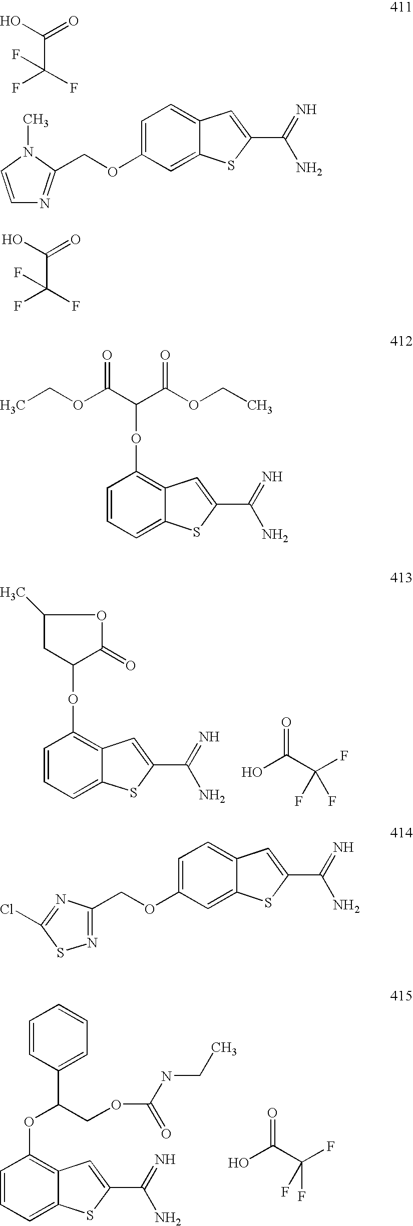

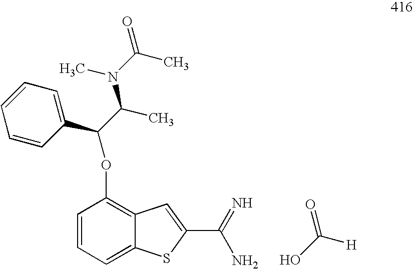

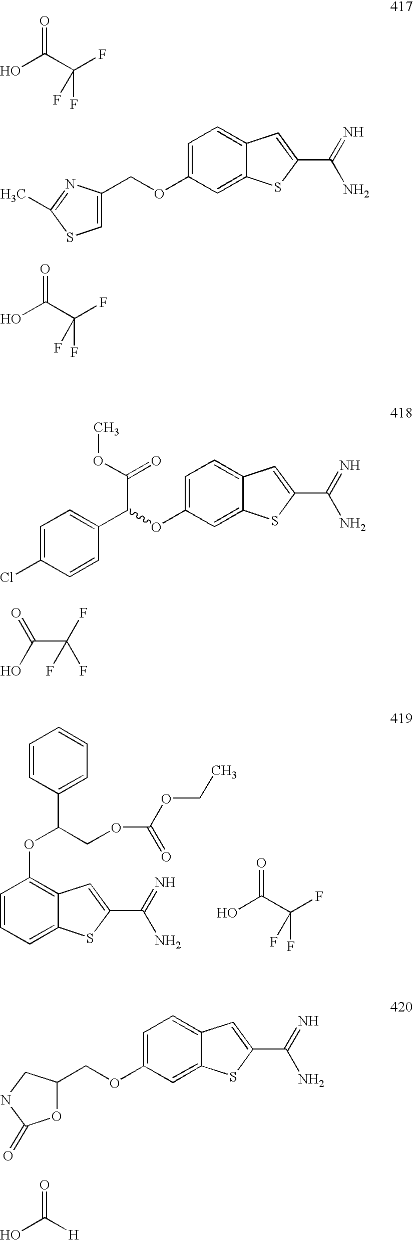

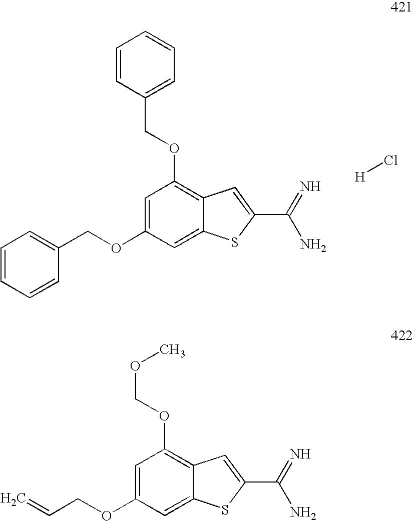

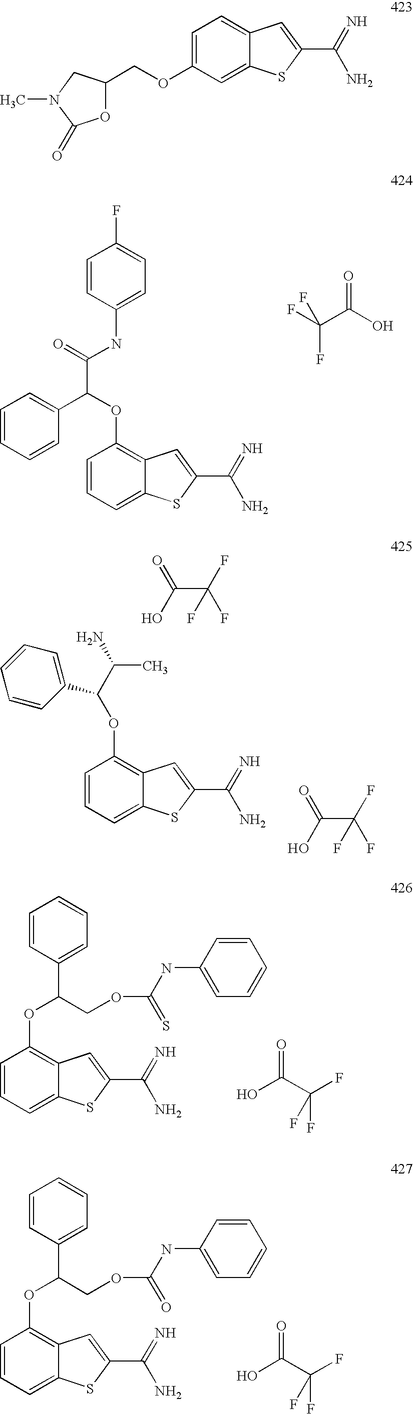

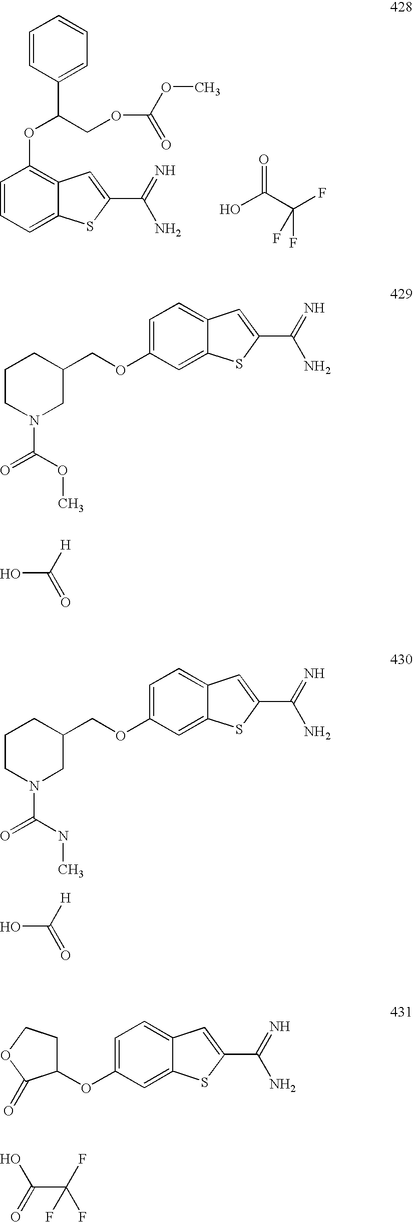

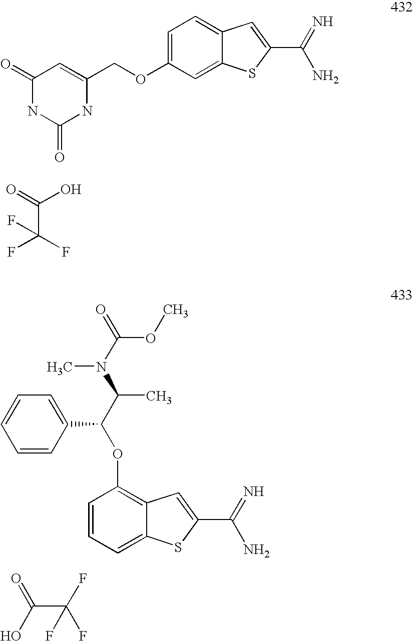

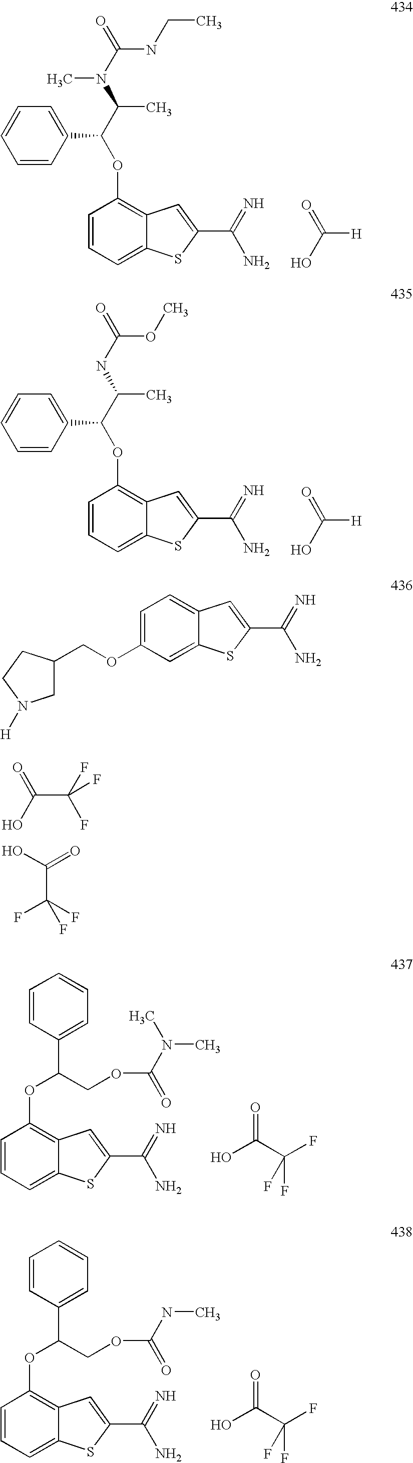

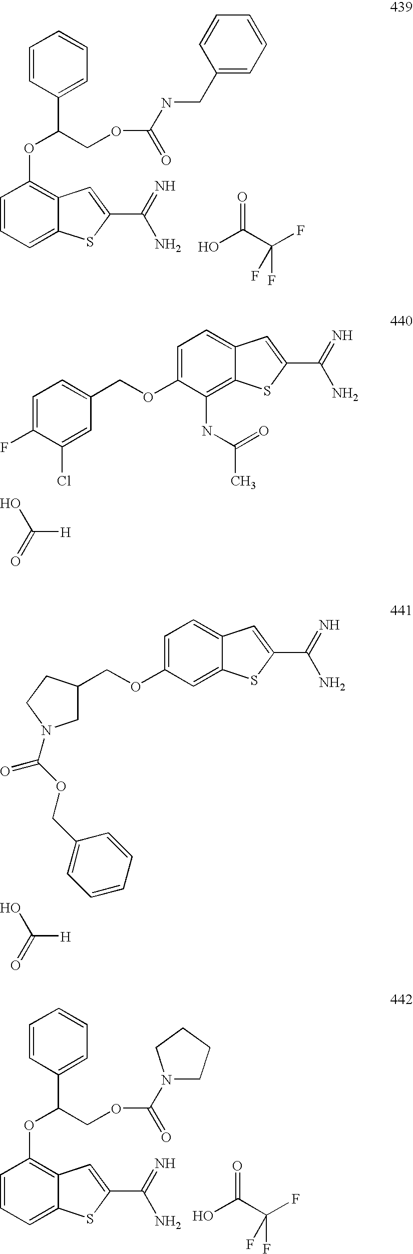

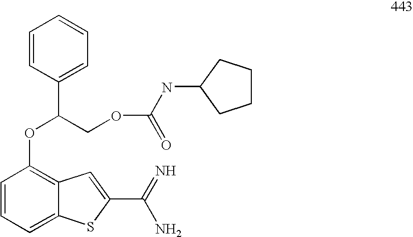

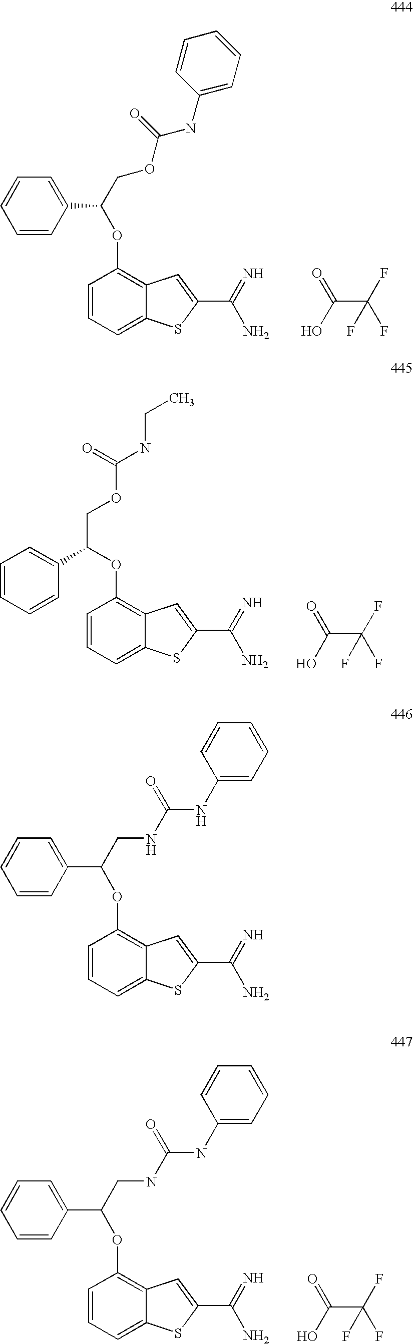

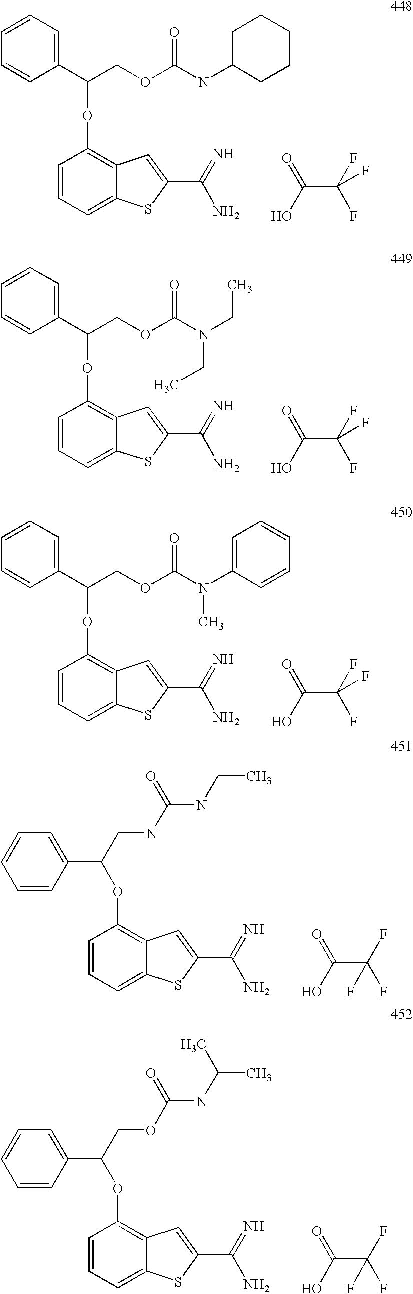

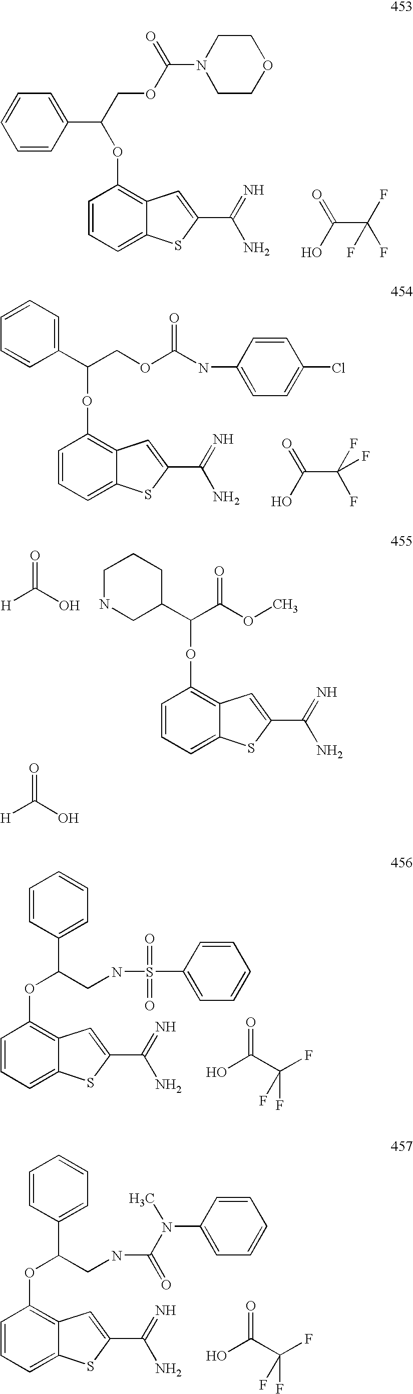

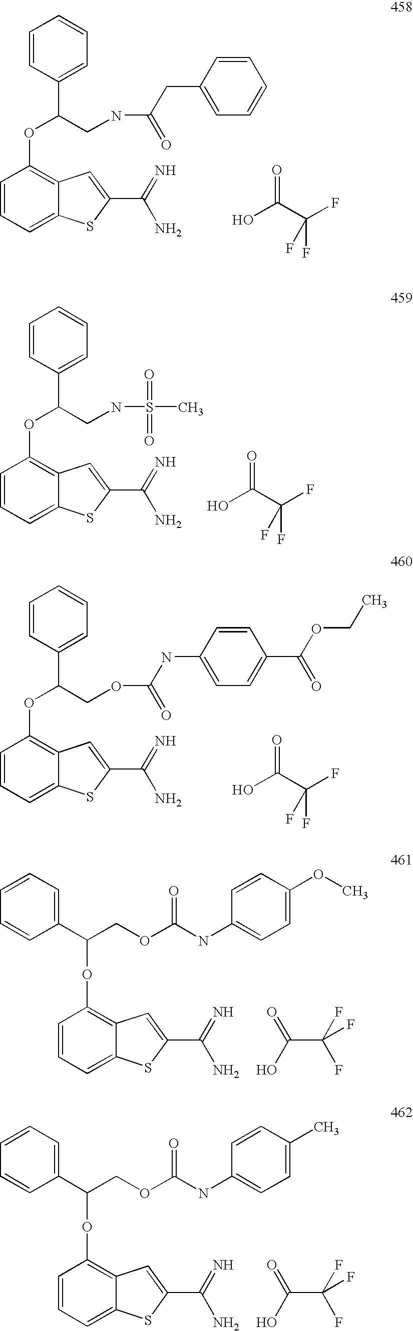

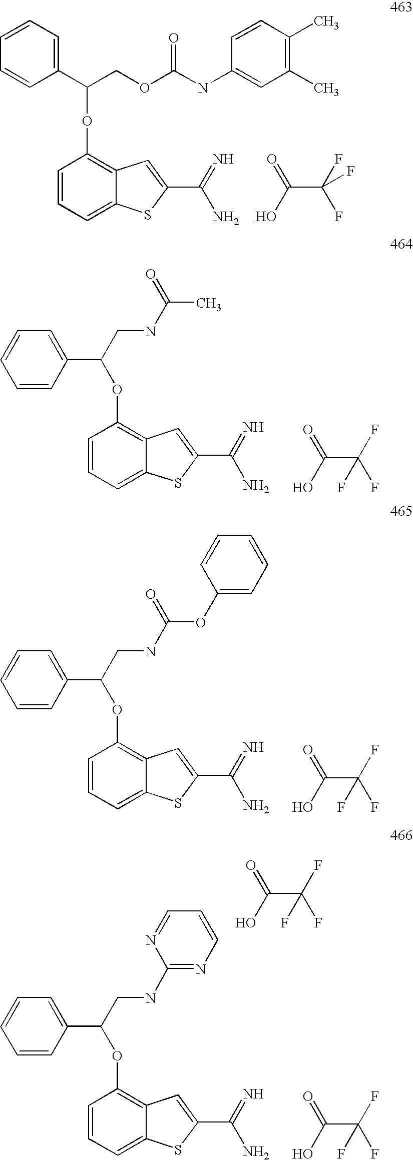

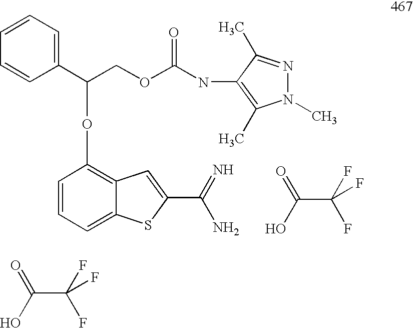

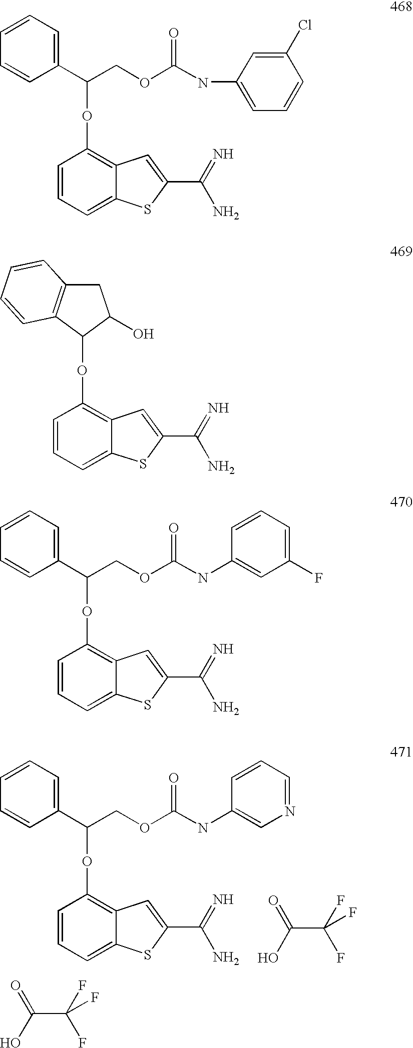

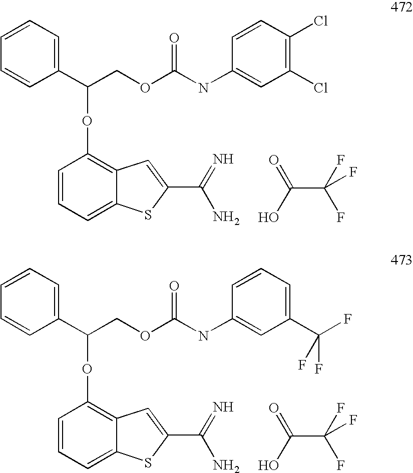

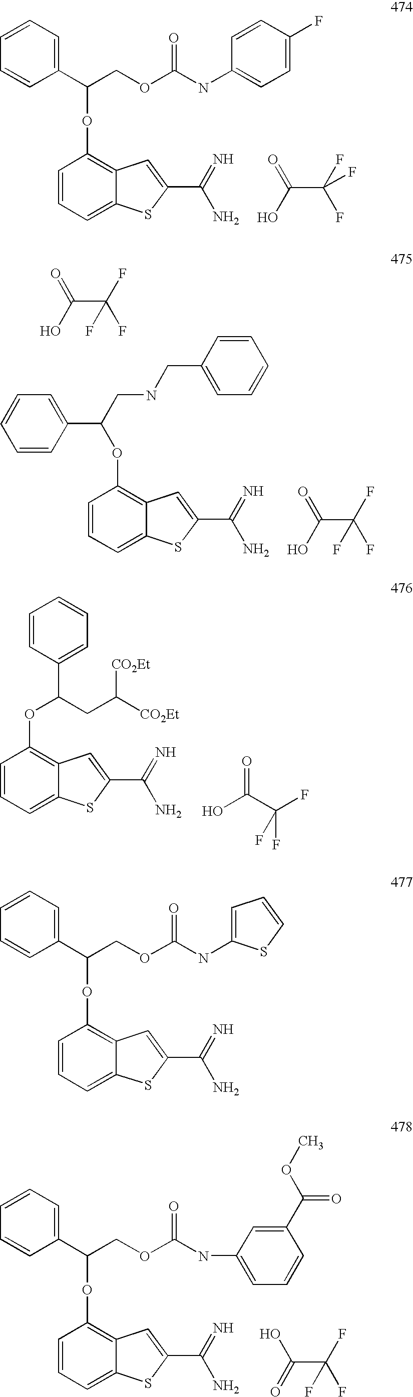

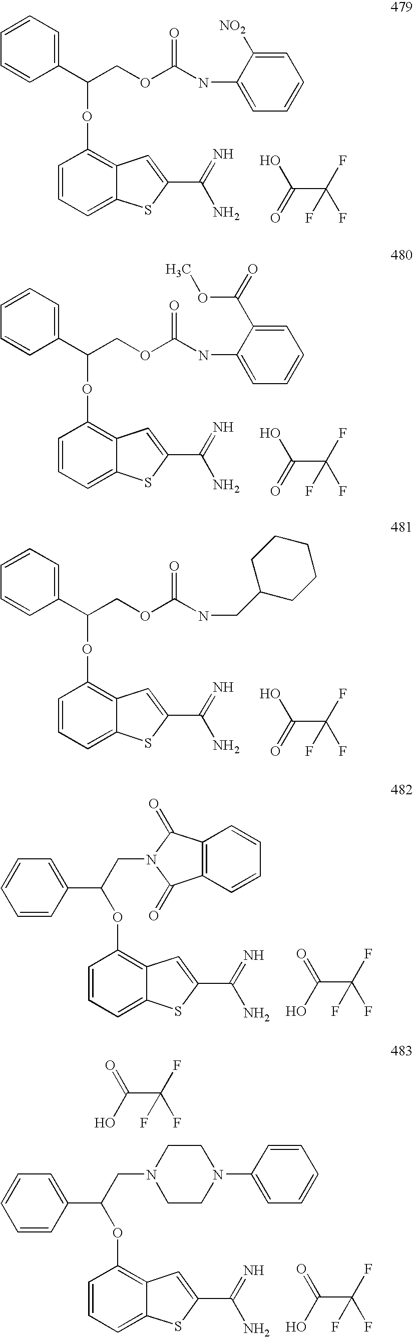

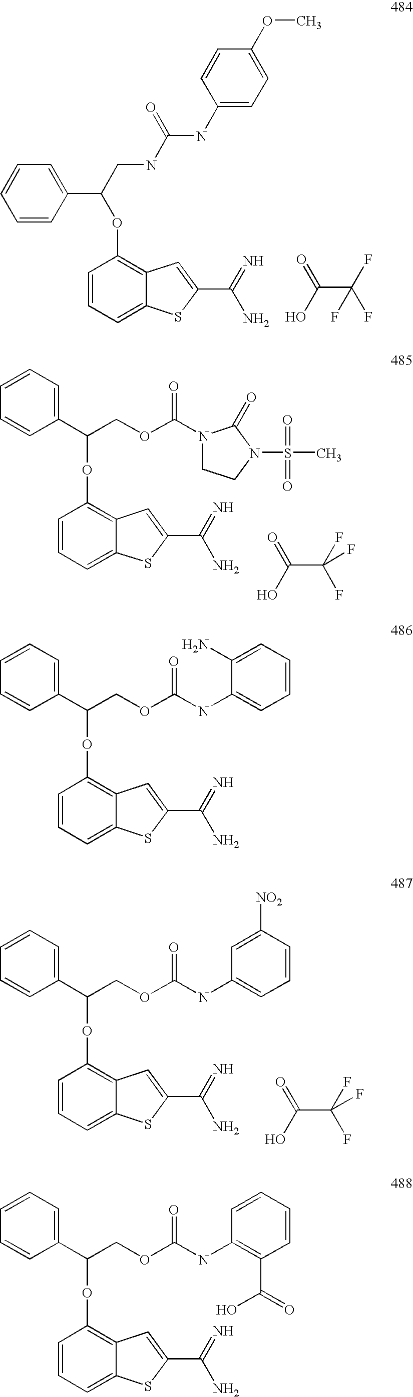

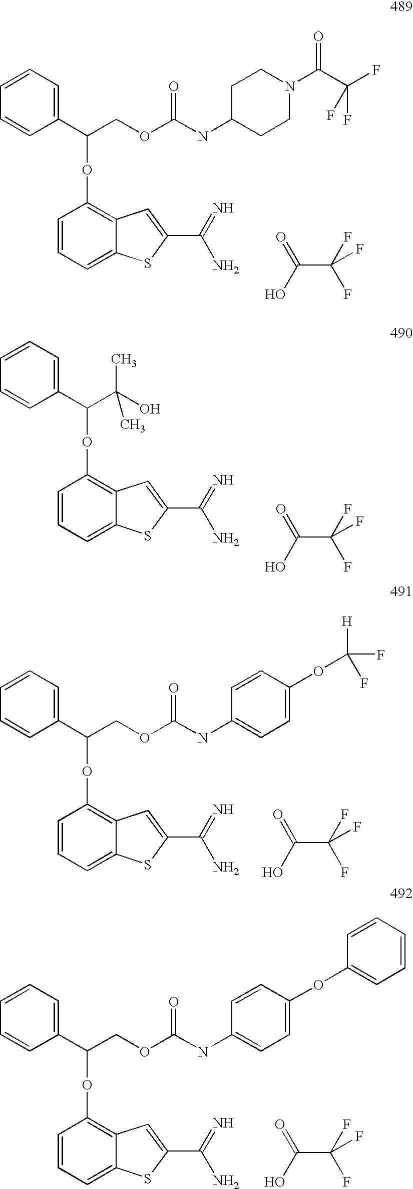

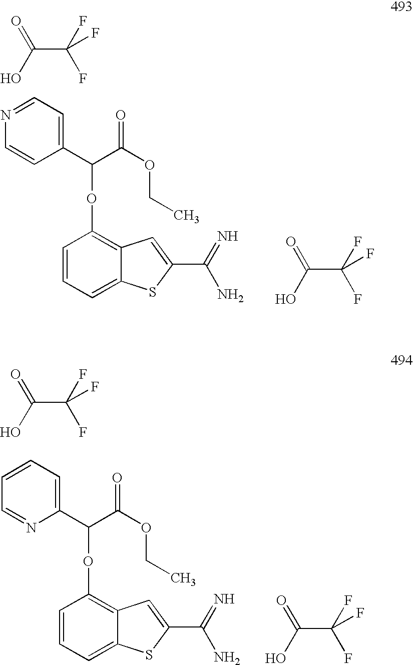

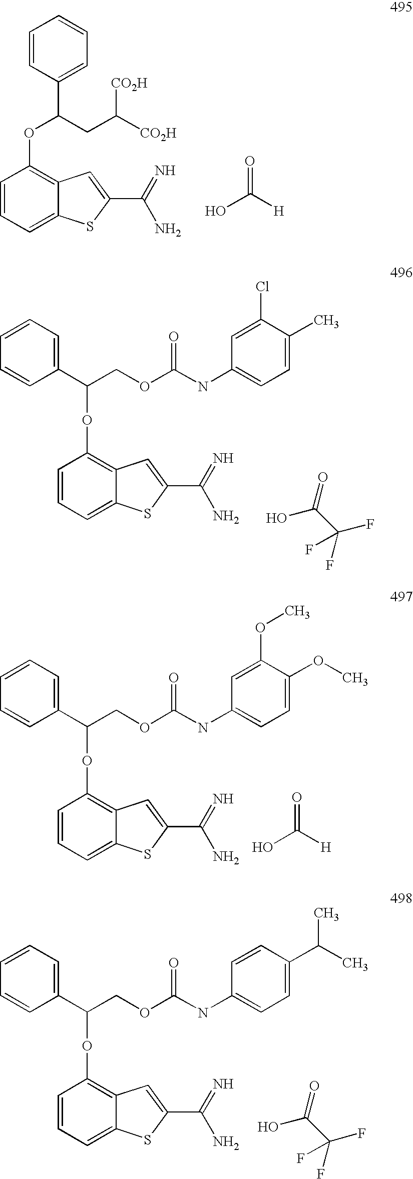

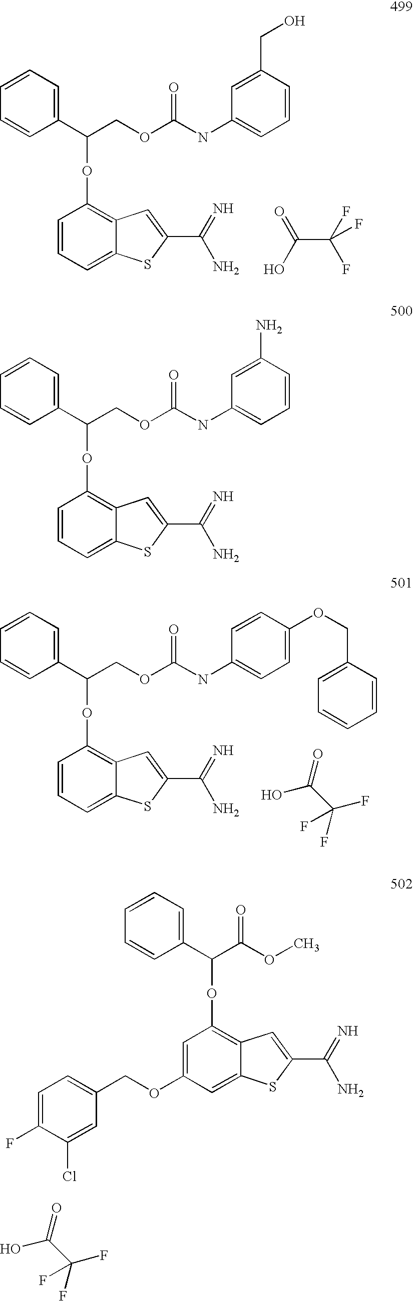

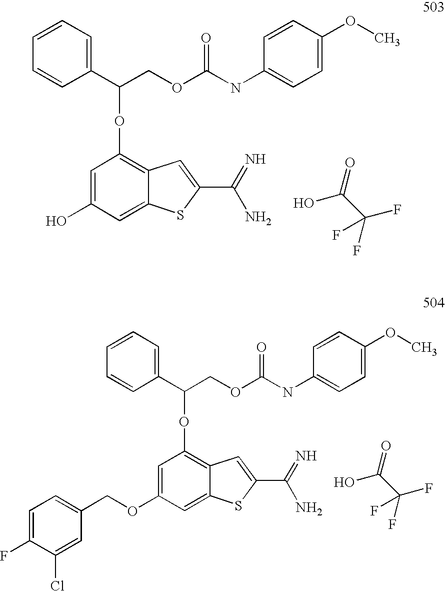

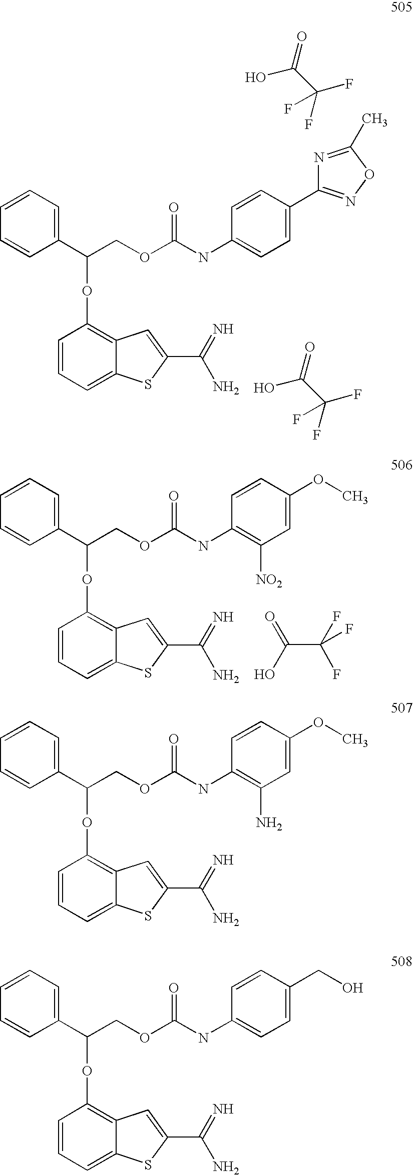

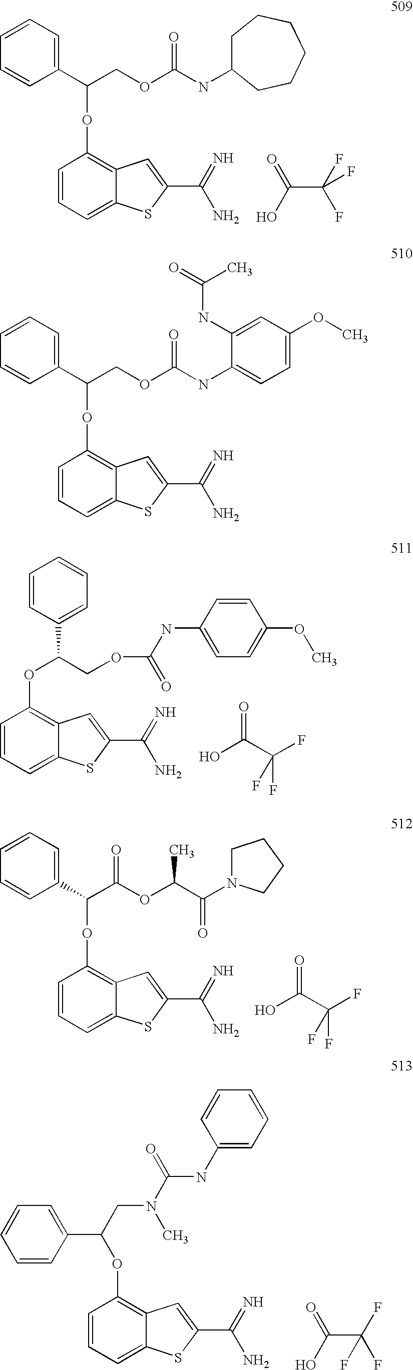

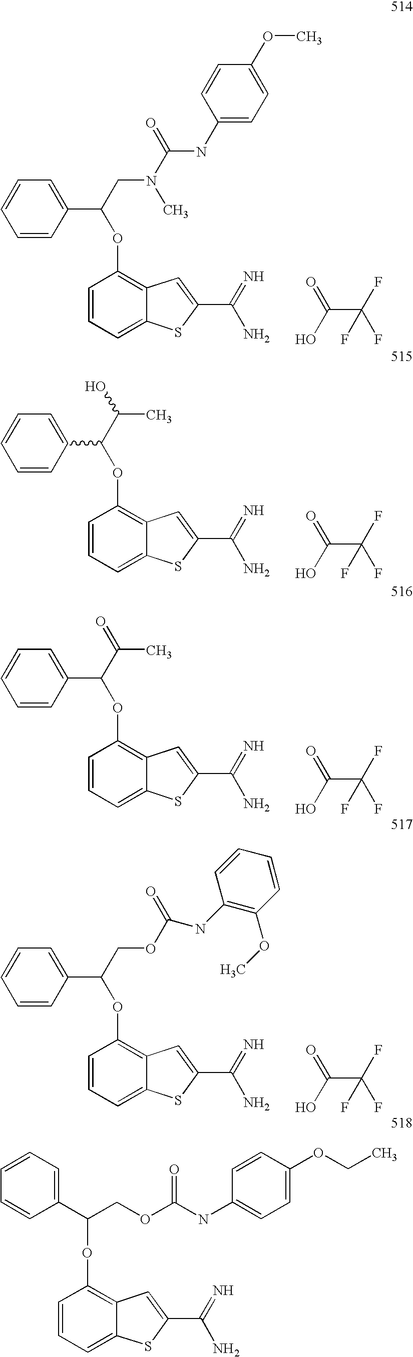

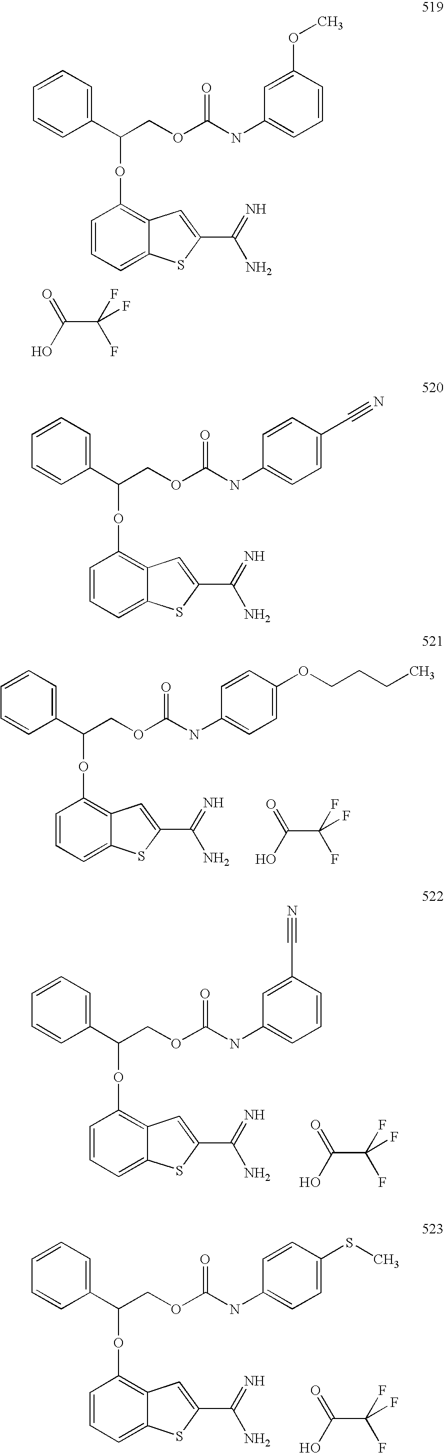

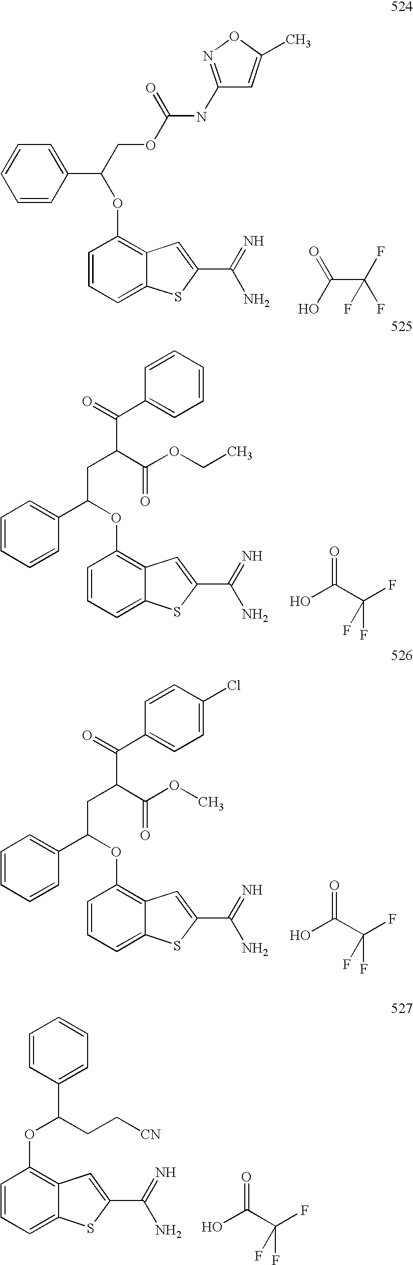

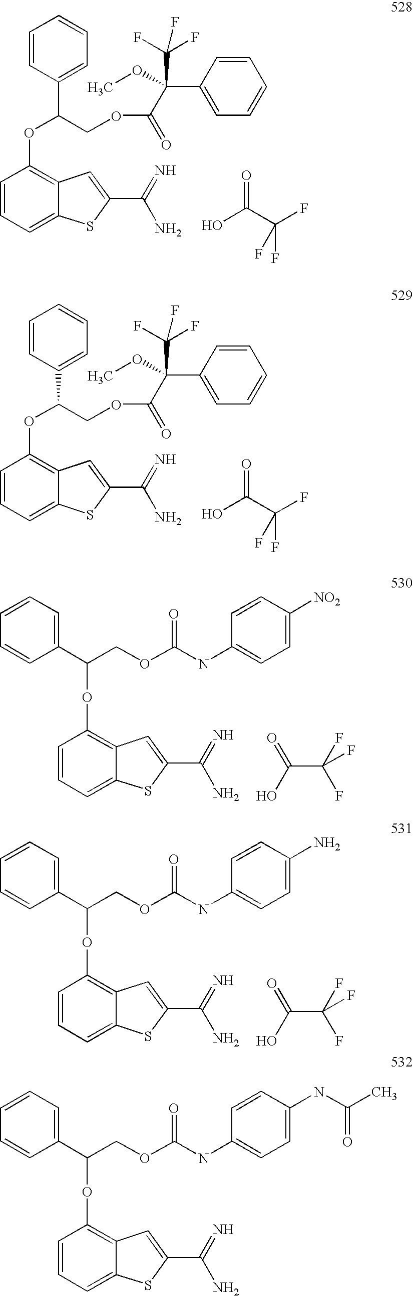

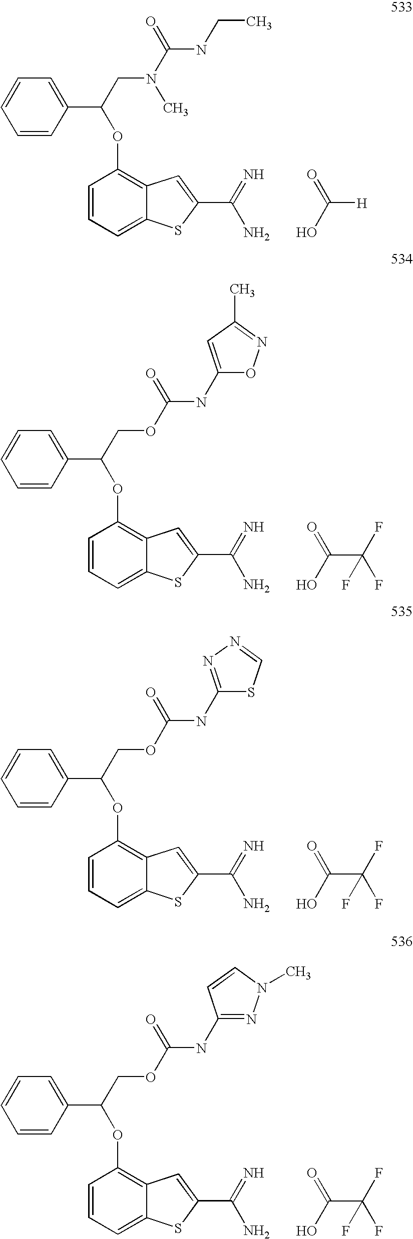

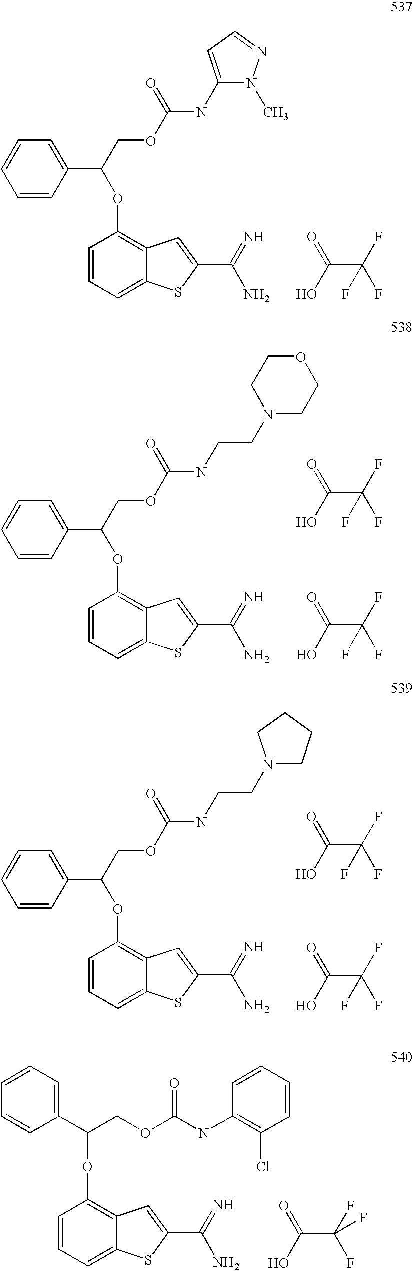

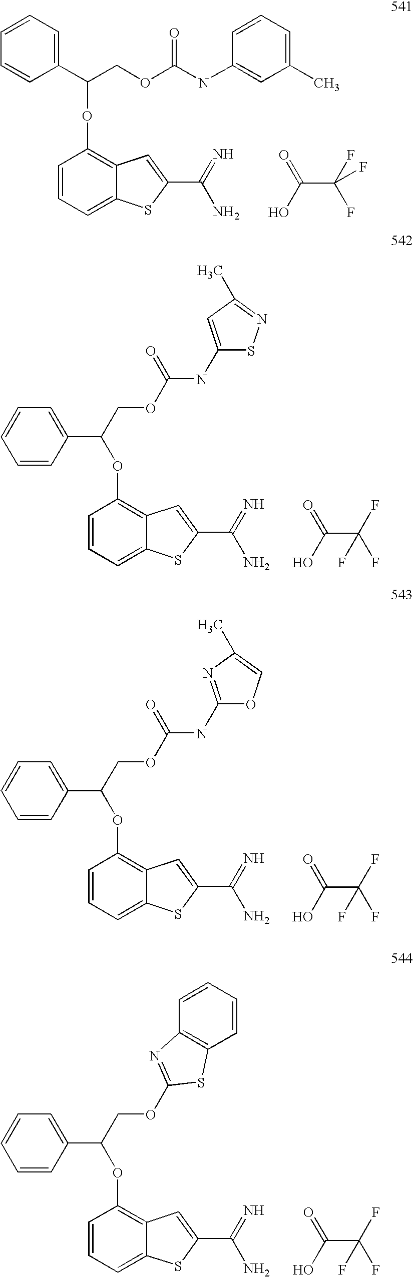

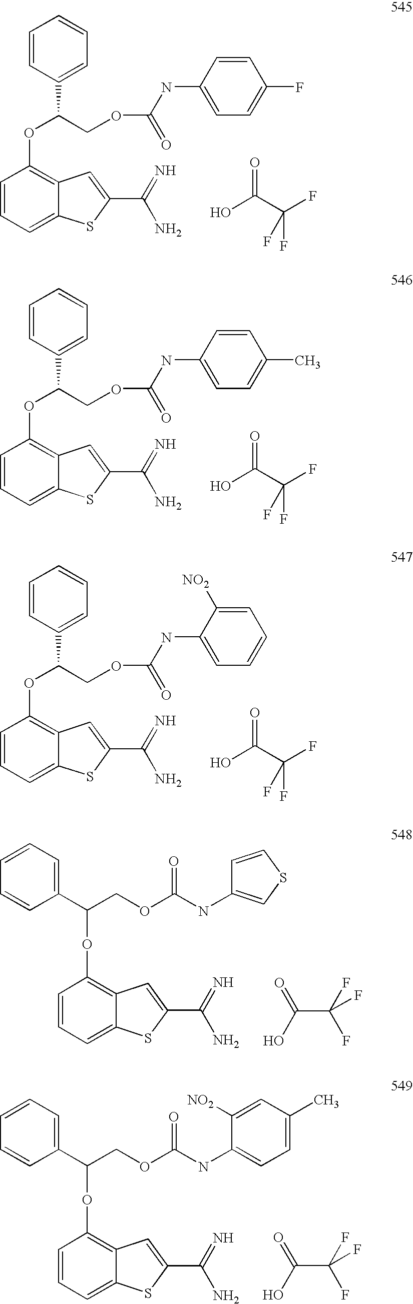

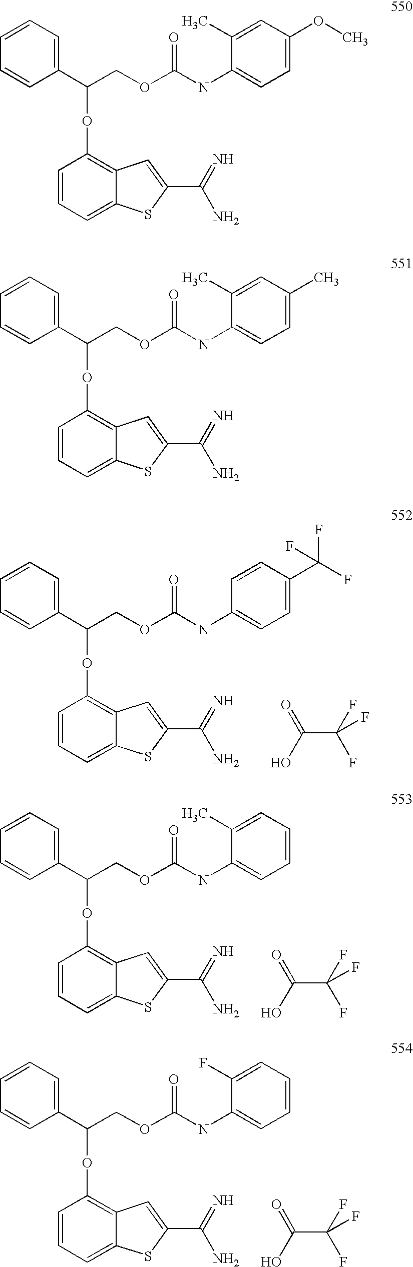

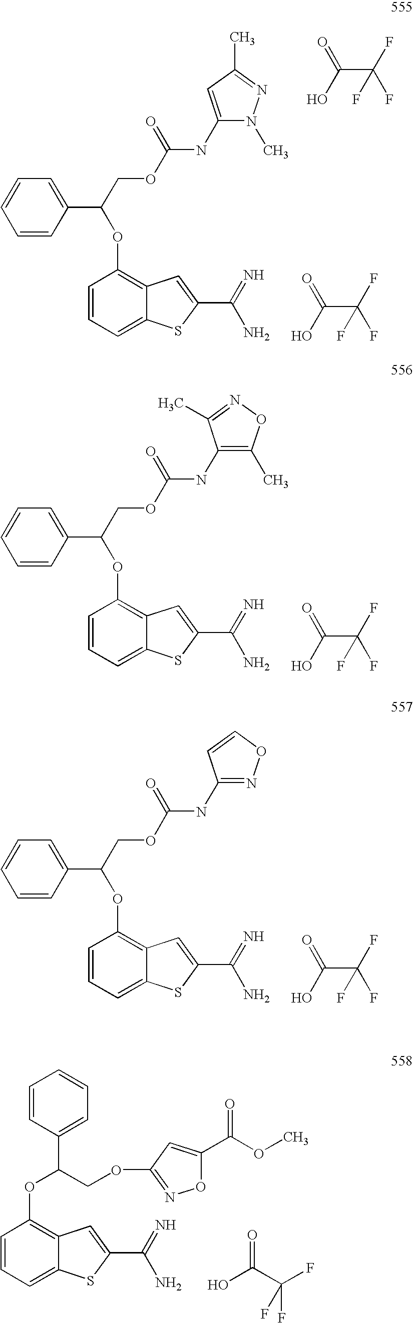

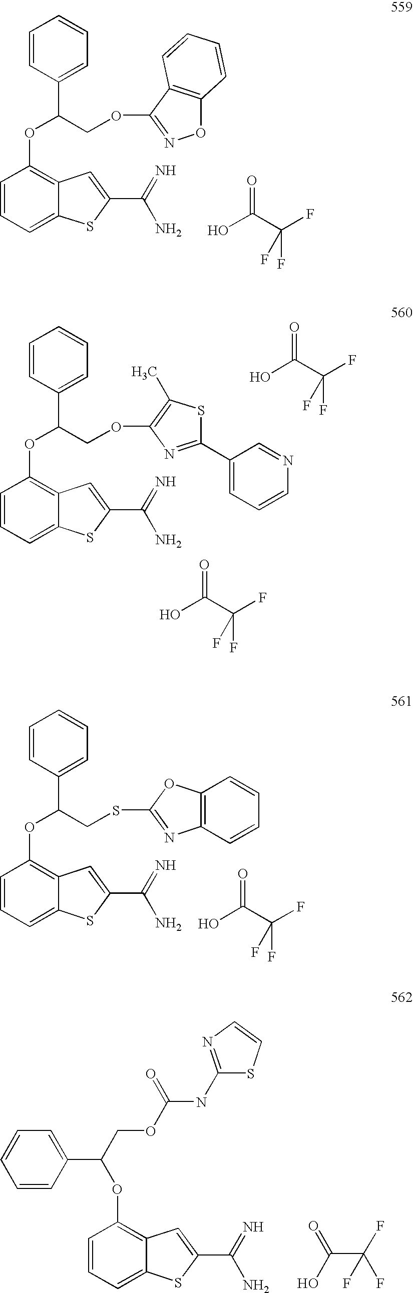

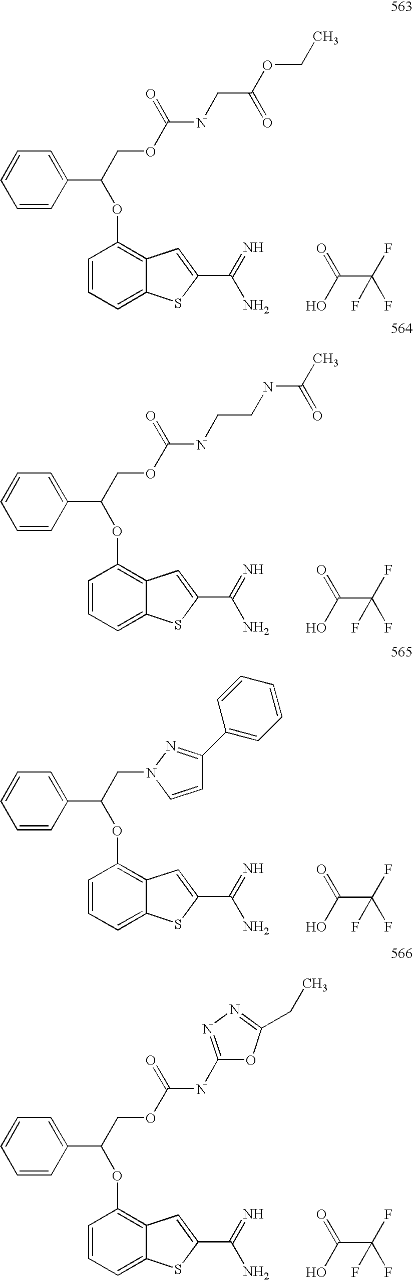

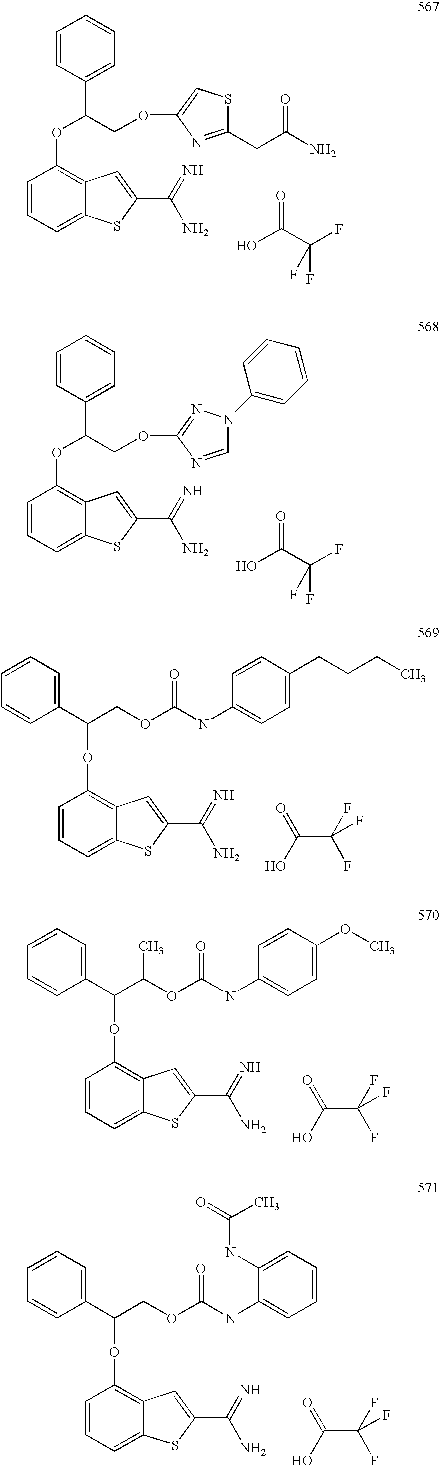

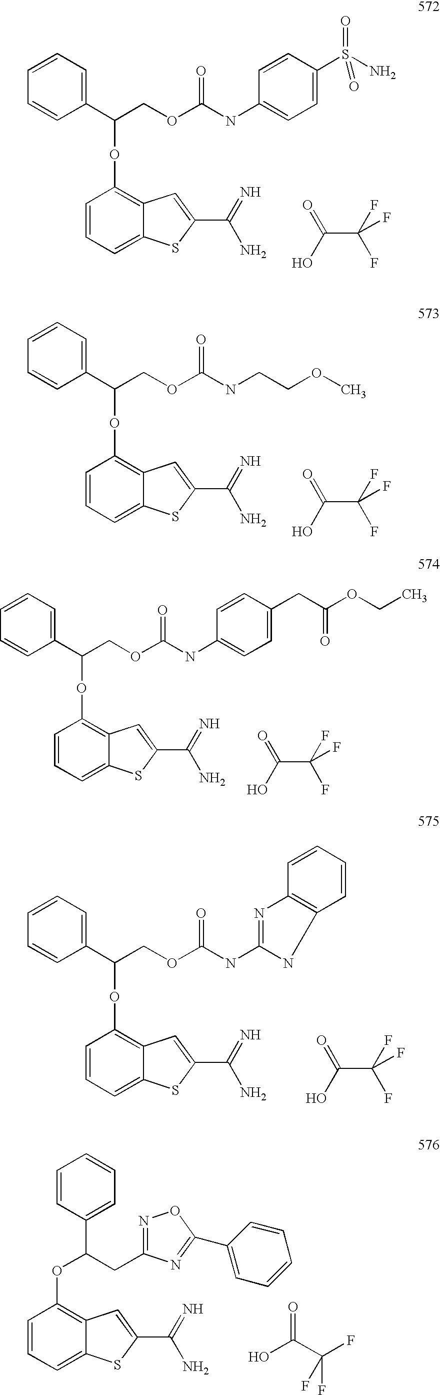

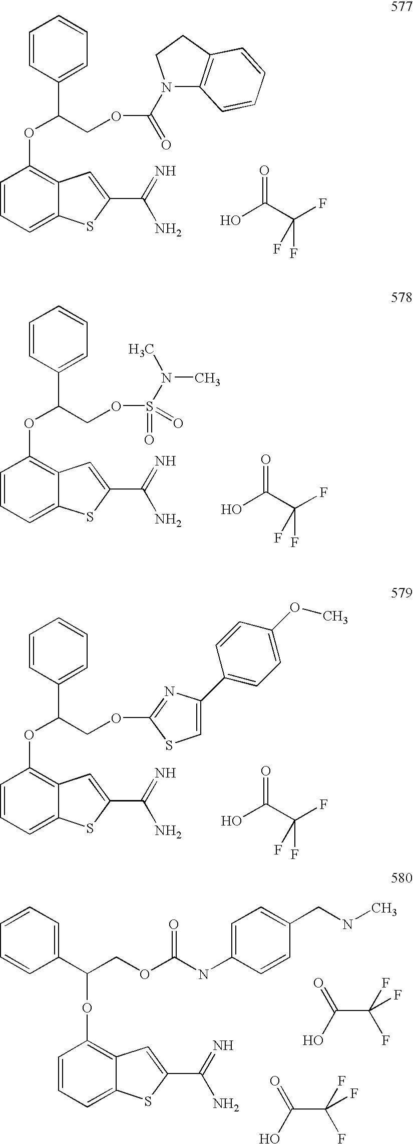

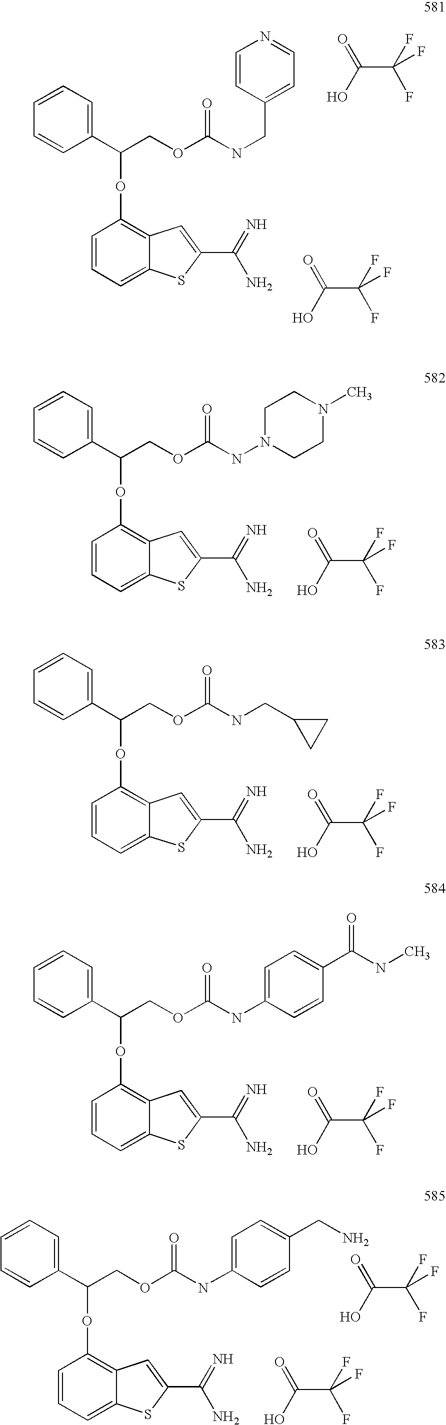

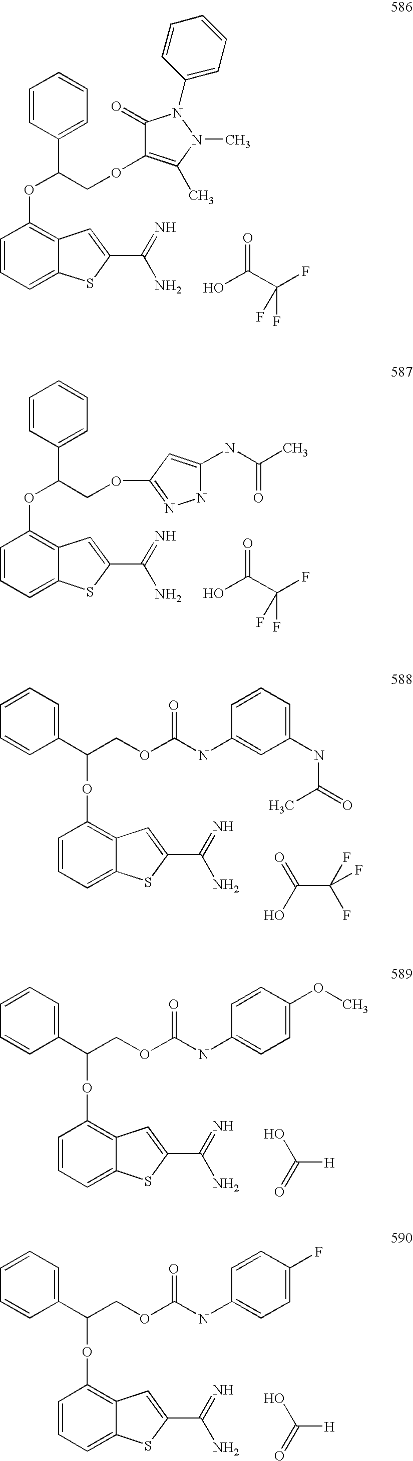

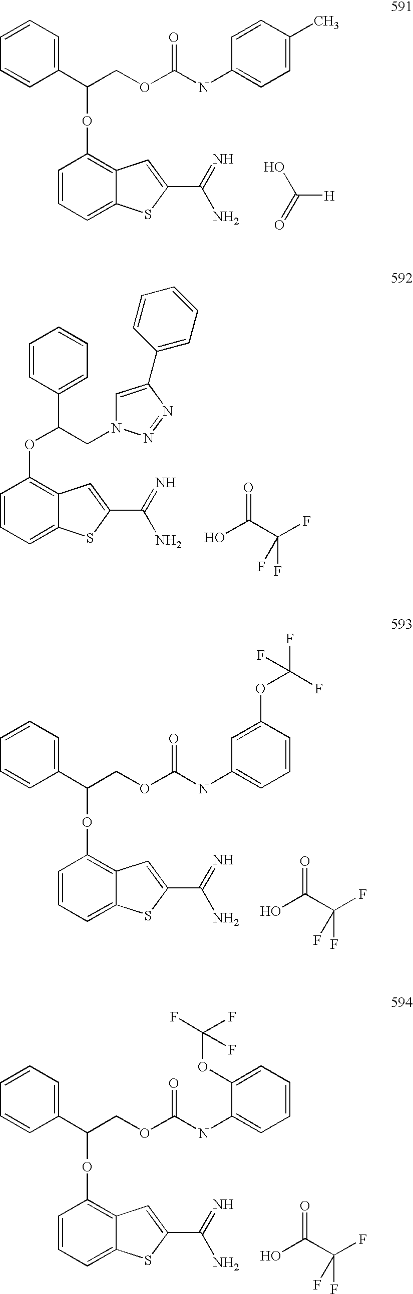

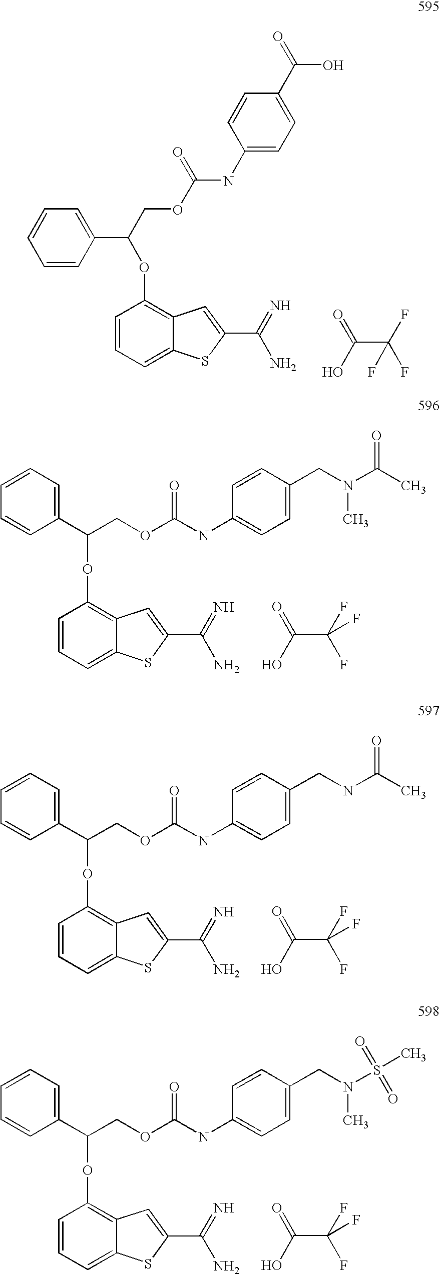

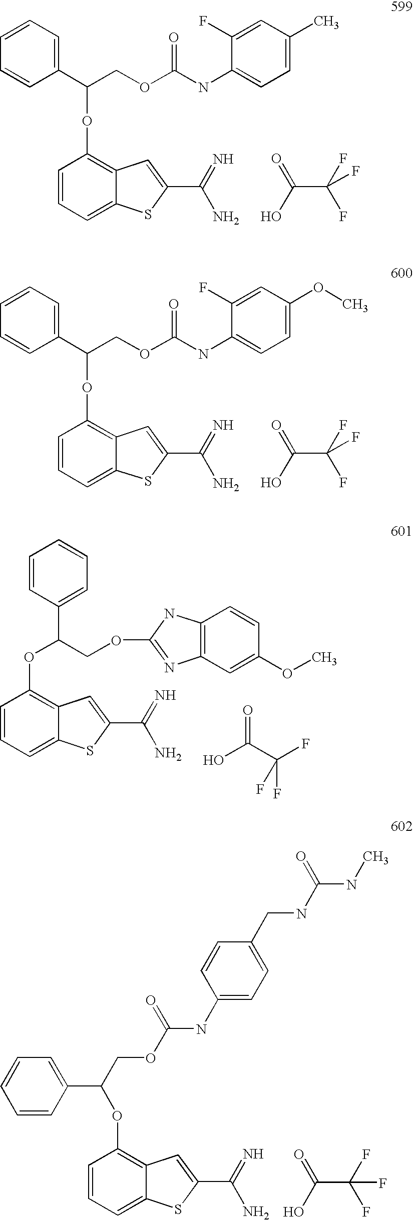

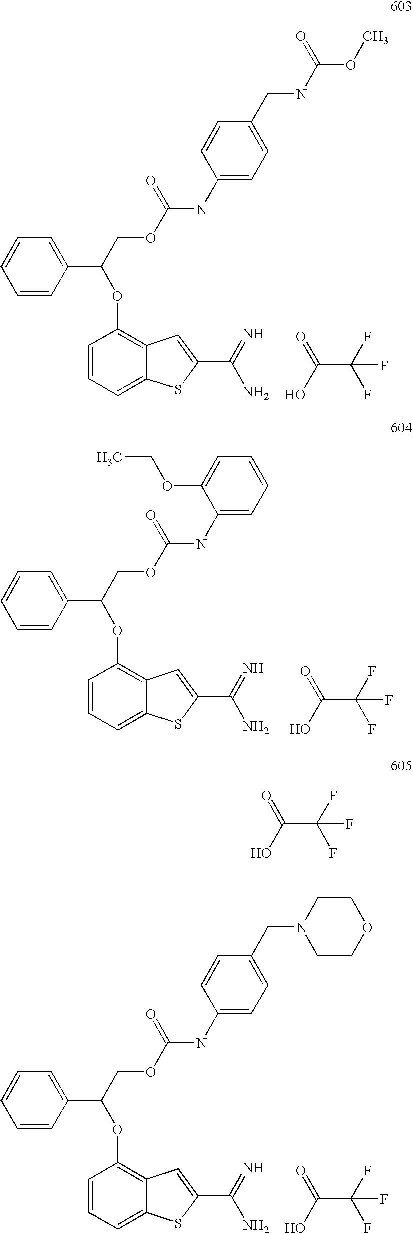

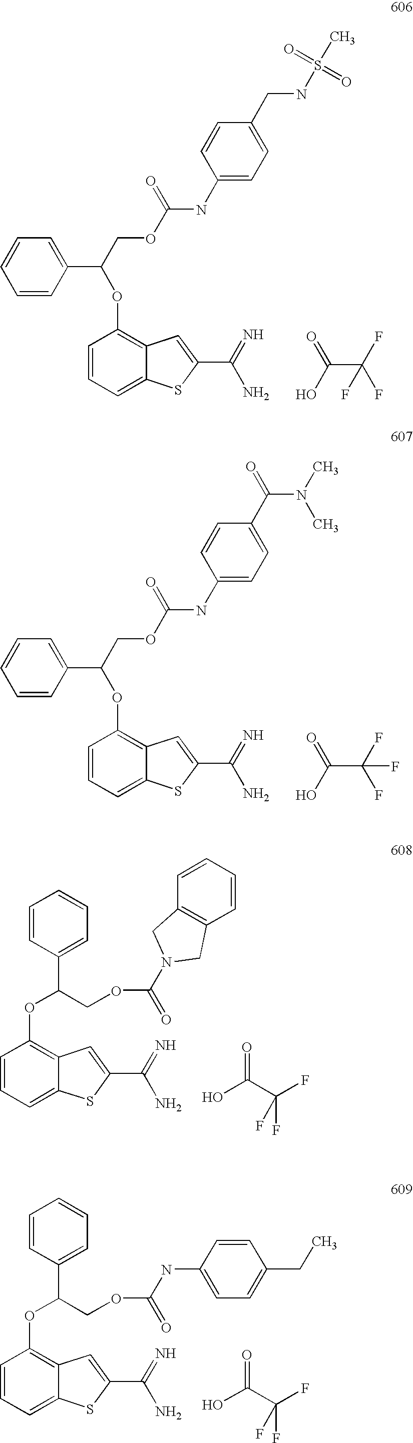

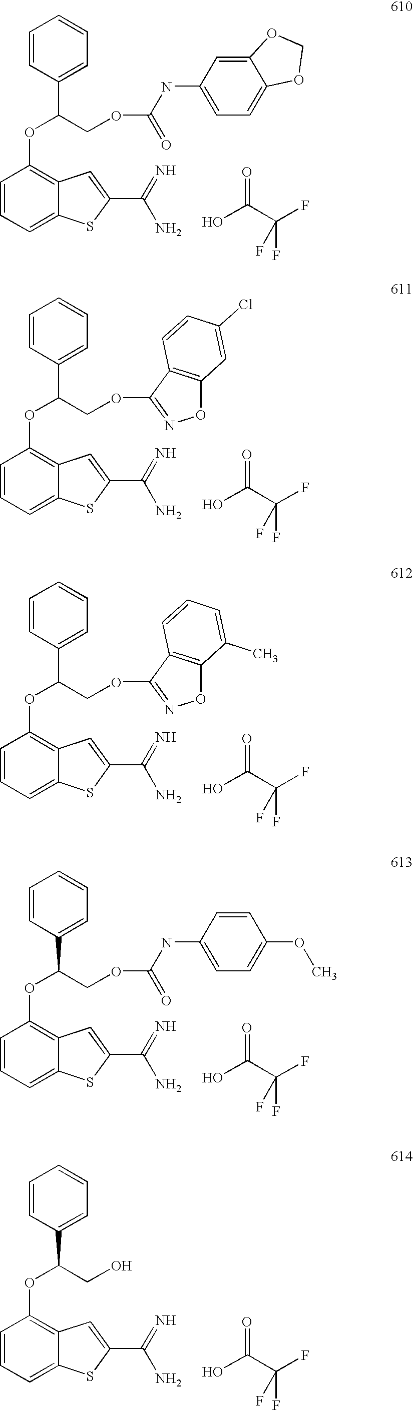

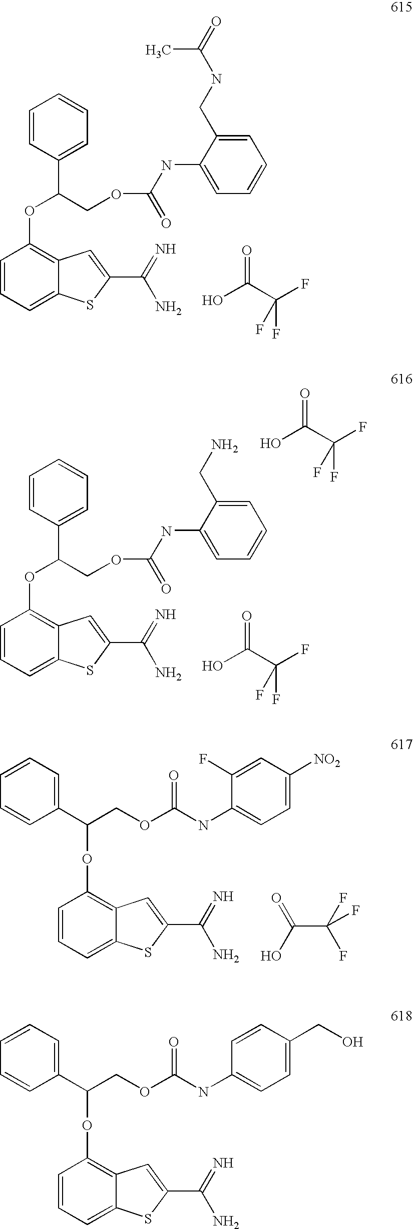

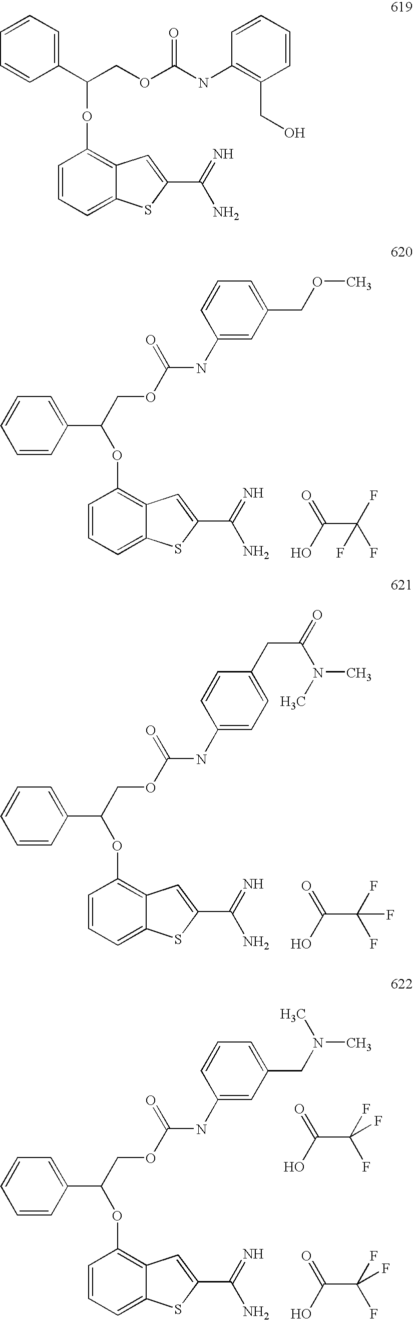

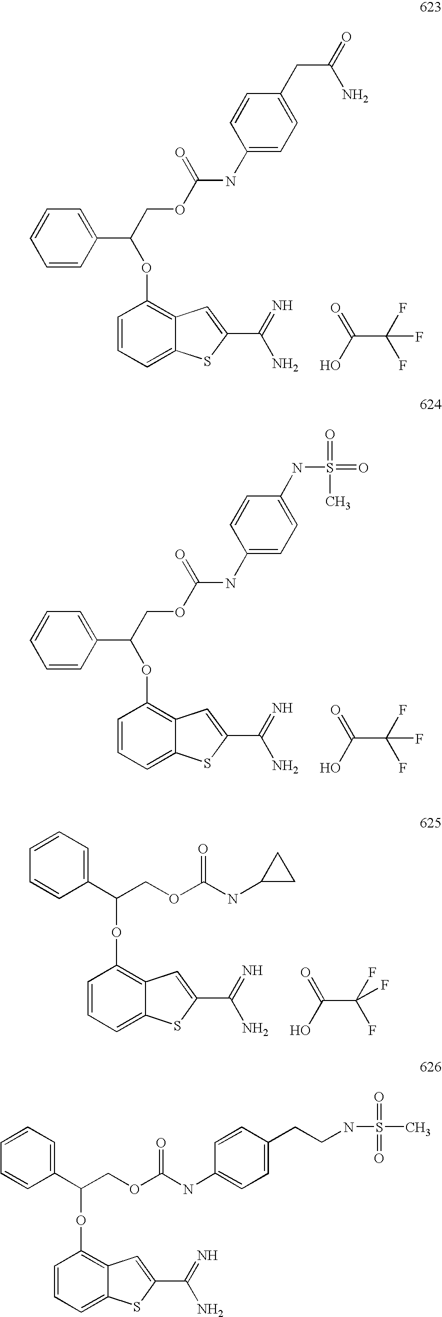

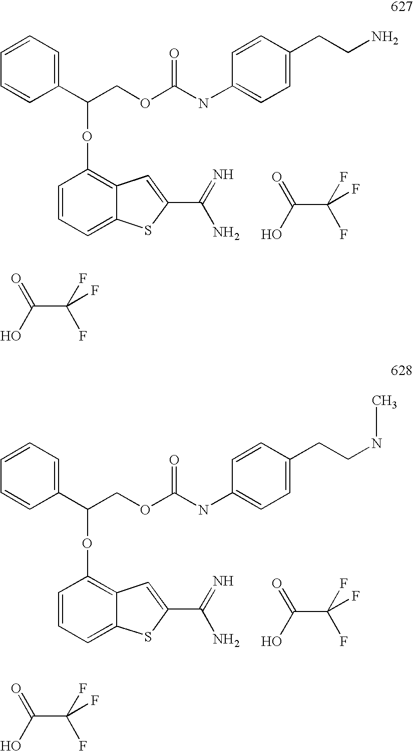

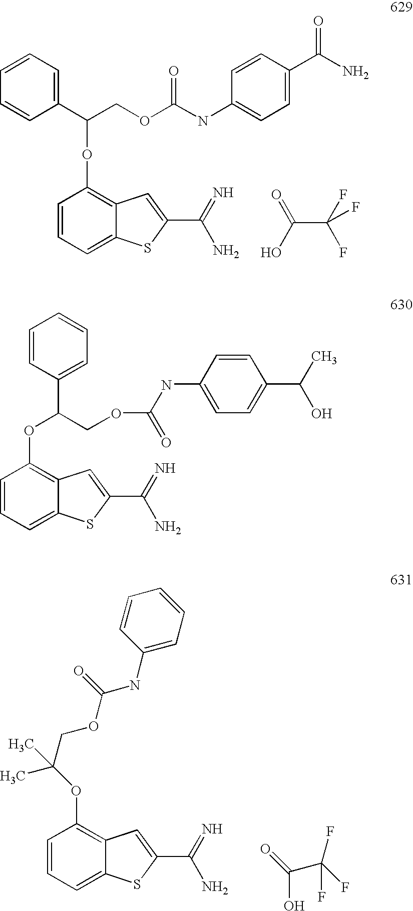

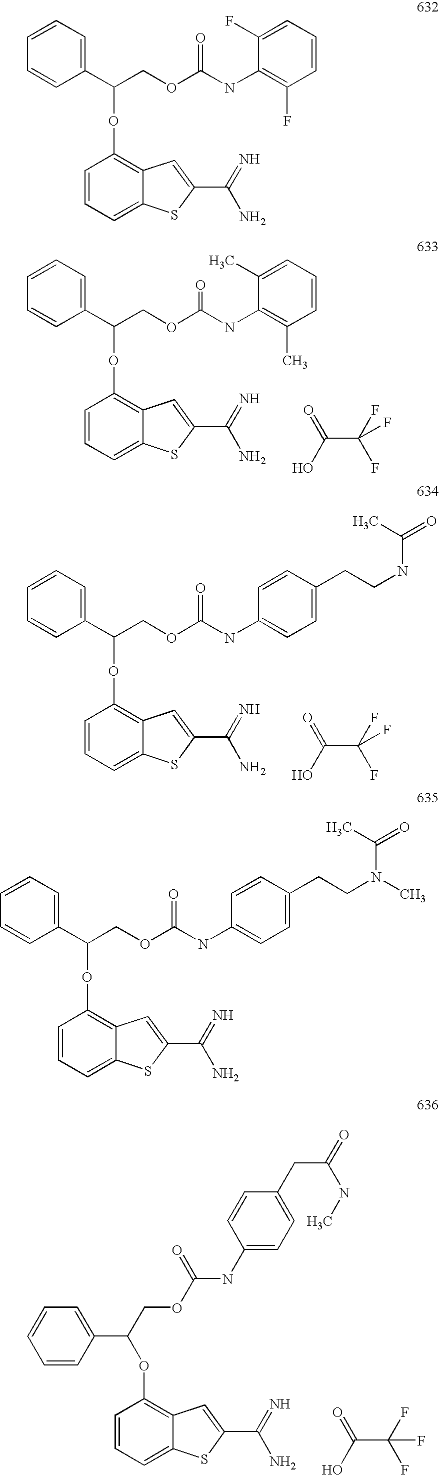

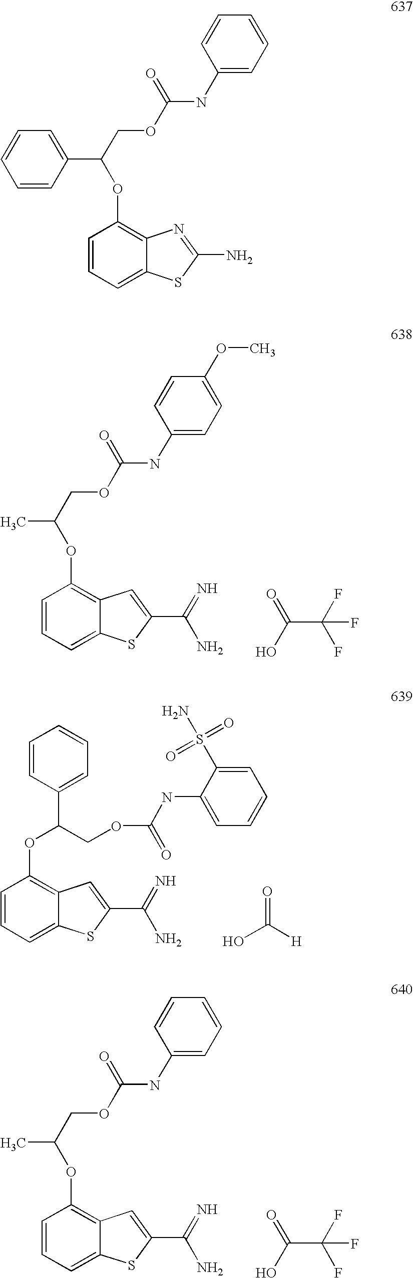

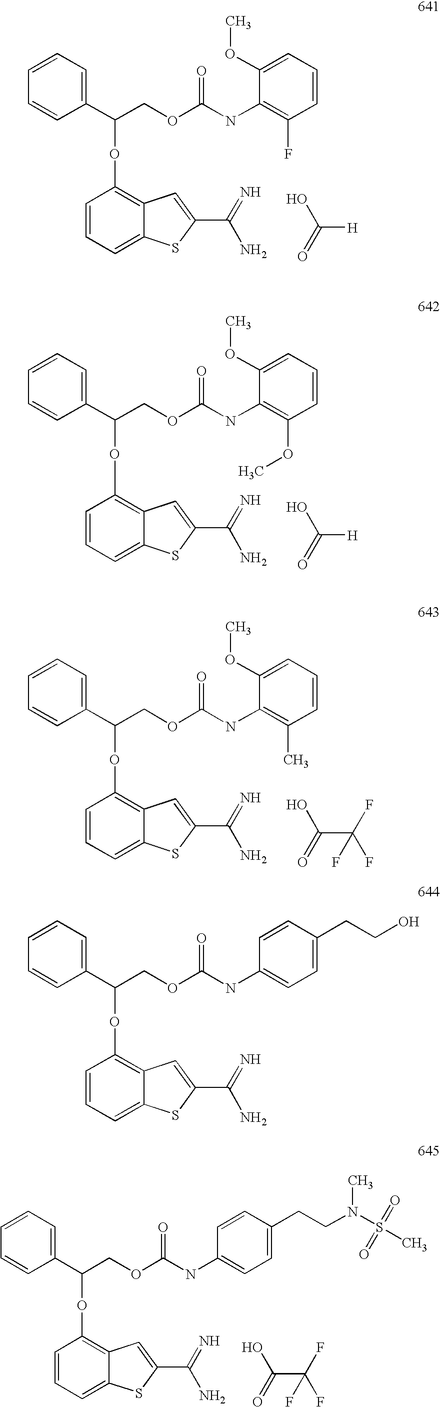

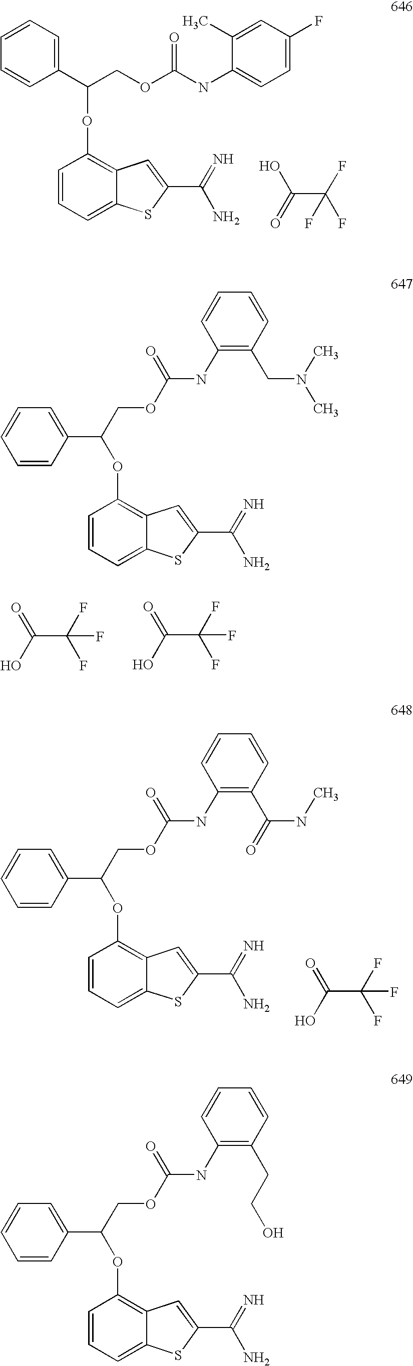

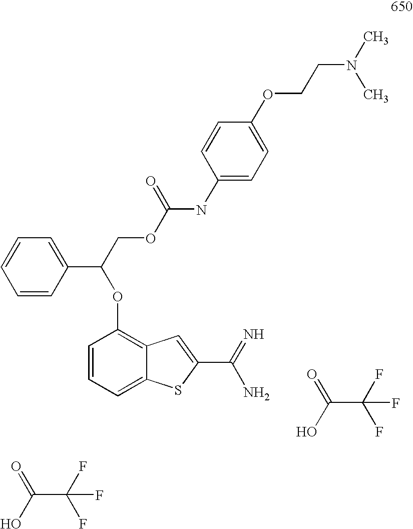

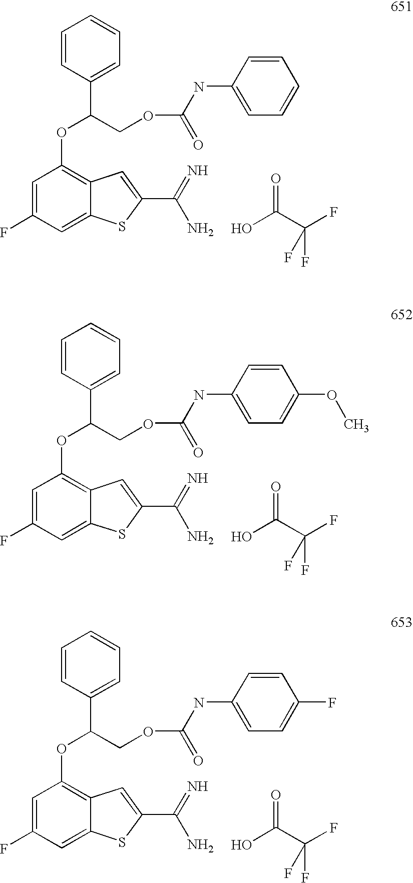

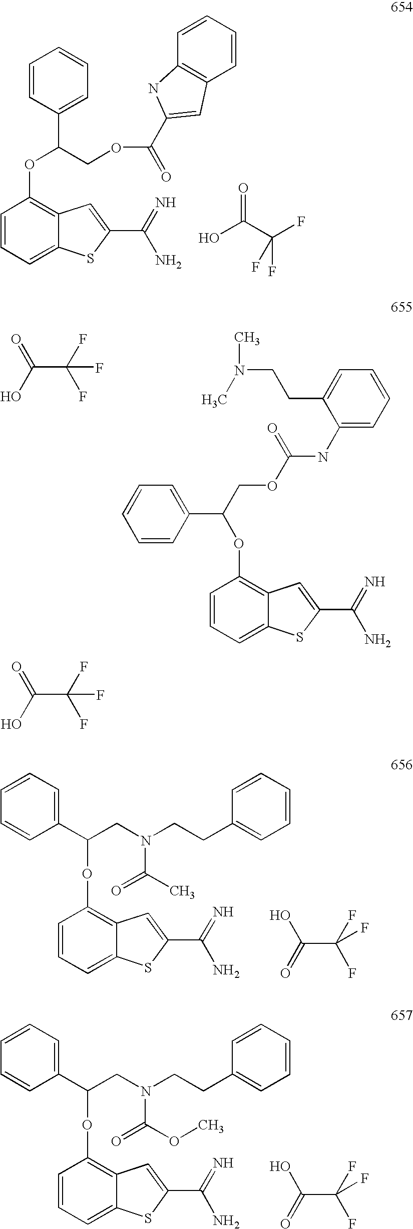

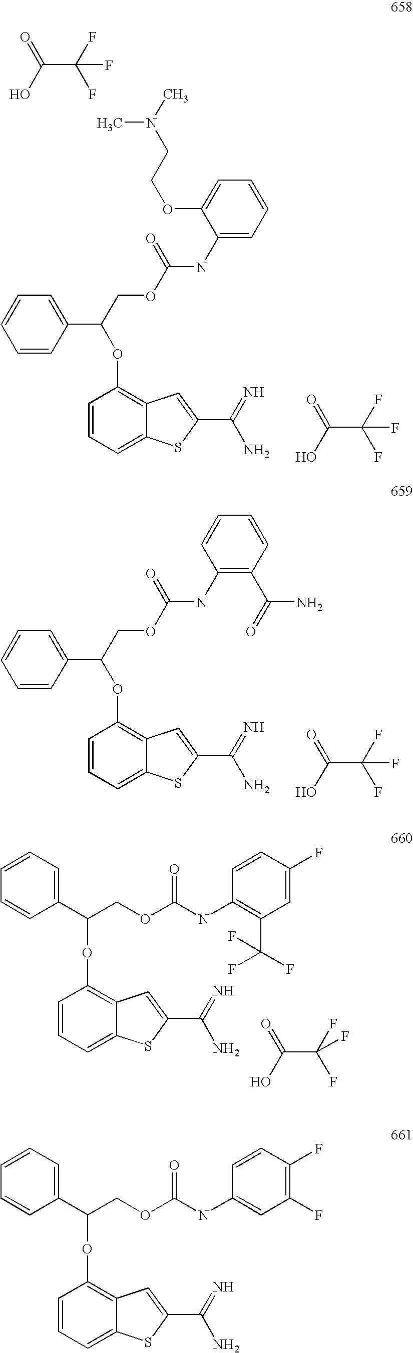

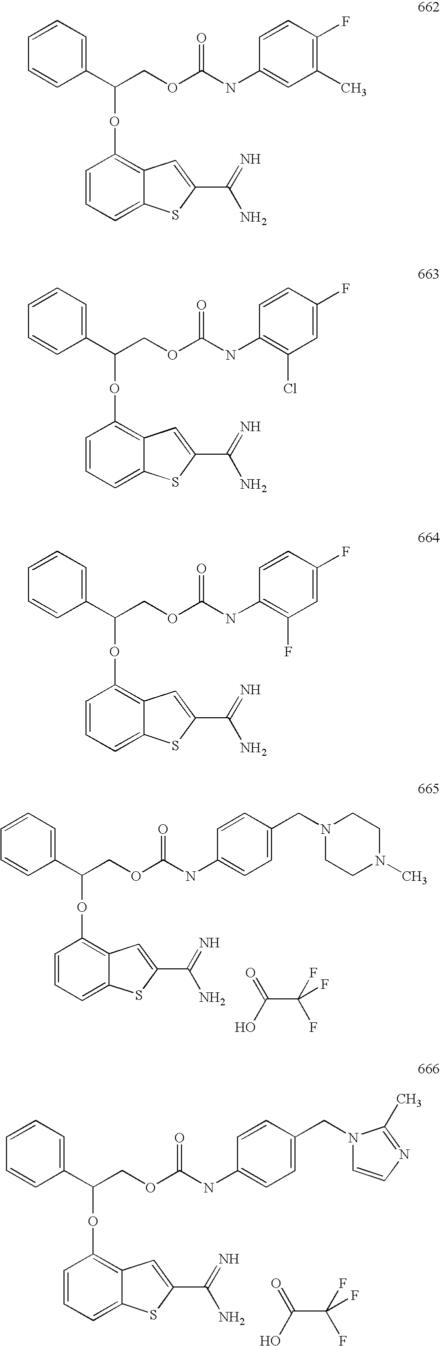

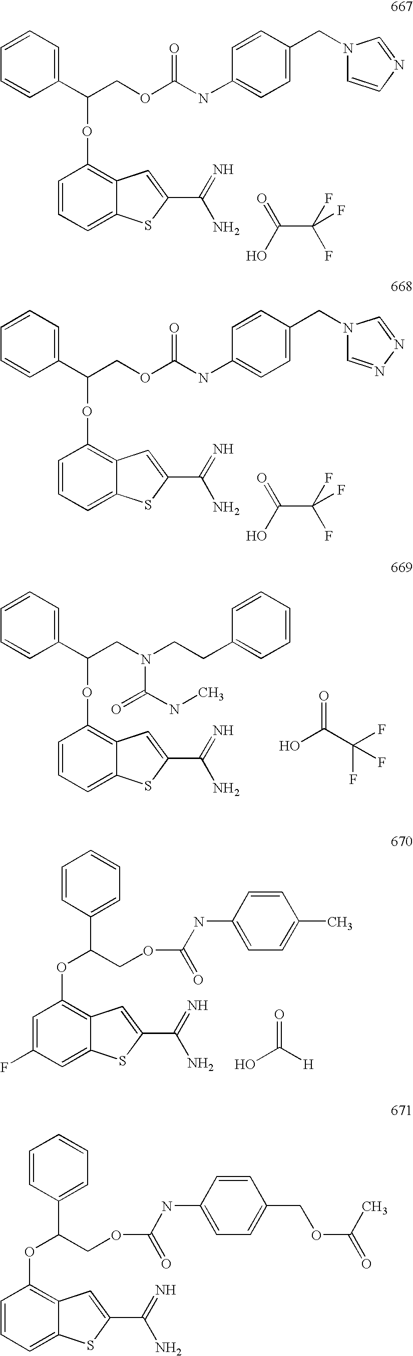

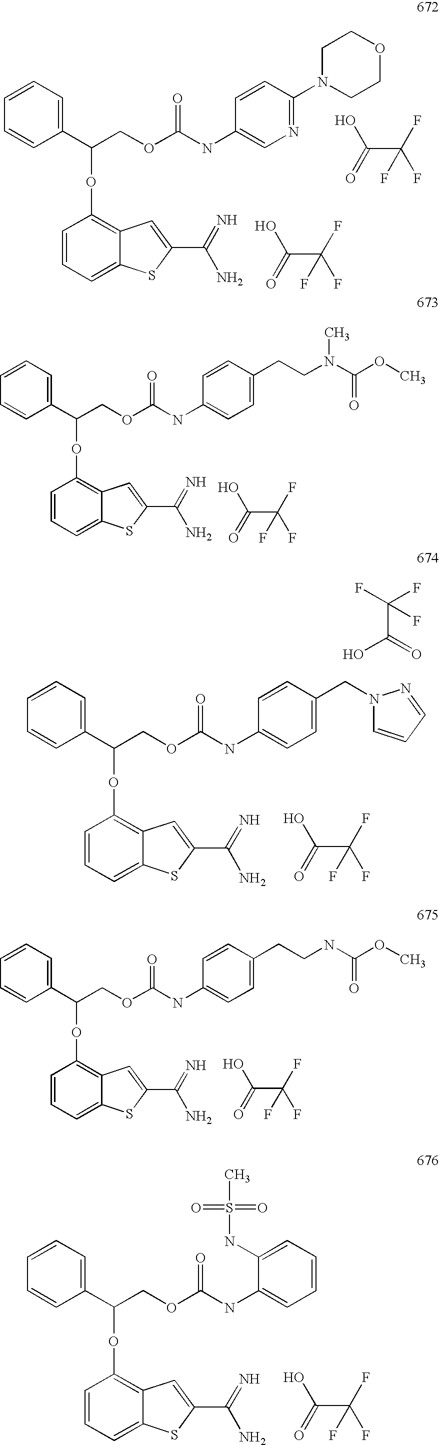

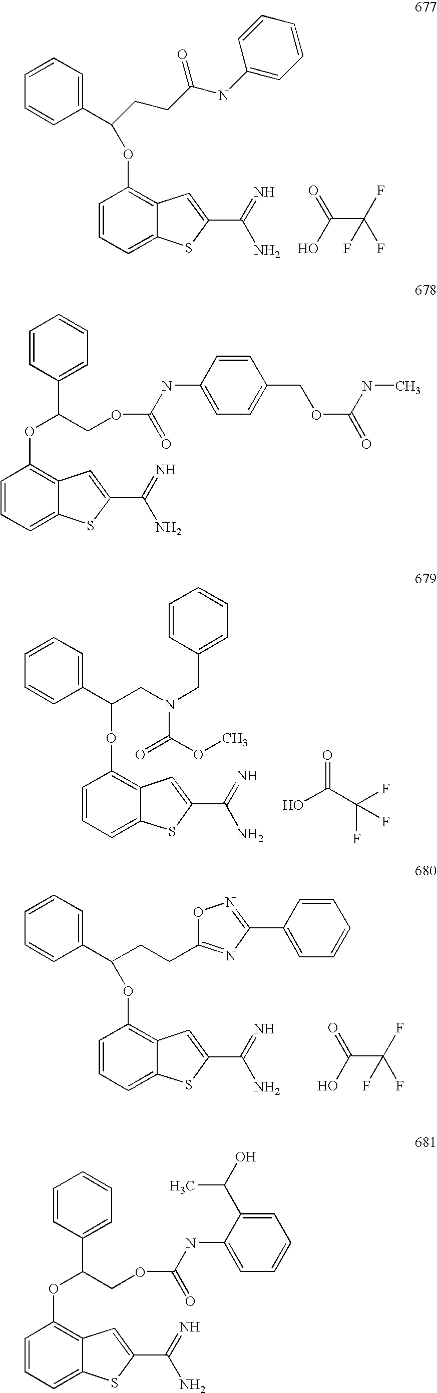

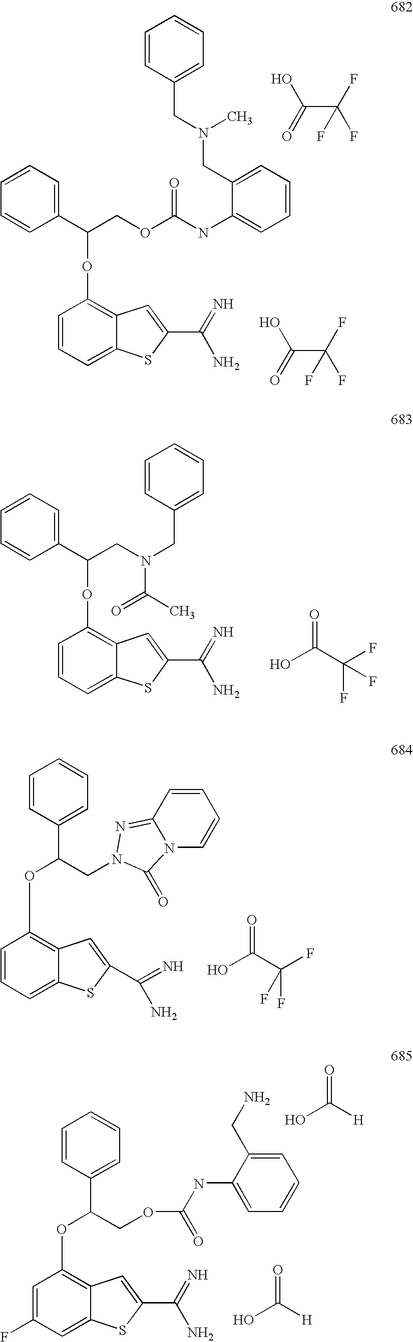

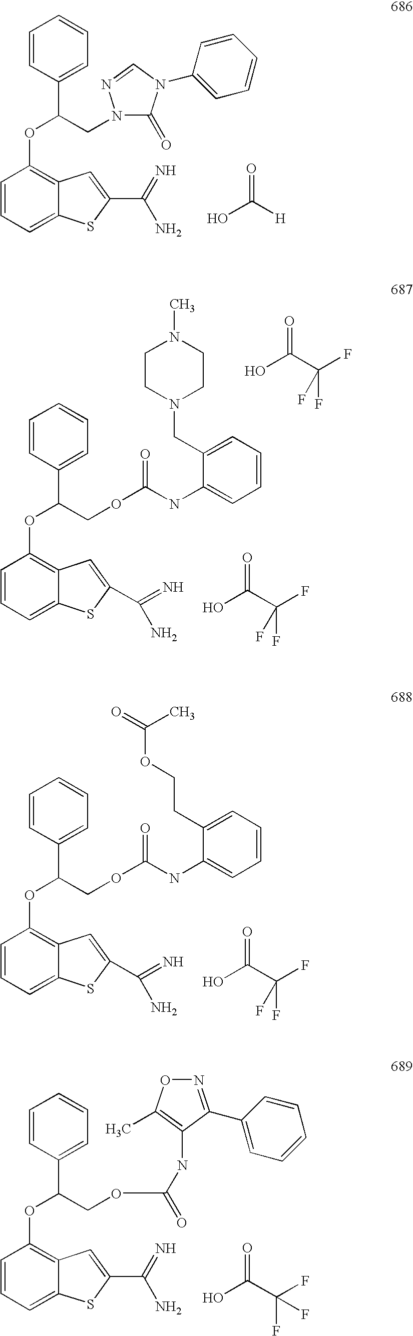

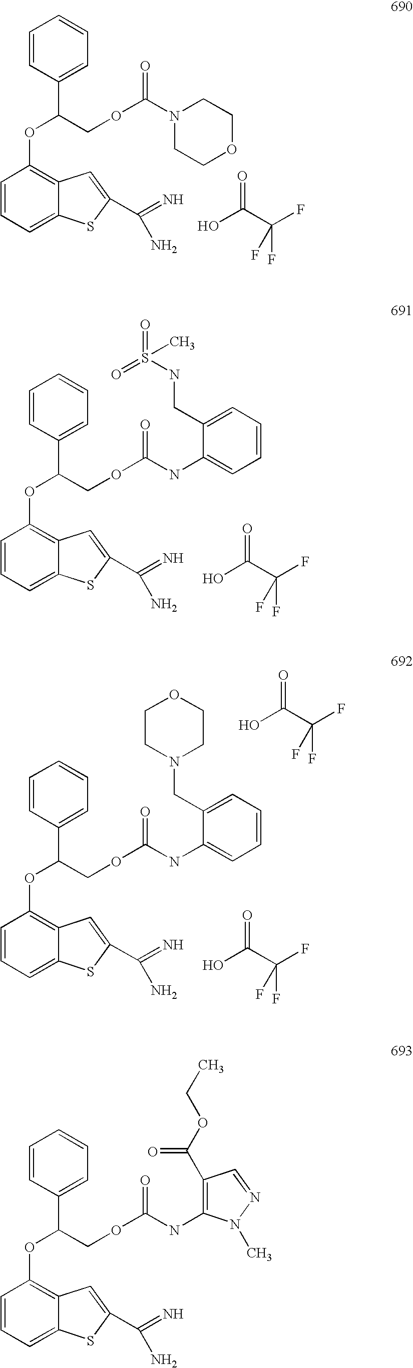

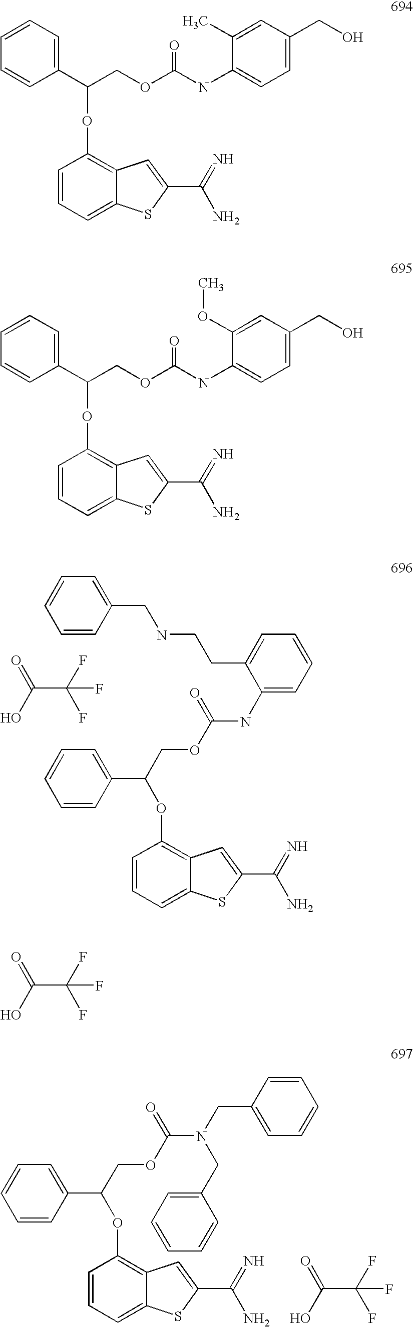

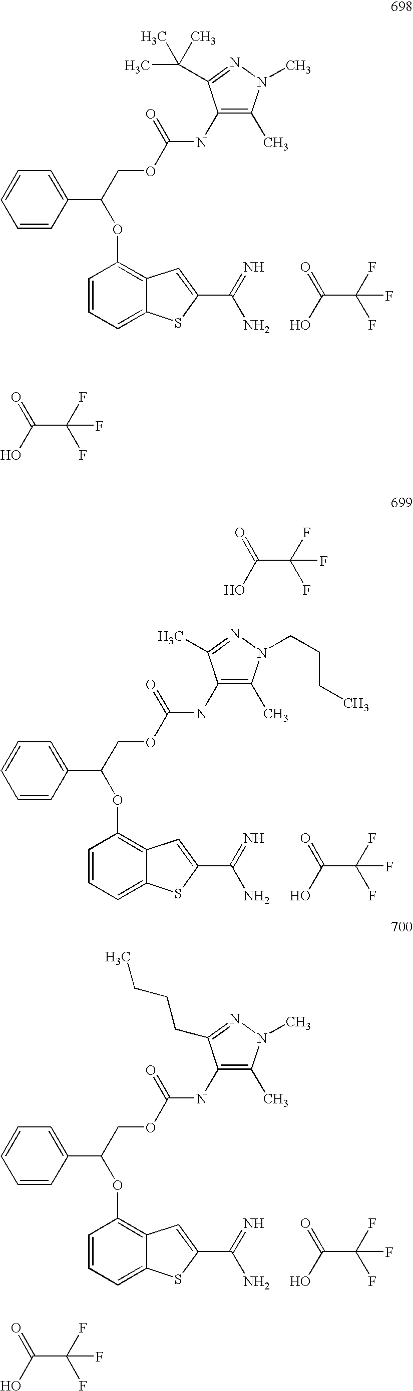

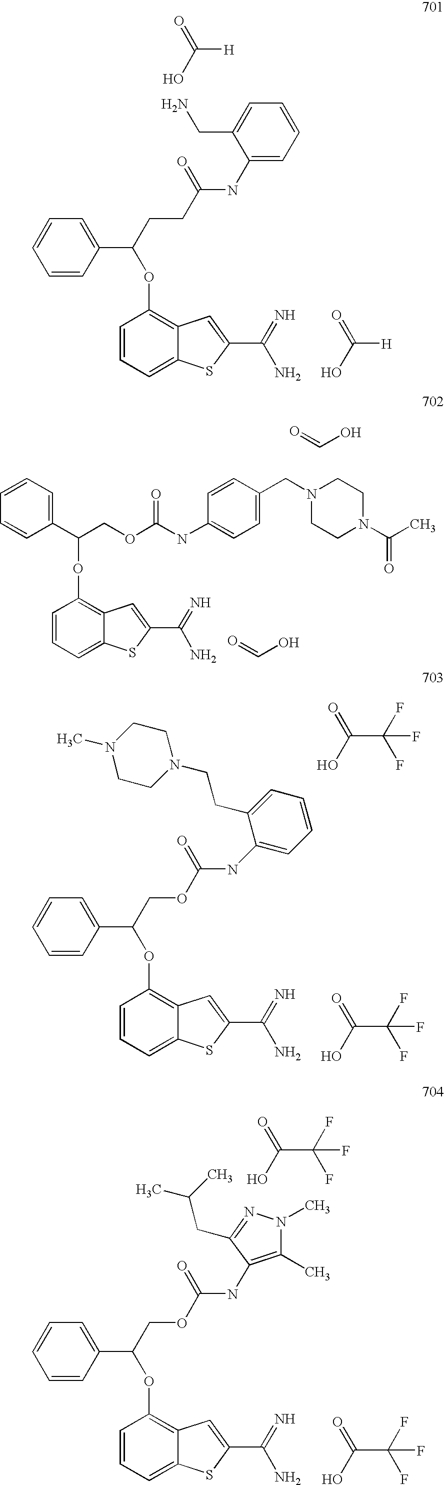

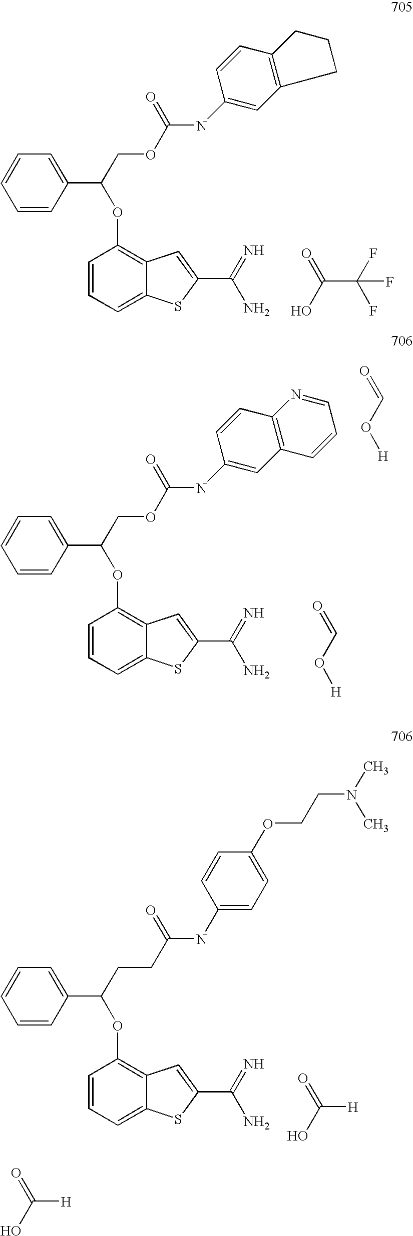

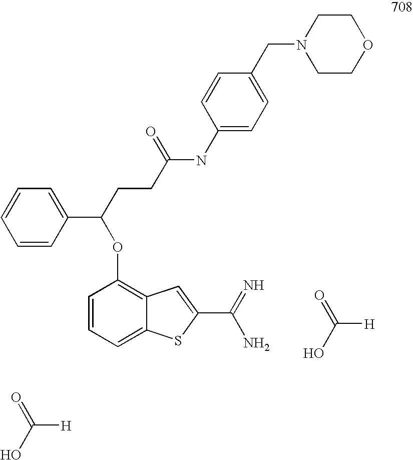

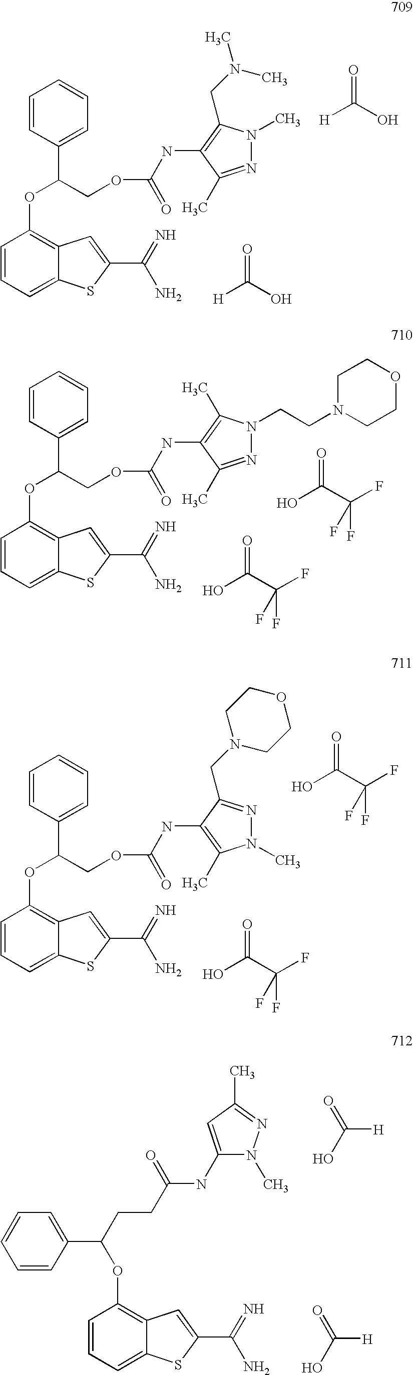

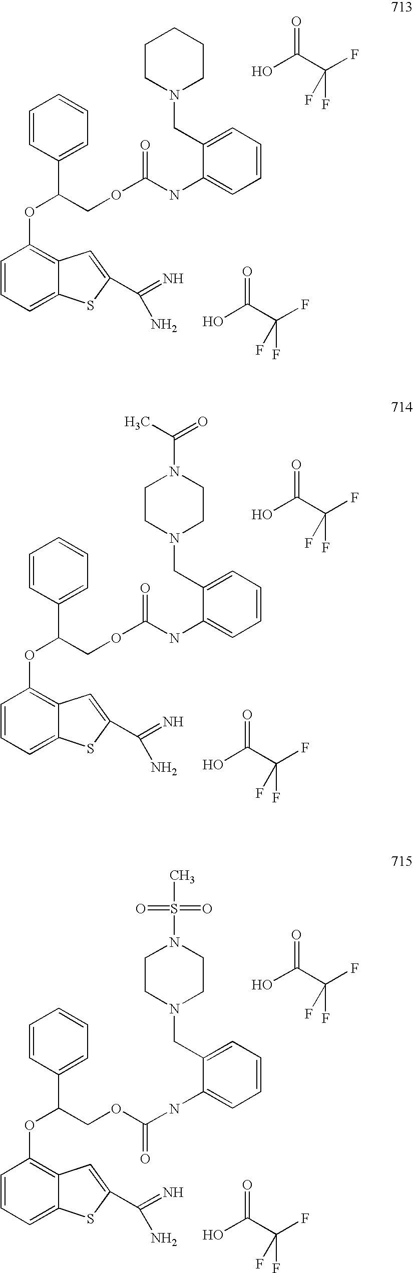

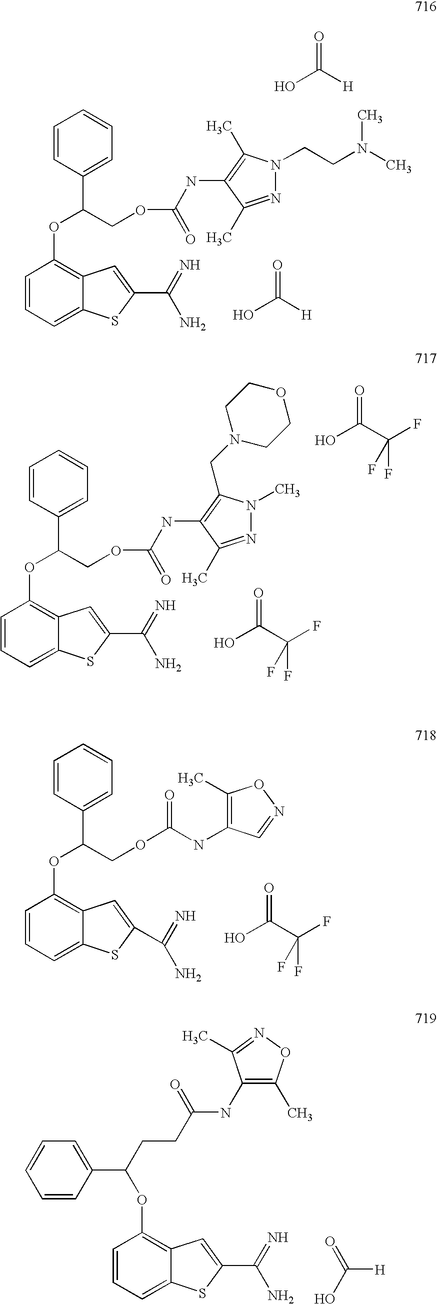

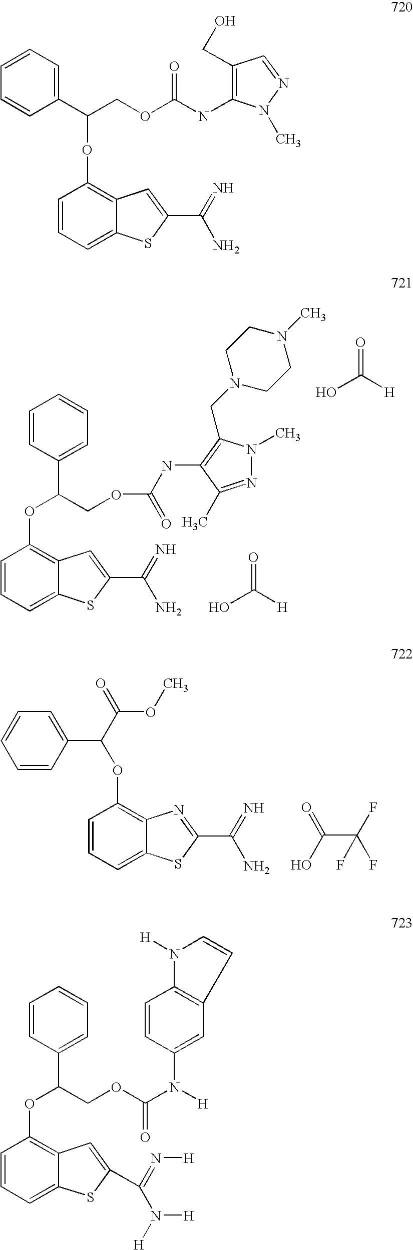

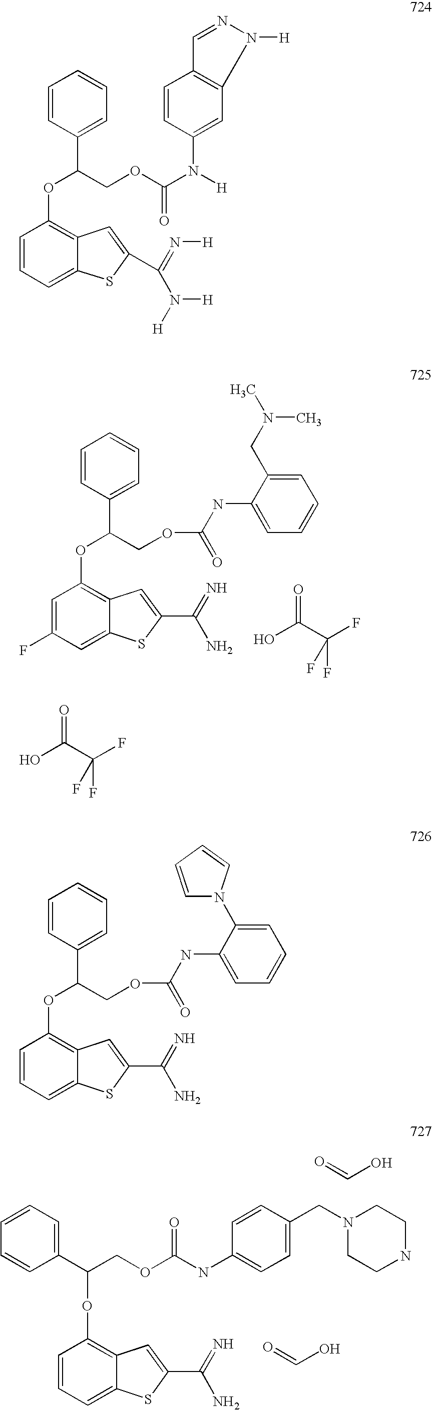

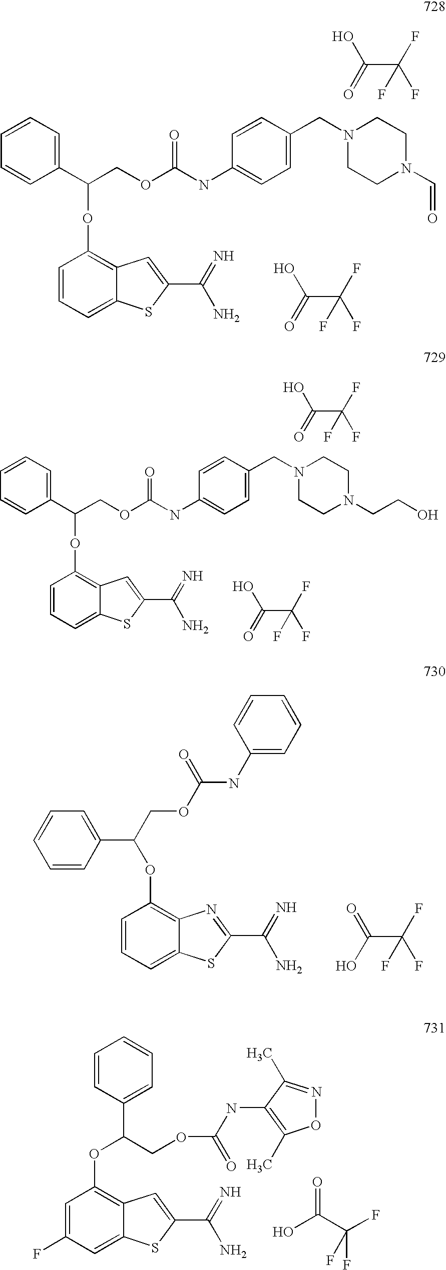

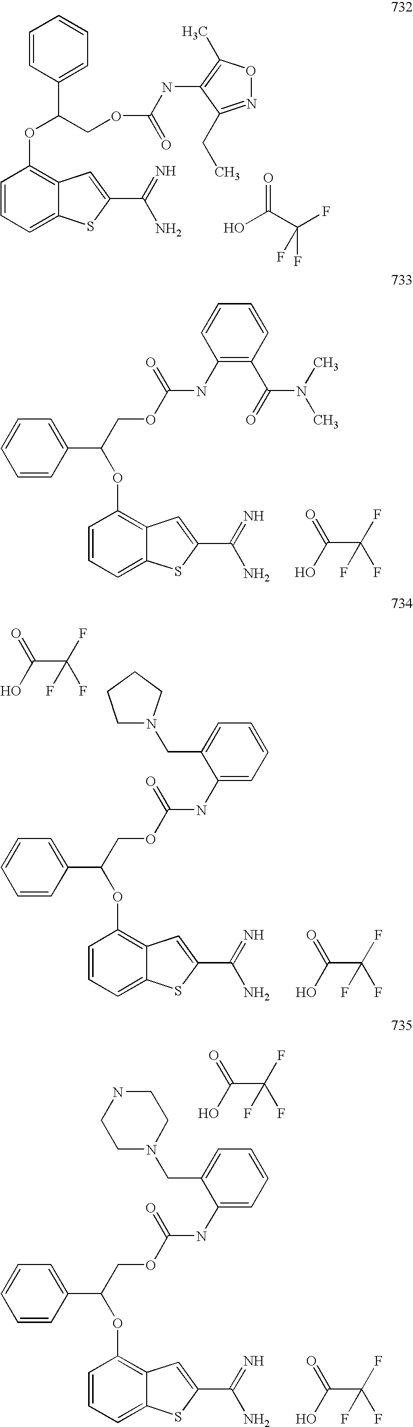

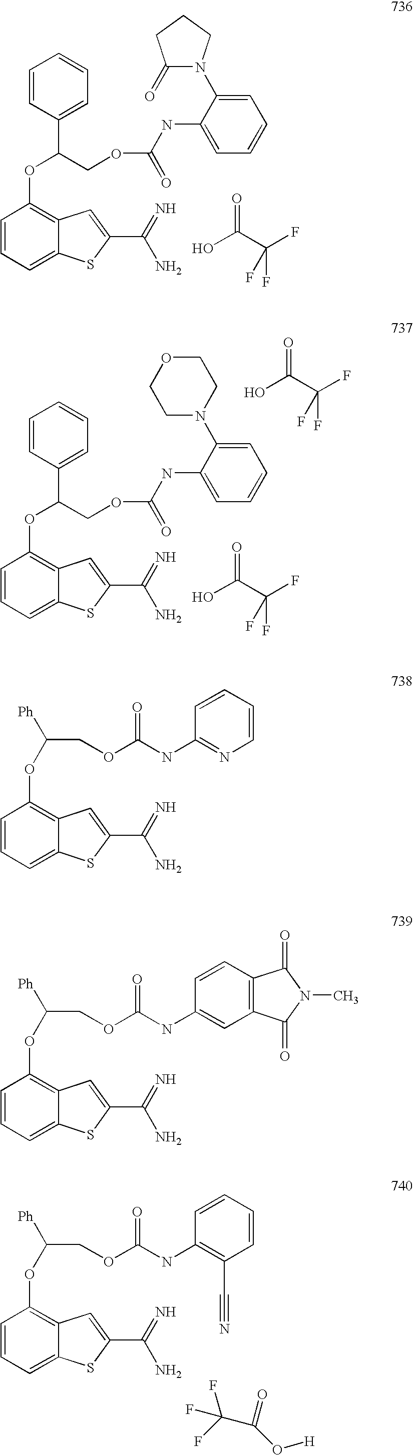

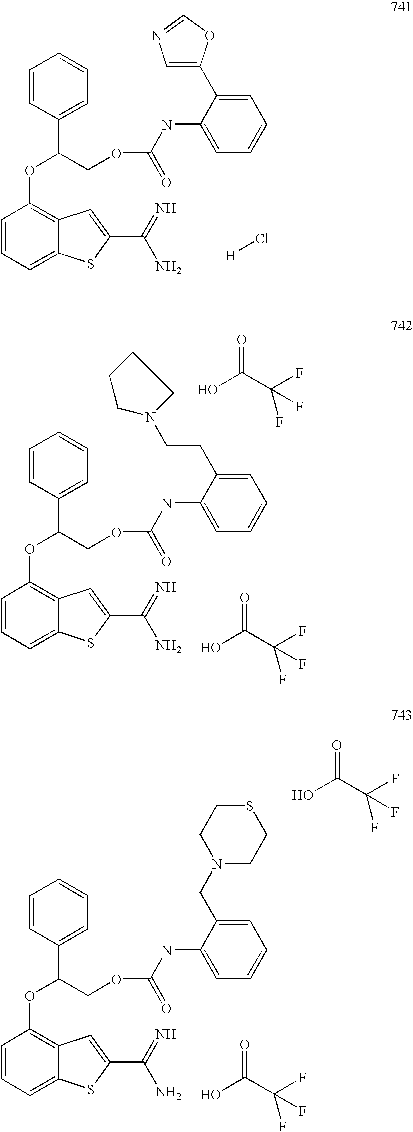

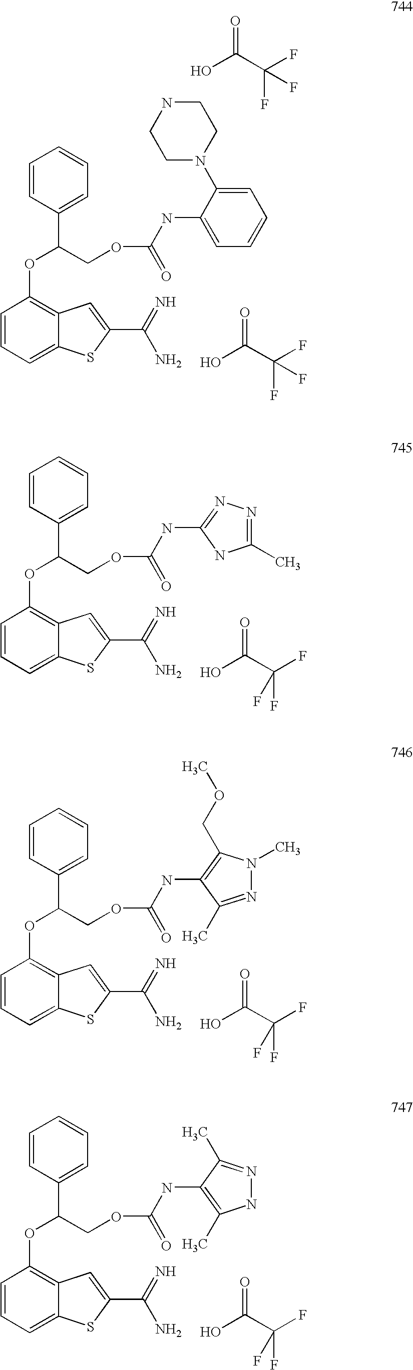

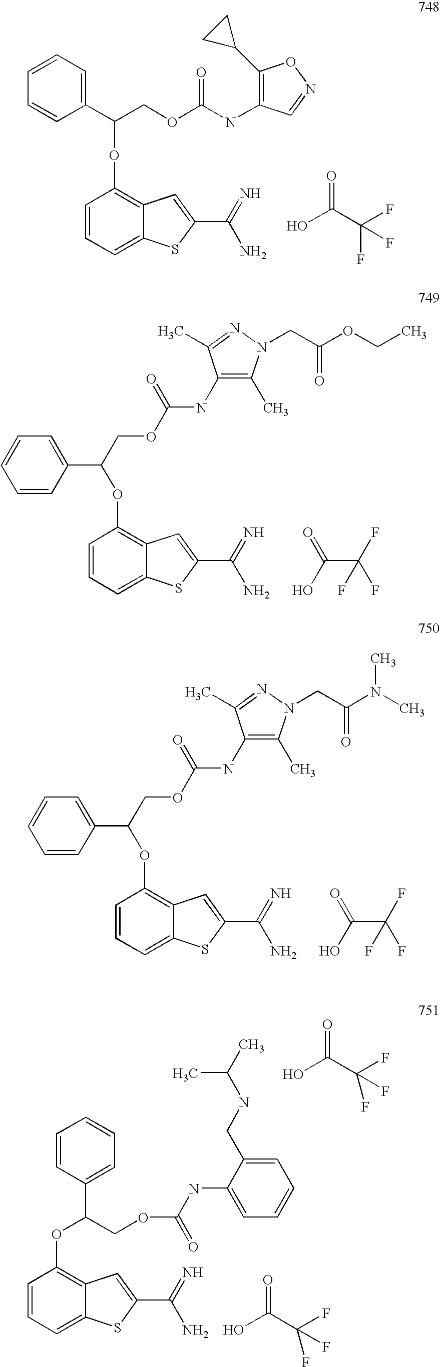

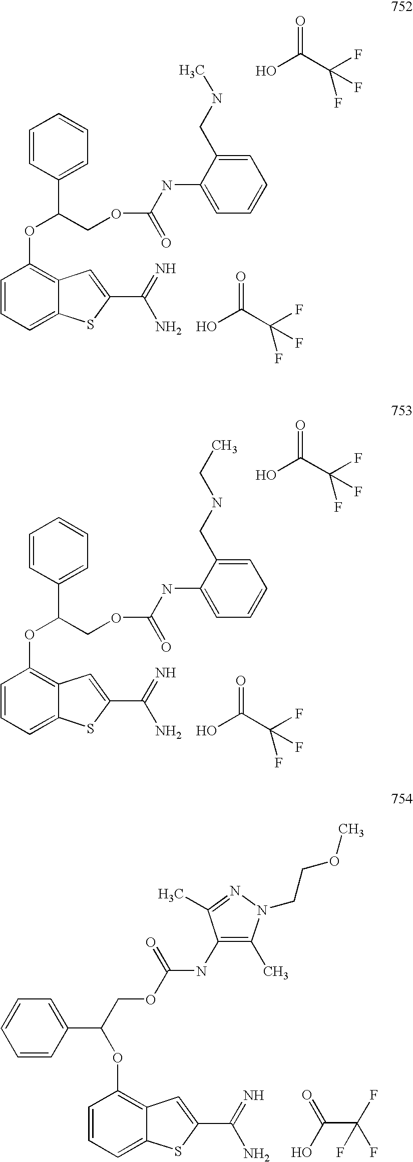

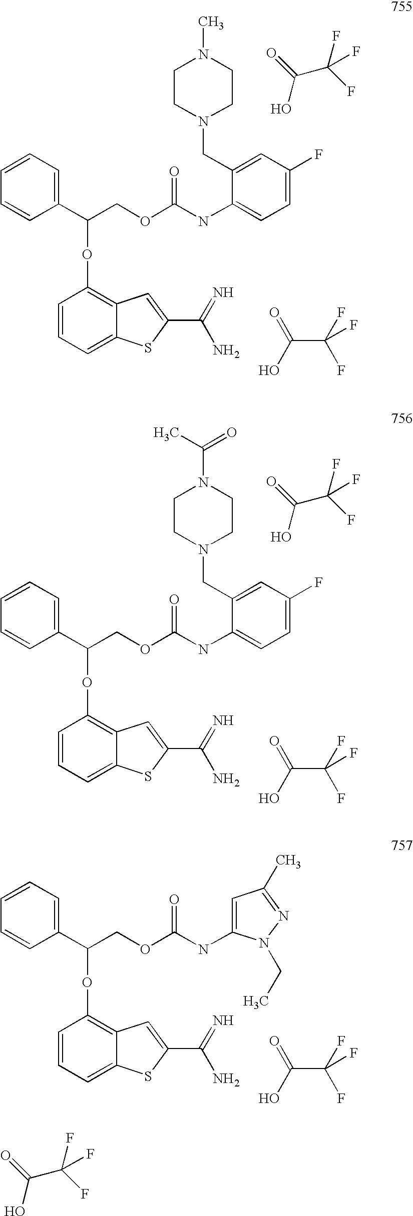

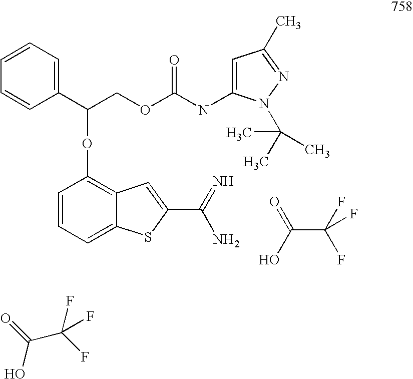

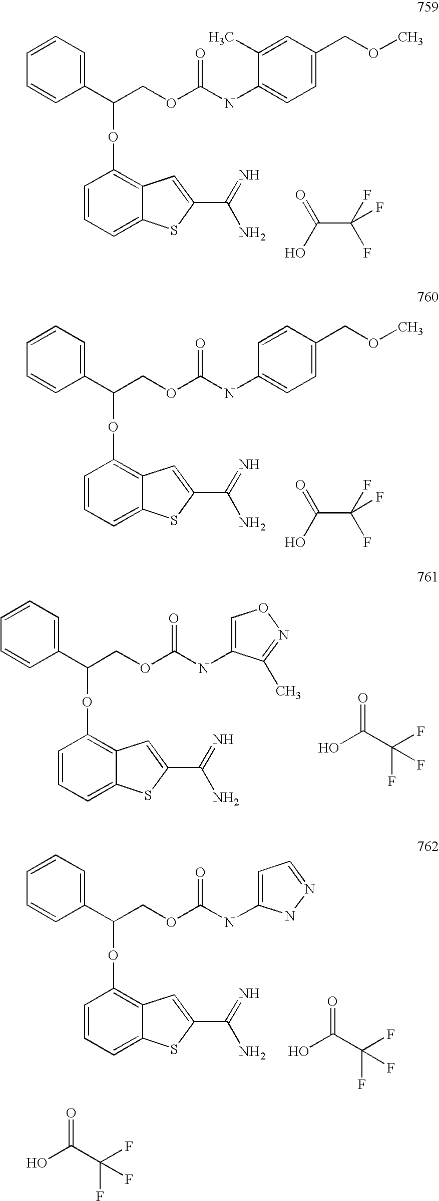

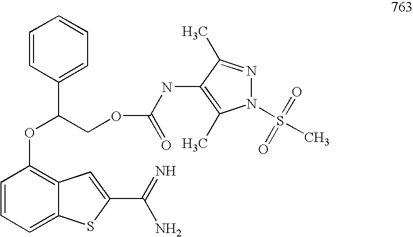

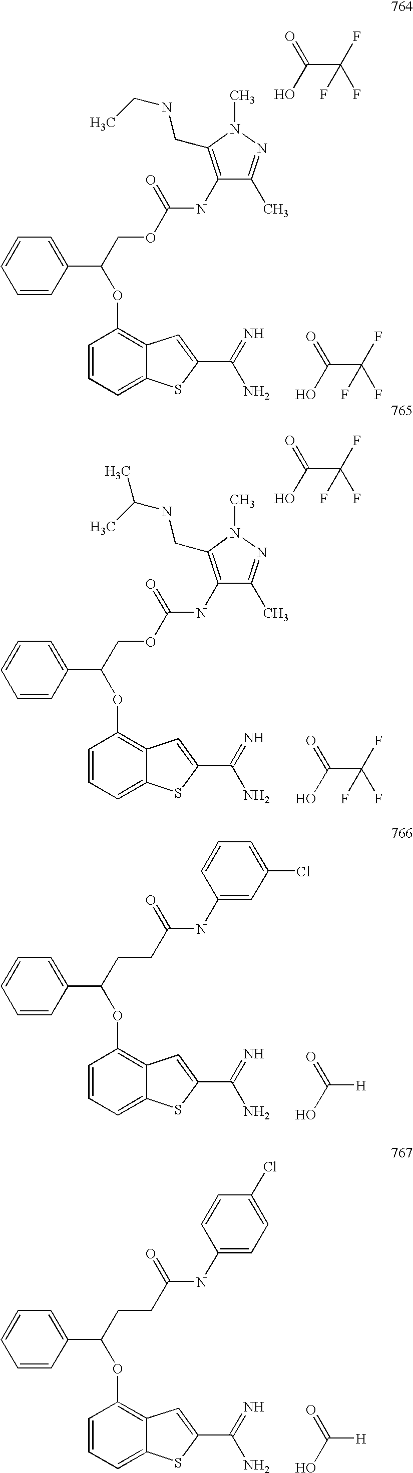

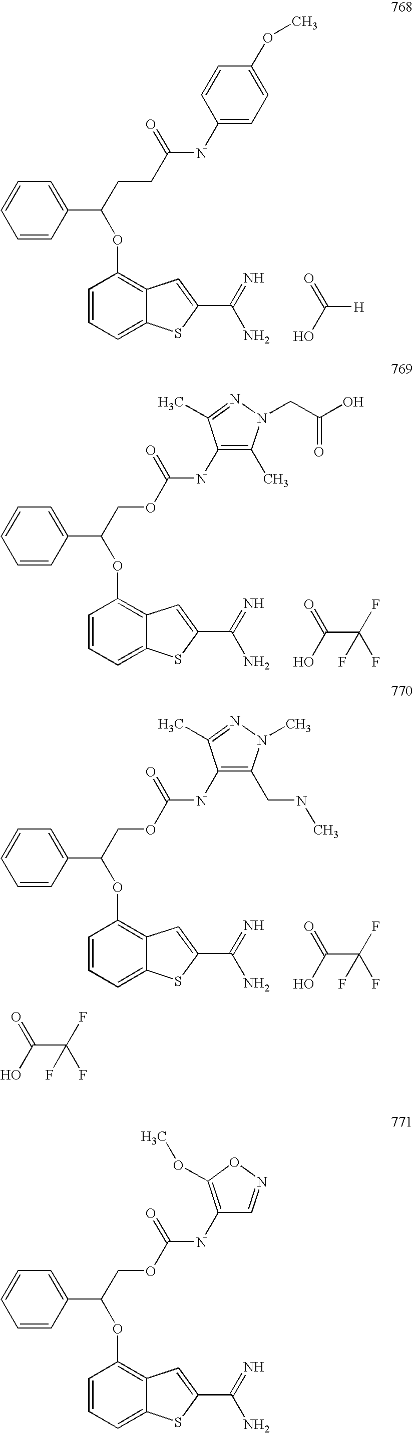

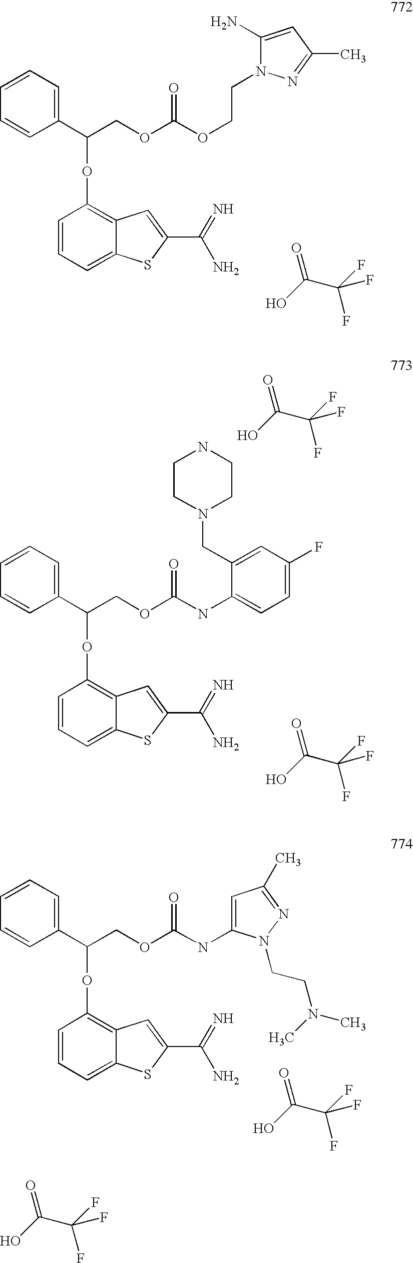

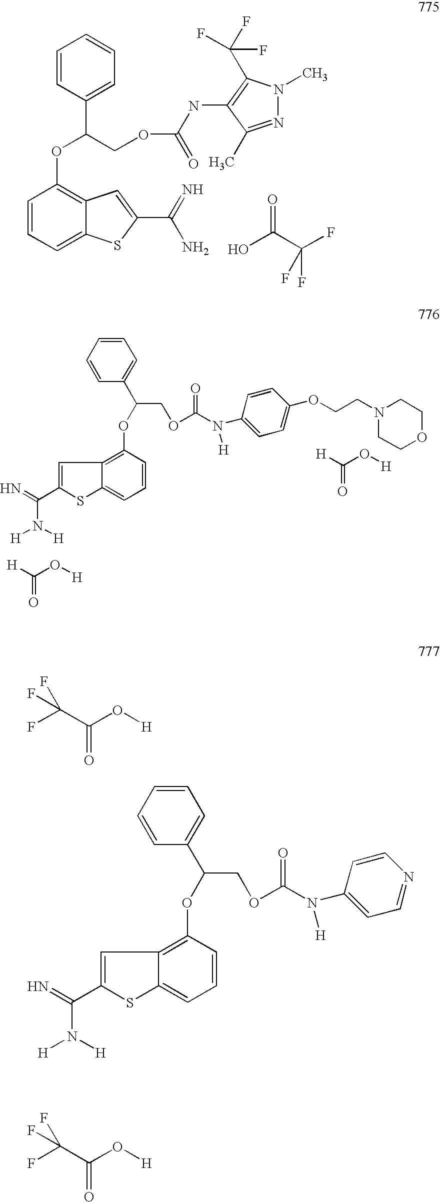

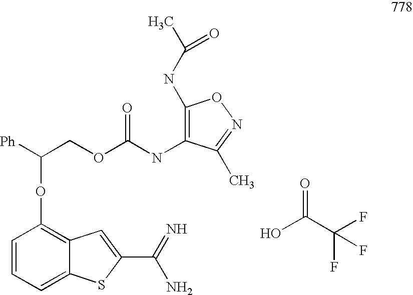

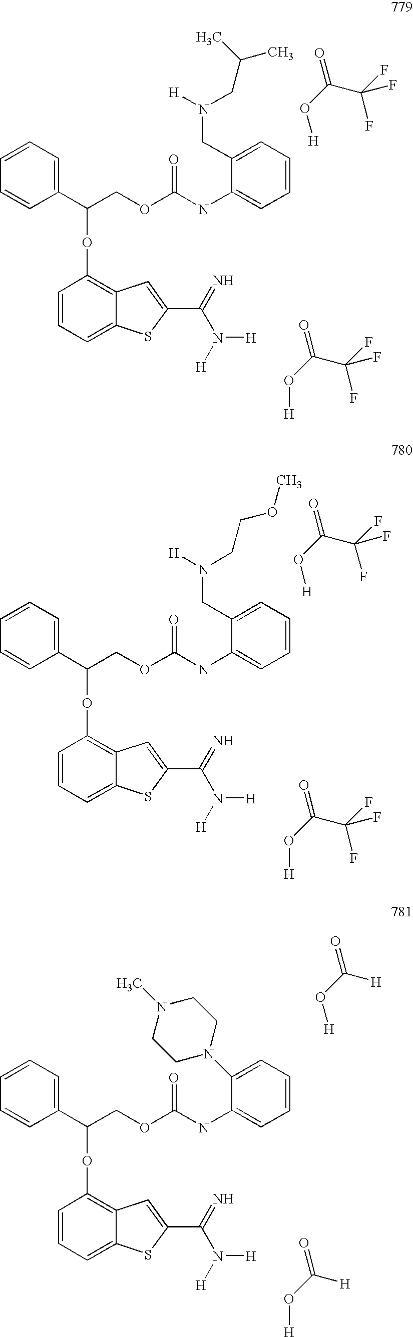

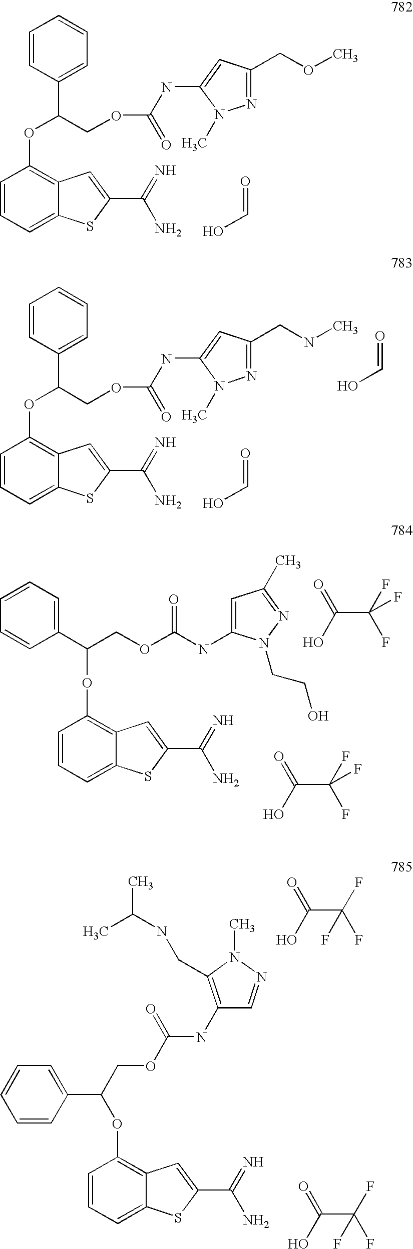

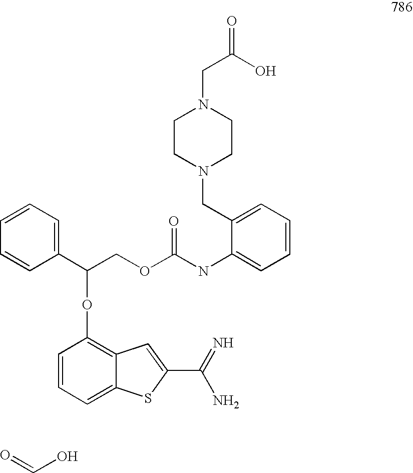

As further compounds may be mentioned:

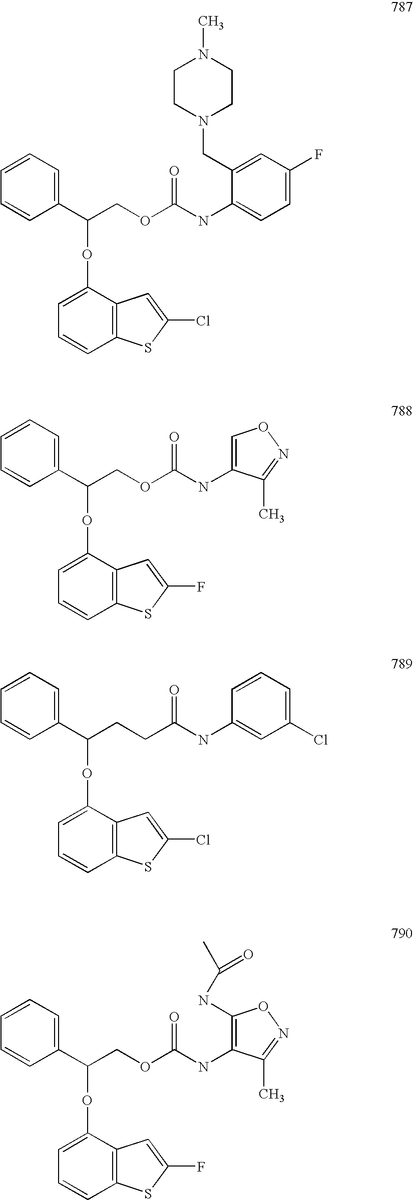

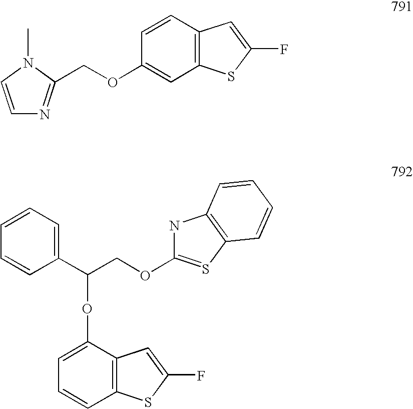

### Form of Compounds

Where applicable, compounds of the invention may exist in open ring or closed ring form, depending on conditions and environment. In particular, where substituents are such that a further ring may form on the benzothiophene ring by intermolecular reaction of substituents, the compound may exist in equilibrium between open and closed ring states. This is particularly the case for closed ring prodrugs and isosteres, such as cyclic esters, for example.

Furthermore, the interactions between substituents such as those described above may be intermolecular electrostatic interactions, such as hydrogen bonding, as well as, or instead of, covalent bonds.

Any asymmetric carbon atoms may be present in (R)-, (S)- or (R,S)-configuration, for example in (R)- or (S)-configuration. Radicals having any unsaturation are present in cis-, trans- or (cis,trans) form. The compounds may thus be present as mixtures of isomers or as pure isomers, preferably as enantiomer-pure diastereomers.

The compounds of the inventions can exist in different forms, such as free acids, free bases, esters and other prodrugs, salts and tautomers, for example, and the disclosure includes all variant forms of the compounds.

Stereoisomeric mixtures, e.g. mixtures of diastereomers, can be separated into their corresponding isomers in a manner known per se by means of suitable separation methods. Diastereomeric mixtures for example may be separated into their individual diastereomers by means of fractionated crystallization, chromatography, solvent distribution, and similar procedures. This separation may take place either at the level of a starting compound or in a compound of the invention. Enantiomers may be separated through the formation of diastereomeric salts, for example by salt formation with an enantiomer-pure chiral acid, or by means of chromatography, for example by HPLC, using chromatographic substrates with chiral ligands.

The compounds may be in the form of pharmaceutically acceptable salts. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The invention therefore also includes pharmaceutically-acceptable salts of the disclosed compounds, such as those in which the parent compound is modified by making acid or base salts thereof. Examples of acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Salts are especially the pharmaceutically acceptable acid addition salts of compounds of the invention. Such salts are formed, for example, by compounds of the invention having a basic nitrogen atom as acid addition salts, preferably with organic or inorganic acids, especially the pharmaceutically acceptable salts. Suitable inorganic acids are, for example, hydrohalic acids, such as hydrochloric acid; sulfuric acid; or phosphoric acid. Suitable organic acids are, for example, carboxylic, phosphonic, sulfonic or sulfamic acids, for example acetic acid, propionic acid, octanoic acid, decanoic acid, dodecanoic acid, glycolic acid, lactic acid, 2-hydroxybutyric acid, gluconic acid, glucosemonocarboxylic acid, fumaric acid, succinic acid, adipic acid, pimelic acid, suberic acid, azelaic acid, malic acid, tartaric acid, citric acid, glucaric acid, galactaric acid, amino acids, such as glutamic acid, aspartic acid, N-methylglycine, acetylaminoacetic acid, N-acetylasparagine, N-acetylcysteine, pyruvic acid, acetoacetic acid, phosphoserine, 2- or 3-glycerophosphoric acid, maleic acid, hydroxymaleic acid, methylmaleic acid, cyclohexanecarboxylic acid, benzoic acid, salicylic acid, 1- or 3-hydroxynaphthyl-2-carboxylic acid, 3,4,5-trimethoxybenzoic acid, 2-phenoxybenzoic acid, 2-acetoxybenzoic acid, 4-aminosalicylic acid, phthalic acid, phenylacetic acid, glucuronic acid, galacturonic acid, methane- or ethane-sulfonic acid, 2-hydroxyethanesulfonic acid, ethane-1,2-disulfonic acid, benzenesulfonic acid, 2-naphthalenesulfonic acid, 1,5-naphthalenedisulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, or ammonium salts with ammonia or suitable organic amines, such as tertiary monoamines.

In the presence of a basic group and an acid group in the same molecule, a compound of the disclosure (or an N-oxide thereof) may also form internal salts.

For isolation or purification it is also possible to use pharmaceutically unacceptable salts, for example picrates or perchlorates. Only the pharmaceutically acceptable salts or the free compounds (optionally in the form of pharmaceutical compositions) are used therapeutically, and those are therefore preferred. Such salts are formed, for example, as acid addition salts, preferably with organic or inorganic acids, from compounds of formula I (or an N-oxide thereof) with a basic nitrogen atom, especially the pharmaceutically acceptable salts.

The invention includes prodrugs of the aforementioned compounds, which can be metabolically converted to the subject compounds by the recipient host. As used herein, a prodrug is a compound that exhibits pharmacological activity after undergoing a chemical transformation in the body. An example of such a prodrug is a pharmaceutically acceptable ester of a carboxylic acid.

The invention therefore includes prodrugs for the active pharmaceutical species of the invention, for example in which one or more functional groups are protected or derivatised but can be converted in vivo to the functional group, as in the case of esters of carboxylic acids convertible in vivo to the free acid, or in the case of protected amines, to the free amino group. The term “prodrug,” as used herein, represents compounds which are transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987; H Bundgaard, ed, Design of Prodrugs, Elsevier, 1985; and Judkins, et al. Synthetic Communications, 26(23), 4351-4367 (1996); and The organic chemistry of drug design and drug action by Richard B Silverman in particular pages 497 to 546; each of which is incorporated herein by reference.

Prodrugs therefore include drugs having a functional group which has been transformed into a reversible derivative thereof. Typically, such prodrugs are transformed to the active drug by hydrolysis. As examples may be mentioned the following:

| Functional Group | Reversible derivative |
|---|---|
| Carboxylic acid | Esters, including e.g. acyloxyalkyl esters, amides |
| Alcohol | Esters, including e.g. sulfates and phosphates as |
| | well as carboxylic acid esters |
| Amidino | Amidoximes, carbamateamidino |
| Amine | Amides, carbamates, imines, enamines, |
| Boronic acid | Diol ester |
| Carbonyl (aldehyde, | Imines, oximes, acetals/ketals, enol esters, |
| ketone) | oxazolidines and thiazoxolidines |

Prodrugs also include compounds convertible to the active drug by an oxidative or reductive reaction. As examples may be mentioned:
Oxidative activation
   N- and O-dealkylation
   Oxidative deamination
   N-oxidation
   Epoxidation
Reductive activation
   Azo reduction
   Sulfoxide reduction
   Disulfide reduction
   Bioreductive alkylation
   Nitro reduction.

Also to be mentioned as metabolic activations of prodrugs are nucleotide activation, phosphorylation activation and decarboxylation activation.

Of particular mention are compounds in which R¹ is an amidino group in prodrug form. Examples of compounds which may be useful as amidino prodrugs include those described by Su et al. (J. Med. Chem., 1997, 40, 4308-4318), who describe the use of N-benzyloxycarbonyl- and N-(acyloxy)methoxycarbonyl amidine derivatives; by Boykin et al. (Bioorg. Med. Chem. Lett., 1996, 6, 3017-3020), who describe the use of amidoximes and O-alkylamidoximes; and by Weller et al. (J. Med. Chem., 1996, 39, 3139-3147) who describe the use of N-alkoxycarbonylamidines as amidine prodrugs.

Alkoxycarbonyl (carbamoyl) and acyloxymethyl carbamate groups may provide a bioconvertable prodrug of amine and amidine nitrogens (see, for example, Saulnier, et al. (1994) Bioorg. Med. Chem. Letts. 4(16):1985 1990). Lower alkoxycarbonyl groups such as methoxycarbonyl and ethoxycarbonyl, optionally substituted phenoxycarbonyl groups, groups such as benzyloxycarbonyl and p-methoxybenzyloxycarbonyl and amide oximes are known as prodrug moieties for these functional groups (see, for example, EP-A-0743320).

In exemplary compounds, R¹ is amidoxime or carbamateamidino.

The prodrugs may be used, for example, to increase solubility, stability, permeability, or to control efflux.

Other prodrugs may be carrier-linked or modified to enhance usability of active transport mechanisms. In particular, the prodrugs are pharmaceutically acceptable salts, esters or solvates.

### Use

Compounds of the invention may be useful as Factor IXa inhibitors. The compounds may be useful as an antagonist or a partial antagonist of Factor IXa. Compounds of the invention have been tested for their Factor IXa inhibitory activity. Included in the tested compounds are those shown to have IC₅₀ values for Factor IXa of less than 50 μm. Some compounds have been found to have an IC₅₀ of less than 10 μm. Particular compounds have been demonstrated to have an IC₅₀ for Factor IXa of less than 1 μm. Suitable methods for determining IC₅₀ values will be apparent to those skilled in the art, and are exemplified herein.

The compounds may show selectivity for Factor IX versus other proteases of the coagulation (e.g. thrombin, FVIIa, FXa) or the fibrinolytic cascades (e.g. plasminogen activators, plasmin) or other trypsin-like enzymes such as trypsin, enterokinase, thrombin, kallikrein, plasmin, urokinase, plasminogen activators and the like. The compounds may also be useful in the inhibition of one or more of Factor XII, Factor XI, Factor X, Factor VII and prothrombin. The compounds may be dual urokinase and Factor IX inhibitors. In particular, compounds of the invention may show selectivity for Factor IXa over uPA. For example, compounds may have IC₅₀ values for Factor IXa which are at least 10 times greater than for uPA.

Compounds of the present invention may be useful in the therapy (i.e. the treatment, prevention or delay of progression of) a cardiovascular disease or condition, for example thrombosis (including arterial, venous, cerebrovascular, coronary or deep vein thrombosis, or thrombosis associated with surgical procedures), stroke, long periods of confinement or other associated states such as pro-coagulant states, for example, which may include anti-phospholipid antibody syndrome, protein C deficiency and protein S deficiency, and inflammation, for example systemic lupus erythmatosis (SLE), diseases associated with the treatment of the kidney by haemodialysis and/or venous haemofiltration, cardiovascular disease, such as, myocardial infarction, arrhythmia, or annurism, for example. In particular, the compounds may be used to treat thrombosis, and to prevent the occurrence or re-occurrence of thrombosis and secondary thrombotic events.

Moreover, products of the disclosure may have utility in prophylaxis of re-occlusion (i.e. thrombosis) after thrombolysis, percutaneous trans-luminal angioplasty (PTA) and coronary bypass operations; the prevention of re-thrombosis after microsurgery and vascular surgery in general. Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis.

The compounds may be useful in the therapy of an arterial disease selected from acute coronary syndromes, cerebrovascular thrombosis, peripheral arterial occlusion and arterial thrombosis resulting from atrial fibrillation, valvular heart disease, arterio-venous shunts, indwelling catheters or coronary stents. According to the invention, such conditions include, for example, any thrombotically mediated acute coronary or cerebrovascular syndrome, any thrombotic syndrome occurring in the venous system, any coagulopathy, and any thrombotic complications associated with extracorporeal circulation or instrumentation. The invention still further provides a method for inhibiting the coagulation of biological samples (e.g. stored blood products and samples).

Examples of conditions involving arterial thrombosis include unstable angina (severe constrictive pain in chest of coronary origin), myocardial infarction (heart muscle cell death resulting from insufficient blood supply), ischemic heart disease (local ischemia due to obstruction, such as by arterial narrowing, of blood supply), reocclusion during or after percutaneous transluminal coronary angioplasty, restenosis after percutaneous transluminal coronary angioplasty, occlusion of coronary artery bypass grafts, and occlusive cerebrovascular disease. Also with regard to arterio-venous (mixed) thrombosis, anti-thrombotic compounds of the invention may be useful for maintaining patency in arteriovenous shunts. Indications involving arterial thrombosis include acute coronary syndromes (especially myocardial infarction and unstable angina), cerebrovascular thrombosis and peripheral arterial occlusion and arterial thrombosis occurring as a result of atrial fibrillation, valvular heart disease, arterio-venous shunts, indwelling catheters or coronary stents. Accordingly, in another aspect there is provided a method of treating a disease or condition selected from this group of indications, comprising administering to a mammal, especially a human patient, a product of the disclosure. The disclosure includes products for use in an arterial environment, e.g. a coronary stent or other arterial implant, having a coating which comprises a product according to the disclosure. The products of the disclosure may be used prophylactically to treat an individual at risk of suffering from arterial thrombosis or a condition or disease involving arterial thrombosis or therapeutically (including to prevent re-occurrence of thrombosis or secondary thrombotic events).

The compounds may be useful for the prevention of thrombosis in procedures involving an extracorporeal blood circuit, for example a surgical procedure such as coronary artery bypass graft (CABG) surgery. The compounds of this disclosure may be incorporated into a cardiopulmonary bypass machine or may be administered externally to the extracorporeal blood circuit. More usually, they may be administered intravenously to the patient by infusion. In one method, the disclosed products may be used to prevent thrombosis in surgery. In particular, the products of the present invention may be used to prevent thrombosis during CABG surgery. Thus the disclosure contemplates medicaments to prevent thrombosis in the extracorporeal circuit during CABG surgery.

It is known that hypercoagulability may lead to thromboembolic diseases. Examples of venous thromboembolism which may be treated or prevented with compounds of the disclosure include obstruction of a vein, obstruction of a lung artery (pulmonary embolism), deep vein thrombosis, thrombosis associated with cancer and cancer chemotherapy, thrombosis inherited with thrombophilic diseases such as Protein C deficiency, Protein S deficiency, antithrombin III deficiency, and Factor V Leiden, and thrombosis resulting from acquired thrombophilic disorders such as systemic lupus erythematosus (inflammatory connective tissue disease). Also with regard to venous thromboembolism, compounds of the disclosure are useful for maintaining patency of indwelling catheters.

Examples of cardiogenic thromboembolism which may be treated or prevented with compounds of the disclosure include thromboembolic stroke (detached thrombus causing neurological affliction related to impaired cerebral blood supply), cardiogenic thromboembolism associated with atrial fibrillation (rapid, irregular twitching of upper heart chamber muscular fibrils), cardiogenic thromboembolism associated with prosthetic heart valves such as mechanical heart valves, and cardiogenic thromboembolism associated with heart disease.

The compounds may be used as modulators of the intrinsic and/or extrinsic clotting pathway by inhibiting the biological activities of one or more enzymes associated therewith. As such, the compounds of the invention may be useful in application such as management, treatment, or control of diseases in humans associated with one or both of the intrinsic or extrinsic clotting pathway, for example, diseases that are contemplated may be any disease, or disease state, associated with one or more of the intrinsic or extrinsic clotting pathway and may be, for example, stroke, myocardial infarction, deep vein thrombosis, inflammation (acute and chronic) and clotting associated with surgery and haemodialysis.

Other conditions associated with hypercoagulability and thromboembolic diseases which may be mentioned inherited or acquired deficiencies in heparin cofactor II, circulating antiphospholipid antibodies (Lupus anticoagulant), homocysteinemia, heparin induced thrombocytopenia and defects in fibrinolysis.

The present compounds may be useful in controlling hemostasis and especially for inhibiting coagulation, for example in the treatment or prevention of secondary events after myocardial infarction.

The compounds may also be used in the treatment of patients by haemodialysis, by providing the product in a dialysis solution. The invention therefore includes dialysing solutions and dialysing concentrates which comprise a product of the disclosure, as well as the use of the compounds in dialysis therapy.

The compounds may be useful in the treatment or prevention of undesirable cell proliferation. The undesirable cell proliferation is typically undesirable hyperplastic cell proliferation, for example proliferation of smooth muscle cells, especially vascular smooth muscle cells. The compounds may particularly find application in the treatment of intimal hyperplasia, one component of which is proliferation of smooth muscle cells. Restenosis can be considered to be due to neointimal hyperplasia; accordingly intimal hyperplasia in the context of the disclosure includes restenosis.

Compounds of the invention may be useful in the treatment of ischemic disorders. More particularly, they may be used in the treatment (whether therapeutic or prophylactic) of an ischemic disorder in a patient having, or at risk of, non-valvular atrial fibrillation (NVAF). Ischemic disorders are conditions whose results include a restriction in blood flow to a part of the body. The term will be understood to include thrombosis and hypercoagulability in blood, tissues and/or organs. Particular uses that may be mentioned include the prevention and/or treatment of ischemic heart disease, myocardial infarction, systemic embolic events in e.g. the kidneys or spleen, and more particularly of cerebral ischemia, including cerebral thrombosis, cerebral embolism and/or cerebral ischemia associated with non-cerebral thrombosis or embolism (in other words the treatment (whether therapeutic or prophylactic) of thrombotic or ischemic stroke and of transient ischemic attack), particularly in patients with, or at risk of, NVAF.

Compounds of the invention may be combined and/or co-administered with another cardiovascular treatment agent. There are large numbers of cardiovascular treatment agents available in commercial use, in clinical evaluation and in pre-clinical development, which could be selected for use with a product of the disclosure for the prevention of cardiovascular disorders by combination drug therapy. Such an agent can be one or more agents selected from, but not limited to several major categories, namely, a lipid-lowering drug, including an IBAT (ileal Na⁺/bile acid cotransporter) inhibitor, a fibrate, niacin, a statin, a CETP (cholesteryl ester transfer protein) inhibitor, and a bile acid sequestrant, an anti-oxidant, including vitamin E and probucol, a IIb/IIIa antagonist (e.g. abciximab, eptifibatide, tirofiban), an aldosterone inhibitor (e.g. spirolactone and epoxymexrenone), an adenosine A2 receptor antagonist (e.g. losartan), an adenosine A3 receptor agonist, a beta-blocker, acetylsalicylic acid, a loop diuretic and an ACE (angiotensin converting enzyme) inhibitor.

Compounds of the invention may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction. The compounds of the disclosure may be combined and/or co-administered with any antithrombotic agent with a different mechanism of action, such as the antiplatelet agents acetylsalicylic acid, ticlopidine, clopidogrel, thromboxane receptor and/or synthetase inhibitors, prostacyclin mimetics and phosphodiesterase inhibitors and ADP-receptor (P₂ T) antagonists.

In particular, compounds of the invention may be combined and/or co-administered with a cardioprotectant, for example an adenosine A1 or A3 receptor agonist. In particular, the compounds may be used in combination or co-administration with a lipid-lowering drug, a fibrate, niacin, a statin, a CETP inhibitor, a bile acid sequestrant, an anti-oxidant, a IIb/IIIa antagonist, an aldosterone inhibitor, an A2 antagonist, an A3 agonist, a beta-blocker, acetylsalicylic acid, a loop diuretic, an ace inhibitor, an antithrombotic agent with a different mechanism of action, an antiplatelet agent, a thromboxane receptor and/or synthetase inhibitor, a fibrinogen receptor antagonist, a prostacyclin mimetic, a phosphodiesterase inhibitor, an ADP-receptor (P₂ T) antagonist, a thrombolytic, a cardioprotectant or a COX-2 inhibitor. More particularly, a compound or product of the invention may be used in conjunction with a non-steroidal anti-inflammatory drug (NSAID), e.g. a COX-2 inhibitor, and used to treat or prevent an inflammatory disease or condition, for example nephritis, systemic lupus, erythematosus, rheumatoid arthritis, glomerulonephritis, vasculitis and sarcoidosis. Accordingly, the compounds of the disclosure may be combined and/or co-administered with an NSAID.

### Formulations & Administration

Compounds of the invention may be administered orally, intravenously, subcutaneously, buccally, rectally, dermally, nasally, tracheally, bronchially, by any other parenteral route, as an oral or nasal spray or via inhalation, The compounds may be administered in the form of pharmaceutical preparations comprising prodrug or active compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

Typically, therefore, the pharmaceutical compounds of the invention may be administered orally or parenterally (“parenterally” as used herein, refers to modes of administration which include intravenous, intramuscular, intraperitoneal, intrasternal, subcutaneous and intraarticular injection and infusion.) to a host to obtain an protease-inhibitory effect. In the case of larger animals, such as humans, the compounds may be administered alone or as compositions in combination with pharmaceutically acceptable diluents, excipients or carriers.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

In the treatment, prevention, control, amelioration, or reduction of risk of conditions which require inhibition of Factor IXa activity, an appropriate dosage level may generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions may be provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0 and 1000.0 milligrams of the active ingredient for the symptomatic adjustment of the dosage to the patient to be treated. The compounds may be administered on a regimen of 1 to 4 times per day, e.g. once or twice per day. The dosage regimen may be adjusted to provide the optimal therapeutic response.

According to a further aspect of the invention there is thus provided a pharmaceutical composition including a compound of the invention, optionally in admixture with a pharmaceutically acceptable adjuvant, diluent or carrier.

Pharmaceutical compositions of this invention for parenteral injection may comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example. Prolonged absorption of the injectable pharmaceutical form may be brought about by the inclusion of agents (for example aluminum monostearate and gelatin) which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms may be made by forming microencapsule matrices of the drug in biodegradable polymers, for example polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations may also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

Solid dosage forms for oral administration include capsules, tablets, pills, powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

Oral formulations may contain a dissolution aid. Examples of dissolution aids include nonionic surface active agents, such as sucrose fatty acid esters, glycerol fatty acid esters, sorbitan fatty acid esters (e.g. sorbitan trioleate), polyethylene glycol, polyoxyethylene hydrogenated castor oil, polyoxyethylene sorbitan fatty acid esters, polyoxyethylene alkyl ethers, methoxypolyoxyethylene alkyl ethers, polyoxyethylene alkylphenyl ethers, polyethylene glycol fatty acid esters, polyoxyethylene alkylamines, polyoxyethylene alkyl thioethers, polyoxyethylene polyoxypropylene copolymers, polyoxyethylene glycerol fatty acid esters, pentaerythritol fatty acid esters, propylene glycol monofatty acid esters, polyoxyethylene propylene glycol monofatty acid esters, polyoxyethylene sorbitol fatty acid esters, fatty acid alkylolamides, and alkylamine oxides; bile acid and salts thereof (e.g. chenodeoxycholic acid, cholic acid, deoxycholic acid, dehydrocholic acid and salts thereof, and glycine or taurine conjugate thereof); ionic surface active agents, such as sodium laurylsulfate, fatty acid soaps, alkylsulfonates, alkylphosphates, ether phosphates, fatty acid salts of basic amino acids; triethanolamine soap, and alkyl quaternary ammonium salts; and amphoteric surface active agents, such as betaines and aminocarboxylic acid salts.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions include polymeric substances and waxes.

The active compounds may also be in micro-encapsulated form, if appropriate, with one or more of the above-mentioned excipients.

The active compounds may be in finely divided form, for example it may be micronised.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration may be in the form of suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolisable lipid capable of forming liposomes can be used. The present compositions in liposome form can contain, in addition to a compound of the present invention, stabilisers, preservatives, excipients and the like. The preferred lipids are the phospholipids and the phosphatidyl cholines (lecithins), both natural and synthetic. Methods to form liposomes are known in the art, for example, Prescott, Ed., Methods in Cell Biology, Volume XIV, Academic Press, New York, N.Y. (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

In a specific aspect, the present invention provides products, particularly kits, for producing a single-dose administration unit. The products (kits) may each contain both a first container having the active compound (optionally combined with additives, for example anti-oxidant, preservative and, in some instances, tonicity agent) and a second container having the carrier/diluent (for example water, optionally containing one or more additives, for example tonicity agent). As examples of such products may be mentioned single and multi-chambered (e.g. dual-chamber) pre-filled syringes; exemplary pre-filled syringes are available from Vetter GmbH, Ravensburg, Germany. Such dual chamber syringes or binary syringes will have in one chamber a dry preparation including or consisting of the active compound and in another chamber a suitable carrier or diluent such as described herein. The two chambers are joined in such a way that the solid and the liquid mix to form the final solution.

The following Examples illustrate the invention.

### EXAMPLES

General procedures for obtaining compounds of the invention are described below, together with specific Examples of the synthesis and inhibitory activities of particular compounds of the invention. It will be appreciated that these processes are provided solely for the purpose of illustrating the invention and should not be construed as limiting. A process utilising similar or analogous reagents and/or conditions known to one skilled in the art may also be used to obtain a compound of the invention.

### Scheme 1: General Procedure for the Synthesis of 6-Substituted Benzothiophene Analogues

A general methodology for synthesising compounds of the invention is shown in Scheme 1 below:

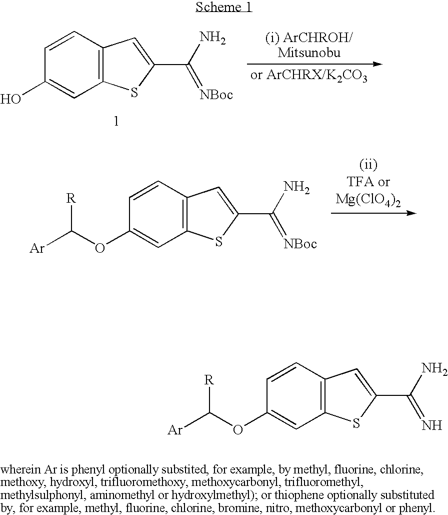

### Step (i)

In step (i) of Scheme 1, intermediate 1 is either coupled with a benzene or thiophene alcohol by Misunobu reaction, or alkylated with a benzene or thiophene halide in the presence of a base.

Intermediate 1 may be synthesised by the route shown in Scheme 2.

Many benzylic halides or alcohols are commercially available. Where they are not commercially available, they may be prepared either by bromination from corresponding phenyl acetic acid (Scheme 3) or from corresponding aldehyde by a cyanohydrin reaction (Scheme 4). Thiophene alcohols can be synthesised by reduction from corresponding aldehydes or ketones (Scheme 5).

In step (ii), the Boc protecting group is then removed with an acid (such as TFA, HCl or formic acid) or by magnesium perchlorate when the products are acid sensitive, such as most thiophene analogues.

By way of example, intermediate 7 was synthesised according to step (i) of Scheme 1.

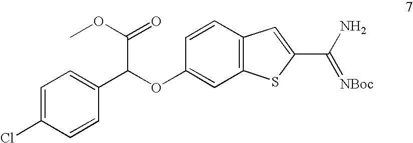

1 (150 mg, 0.51 mmol) was stirred with 4-chloromandelate (155 mg, 0.77 mmol) in dry THF at room temperature under argon followed by triphenylphosphine (202.1 mg, 0.77 mmol). A solution (1 ml) of DEAD in dry THF was then added dropwise over a period of 15 minutes. The reaction mixture was stirred for 15 hours and purified through a column (silica gel, 2:8 ethyl acetate:hexane) to furnish 198 mg of the title compound 7 (81%). ¹H NMR (DMSO-d6): δ 3.73 (s, 3H, CH₃), 6.31 (s, 1H, alpha H), 7.43 (d, 1H, ArH), 7.58 (m, 2H, m-Cl—ArH), 7.65 (m, 2H, o-Cl—ArH), 7.84 (s, 1H, ArH), 8.04 (d, 1H, ArH), 8.33 (s, 1H, ArH), 9.4 (NH, bs). MS: ES+ 476 (M+H).

### Step (ii)

In step (ii) of Scheme 1, the Boc protecting group is then removed with an acid (such as TFA, HCl or formic acid) or by magnesium perchlorate when the products are acid sensitive, such as most thiophene analogues.

### Scheme 2: Synthesis of Intermediate 1

Intermediate 1 of Scheme 1 may be obtained according to the procedure of Scheme 2:

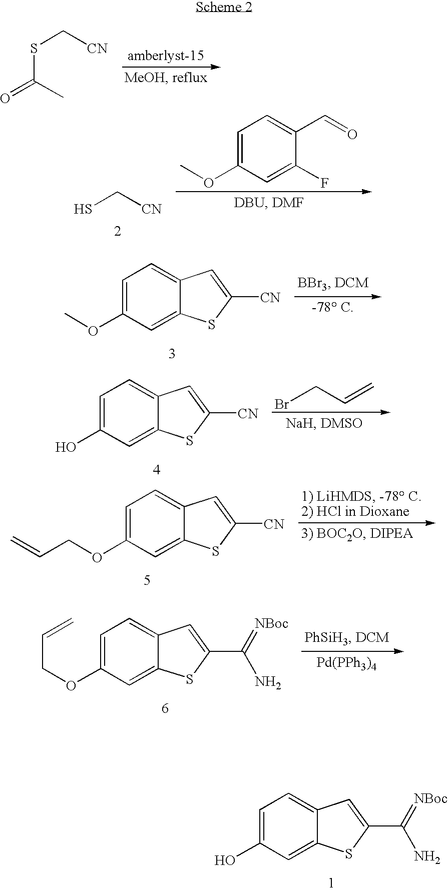

a. Synthesis of Thioacetonitrile 2

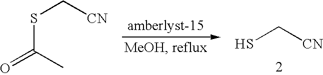

Acetylthioacetonitrile (23.00 g, 0.198 mol) was added to a solution of dry methanol (150 ml) containing amberlyst-15 (5 g) and the reaction mixture was refluxed at 85° C. under argon overnight. The reaction mixture was then allowed to cool to room temperature, and then filtered into a vessel containing dry amberlyst-15 (5 g). Solvent removal under vacuum gave a brown oil (12.13 g, 84%). ¹H NMR (DMSO-d6): δ 3.49 (s, 2H), 3.98 (s, 1H).

b. Synthesis of 2-cyano-6-methoxybenzothiophene 3

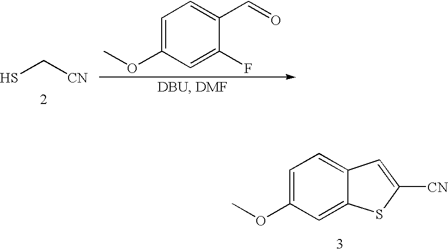

Thioacetonitrile 1 was added dropwise to a solution of 2-fluoro-4-methoxybenzaldehyde and DBU in anhydrous DMF under argon at room temperature. The reaction mixture was stirred for 20 minutes prior to heating to 60° C. and stirring at 60° C. overnight. Solvent was removed under vacuum, and the residue was dissolved in DCM, and then washed with 1M HCl, water. The aqueous layer was extracted with DCM. Combined organic extracts were dried over magnesium sulphate and concentrated under reduced pressure. Part of the pure product was obtained by recrystallising the crude residue from EtOAc/hexane (11.40 g), and more product was obtained by flash column chromatography of the concentrated mother liquors (silica gel, 2:8 EtOAc/hexane) to yield a yellow solid (9.23 g), yield: 68.1%. ¹H NMR (DMSO-d6): δ 3.76 (s, 3H), 7.07 (d, 1H), 7.65 (s, 1H), 7.81 (d, 1H), 8.18 (s, 1H). MS: ES+ 189.8 (M+H).

c. Synthesis of 2-cyano-6-hydroxybenzothiophene 4

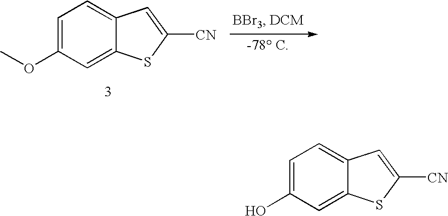

1.0 M Boron tribromide solution in DCM (328 ml, 0.328 mol) was added dropwise to a cooled solution of 2-cyano-6-methoxybenzothiophene (20.6 g, 0.109 mol) in DCM (350 ml) at −78° C. under argon. The reaction mixture was stirred at −78° C. for 1 hour, then allowed to warm to room temperature and stirring at room temperature overnight. The excess reagent was quenched by ice and saturated NaHCO₃ solution. The organic layer was separated and aqueous layer was extracted with EtOAc. The combined organic layers were dried over magnesium sulphate, and concentrated to afford a tan coloured solid (12.90 g, 67%). ¹H NMR (DMSO-d6): δ 7.09 (d, 1H), 7.49 (s, 1H), 7.90 (d, 1H), 8.29 (s, 1H), 10.40 (s, 1H, D₂O labile). MS: ES+ 175.81 (M+H).

d. Synthesis of 6-allyloxy-2-cyanobenzothiophene 5

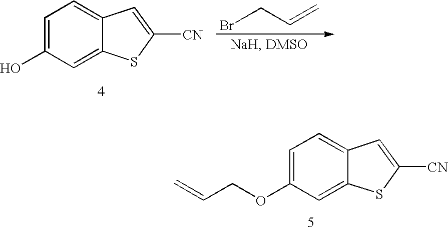

Sodium hydride (60% in mineral oil) (3.25 g, 0.081 mol) was added in several portions to a stirred cold solution of 2-cyano-6-hydroxybenzothiophene (12.86 g, 0.0735 mol) in DMSO at 5° C. under argon, and the reaction mixture was stirred for 20 minutes. Allyl bromide (6.96 ml, 0.081 mol) was added dropwise, and the reaction mixture was allowed to warm to room temperature and stirred at room temperature overnight. Reaction mixture was poured onto iced water (250 ml) and extracted with EtOAc. The combined organic extracts were washed with 1M NaOH solution, dried over magnesium sulphate and concentrated under reduced pressure to afford a light brown solid (15.81 g, 100%). ¹H NMR (DMSO-d6): δ 4.46 (d, 2H), 5.31 (d, 1H), 5.41 (d, 1H), 6.04 (m, 1H), 7.15 (d, 1H), 7.71 (s, 1H), 7.89 (d, 1H), 8.26 (s, 1H). MS: ES+ 215.89 (M+H).

e. Synthesis of 6-allyloxy-2-amidinebenzothiophene 6a

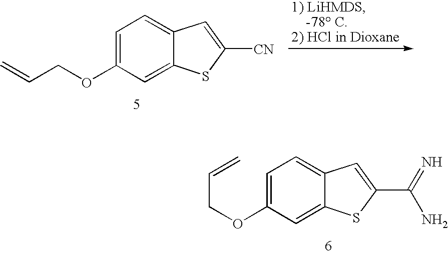

1M Lithiumtrimethylsilylamide solution in THF (95.60 ml, 0.0956 mol) was added to a stirred cooled solution of 6-allyloxy-2-cyanobenzothiophene (15.81 g, 0.0735 mol) in THF (200 ml) at −78° C. under argon. The reaction mixture was stirred for 10 minutes at −78° C. then allowing the temperature to rise to room temperature and stirred for 1.5 hours at room temperature. 4M HCl solution in dioxane (20 ml, 0.08 mol) was added and stirred at room temperature for 16 hours. Solvent removal under vacuum yielded a brown semi-solid (40.485 g), no further purification was undertaken. ¹H NMR (DMSO-d6): δ 4.69 (s, 2H), 5.30 (d, 1H), 5.45 (d, 1H), 6.05-6.12 (m, 1H), 7.16 (d, 1H), 7.77 (s, 1H), 7.94 (d, 1H), 8.34 (s, 1H), 9.30 (b, 3H). MS: ES+ 232.91 (M+H).

f. Synthesis of 6-allyloxy-2-(t-butyloxycarbonyl)amidinebenzothiophene 6b

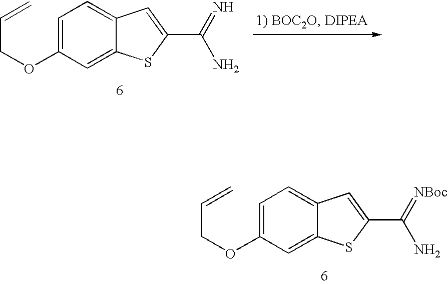

To a stirred solution of crude 6-allyloxy-2-amidinebenzothiophene (40.495 g) and diisopropylethylamine (38.33 ml, 0.22 mol) in dioxane and water (1:1, 40 ml) at room temperature, di-tert-butyl-dicarbonate (19.23 g, 0.088 mol) was added and stirred for 2 hours. Reaction mixture was diluted with EtOAc, then the organic layer was separated, and aqueous layer was extracted with EtOAc. The combined organic layers were washed with saturated sodium bicarbonate solution, dried over magnesium sulphate and concentrated. The crude residue was purified by crystallisation from ether to yield a light brown solid (15.98 g). More product was obtained from the purification of concentrated mother liquid by flash chromatography (silica gel, 8:2 EtOAc/hexane) (2.316 g). Yield: 74.5%. ¹H NMR (DMSO-d6): δ 1.44 (s, 9H), 4.64 (d, 2H), 5.25 (d, 1H), 5.45 (d, 1H), 6.20 (m, 1H), 7.03 (d, 1H), 7.54 (s, 1H), 7.77 (d, 1H), 8.22 (s, 1H), 9.07 (s, 2H). MS: ES+ 333 (M+H).

g. Synthesis of 2-(N-t-butyloxycarbonyl)amidine-6-hydroxybenzothiophene 1

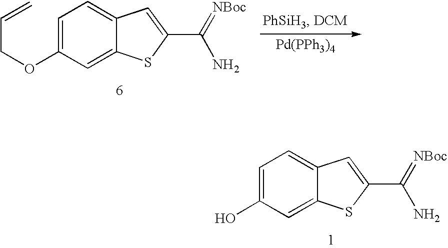

To a stirred cooled solution of tetrakis(triphenylphosphine)palladium(0) (1.27 g, 0.11% mol) and 6-allyloxy-2-(t-butyl)amidinebenzothiophene (18.296 g, 0.055 mol) in DCM (250 ml) at 0° C. under argon, phenylsilane (7.45 ml, 0.0605 mol) was added dropwise and the reaction mixture was stirred at room temperature for one hour. Dropwise addition of water was undertaken until H₂ (g) evolution ceased and stirred for 30 minutes. The organic layer was separated; aqueous layer was extracted with DCM. The combined organic layers were dried over magnesium sulphate and concentrated. Bi-phase recrystallisation using DCM and hexane was conducted on the crude residue to afford a yellow solid (4.494 g), and flash chromatography on concentrated mother liquors gave more product as a yellow solid (silica: 20% EtOAc/hexane) (3.692 g). Yield: 58%. ¹H NMR (DMSO-d6): δ 1.67 (s, 9H), 7.12 (d, 1H), 7.46 (s, 1H), 7.92 (d, 1H), 8.40 (s, 1H), 9.27 (s, 2H), 10.19 (s, 1H). MS: ES+ 292.94 (M+H).

### Scheme 3: Synthesis of Benzyl Halides

Benzyl halides (for use in step (i) of Scheme 1) may be synthesised according to Scheme 3:

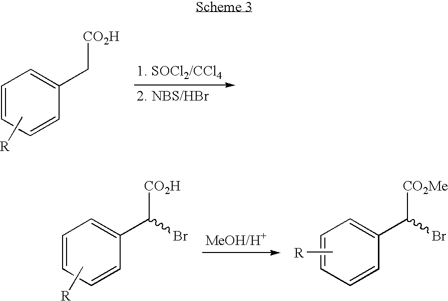

By way of example, methyl α-bromo-3,4-difluorophenylacetate 8 was synthesised according to Scheme 3.

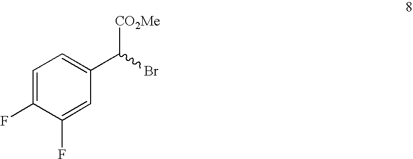

3,4-Difluoroacetic acid (500 mg, 2.90 mmol) and thionyl chloride (848 μl, 11.62 mmol) were stirred at 65° C. for 30 minutes. (N.B. NMR showed a slight shift downfield of the a protons from 3.53 to 3.63 ppm). To this solution, NBS (1.185 g, 6.66 mmol) and 7 drops of HBr/HOAc were added with more CCl₄ and heated at 70° C. for 10 minutes and then 85° C. for 2.5 hrs. The succinimide precipitate was filtered and the reaction mixture concentrated. The latter was heated with methanol with a few drops of HCl (4M in 1,4-dioxane) at 65° C. for 30 minutes. The product was purified through a column (silica gel, 1:9 MeOH:DCM) to provide the desired compound. ¹H NMR (DMSO-d6): δ 3.74 (s, CO₂Me, 3H), 5.99 (s, α-H, 1H), 7.11 (d, o-H, 1H), 7.34-7.31 (m, o- & p-Hs, 2H). MS: ES+ 266.95 (M+H).

### Scheme 4: Synthesis of Benzyl Alcohols

Benzyl alcohols (for use in step (i) of Scheme 1) may be synthesised according to Scheme 4:

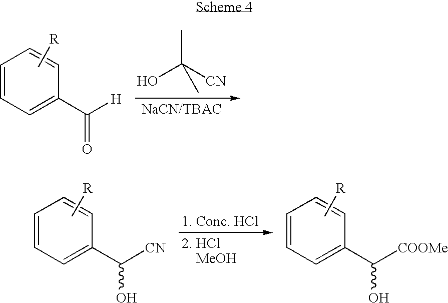

By way of example, methyl 3-methoxycarbonylmandelate 9 was synthesised according to Scheme 4.

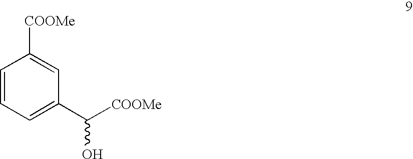

A mixture of methyl 3-formylbenzoate (1 g, 6 mmol), acetone cyanohydrin (0.7 mL, 9 mmol), sodium cyanide (7.1 mg, 0.145 mmol) and tetrabutylammonium chloride (11 mg, 0.04 mmol) in DCM (5 mL) and water (2.5 mL) was stirred at room temperature for 18 hours. The organic layer was separated, and aqueous layer was extracted with DCM. The combined organic layers were washed with water, dried over magnesium sulfate. Solvent removal gave a yellow oil. Concentrated hydrochloric acid (5 mL) was added to this oil, and the mixture is stirred at 100° C. for 2 hours. After cooling, the reaction mixture was extracted with EtOAc, and organic layer was washed with water, then dried over magnesium sulphate, and concentrated to give an off-white solid (1.08 g). Methanol (30 mL) and 0.5 mL 4M HCl solution in dioxane were added to this solid, and the reaction solution was stirred at refux temperature for one hour. Solvent was removed, and the residue was purified by column chromatography (silica gel, 1:1 hexane:EtOAc) to afford 9 as a white solid (0.69 g, 51.3% over the three steps). ¹H NMR (CD₃OD): δ 3.59 (s, 3H, ArCOOCH₃), 3.80 (s, 3H, COOCH₃), 5.17 (s, 1H, CH), 7.38 (t, 1H, ArH), 7.58 (d, 1H, ArH), 7.86 (d, 1H, ArH), 8.00 (s, 1H, ArH). MS: ES+ 225 (M+H).

### Schemes 5 and 6: Synthesis of Substituted Thiophene Alcohols

Thiophene alcohols (for use in step (i) of Scheme 1) may be synthesised according to Scheme 5 or Scheme 6:

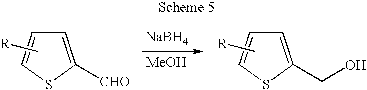

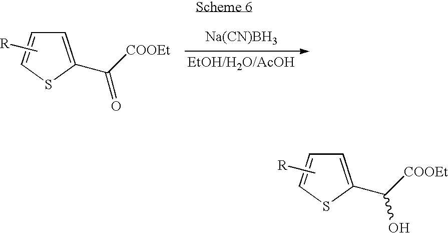

By way of example, 5-chloro-2-thienylmethanol 10 was synthesised according to Scheme 5.

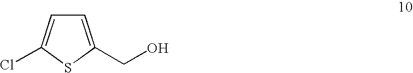

Sodium borohydride (0.19 g, 5 mmol) was added slowly to the solution of 5-chloro-2-thiophenecarboxaldehyde (0.53 mL, 5 mmol) in methanol (10 mL) at room temperature. The reaction mixture was stirred at room temperature for 2 hours, then diluted into ethyl acetate, and washed 0.5M HCl. The organic layer was dried and concentrated to give a yellow liquid as desired product (W=0.68 g, 92%). ¹H NMR (CDCl₃): δ 4.73 (s, 2H), 6.78-6.80 (m, 2H).

As another example, ethyl 2-hydroxy, 2-thienylacetate 11 was synthesised according to Scheme 6.

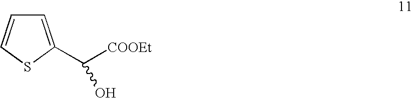

Sodium cyanoborohydride (0.63 g, 10 mmol) was added slowly to a solution of ethyl thiophene-2-glyoxylate (1.84 g, 10 mmol) in ethanol, water and acetic acid (8:3:1, total 70 mL). The reaction mixture was stirred at room temperature for 8 hours, then acidified with 1M HCl to a pH of 1, and extracted with ethyl acetate. The combined extracts were washed with saturated sodium bicarbonate, water, then dried over magnesium sulphate, and concentrated to afford 11 as a pale yellow oil (W=1.94 g, 100%). ¹H NMR (CDCl₃): δ 1.29 (t, 3H, CH₃), 3.59 (b, 1H, OH), 4.28-4.34 (m, 2H, COOCH2), 5.42 (d, 1H, CH), 6.99-7.02 (m, 1H), 7.12 (d, 1H), 7.29 (dd, J=2 Hz, 1H).

### Example 1

### Compound 219

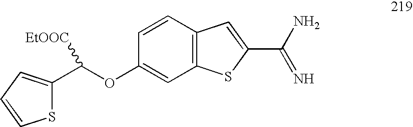

Compound 219 was obtained according to the procedure recited in steps (i) and (ii) of Scheme 1.

### Step (i)

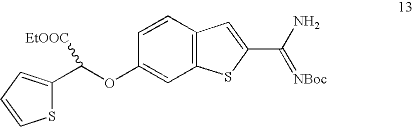

2-(N-tert-butoxycarbonyl) amidino-6-(2-thienyl-2-ethoxycarbonyl)methyl benzothiophene ether 13 was obtained from intermediate 1 according to the procedure given in step (i) of Scheme 1.

### Step (ii)

Magnesium perchlorate (0.03 g, 0.13 mmol) was added to a solution of 13 (56 mg, 0.119 mmol) in acetonitrile (2 mL), and the reaction solution was stirred at 80° C. for 3 hours, then concentrated. The crude product was purified by column (silica gel, 9:1 DCM: MeOH) to give compound 219 as an off-white solid (18 mg, 47%). ¹H NMR (DMSO-d6): δ 1.14 (t, 3H, CH₃, J=7.8 Hz), 4.10-4.18 (m, 2H, COOCH₂), 6.21 (s, 1H, CHCOO), 6.94 (2d, J=5 Hz, 1H, ArH), 7.14 (dd, J1=2.3 Hz, J2=8.9 Hz, 1H, ArH), 7.20 (d, J=3.3 Hz, 1H, ArH), 7.38 (dd, J1=1 Hz, J2=5.1 Hz, 1H, ArH), 7.49 (d, J=2.3 Hz, 1H, ArH), 7.82 (d, J=9 Hz, 1H, ArH), 8.06 (s, 1H, ArH). MS: ES+361 (M+H).

### Example 2

### Compound 244

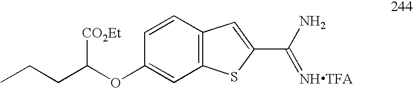

Compound 244 was obtained by Boc deprotection of Intermediate 1 using TFA. Intermediate 1 (100 mg, 0.34 mmol) was heated at 65° C. with DL-ethyl-2-bromovalerate (64.55111, 0.37 mmol) and K₂CO₃ (52 mg, 0.37 mmol) in acetonitrile for 10 hours. This was subsequently purified through a small flash chromatography (SiO₂, 20:80 ethyl acetate:hexane) to furnish 131 mg of the title compound 244 as a yellow solid (yield: 90%). ¹H NMR (DMSO-d6): δ 9.5 (bs, NH, 3H), 8.28 (s, ArH, 1H), 7.97 (dd, ArH, 1H), 7.68 (bs, ArH, 1H), 7.16 (d, ArH, 1H), 5.03 (t, α-H, 1H), 4.15 (q, ester CH₂, 2H), 1.90 (q, α-CH₂, 2H), 1.5 (dq, β-CH₂, 2H), 1.16 (t, ester CH₃, 3H) and 0.94 (t, χ-CH₃, 3H). MS: ES+ 321 (M+H).

### Example 3

### Compound 444

Compound 444 was obtained according to the procedure described in Scheme 6 below:

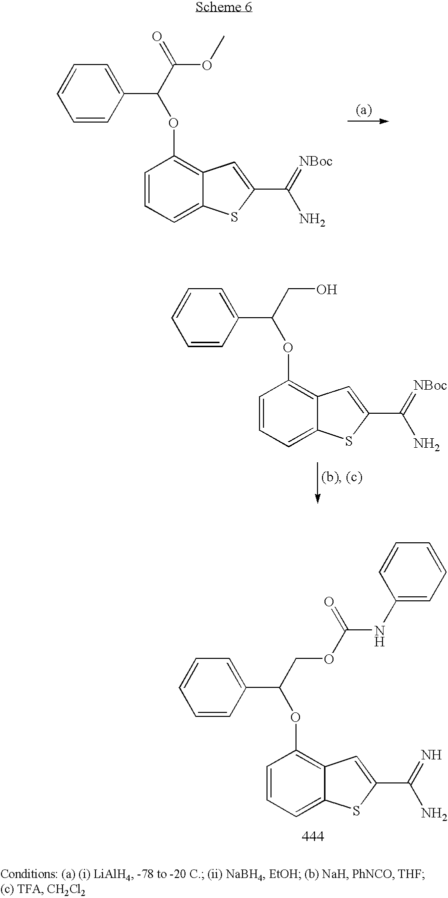

### Example 4

### Compound 446

Compound 446 was obtained according to the procedure described in Scheme 7 below:

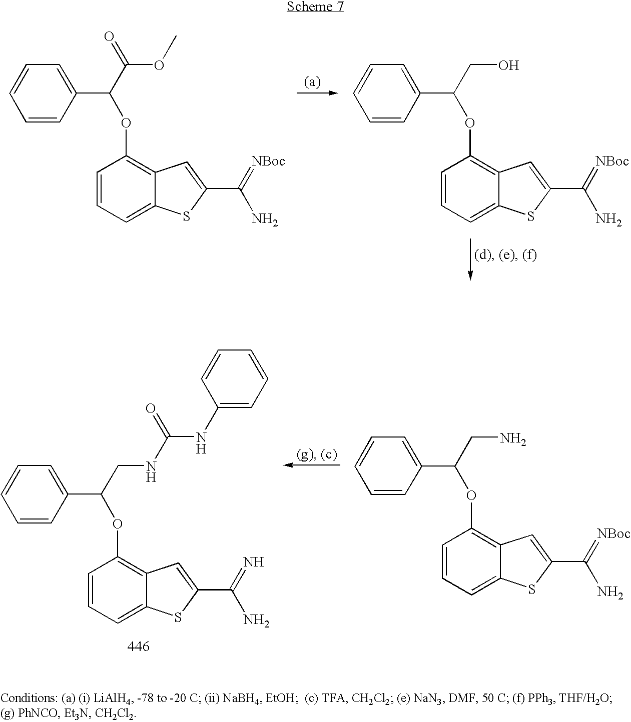

### Example 5

### Activity Assay

Various compounds of the disclosure were tested for their activity against Factor IXa, Factor Xa, uPA, plasmin, and thrombin. Examples of the testing procedures are described below:

### Method A

100 μl Factor IXa (5 μg/ml in assay buffer) and 20 μl vehicle or compound solution were added to 60 μl assay buffer (50 mM Tris, 100 mM NaCl, 5 mM CaCl₂, 0.5% PEG 6000, pH 7.4) and incubated for 5 minutes at 37° C. After the incubation period, 20 μl of factor IXa substrate (S2366, 5 mM in assay buffer) was added and changes in Vmax monitored on a plate reader for 10 minutes using a wavelength of 405 nm at 37° C.

### Method B

50 μl Factor Xa (40 ng/ml in assay buffer) and 20 μl vehicle or compound solution were added to 110 μl assay buffer (100 mM Na orthophosphate (80% Na₂HPO₄ and 20% NaH₂PO₄), 200 mM NaCl, 0.5% PEG 6000, 0.02% Na azide, pH 7.5) and incubated for 5 minutes at 37° C. After the incubation period, 20 μl of factor Xa substrate (50 μM, S2765) was added and changes in Vmax monitored on a plate reader for 10 minutes using a wavelength of 405 nm at 37° C.

### Method C

50 μl Plasmin (75 ng/ml in assay buffer) and 20 μl vehicle or compound solution were added to 110 μl assay buffer (100 mM Na orthophosphate (80% Na₂HPO₄ and 20% NaH₂PO₄), 200 mM NaCl, 0.5% PEG 6000, 0.02% Na azide, pH 7.5) and incubated for 5 minutes at 37° C. After the incubation period, 20 μl of plasmin substrate (7 mM, S2366) was added and changes in Vmax monitored on a plate reader for 10 minutes using a wavelength of 405 nm at 37° C.

### Method D

50 μl Urokinase (33.3 ng/ml in assay buffer) and 20 μl vehicle or compound solution were added to 110 μl assay buffer (100 mM Na orthophosphate (80% Na₂HPO₄ and 20% NaH₂PO₄), 200 mM NaCl, 0.5% PEG 6000, 0.02% Na azide, 0.01% BSA, pH 7.5) and incubated for 5 minutes at 37° C. After the incubation period, 20 μl of urokinase substrate (1.6 mM, S2444) was added and changes in Vmax monitored on a plate reader for 10 minutes using a wavelength of 405 nm at 37° C.

The results of the tests showed that the listed compounds include those which have an IC₅₀ for Factor IXa of 50 μm or less.

1. A method of inhibiting Factor IXa in the treatment, prevention or delay in progression of a thrombotic disorder, the method comprising administering to a patient a therapeutic amount of a compound of Formula (i): 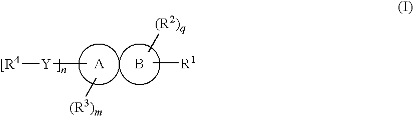
wherein
ring A is a 5-, 6- or 7-membered ring which is fused with ring B;
ring B is a 5-, 6- or 7-membered ring having at least one in-ring atom which is —O— or —S—;
each Y is independently a bond or a linker having 1 to 20 in-chain atoms and comprising, for example, one or more linkages selected from —O—, —N(R⁵)—, —N(R⁶)—, —C(O)—, —C(S)—, —S(O)ₗ—, —(CH₂)ₖ—, —C(R⁶)(R⁷)—, —C(R⁵)═C(R⁵)—, —C≡C—, carbocyclylene optionally substituted with 1, 2, 3, 4 or 5 R¹¹, and heterocyclylene optionally substituted with 1, 2, 3, 4 or 5 R¹¹;
R¹ is hydrogen R¹¹, or a basic moiety;
R² and R³ are each independently selected from R¹¹;
each R⁴ is independently hydrogen, except when Y is a bond; or is hydrocarbyl or heterocyclyl, either of which is optionally substituted with 1, 2, 3, 4 or 5 R¹¹;
each R⁵ is independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹;
R⁶ and R⁷ are each independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃, with the proviso that R⁷ is not hydrogen;
R⁸, R⁹ and R¹⁰ are each independently selected from hydrogen, R⁸, hydrocarbyl optionally substituted with 1, 2, 3, 4 or 5 R¹¹; heterocyclyl optionally substituted with 1, 2, 3, 4 or 5 R¹; and —(CH₂)ⱼ-heterocyclyl, the heterocyclyl part of which is optionally substituted with 1, 2, 3, 4 or 5 R¹¹;
each R¹¹ is independently selected from halogen, hydroxy, trifluoromethyl, cyano, nitro, oxo, amidino, —B(OH)₂, ═NR¹², —OR¹², —SR¹², —C(O)R¹², —C(O)OR¹², —OC(O)R¹², —N(R¹²)R¹², —C(O)N(R¹²)R¹³, —OC(O)N(R¹²)R¹³, —S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹², —NR¹³S(O)ₗR¹², —NR¹³C(O)NR¹²R¹³, —C(R¹²)₃ and R¹⁴;
R¹² and R¹³ are the same or different and are each hydrogen or are selected from C₁₋₆ acyclic aliphatic groups, carbocyclyl optionally substituted by a C₁₋₆ acyclic aliphatic group and bonded to the remainder of the molecule either directly or through a C₁₋₆ acyclic aliphatic group, and heterocyclyl optionally substituted by a C₁₋₆ acyclic aliphatic group and bonded to the remainder of the molecule either directly or through a C₁₋₆ acyclic aliphatic group, any of which is optionally substituted with 1, 2, 3, 4 or 5 substituents independently selected from halogen, hydroxy, trifluoromethyl, cyano, nitro, oxo, amidino, —B(OH)₂, ═NR^{u}, —OR^{v}, —SR^{v}, —C(O)R^{v}, —C(O)OR^{v}, —OC(O)R^{v}, —N(R^{u})R^{v}, —C(O)N(R^{u})R^{v}, —OC(O)N(R^{u})R^{v}, —S(O)ₗRV, —S(O)ₗNR^{u}R^{v}, —S(O)ₗNR^{u}C(O)R^{v}, —S(O)ₗNR^{u}C(O)OR^{v}, —NR^{u}C(O)R^{v}, —NR^{u}C(O)OR^{v}, —NR^{u}S(O)ₗR^{v}, —NR^{u}C(O)NR^{v}R^{u}, —C(R^{v})₃, and C₁₋₆ alkyl optionally substituted by 1, 2, 3, 4 or 5 halogens, where R^{u} is H, OH or C₁₋₆ alkyl optionally substituted by up to 5 halogens and R^{v} is H or C₁₋₆ alkyl optionally substituted by up to 5 halogens;
R¹³ additionally may be hydroxy or C₁₋₆ alkoxy;
R¹⁴ is selected from C₁₋₆ acyclic aliphatic groups, C₁₋₆ acyclic aliphatic-oxy, —(CH₂)ᵢ—O—(CH₂)ⱼ-carbocyclyl, —(CH₂)ᵢ—O—(CH₂)ⱼ-heterocyclyl, —(CH₂)ᵢ—O—(CH₂)ⱼ-carbocyclyl(C₁-C₆)alkyl and —(CH₂)ᵢ—O—(CH₂)ⱼ-heterocyclyl(C₁-C₆)alkyl, any of which is optionally substituted with 1, 2, 3, 4 or 5 substituents independently selected from halogen, hydroxy, trifluoromethyl, cyano, nitro, oxo, amidino, —B(OH)₂, ═NR¹², —OR¹², —SR¹², —C(O)R¹², —C(O)OR¹², —OC(O)R¹², —N(R¹²)R¹³, —C(O)N(R¹²)R¹³, —OC(O)N(R¹²)R¹³, —S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹²—NR¹³S(O)ₗR¹², —NR¹³C(O)NR¹²R¹³, and —C(R¹²)₃;
i is 0, 1, 2, 3, 4, 5 or 6
j is 0, 1, 2, 3, 4, 5 or 6;
k is 1, 2, 3, 4, 5 or 6;
l is 0, 1 or 2;
m is 0, 1, 2, 3 or 4;
n is 0, 1 or 2;
p is 0 or 1; and
q is 0, 1, 2, 3 or 4;
or a pharmaceutically acceptable salt or prodrug thereof.

2-4. (canceled)

5. The method according to claim 1, wherein the inhibition of the factor IXa comprises inhibiting the formation of a tenase complex; and further wherein R¹ is —C(NH)NH₂ or a prodrug or salt form thereof, wherein the prodrug is selected from an N-benzyloxycarbonyl- or an N-(acyloxy)methoxycarbonyl amidine derivative, an amidoxime, an O-alkylamidoxime, an N-alkoxycarbonylamidine, an alkoxycarbonyl derivative (carbamoyl) and an acyloxymethyl carbamate group.

6-7. (canceled)

8. The method according to claim 1, wherein ring A is aromatic and the compound has one or both of the features that ring A is a 6-membered ring and ring B is a 5-membered ring.

9-10. (canceled)

11. The method according to claim 1, wherein the compound is a compound of Formula IV or a compound of Formula (VI), or a pharmaceutically acceptable salt or prodrug thereof: 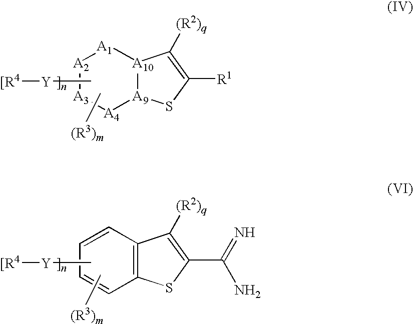
wherein
A₁, A₂, A₃ and A₄ may be the same or different and are each independently selected from —N═, —NH—, —O—, —S—, —CH═ and —CH₂— and
A₉ and A₁₀ are each independently C, CH or N.

12-13. (canceled)

14. The method according to claim 1, wherein n is 1 and Y is selected from:
a bond
-Y¹-;
-Y¹-Y²-;
-Y¹-Y²-Y³-;
-Y¹-Y²-Y³-Y⁴-;
-Y¹-Y²-Y³-Y⁴-Y⁵-;
-Y¹-Y²-Y³-Y⁴-Y⁵-Y⁶-;
-Y¹-Y²-Y³-Y⁴-Y⁵-Y⁶-Y⁷-Y⁸-;
-Y¹-Y²-Y³-Y⁴-Y⁵-Y⁶-Y⁷-Y⁸-Y⁹-; and
-Y¹-Y²-Y³-Y⁴-Y⁵-Y⁶-Y⁷-Y⁸-Y⁹-Y¹⁰-;
wherein Y¹, Y², Y³, Y⁴, Y⁵, Y⁶, Y⁷, Y⁸, Y⁹ and Y¹⁰ are each independently selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C≡C—, carbocyclylene optionally substituted with 1, 2, 3, 4 or 5 R¹¹, and heterocyclylene optionally substituted with 1, 2, 3, 4 or 5 R¹¹.

15. The method according to claim 14, wherein R³ is:
(i) selected from halogen, hydroxy, trifluoromethyl, cyano and nitro when m is 1; or
(ii) C₁₋₆ alkyl or C₁₋₆ alkoxy.

16. (canceled)

17. The method according to claim 15, wherein the compound is of Formula (VII, 1.1, 1) or Formula (VII, 1.3, 1):

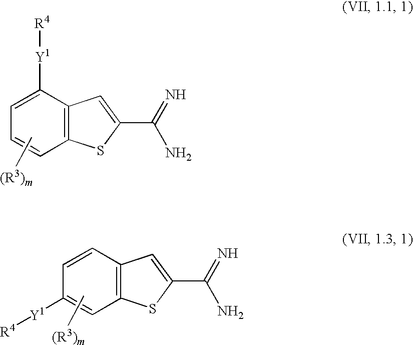

18-20. (canceled)

21. The method according to claim 17, wherein the compound is a compound of Formula (X, 1) or Formula (X, 3): 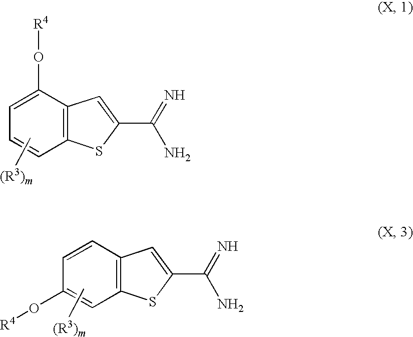
or a pharmaceutically acceptable salt of prodrug thereof.

22-23. (canceled)

24. The method according to claim 15, wherein the compound is a compound of Formula (VII, 1.1, 2) or Formula (VII, 1.3, 2): 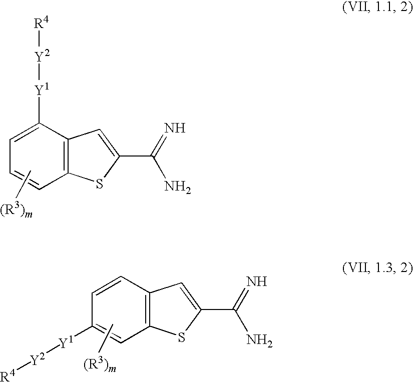
or a pharmaceutically acceptable salt of prodrug thereof.

25. (canceled)

26. The method according to claim 24, wherein the compound is a compound of Formula (XI, 1) or (XI, 3): 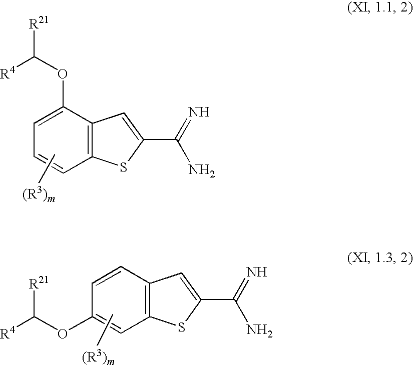
wherein R²¹ is hydrogen or R⁷, or is selected from C₁₋₆ alkyl cycloalkyl, aryl and heterocyclyl, any of which is optionally substituted with 1, 2, 3, 4 or 5 R¹¹;
or a pharmaceutically acceptable salt of prodrug thereof.

27-29. (canceled)

30. The method according to claim 15, wherein the compound is a compound of the Formula (VII, 1.1, 3) or Formula (VII, 1.3, 3): 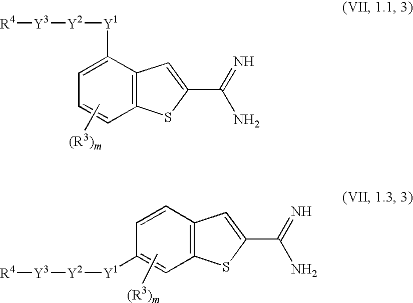
or a pharmaceutically acceptable salt of prodrug thereof, or wherein the compound is a compound of the Formula (XII. 1) or Formula (XII. 3): 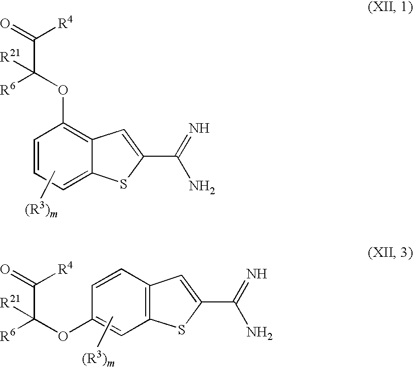
or a pharmaceutically acceptable salt or prodrug thereof, wherein
R⁶ is hydrogen or C₁₋₆ alkyl optionally substituted with 1, 2, 3, 4 or 5 R¹¹ and
R²¹ is hydrogen or is selected from C₁₋₆ alkyl, cycloalkyl aryl and heterocyclyl, any of which is optionally substituted with 1, 2, 3, 4 or 5 R¹¹.

31-36. (canceled)

37. The method according to claim 15, wherein the compound is a compound of the Formula (VII, 1.1, 4) or Formula (VII, 1.3, 4): 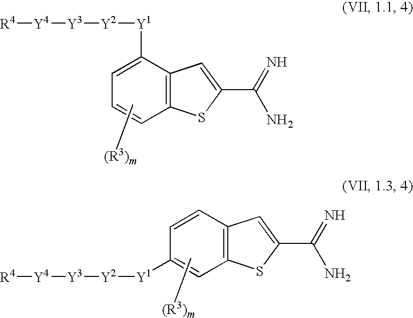
or a pharmaceutically acceptable salt or prodrug thereof.

38. (canceled)

39. The method according to claim 37, wherein the compound is a compound of Formula (XIII, 1) or (XIII, 3): 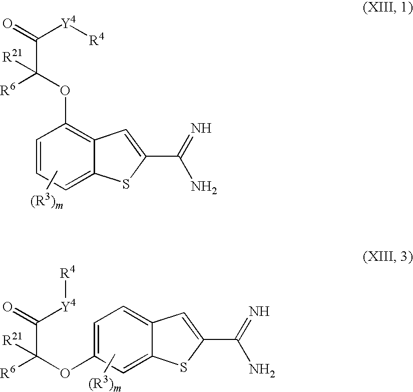
or a pharmaceutically acceptable salt or prodrug thereof, wherein:
Y⁴ is —O—, —N(R⁵)— or —CH₂—;
R⁶ is hydrogen or C₁₋₆ alkyl optionally substituted with 1, 2, 3, 4 or 5 R¹¹; and
R²¹ is hydrogen or is selected from C₁₋₆ alkyl, cycloalkyl, aryl and heterocyclyl, any of which is optionally substituted with 1, 2, 3, 4 or 5 R¹¹.

40-44. (canceled)

45. The method according to claim 15, wherein the compound is a compound of the Formula (VII, 1.1, 5) or Formula (VII, 1.3, 5): 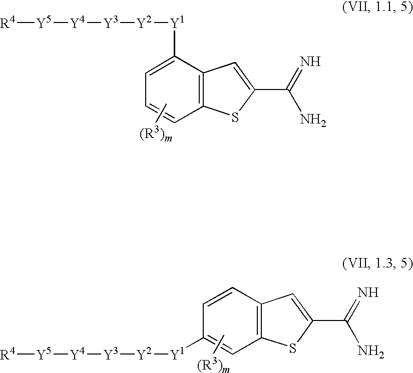
or a pharmaceutically acceptable salt or prodrug thereof; or
wherein the compound is a compound of the Formula (XIV, 1) or (XIV, 3): 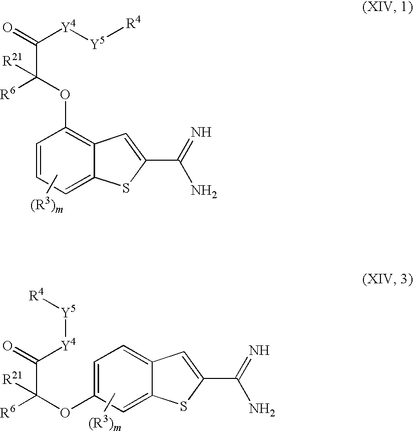
or a pharmaceutically acceptable salt or prodrug thereof wherein:
Y⁴ is —O—, —N(R⁵)— or —CH₂—;
Y⁵ is —CH₂—, carbocyclylene or heterocyclylene;
R⁶ is hydrogen or C₁₋₆ alkyl optionally substituted with 1, 2, 3, 4 or 5 R¹¹; and
R²¹ is hydrogen or is selected from C₁₋₆ alkyl, cycloalkyl, aryl and heterocyclyl, any of which is optionally substituted with 1, 2, 3, 4 or 5 R¹¹.

46-55. (canceled)

56. The method according to claim 15, wherein the compound is a compound of Formula (VII, 1.1, 6) or Formula (VII, 1.3, 6): 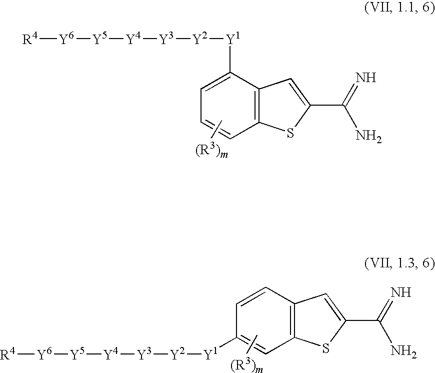
or a pharmaceutically acceptable salt or prodrug thereof, or is a compound of Formula (VII. 1.1, 6) or Formula (VII, 1.3, 6) wherein R⁴ is selected from C₁₋₆ alkyl, cycloalkyl, aryl and heterocyclyl, any of which is optionally substituted with 1, 2, 3, 4 or 5 R¹¹.

57. (canceled)

58. The method according to claim 56, wherein the compound is a compound of Formula (XVI, 1): 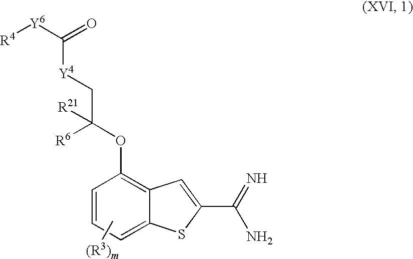
wherein R²¹ is hydrogen or R⁷, or is selected from C₁₋₆ alkyl, cycloalkyl, aryl and heterocyclyl, any of which is optionally substituted with 1, 2, 3, 4 or 5 R¹¹;
or a pharmaceutically acceptable salt or prodrug thereof.

59. The method according to claim 58, wherein Y⁴ and Y⁶ are each independently —O— or —N(R⁵)—; and further wherein R⁶ is hydrogen and R²¹ is hydrogen, or is C₁₋₆ alkyl or phenyl, either of which is optionally substituted with 1, 2, 3, 4 or 5 R¹¹.

60-61. (canceled)

62. The method according to claim 14, wherein R⁴ is selected from C₁₋₆ alkyl, cycloalkyl, aryl and heterocyclyl, any of which is optionally substituted with 1, 2, 3, 4 or 5 R¹¹.

63-67. (canceled)

68. A method of inhibiting Factor IXa in the treatment, prevention or delay in progression of a thrombotic disorder, the method comprising administering to a patient a therapeutic amount of a compound of the following formula: 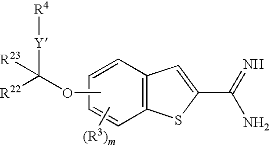
wherein
Y¹ is a bond or a linker having 1 to 18 in-chain atoms and comprising, for example, one or more linkages selected from —O—, —N(R⁵)—, —C(O)—, —C(S)—, —S(O)ₗ—, —(CH₂)ₖ—, —C(R⁶)(R⁷)—, —C(R⁵)═C(R⁵)—, —C≡C—, carbocyclylene optionally substituted with 1, 2, 3, 4 or 5 R¹¹, and heterocyclylene optionally substituted with 1, 2, 3, 4 or 5 R¹¹;
each R³ is independently selected from R¹¹;
R⁴ is hydrogen, except when Y is a bond; or is hydrocarbyl or heterocyclyl, either of which is optionally substituted with 1, 2, 3, 4 or 5 R¹¹;
each R⁵ is independently selected from hydrogen, R¹¹, hydrocarbyl optionally substituted with 1, 2, 3, 4 or 5 R¹¹, heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
R⁶ and R⁷ are each independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃, with the proviso that R⁷ is not hydrogen;
R⁸, R⁹ and R¹⁰ are each independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
each R¹¹ is independently selected from halogen, hydroxy, trifluoromethyl, cyano, nitro, oxo, amidino, —B(OH), ═NR¹², —OR¹², —SR¹², —C(O)R¹², —C(O)OR¹², —OC(O)R¹², —N(R¹²)R¹³, —C(O)N(R¹²)R¹³, —OC(O)N(R¹²)R¹³, —S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹², —NR¹³S(O)ₗR¹², —NR³C(O)NR¹²R¹³, —C(R¹²)₃ and R¹⁴;
R¹² and R¹³ are the same or different and are each hydrogen or are selected from C₁₋₆ acyclic aliphatic groups, carbocyclyl optionally substituted by a C₁₋₆ acyclic aliphatic group and bonded to the remainder of the molecule either directly or through a C₁₋₆ acyclic aliphatic group, and heterocyclyl optionally substituted by a C₆ acyclic aliphatic group and bonded to the remainder of the molecule either directly or through a C₁₋₆ acyclic aliphatic group, any of which is optionally substituted with 1, 2, 3, 4 or 5 substituents independently selected from halogen, hydroxy, trifluoromethyl, cyano, nitro, oxo, amidino, —B(OH)—, ═NR^{u}, —OR^{v}, —SR^{v}, —C(O)R^{v}, —C(O)OR^{v}, —OC(O)R^{v}, —N(R^{u})R^{v}, —C(O)N(R^{u})R^{v}, —OC(O)N(R^{u})R^{v}, —S(O)ₗR^{v}, —S(O)ₗNR^{u}R^{v}, —S(O)ₗNR^{u}C(O)R^{v}, —S(O)ₗNR^{u}C(O)OR^{v}, —NR^{u}C(O)R^{v}, —NR^{u}C(O)OR^{v}, —NR^{u}S(O)ₗR^{v}, —NR^{u}C(O)NR^{v}R^{u}, —C(R^{v})₃, and C₁₋₆ alkyl optionally substituted by 1, 2, 3, 4 or 5 halogens, where R^{u} is H, OH or C₁₋₆ alkyl optionally substituted by up to 5 halogens and R^{v} is H or C₁₋₆ alkyl optionally substituted by up to 5 halogens,
R¹³ additionally may be hydroxy or C₁₋₆ alkoxy,
R¹⁴ is selected from C₁₋₆ acyclic aliphatic groups, C₁₋₆ acyclic aliphatic-oxy, —(CH₂)ᵢ—O—(CH₂)ⱼ-carbocyclyl, —(CH₂)ᵢ—O—(CH₂)ⱼ-heterocyclyl, —(CH₂)ᵢ—O—(CH₂)ⱼ-carbocyclyl(C₁-C₆)alkyl and —(CH₂)ᵢ—O—(CH₂)ⱼ-heterocyclyl(C₁-C₆)alkyl, any of which is optionally substituted with 1, 2, 3, 4 or 5 substituents independently selected from halogen, hydroxy, trifluoromethyl, cyano, nitro, oxo, amidino, —B(OH)₂, ═NR¹², —OR¹², —SR¹², —C(O)R¹², —C(O)OR¹², —OC(O)R¹², —N(R¹²)R¹³, —C(O)N(R¹²)R¹³, —OC(O)N(R¹²)R¹³, —S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹², —NR¹³S(O)ₗR¹², —NR¹³C(O)NR¹²R¹³, and —C(R¹²)₃;
i is 0, 1, 2, 3, 4, 5 or 6
j is 0, 1, 2, 3, 4, 5 or 6;
k is 1, 2, 3, 4, 5 or 6,
l is 0, 1 or 2;
m is 0, 1, 2, 3 or 4;
R²² is carbocyclyl or heterocyclyl, either of which is optionally substituted with 1, 2, 3, 4 or 5 R¹¹; and
R²³ is independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃;
or a pharmaceutically acceptable salt or prodrug thereof.

69. (canceled)

70. The method according to claim 68, wherein R²² is selected from phenyl, cyclopropyl and pyridinyl, any of which is optionally substituted with 1, 2, 3, 4 or 5 R¹¹; and R²³ is hydrogen or C₁₋₆ alkyl optionally substituted with 1, 2, 3, 4 or 5 R¹¹.

71-74. (canceled)

75. A compound of the following Formula: 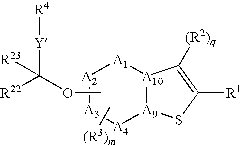
wherein
A₁, A₂, A₃, and A₄ may be the same or different and are each independently selected from —N═, —NH—, —O—, —S—, —CH═ and —CH₂—;
A₉ and A₁₀ are each independently C, CH or N;
Y′ is a bond or a linker having 1 to 18 in-chain atoms and comprising, for example, one or more linkages selected from —O—, —N(R⁵)—, —C(O)—, —C(S)—, —S(O)ₗ—, —(CH₂)ₖ—, —C(R⁶(R⁷)—, —C(R⁵)═C(R⁵)—, —C═C—, carbocyclylene optionally substituted with 1, 2, 3, 4 or 5 R¹¹ and heterocyclylene optionally substituted with 1, 2, 3, 4 or 5 R¹¹;
R²² is carbocyclyl or heterocyclyl, either of which is optionally substituted with 1, 2, 3, 4 or 5 R¹¹; and
R²³ is independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃;
R¹ is a group of Formula (i): 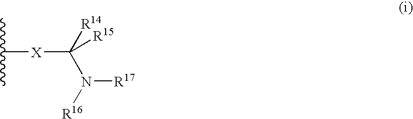
wherein
X is a bond, —NR³⁰— or —C(O)—;
R¹⁴, R¹⁵ and R³⁰ are each independently selected from R¹⁸, —OR¹⁸, —C(O)R¹⁸—C(O)OR¹⁸, —OC(O)R¹⁸, —N(R¹⁸)R¹⁹, —C(O)N(R¹⁹)R²⁰, —S(O)ₗR¹⁸ and —C(R¹⁸)₃;
or R¹⁴ and R¹⁵ taken together form ═NR²⁰, ═O or ═S,
R¹⁶ and R¹⁷ are each independently selected from hydrogen, C₁₋₆ alkyl, —OR²¹ and —NR¹⁸R¹⁹;
R¹⁸ and R¹⁹ are each independently selected from hydrogen, R¹¹, hydrocarbyl optionally substituted with 1, 2, 3, 4 or 5 R¹¹; and heterocyclyl optionally substituted with 1, 2, 3, 4 or 5 R¹¹;
R²⁰ hydrogen, hydroxy, C₁₋₆ alkoxy or C₁₋₆ alkyl and
R²¹ is hydrogen or R⁷;
R² and R³ are each independently selected from R¹¹;
each R⁴ is independently hydrogen, except when Y′ is a bond; or is hydrocarbyl or heterocyclyl, either of which is optionally substituted with 1, 2, 3, 4 or 5 R¹¹;
each R⁵ is independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
R⁶ and R⁷ are each independently selected from R⁸, —OR⁸, —C(O)R⁸, —C(O)OR⁸, —OC(O)R⁸, —N(R⁹)R¹⁰, —C(O)N(R⁹)R¹⁰, —S(O)ₗR⁸ and —C(R⁸)₃, with the proviso that R⁷ is not hydrogen;
R⁸, R⁹ and R¹⁰ are each independently selected from hydrogen, R¹¹, hydrocarbyl optionally substituted with 1, 2, 3, 4 or 5 R¹¹; heterocyclyl optionally substituted with 1, 2, 3, 4 or 5 R¹¹; and —(CH₂)ⱼ-heterocyclyl, the heterocyclyl part of which is optionally substituted with 1, 2, 3, 4 or 5 R¹¹;
each R¹¹ is independently selected from halogen, hydroxy, trifluoromethyl, cyano, nitro, oxo, amidino, —B(OH)₂, ═NR¹², —OR¹², —SR¹², —C(O)R¹², —C(O)OR¹², —OC(O)R¹², —N(R¹²)R¹³, —C(O)N(R¹²)R¹³, —OC(O)N(R¹²)R¹³, —S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹², —NR¹³S(O)ₗR¹², —NR¹³C(O)NR¹²R¹³, —C(R¹²)₃ and R¹⁴;
R¹² and R¹³ are the same or different and are each hydrogen or are selected from C₁₋₆ acyclic aliphatic groups, carbocyclyl optionally substituted by a C₁₋₆ acyclic aliphatic group and bonded to the remainder of the molecule either directly or through a C₁₋₆ acyclic aliphatic group, and heterocyclyl optionally substituted by a C₁₋₆ acyclic aliphatic group and bonded to the remainder of the molecule either directly or through a C₁₋₆ acyclic aliphatic group, any of which is optionally substituted with 1, 2, 3, 4 or 5 substituents independently selected from halogen, hydroxy, trifluoromethyl, cyano, nitro, oxo, amidino, —B(OH)₂, ═NR^{u}, —OR^{v}, —SR^{v}, —C(O)R^{v}, —C(O)OR^{v}, —OC(O)R^{v}, —N(R^{u})R^{v}, —C(O)N(R^{u})R^{v}, —OC(O)N(R^{u})R^{v}, —S(O)ₗR^{v}, —S(O)ₗNR^{u}R^{v}, —S(O)ₗNR^{u}C(O)R^{v}, —S(O)ₗNR^{u}C(O)OR^{v}, —NR^{u}C(O)R^{v}, —NR^{u}C(O)OR^{v}, —NR^{u}S(O)ₗRV, —NR^{u}C(O)NR^{v}R^{u}, —C(R^{v})₃, and C₁₋₆ alkyl optionally substituted by 1, 2, 3, 4 or 5 halogens, where R^{u} is H, OH or C₁₋₆ alkyl optionally substituted by up to 5 halogens and R^{v} is H or C₁₋₆ alkyl optionally substituted by up to 5 halogens;
R¹³ additionally may be hydroxy or C₁₋₆ alkoxy;
R¹⁴ is selected from C₁₋₆ acyclic aliphatic groups, C₁₋₆ acyclic aliphatic-oxy, —(CH₂)ᵢ—O—(CH₂)ⱼ-carbocyclyl, —(CH₂)ᵢ—O—(CH₂)ⱼ-heterocyclyl, —(CH₂)ᵢ—O—(CH₂)ⱼ-carbocyclyl(C₁-C₆)alkyl and —(CH₂)ᵢ—O—(CH₂)ⱼ-heterocyclyl(C₁-C₆)alkyl, any of which is optionally substituted with 1, 2, 3, 4 or 5 substituents independently selected from halogen, hydroxy, trifluoromethyl, cyano, nitro, oxo, amidino, —B(OH)₂, ═NR¹², —OR¹², —SR¹², —C(O)R¹², —C(O)OR¹², —OC(O)R¹², —N(R¹²)R¹³, —C(O)N(R¹²)R¹³, —OC(O)N(R¹²)R¹³, —S(O)ₗR¹², —S(O)ₗNR¹²R¹³, —S(O)ₗNR¹³C(O)R¹², —S(O)ₗNR¹³C(O)OR¹², —NR¹³C(O)R¹², —NR¹³C(O)OR¹², —NR¹³S(O)R¹², —NR¹³C(O)NR¹²R¹³, and —C(R¹²)₃;
i is 0, 1, 2, 3, 4, 5 or 6
j is 0, 1, 2, 3, 4, 5 or 6;
k is 1, 2, 3, 4, 5 or 6;
l is 0, 1 or 2;
m is 0, 1, 2, 3 or 4;
n is 0, 1 or 2;
p is 0 or 1; and
q is 0, 1, 2, 3 or 4;
or a pharmaceutically acceptable salt or prodrug thereof.

76. (canceled)

77. A compound according to claim 75, wherein the compound is as defined in claim 68.

78. A 2-amidinobenzothiophene compound which is an inhibitor of Factor IXa, the compound being characterised by a substituent at one or both of the 4- and 6-positions which substituent comprises a fragment of the following Formula: 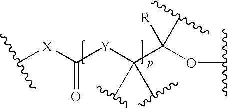
wherein
R is a moiety comprising an optionally substituted carbocyclic or heterocyclic group;
X and Y are each independently O or N;
p is 0 or 1; and
the oxygen atom on the right hand side of the fragment as drawn is bound directly to the 4- or 6-carbon atom of the benzothiophene ring;
or a pharmaceutically acceptable salt or prodrug thereof.

79-80. (canceled)

81. A compound according to claim 78, wherein said substituent is of the Formula (i.6) or the Formula (i.7): 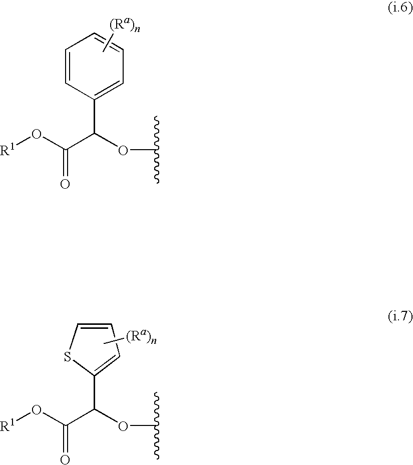
wherein
R¹ is hydrogen or selected from C₁₋₆ alkyl, —(CH₂)ₖ-carbocyclyl and —(CH₂)ₖ-heterocyclyl, any of which is optionally substituted with 1, 2, 3, 4 or 5 R^{a};
each R^{a} is independently selected from selected from (i) halogen; and (ii) moieties having from 1 to 30 plural valent atoms selected from C, N, O and S as well as monovalent atoms selected from hydrogen and halogen, for example is selected from halogen, hydroxy, trifluoromethyl, cyano, nitro, oxo, amidino, —B(OH)₂, ═NR^{b}, —OR^{b}, —C(O)R^{b}, —C(O)OR^{b}, OC(O)R^{b}, N(R^{b})R^{c}, —C(O)N(R^{b})R^{c}, —S(O)R^{b}, —C(R^{b})₃ and R^{d};
wherein:
R^{b} and R^{c} are each independently hydrogen or selected from C₁₋₆ alkyl, —(CH₂)ₖ-carbocyclyl and —(CH₂)ₖ-heterocyclyl, any of which is optionally substituted with 1, 2, 3, 4 or 5 substituents independently selected from halogen, hydroxy and C₁₋₆ alkyl,
R^{d} is selected from C₁₋₆ alkyl, C₁₋₆ alkoxy, —(CH₂)ₖ-carbocyclyl and —(CH₂)ₖ-heterocyclyl, any of which is optionally substituted with 1, 2, 3, 4 or 5 substituents independently selected from halogen, hydroxy, C₁₋₆ alkyl and C₁₋₆ alkoxy,
k is 0, 1, 2, 3, 4, 5 or 6, and
l is 0, 1 or 2; and
n is 0, 1, 2, 3, 4 or 5 or
wherein said substituent is of the Formula (ii.7): 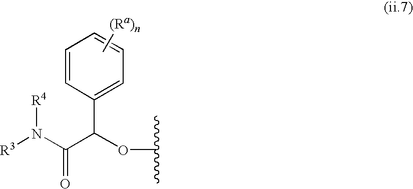
R³ and R⁴ are each independently hydrogen or selected from C₁₋₆ alkyl, —(CH₂)ₖ-carbocyclyl and —(CH₂)ₖ-heterocyclyl any of which is optionally substituted with 1, 2, 3, 4 or 5 R^{a};
or R³ and R⁴ together with the nitrogen atom to which they are attached form heterocyclyl optionally substituted with 1, 2, 3, 4 or 5 R^{a};
and R^{a}, R^{b}, R^{c}, R^{d}, k, l and n are as defined above; or wherein said substituent is of the Formula (iii.13): 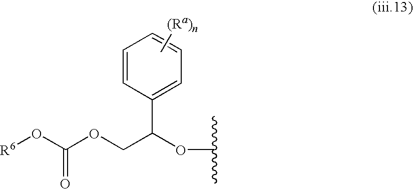
wherein
R⁶ is hydrogen or selected from C₁₋₆ alkyl —(CH₂)ₖ-carbocyclyl and —(CH₂)ₖ-heterocyclyl, any of which is optionally substituted with 1, 2, 3, 4 or 5 R^{a};
and R^{a}, R^{b}, R^{c}, R^{d}, k, l and n are as defined above; or wherein said substituent is of the Formula (iv.17): 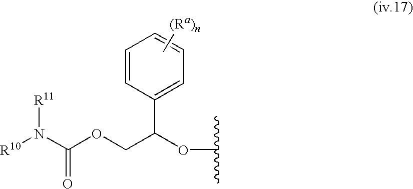
wherein
R¹⁰ and R¹¹ are each independently hydrogen or selected from C₁₋₆ alkyl, —(CH₂)ₖ-carbocyclyl and —(CH₂)ₖ-heterocyclyl, any of which is optionally substituted with 1, 2, 3, 4 or 5 R^{a};
or R¹⁰ and R¹¹ together with the nitrogen atom to which they are attached form heterocyclyl optionally substituted with 1, 2, 3, 4 or 5 R^{a};
and R^{a}, R^{b}, R^{c}, R^{d}, k, l and n are as defined above; or wherein said substituent is of the Formula (v.25): 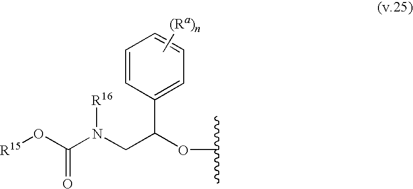
wherein
R¹⁵ is hydrogen or selected from C₁₋₆ alkyl, —(CH₂)ₖ-carbocyclyl and —(CH₂)ₖ-heterocyclyl, any of which is optionally substituted with 1, 2, 3, 4 or 5 R^{a};
R¹⁶ is hydrogen or C₁₋₆ alkyl optionally substituted with 1, 2, 3, 4 or 5 R^{a};
and R^{a}, R^{b}, R^{c}, R^{d}, k, l and n are as defined above; or
wherein said substituent is of the Formula (vi.37): 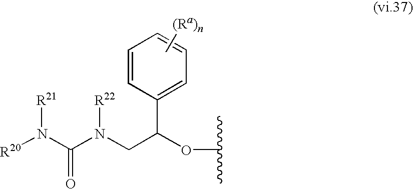
wherein
R²⁰ and R²¹ are each independently hydrogen or selected from C₁₋₆ alkyl, —(CH₂)ₖ-carbocyclyl and —(CH₂)ₖ-heterocyclyl, any of which is optionally substituted with 1, 2, 3, 4 or 5 R^{a};
or R²⁰ and R²¹ together with the nitrogen atom to which they are attached form heterocyclyl optionally substituted with 1, 2, 3, 4 or 5 R^{a};
R²² is hydrogen or C₁₋₆ alkyl optionally substituted with 1, 2, 3, 4 or 5 R^{a};
R^{a} is independently selected from each halogen, hydroxy, trifluoromethyl, cyano, nitro, oxo, amidino, —B(OH)₂, ═NR^{b}, —OR^{b}, —C(O)R^{b}, —C(O)OR^{b}, —OC(O)R^{b}, —N(R^{b})R^{c}, —C(O)N(R^{b})R^{c}, —S(O)ₗR^{b}, —C(R^{b}), and R^{d};
and R^{b}, R^{c}, R^{d}, k, l and n are as defined above.

82. A compound according to claim 81, wherein the 2-amidinobenzothiophene ring is substituted as follows:
(i) at the 4-position by a substituent of claim 24; and
(ii) on the benzene part of the ring by a single further substituent selected from halogen, a C₁₋₆ alkyl and a C₁₋₆ alkoxy, either of which is optionally substituted by halogen.

83-100. (canceled)

101. A pharmaceutical formulation comprising a compound of claim 75, which further comprises a pharmaceutically acceptable carrier, excipient or diluent.

102-106. (canceled)

107. A compound having a formula (VI, 1.1)-(VI, 1.4), or a pharmaceutically acceptable salt or prodrug thereof: 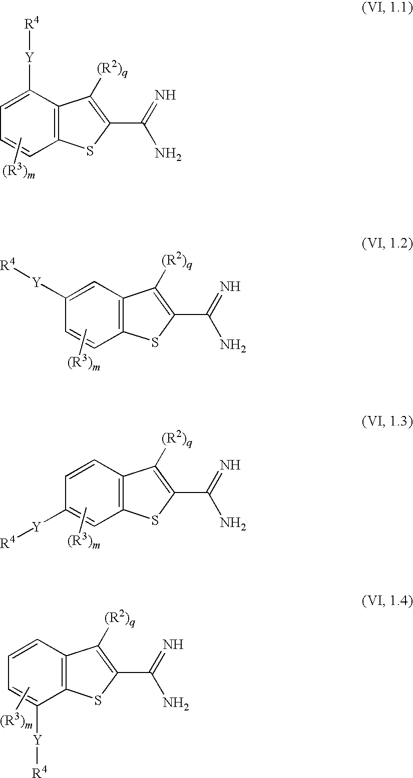
wherein R², R³, R⁴, q and m are as defined in claim 1.
